(19) United States
(12) Patent Application Publication
(10) Pub. No.: US 2006/0205761 A1
(43) Pub. Date: Sep. 14, 2006

(54) Ccr-2 antagonists for treatment of neuropathic pain

(76) Inventors: Catherine Abbadie, Summit, NJ (US); Jill Ann Lindia, South Plainfield, NJ (US); Hao Wang, Fort Washington, PA (US)

Correspondence Address: MERCK AND CO., INC, P O BOX 2000, RAHWAY, NJ, 07065-0907, US

(21) Appl. No.: 10/559,701

(22) PCT Filed: Jun. 2, 2004
(86) PCT No.: PCT/US04/17499

§ 371 Date: Dec. 6, 2005

Related U.S. Application Data
(60) Provisional application No. 60/476,391, filed on Jun. 6, 2003.
(60) Provisional application No. 60/531,637, filed on Dec. 22, 2003.

(51) Int. Cl. A61K 31/4745; A61K 31/4709

(52) U.S. Cl. 514/300; 514/310

(57) ABSTRACT

The invention is directed to methods of treating neuropathic pain and other neuropathic diseases and conditions with CCR-2 antagonists and pharmaceutical composition containing CCR-2 antagonists.

### BACKGROUND OF THE INVENTION

Neuropathic pain refers to a group of chronic pain syndromes which share the common feature that they are caused initially by nerve damage which subsequently results in an abnormal sensory processing in the central and peripheral nervous system. Neuropathic pain conditions are the consequence of a number of diseases and conditions, including diabetes, AIDS, multiple sclerosis, stump and phantom pain after amputation, cancer-related neuropathy, post-herpetic neuralgia, traumatic nerve injury, ischemic neuropathy, nerve compression, stroke, spinal cord injury. Available analgesic drugs often produce insufficient pain relief. Although tricyclic antidepressants and some antiepileptic drugs, for example gabapentin, lamotrigine and carbamazepine, are efficient in some patients, there remains a large unmet need for efficient drugs for the treatment of these conditions.

The role off chemokines, chemokine receptors and antagonists of chemokine receptors in the regulation of inflammation and inflammation related pain is currently of significant interest. The chemokines are a family of small (70-120 amino acids) peptides, proinflammatory cytokines,. Chemokines are chemotactic cytokines that are released by a wide variety of cells to attract various cells, such as monocytes, macrophages, T cells, eosinophils, basophils and neutrophils to sites of inflammation (reviewed in Schall, Cytokine, 3, 165-183 (1991) and Murphy, Rev. Immun., 12, 593-633 (1994)). These molecules were originally defined by four conserved cysteines and divided into two subfamilies based on the arrangement of the first cysteine pair. In the CXC-chemokine family, which includes IL-8, GROα, NAP-2 and IP-10, these two cysteines are separated by a single amino acid, while in the CC-chemokine family, which includes RANThS, MCP-1, MCP-2, MCP-3, MIP-1α, MIP-18 and eotaxin, these two residues are adjacent.

The α-chemokines, such as interleukin-8 (IL-8), neutrophil-activating protein-2 (NAP-2) and melanoma growth stimulatory activity protein (MGSA) are chemotactic primarily for neutrophils, whereas β-chemokines, such as RANTES, MIP-1α, MIP-1β, monocyte chemotactic protein-1 (MCP-1), MCP-2, MCP-3 and eotaxin are chemotactic for macrophages, monocytes, T-cells, eosinophils and basophils (Deng, et al., Nature, 381, 661-666 (1996)).

Chemokines are secreted by a wide variety of cell types and bind to specific G-protein coupled receptors (GPCRs) (reviewed in Horuk, Trends Pharm. Sci., 15, 159-165 (1994)) present on leukocytes and other cells. These chemokine receptors form a sub-family of GPCRs, which, at present, consists of fifteen characterized members and a number of orphans. Unlike receptors for promiscuous chemoattractants such as C5a, fMLP, PAF, and LTB4, chemokine receptors are more selectively expressed on subsets of leukocytes. Thus, generation of specific chemokines provides a mechanism for recruitment of particular leukocyte subsets.

On binding their cognate ligands, chemokine receptors transduce an intracellular signal though the associated trimeric G protein, resulting in a rapid increase in intracellular calcium concentration. There are at least seven human chemokine receptors that bind or respond to β-chemokines with the following characteristic pattern: CCR-1 (or “CKR-1” or “CC-CKR-1”) [MIP-1α, MIP-1β, MCP-3, RANTES] (Ben-Barruch, et al., J. Biol. Chem., 270, 22123-22128 (1995); Beote, et al, Cell, 72, 415-425 (1993)); CCR-2A and CCR-2B (or “CKR-2A”/“CKR-2A” or “CC-CKR-2A”/“CC-CY-R-2A”) [MCP-1, MCP-2, MCP-3, MCP4]; CCR-3 (or “CKR-3” or “CC-CKR-3”) [Eotaxin, Eotaxin 2, RANTES, MCP-2, MCP-3] (Rollins, et al., Blood, 90, 908-928 (1997)); CCR-4 (or “CKR-4” or “CC-CKR4”) [MIP-1α, RANTES, MCP-1] (Rollins, et al., Blood, 90, 908-928 (1997)); CCR-5 (or “CKR-5” or “CC-CKR-5”) [MIP-1α, RANTES, MIP-1β] (Sanson, et al., Biochemistry, 35,3362-3367 (1996)); and the Duffy blood-group antigen [RANTES, MCP-1] (Chaudhun, et al., J. Biol. Chem., 269,7835-7838 (1994)). The β-chemokines include eotaxin, MIP (“macrophage inflammatory protein”), MCP (“monocyte chemoattractant protein”) and RANTES (“regulation-upon-activation, normal T expressed and secreted”) among other chemokines. Chemokine receptors, such as CCR-1, CCR-2, CCR-2A, CCR-2B, CCR-3, CCR4, CCR-5, CXCR-3, CXCR-4, have been implicated as being important mediators of inflammatory and immunoregulatory disorders and diseases.

Despite this current interest in; chemokine receptors and chemokine receptor antagonists in connection with inflammatory disorders and diseases, the role of chemokines, chemokine receptors and chemokine receptors antagonists in the mediation of neuropathic pain conditions and diseases has yet to be established and remains largely unexplored.

### SUMMARY OP THE INVENTION

The invention is directed to methods of treating neuropathic pain and other neuropathic diseases and conditions with CCR-2 antagonists and with pharmaceutical composition containing CCR-2 antagonists.

### DETAILED DESCRIPTION OF THE INVENTION

The invention includes methods by which CCR-2 antagonists are used to treat neuropathic pain and neuropathic diseases and conditions. The invention lies in the discovery that CCR-2 chemokine receptor activity plays an important role in mediating neuropathic pain, and that CCR-2 antagonists treat, ameliorate and/or prevent neuropathic pain by blocking or altering the activity of CCR-2 in the peripheral and central nervous system.

Although the inventive methods and uses are directed to CCR-2 antagonists generally, and thus are not limited to particular CCR-2 antagonists, CCR-2 antagonists useful in connection with the invention include those specific compounds and classes of compounds which are known to antagonize CCR-2. The present invention therefore includes methods for treating neuropathic pain, and other neuropathic diseases and conditions, by administering a therapeutically effective amount of one or more of the compounds of Formulae I through XII. Recited below are CCR-2 antagonists and classes of CCR-2 antagonists useful in connection with the inventive methods. 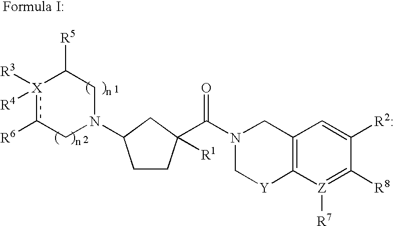
or a pharmaceutically acceptable salt thereof, or an individual diastereomer thereof, wherein:

X is C, N, O or S;

Y is O, S, SO, SO₂, or NR⁹;

Z is C or N;

R¹ is hydrogen, —C₀₋₆alkyl-W—(C₁₋₆alkyl)-, —(C₀₋₆alkyl)-W—(C₀₋₆alkyl)-(C₃₋₇cycloalkyl)-(C₀₋₆alkyl), —(C₀₋₆alkyl)-W-phenyl, or —(C₀₋₆alkyl)-W-heterocycle, wherein the alkyl, phenyl, heterocycle and the cycloalkyl are optionally substituted with 1-7 independent halo, hydroxy, —O—C₁₋₃alkyl, trifluoromethyl, C₁₋₃alkyl, —O—C₁₋₃alkyl, —CO₂R¹⁰, —CN, —NR¹⁰R¹⁰, —NR¹⁰COR¹⁰, —NR¹⁰SO₂R¹¹, or —CONR¹⁰R¹⁰ substituents;

W is a single bond, —O—, —S—, —SO—, —SO₂—, —CO—, —CO₂—, —CONR¹⁰— or —NR⁹—;

R² is -halo, —C₀₋₆alkyl, C₀₋₆alkyl-W—C₁₋₆alkyl, C₀₋₆alkyl-W—C₃₋₇cycloalkyl, C₀₋₆alkyl-W-phenyl, or C₀₋₆alkyl-W-heterocycle, wherein the C₁₋₆alkyl, C₃₋₇cycloalkyl, phenyl and heterocycle optionally are independently substituted with 1-6 halo, trifluoromethyl, —CN, —C₁₋₆alkyl, or hydroxy substituents;

R³ is hydrogen, —(C₀₋₆alkyl)-phenyl, —(C₀₋₆alkyl)-heterocycle, —(C₀₋₆alkyl)-C₃₋₇cycloalkyl, —(C₀₋₆alkyl)-CO₂R¹⁰, —(C₀₋₆alkyl)-(C₂₋₆alkenyl)-CO₂R¹⁰, —(C₀₋₆alkyl)-SO₃H, —(C₀₋₆alkyl)-W-C₀₋₄alkyl, —(C₀₋₆alkyl)-CONR¹⁰-phenyl, —(C₀₋₆alkyl)-CONR¹²—V—CO₂R¹⁰, and wherein R³ is nothing when X is O, and wherein C₀₋₆alkyl is optionally substituted with 1-5 independent halo, hydroxy, —C₀₋₆alkyl, —O—C₁₋₃alkyl, trifluoromethyl, or —C₀₋₂alkyl-phenyl substituents, and wherein the phenyl, pyridyl, diazolyl, tetrazolyl, thiadiazolonyl, oxadiazolonyl, thiazolphenyl, N-oxide pyridyl, heterocycle, cycloalkyl, or C₀₋₄alkyl is optionally substituted with 1-5 independent halo, trifluoromethyl, hydroxy, C₁₋₃alkyl, —O—C₁₋₃alkyl, —C₀₋₃—CO₂R¹⁰, —CN, —(C₀₋₆alkyl)-C(O)—(C₀₋₆alkyl), —NR¹⁰R¹⁰, —CONR¹⁰R¹⁰, or —(C₀₋₃alkyl)-heterocycle substituents, and wherein the phenyl and heterocycle may be fused to another heterocycle, which itself optionally may be substituted with 1-2 independently hydroxy, halo, —CO₂R¹⁰, or —C₁₋₃alkyl substituents, and where alkenyl is optionally substituted with 1-3 independently halo, trifluoromethyl, C₁₋₃alkyl, phenyl, or heterocycle substituents;

V is C₁₋₆alkyl or phenyl;

R¹² is hydrogen, C₁₋₄alkyl, or R¹² is joined via a 1-5 carbon tether to one of the carbons of V to form a ring;

R⁴ is nothing when X is either O, or N or when a double bond joins the carbons to which R³ and R⁶ are attached, or R⁴ is hydrogen, hydroxy, C₀₋₆alkyl, C₁₋₆alkyl-hydroxy, —O—C₁₋₃alkyl, —CO₂R¹⁰, —CONR¹⁰R¹⁰, or —CN;

or R³ and R⁴ are joined together to form a 1H-indenyl, 2,3-dihydro-1H-indenyl, 2,3-dihydro-benzofuranyl, 1,3-dihydro-isobenzofuranyl, 2,3-dihydro-benzothiofuranyl, 1,3-dihydro-isobenzothiofuranyl, 6H-cyclopenta[d]isoxazol-3-olyl, cyclopentanyl, or cyclohexanyl ring, wherein the ring formed optionally is substituted with 1-5 independently halo, trifluoromethyl, hydroxy, C₁₋₃alkyl, —O—C₁₋₃alkyl, —C₀₋₃—CO₂R¹⁰, —CN, —NR¹⁰R¹⁰, CONR¹⁰R¹⁰, or —C₀₋₃-heterocyclyl substituents;

or R³ and R⁵ or R⁴ and R⁶ are joined together to form a phenyl or heterocyclyl ring, wherein the ring is optionally substituted with 1-7 independent halo, trifluoromethyl, hydroxy, C₁₋₃alkyl, —O—C₁₋₃alkyl, —CO₂R₁₀, —CN, —NR¹⁰R¹⁰, or —CONR¹⁰R¹⁰ substituents;

R⁵ and R⁶ are independently hydrogen, hydroxy, C₁₋₆alkyl, C₁₋₆alkyl-CO₂R¹⁰, C₁₋₆alkyl-hydroxy, —O—C₁₋₃alkyl, or halo; or ═O, when R⁵ or R⁶ is connected to the ring via a double bond;

when Z=C, R⁷ is hydrogen, hydroxy, halo, C₁₋₆alkyl optionally substituted with 1-6 fluro, —O—C₁₋₆alkyl optionally substituted with 1-6 fluro, —NR¹⁰R¹⁰, —NR¹⁰CO₂R¹¹, —NR¹⁰CONR¹⁰R₁₀, —NR¹⁰—SO₂—NR¹⁰R¹⁰, —NR¹⁰—SO₂—R¹¹, heterocycle, —CN, —CONR¹⁰R¹⁰, —CO₂R¹⁰, —NO₂, —S—R¹⁰, —SO—R¹¹, —SO₂—R¹¹, or —SO₂—NR¹¹R¹¹;

when Z=N, R⁷ is nothing or oxide (resulting in a pyridine N-oxide);

R⁸ is hydrogen, C₁₋₆alkyl, trifluoromethyl, trifluoromethoxy, chloro, fluoro, bromo, or phenyl;

R⁹ is So₂R¹¹, COR¹⁰, CONHR¹⁰, CO₂R¹¹, or SO₂NHR¹⁰;

R¹⁰ is hydrogen, —C₁₋₆ alkyl, benzyl, phenyl, or —C₀₋₆ alkyl-C₃₋₆ cycloalkyl, optionally substituted with 1-3 independent halo, C₁₋₃alkyl, C₁₋₃alkoxy or trifluoromethyl substituents;

R¹¹ is C₁₋₆alkyl, —C₀₋₆alkyl-C₃₋₆cycloalkyl, benzyl or phenyl, optionally substituted with 1-3 independent halo, C₁₋₃alkyl, C₁₋₃alkoxy or trifluoromethyl substituents;

n¹ and n² are independently 0, 1 or 2, wherein the sum of n¹ and n² is 0, 1, 2, or 3; and

the dashed line represents an optional bond.

### Formula I Compounds

### EXAMPLES

Examples of the compounds of Formula I include the following:

### Example I-1

### 44363-64

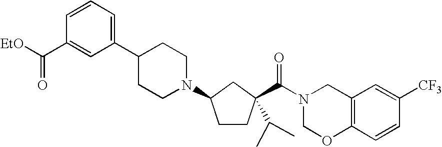

### Example I-2

### 44363-70, L-392018-001R005

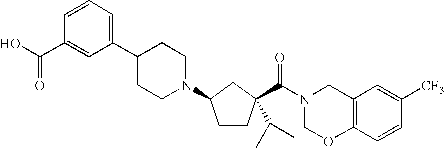

### Example I-3

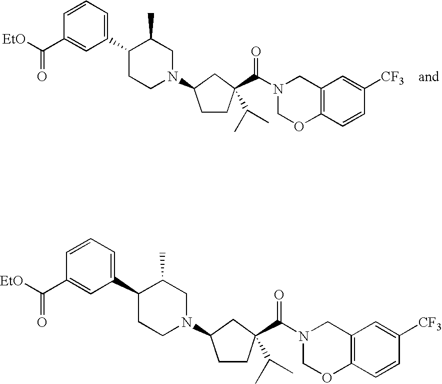

### Example I-4

### (Steve Goble, NB#)

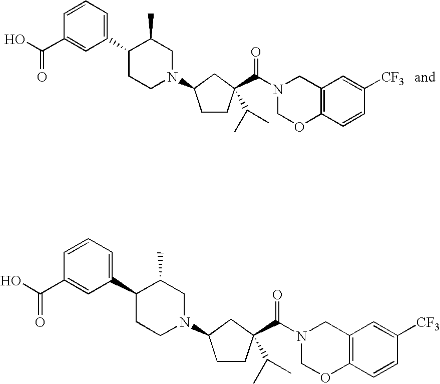

### Example I-5

### 44363-67, L-458295; L-458296, L-459541, and L-459545

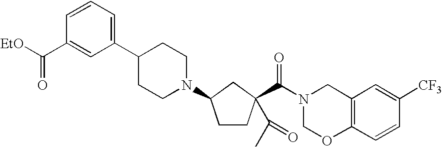

### Example I-6

### 44363-75 and 113, L-464123 and L-464129

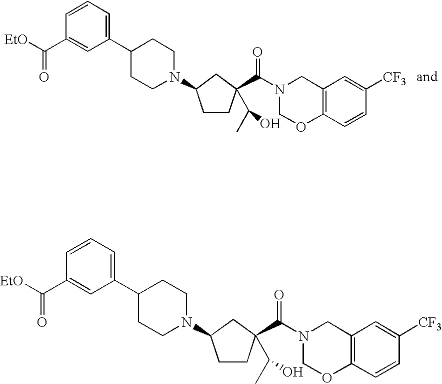

### Example I-7

### 44363-83, L-464946 and L-464962

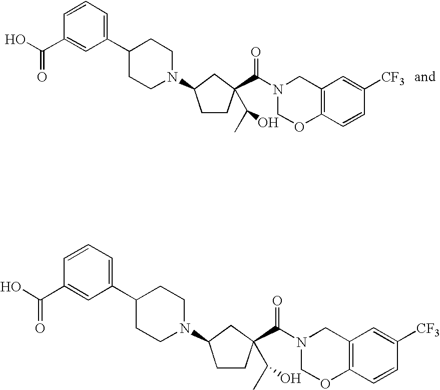

### Example I-8

### 44363-103

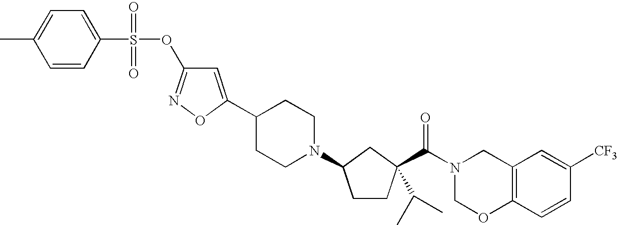

### Example I-9

### L-472057-001B001, 44363-106

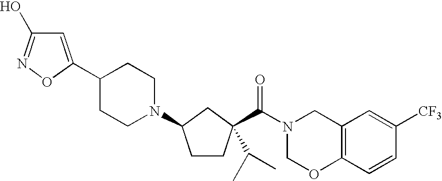

### Examples I-10 to I-46 I-3A and I-3B

Examples I-10 through I-46, I-3A and I-3B, in Table 1, below, are based on the formula:

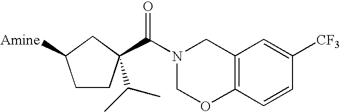

| | | | ESI-MS observed |
|---|---|---|---|
| EX. | Amine | Formula/calc. MW | M + H⁺ (M + 1) |
| I-10 | 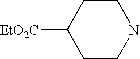 | C26H35F3N2O4 496 | 497 |
| I-11 | 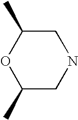 | C24H33F3N2O3 454 | 455 |
| I-12 | 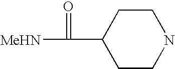 | C25H34F3N3O3 481 | 482 |
| I-13 | 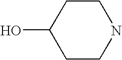 | C23H31F3N2O3 440 | 441 |
| I-14 | 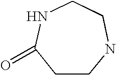 | C23H30F3N3O3 453 | 454 |
| I-15 | 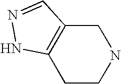 | C24H29F3N4O2 462 | 463 |
| I-16 | 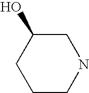 | C23H31F3N2O3 440 | 441 |
| I-17 | 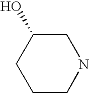 | C23H31F3N2O3 440 | 441 |
| I-18 | 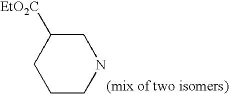 | C26H35F3N2O4 496 | 497 |
| I-19 | 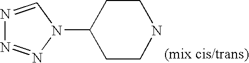 | C24H31F3N6O2 492 | 493 |
| I-20 | 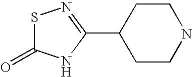 | C25H31F3N4O3S 524 | 525 |
| I-21 | 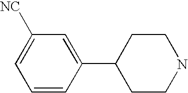 | C30H34F3N3O2 525 | 526 |
| I-22 | 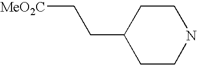 | C27H37F3N2O4 510 | 511 |
| I-23 | 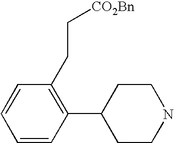 | C39H45F3N2O4 662 | 663 |
| I-24 | 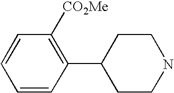 | C31H37F3N2O4 558 | 559 |
| I-25 | 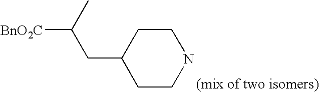 | C34H43F3N2O4 600 | 601 |
| I-26 | 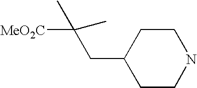 | C29H41F3N2O4 538 | 539 |
| I-27 | 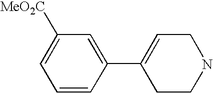 | C31H35F3N2O4 556 | 557 |
| I-28 | 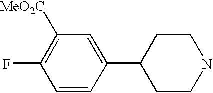 | C31H36F4N2O4 576 | 577 |
| I-29 | 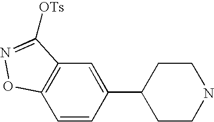 | C37H40F3N3O6S 711 | 712 |
| I-30 | 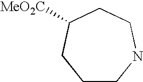 | C26H35F3N2O4 496 | 497 |
| I-31 | 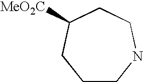 | C26H35F3N2O4 496 | 497 |
| I-32 | 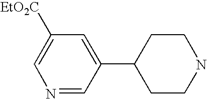 | C31H38F3N3O4 573 | 574 |
| I-33 | 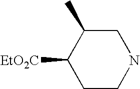 | C27H37F3N2O4 510 | 511 |
| I-34 | 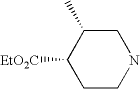 | C27H37F3N2O4 510 | 511 |
| I-35 | 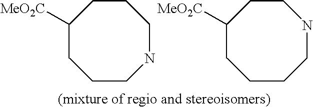 | C27H37F3N2O4 510 | 511 |
| I-3A | 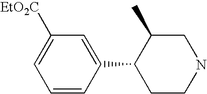 | C33H41F3N2O4 586 | 587 |
| I-3B | 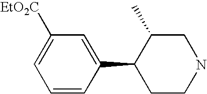 | C33H41F3N2O4 586 | 587 |
| I-36 | 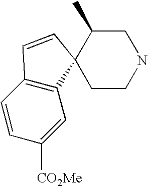 | C34H39F3N2O4 596 | 597 |
| I-37 | 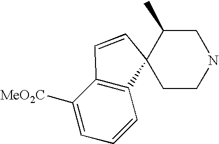 | C34H39F3N2O4 596 | 597 |
| I-38 | 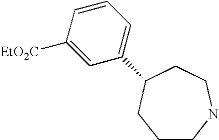 | C33H41F3N2O4 586 | 587 |
| I-39 | 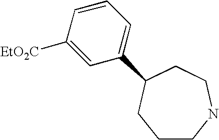 | C33H41F3N2O4 586 | 587 |
| I-40 | 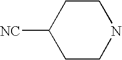 | C24H30F3N3O2 449 | 450 |
| I-41 | 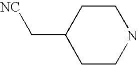 | C25H32F3N3O2 463 | 464 |
| I-42 | 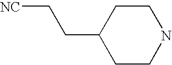 | C26H34F3N3O2 477 | 478 |
| I-43 | 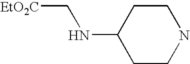 | C27H38F3N3O4 525 | 526 |
| I-44 | 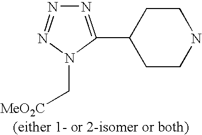 | C27H35F3N6O4 564 | 565 |
| I-45 | 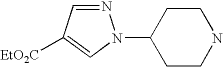 | C29H37F3N4O4 562 | 563 |
| I-46 | 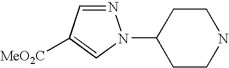 | C28H35F3N4O4 548 | 549 |

In may cases the analogs listed in Table 1 could be further modified to generate new target chemokine receptor modulators. For example, the ester groups of the analogs in this table were hydrolyzed to give the corresponding carboxylic acids which were themselves potent modulators. Alternatively, in the case of benzyl esters, the carboxylic acid could be generated by hydrogenolysis. A representative list of the resulting carboxylic acid containing chemokine receptor modulators is presented below in Table 2.

### Examples I-47 to I-69, I-4A and I-4B

Examples I-47 through I-69, I-4A and I-4B, in-Table 2, below, are based on the formula:

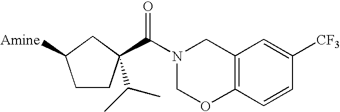

| | | | ESI-MS observed |
|---|---|---|---|
| EX. | Amine | Formula/calc. MW | M + H⁺ (M + 1) |
| I-47 | 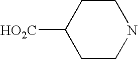 | C24H31F3N2O4 468 | 469 |
| I-48 | 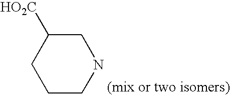 | C24H31F3N2O4 468 | 469 |
| I-49 | 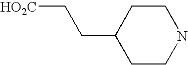 | C26H35F3N2O4 496 | 497 |
| I-50 | 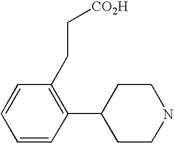 | C32H39F3N2O4 572 | 573 |
| I-51 | 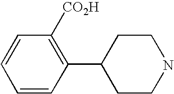 | C30H35F3N2O4 544 | 545 |
| I-52 | 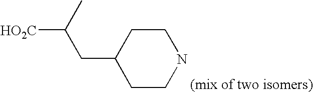 | C27H37F3N2O4 510 | 511 |
| I-53 | 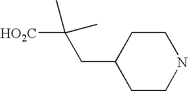 | C28H39F3N2O4 524 | 525 |
| I-54 | 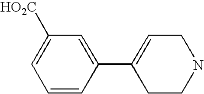 | C30H33F3N2O4 542 | 543 |
| I-55 | 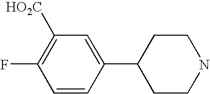 | C30H34F4N2O4 562 | 563 |
| I-56 | 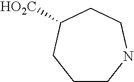 | C25H33F3N2O4 482 | 483 |
| I-57 | 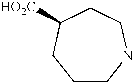 | C25H33F3N2O4 482 | 483 |
| I-58 | 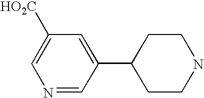 | C29H34F3N3O4 545 | 546 |
| I-59 | 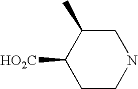 | C25H33F3N2O4 482 | 483 |
| I-60 | 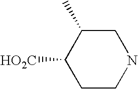 | C25H33F3N2O4 482 | 483 |
| I-61 | 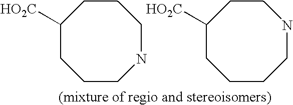 | C26H35F3N2O4 496 | 497 |
| I-4A | 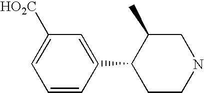 | C31H37F3N2O4 558 | 559 |
| I-4B | 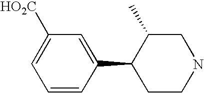 | C31H37F3N2O4 558 | 559 |
| I-62 | 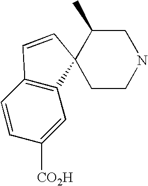 | C33H37F3N2O4 582 | 583 |
| I-63 | 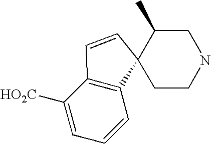 | C33H37F3N2O4 582 | 583 |
| I-64 | 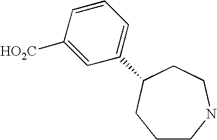 | C31H37F3N2O4 558 | 559 |
| I-65 | 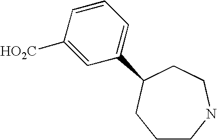 | C31H37F3N2O4 558 | 559 |
| I-66 | 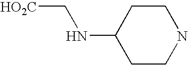 | C25H34F3N3O4 497 | 498 |
| I-67 | 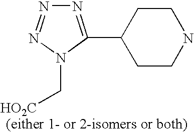 | C26H33F3N6O4 550 | 551 |
| I-68 | 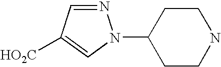 | C27H33F3N4O4 534 | 535 |
| I-69 | 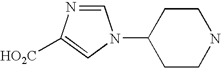 | C27H33F3N4O4 534 | 535 |

### Example I-70

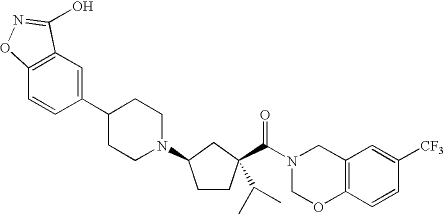

Additional potent chemokine receptor modulators may be created by converting of the nitrile groups found in some of the analogs in Table 1 into tetrazole groups, as described for EXAMPLE I-71 below:

### Example I-71

### L-415175-001C001, 44363-14

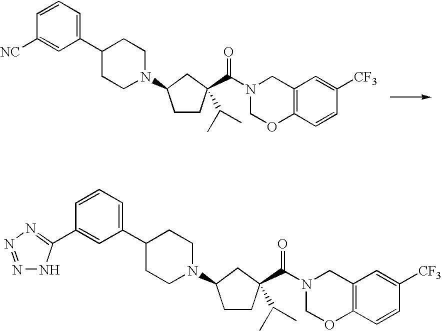

### Examples I-72 to I-74

In a similar fashion to that described immediately above, the Examples in Table 3, below, were prepared by conversion of nitrile containing analogs into the corresponding tetrazole containing analogs. Examples I-72 through I-74, in Table 3, below, are based on the formula:

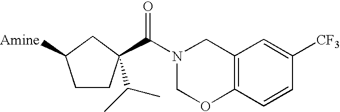

| | | | ESI-MS observed |
|---|---|---|---|
| EX. | Amine | Formula/calc. MW | M + H⁺(M + 1) |
| 1-72 | 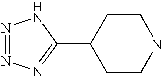 | C24H31F3N6O2 492 | 493 |
| 1-73 | 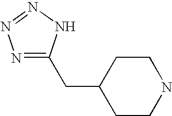 | C25H33F3N6O2 506 | 507 |
| 1-74 | 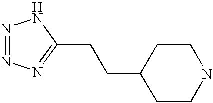 | C26H35F3N6O2 520 | 521 |

### Example I-75

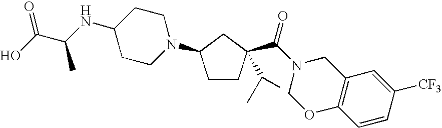

### Example I-76

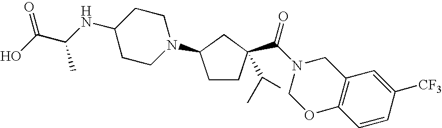

### Example I-77

### L-441092-001R001, 44363-51

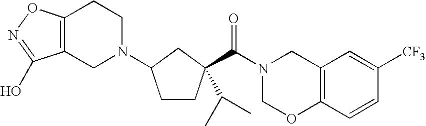

### Examples I-78 to I-81

Examples I-78 through I-81, in Table 4, below, are based on the formula:

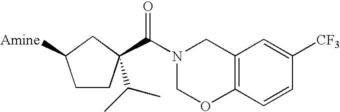

| | | | ESI-MS |
|---|---|---|---|
| | | | ob- |
| | | | served |
| | | | M + H⁺ |
| EX. | Amine | Formula/calc. MW | (M + 1) |
| I-78 | 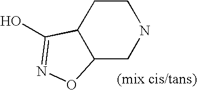 | C24H28F3N3O4 479 | 480 |
| I-79 | 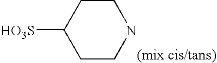 | C23H31F3N2O5S 504 | 505 |
| I-80 | 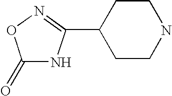 | C25H31F3N4O4 508 | 509 |
| I-81 | 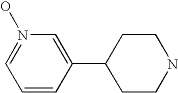 | C28H34F3N3O3 517 | 518 |

Additional CCR-2 antagonists useful in the methods of the invention are those of Formula II. 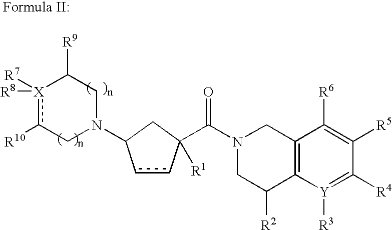
wherein:

X is selected from:
C, N, O, S and SO₂;

Y is selected from N or C.

R¹ is selected from:
hydrogen, —C₁₋₆alkyl, —C₀₋₆alkyl-O—C₁₋₆alkyl, —C₀₋₆alkyl-S—C₁₋₆alkyl, —(C₀₋₆alkyl)-(C₃₋₇cycloalkyl)-(C₀₋₆alkyl), hydroxy, heterocycle, —CN, —NR¹²R¹², —NR₁₂COR¹³, —NR¹²SO₂R¹⁴, —COR¹¹, —CONR¹²R¹², and phenyl,
where R¹¹ is independently selected from: hydroxy, hydrogen, C₁₋₆ alkyl, —O—C₁₋₆alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂-C₁₋₆ alkyl, and trifluoromethyl, and
where R¹² is selected from: hydrogen, C₁₋₆ alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂-C₁₋₆ alkyl, and trifluoromethyl, and
where R¹³ is selected from: hydrogen, C₁₋₆ alkyl, —O—C₁₋₆alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂-C₁₋₆ alkyl, and trifluoromethyl, and
where R¹⁴ is selected from: hydroxy, C₁₋₆ alkyl, —O—C₁₋₆-alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl, and
where the alkyl and the cycloalkyl are unsubstituted or substituted with 1-7 substituents where the substituents are independently selected from:
   (a) halo,
   (b) hydroxy,
   (c) —O—C₁₋₃alkyl,
   (d) trifluoromethyl,
   (f) C₁₋₃alkyl,
   (g) —O—C₁₋₃alkyl,
   (h) —COR¹¹,
   (i) —SO₂R¹⁴,
   (j) —NHCOCH₃,
   (k) —NHSO₂CH₃,
   (l) -heterocycle,
   (m) ═O,
   (n) —CN,
and where the phenyl and heterocycle are unsubstituted or substituted with 1-3 substituents where the substituents are independently selected from: halo, hydroxy, C₁₋₃ alkyl, C₁₋₃alkoxy and trifluoromethyl;

R² is selected from:
(a) hydrogen,
(b) hydroxy,
(c) halo,
(d) C₁₋₃alkyl, where the alkyl is unsubstituted or substituted with 1-6 substituents independently selected from: fluoro, and hydroxy,
(e) —NR¹²R¹²,
(f) —COR¹¹,
(g) —CONR¹²R¹²,
(h) —NR¹²COR¹³,
(i) —OCONR¹²R¹²,
(j) —NR¹²CONR¹²R¹²,
(k) -heterocycle,
(l) —CN,
(m) —NR¹²—SO₂—NR¹²R¹²,
(n) —NR¹²—SO₂—R¹⁴,
(o) —SO₂—NR¹²R¹², and
(p) ═O, where R² is connected to the ring via a double bond;

R³ is oxygen or is absent when Y is N;

R³ is selected from the following list when Y is C:
(a) hydrogen,
(b) hydroxy,
(c) halo,
(d) C₁₋₃alkyl, where the alkyl is unsubstituted or substituted with 1-6 substituents independently selected from: fluoro, hydroxy, and —COR¹¹,
(e) —NR¹²R¹²,
(f) —COR¹¹,
(g) —CONR¹²R¹²,
(h) —NR¹²COR¹³,
(i) —OCONR¹²R¹²,
(j) —NR¹²CONR¹²R¹²,
(k) -heterocycle,
(l) —CN,
(m) —NR¹²—SO₂—NR¹²R¹²,
(n) —NR¹²—SO₂—R¹⁴,
(o) —SO₂—NR¹²R¹² and
(p) nitro;

R⁴ is selected from:
(a) hydrogen,
(b) C₁₋₆alkyl,
(c) trifluoromethyl,
(d) trifluoromethoxy,
(e) chloro,
(f) fluoro,
(g) bromo, and
(h) phenyl;

R⁵ is selected from:
(a) C₁₋₆alkyl, where alkyl may be unsubstituted or substituted with 1-6 fluoro and optionally substituted with hydroxyl,
(b) —O—C₁₋₆alkyl, where alkyl may be unsubstituted or substituted with 1-6 fluoro,
(c) —CO—C₁₋₆alkyl, where alkyl may be unsubstituted or substituted with 1-6 fluoro,
(d) —S—C₁₋₆alkyl, where alkyl may be unsubstituted or substituted with 1-6 fluoro,
(e) -pyridyl, which may be unsubstituted or substituted with one or more substituents selected from the group consisting of: halo, trifluoromethyl, C₁₋₄alkyl, and COR¹¹,
(f) fluoro,
(g) chloro,
(h) bromo,
(i) —C₄₋₆cycloalkyl,
(j) —O—C₄₋₆cycloalkyl,
(k) phenyl, which may be unsubstituted or substituted with one or more substituents selected from the group consisting of: halo, trifluoromethyl, C₁₋₄alkyl, and COR¹¹,
(l) —O-phenyl, which may be unsubstituted or substituted with one or more substituents selected from the group consisting of: halo, trifluoromethyl, C₁₋₄alkyl, and COR¹¹,
(m) —C₃₋₆cycloalkyl, where alkyl may be unsubstituted or substituted with 1-6 fluoro,
(n) —O—C₃₋₆cycloalkyl, where alkyl may be unsubstituted or substituted with 1-6 fluoro,
(o) -heterocycle,
(p) —CN, and
(q) —COR¹¹;

R⁶ is selected from:
(a) hydrogen,
(b) C₁₋₆alkyl, and
(c) trifluoromethyl
(d) fluoro
(e) chloro, and
(f) bromo;

R⁷ is selected from:
nothing (when X═O), hydrogen, (C₀₋₆alkyl)-phenyl, (C₀₋₆alkyl)-heterocycle, (C₀₋₆alkyl)-C₃₋₇cycloalkyl, (C₀₋₆alkyl)-COR¹¹, (C₀₋₆alkyl)-(alkene)-COR¹¹, (C₀₋₆alkyl)-SO₃H, (C₀₋₆alkyl)-W—C₀₋₄alkyl, (C₀₋₆alkyl)-CONR¹²-phenyl, (C₀₋₆alkyl)-CONR¹⁵—V—COR¹¹, and nothing (when X is O, S, or SO₂), where V is selected from C₁₋₆ alkyl or phenyl, and
   where W is selected from: a single bond, —O—, —S—, —SO—, —SO₂—, —CO—, —CO₂—, —CONR¹² and —NR¹²—, and
   where the R¹⁵ can be hydrogen, C₁₋₄alkyl, or where R¹⁵ is joined via a 1-5 carbon tether to one of the carbons of V to form a ring, and
   where the C₀₋₆alkyl is unsubstituted or substituted with 1-5 substituents, where the substituents are independently selected from:
   (a) halo,
   (b) hydroxy,
   (c) —C₀₋₆alkyl
   (d) —O—C₁₋₃alkyl,
   (e) trifluoromethyl, and
   (f) —C₀₋₂alkyl-phenyl,
and where the phenyl, heterocycle, cycloalkyl, and C₀₋₄alkyl is unsubstituted or substituted with 1-5 substituents where the substituents are independently selected from:
   (a) halo,
   (b) trifluoromethyl,
   (c) hydroxy,
   (d) C₁₋₃alkyl,
   (e) —O—C₁₋₃alkyl,
   (f) —C₀₋₃—COR¹¹,
   (g) —CN,
   (h) —NR¹²R¹²,
   (i) —CONR¹²R¹², and
   (l) —C₀₋₃-heterocycle,
   or where the phenyl and heterocycle may be fused to another heterocycle, which itself may be unsubstituted or substituted with 1-2 substituents independently selected from hydroxy, halo, —COR¹¹, and —C₁₋₃alkyl,
and where alkene is unsubstituted or substituted with 1-3 substituents which are independently selected from:
   (a) halo,
   (b) trifluoromethyl,
   (c) C₁₋₃alkyl,
   (d) phenyl, and
   (e) heterocycle;

R⁸ is selected from:
(a) hydrogen,
(b) nothing when X is either O, S, SO₂ or N or when a double bond joins the carbons to which R⁷ and R¹⁰ are attached,
(c) hydroxy,
(d) C₁₋₆alkyl,
(e) C₁₋₆alkyl-hydroxy,
(f) —O—C₁₋₃alkyl,
(g) —COR¹¹,
(h) —CONR¹²R¹², and
(i) —CN;

or where R⁷ and R⁸ may be joined together to form a ring which is selected from:
(a) 1H-indene,
(b) 2,3-dihydro-1H-indene,
(c) 2,3-dihydro-benzofuran,
(d) 1,3-dihydro-isobenzofuran,
(e) 2,3-dihydro-benzothiofuran,
(f) 1,3-dihydro-isobenzothiofuran,
(g) 6H-cyclopenta[d]isoxazol-3-ol
(h) cyclopentane, and
(i) cyclohexane,
where the ring formed may be unsubstituted or substituted with 1-5 substituents independently selected from:
(a) halo,
(b) trifluoromethyl,
(c) hydroxy,
(d) C₁₋₃alkyl,
(e) —O—C₁₋₃alkyl,
(f) —C₀₋₃—COR¹¹,
(g) —CN,
(h) —NR¹²R¹²,
(i) —CONR¹²R¹², and
(j) —C₀₋₃-heterocycle,

or where R⁷ and R⁹ or R⁸ and R¹⁰ may be joined together to form a ring which is phenyl or heterocycle,
wherein the ring is unsubstituted or substituted with 1-7 substituents where the substituents are independently selected from:
(a) halo,
(b) trifluoromethyl,
(c) hydroxy,
(d) C₁₋₃alkyl,
(e) —O—C₁₋₃alkyl,
(f) —COR¹¹,
(g) —CN,
(h) —NR¹²R¹², and
(i) —CONR¹²R¹²;

R⁹ and R¹⁰ are independently selected from:
(a) hydrogen,
(b) hydroxy,
(c) C₁₋₆alkyl,
(d) C₁₋₆alkyl-COR¹¹,
(e) C₁₋₆alkyl-hydroxy,
(f) —O—C₁₋₃alkyl,
(g) ═O, when R⁹ or R¹⁰ is connected to the ring via a double bond
(h) halo;

n is selected from 0, 1 and 2;

the dashed line represents a single or a double bond;

and pharmaceutically acceptable salts thereof and individual diastereomers thereof.

### Formula II Compounds

### EXAMPLES

Examples of the compounds of Formula II include the following:

### Example II-1

### L-070912

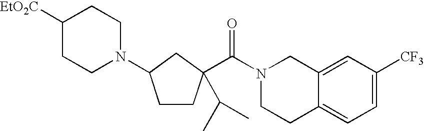

### Examples II-2 to II-6

### (L-070913/914/915/922/923

Examples II-2 through II-6, in Table 5, below, are based on the formula:

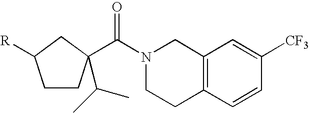

| Ex- | | Molecular | Calculated | Found |
|---|---|---|---|---|
| ample | R | Formula | [M⁺H⁺] | [M⁺H⁺] |
| II-2 | 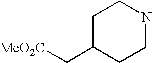 | C₂₇H₃₈F₃N₂O₃ | 495.28 | 495.15 |
| II-3 | 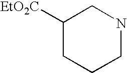 | C₂₇H₃₈F₃N₂O₃ | 485.28 | 495.15 |
| II-4 | 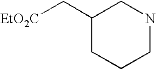 | C₂₈H₄₀F₃N₂O₃ | 509.29 | 509.35 |
| II-5 | 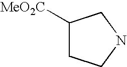 | C₂₅H₃₄F₃N₂O₃ | 467.24 | 467.1 |
| II-6 | 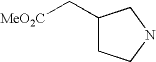 | C₂₆H₃₆F₃N₂O₃ | 481.26 | 481.2 |

### Example II-7

### L-070927

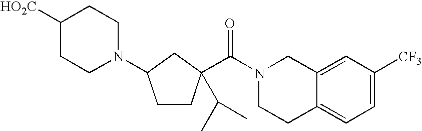

### Examples II-8 to II-12

### L-070928/929/930/932/???

Examples II-8 through II-12, in Table 6, below, are based on the formula:

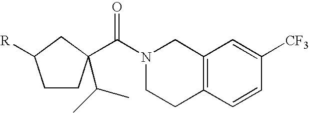

| Ex- | | Molecular | Calculated | Found |
|---|---|---|---|---|
| ample | R | Formula | [M⁺H⁺] | [M⁺H⁺] |
| II-8 | 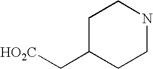 | C₂₆H₃₆F₃N₂O₃ | 481.26 | 481.3 |
| II-9 | 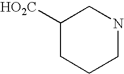 | C₂₅H₃₄F₃N₂O₃ | 467.24 | 467.3 |
| II-10 | 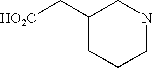 | C₂₆H₃₆F₃N₂O₃ | 481.26 | 481.3 |
| II-11 | 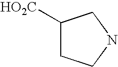 | C₂₄H₃₂F₃N₂O₃ | 453.23 | 453.25 |
| II-12 | 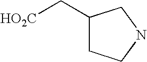 | C₂₅H₃₃F₃N₂O₃ | 467.24 | 467.25 |

### Example II-13

### L-310727; M. Lombardo; 31995-91 #3

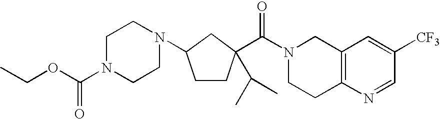

### Examples II-14 to II-16

### L-071082, L-071083, L-310729

Examples II-14 through II-16, in Table 7, below, are based on the formula:

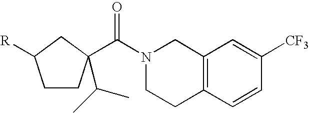

| | | | Calcu- | |
|---|---|---|---|---|
| Ex- | | Molecular | lated | Found |
| ample | R | Formula | [M⁺H⁺] | [M⁺H⁺] |
| II-14 | 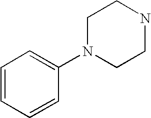 | C₂₈H₃₆F₃N₄O | 501.28 | 501.25 |
| II-15 | 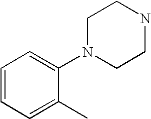 | C₂₉H₃₇F₃N₄O | 515.29 | 515.3 |
| II-16 | 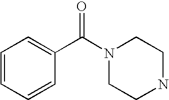 | C₂₉H₃₅F₃N₄O | 528.27 | 529.25 |

### Example II-17

### (L-310728; M. Lombardo; 31995-91 #2

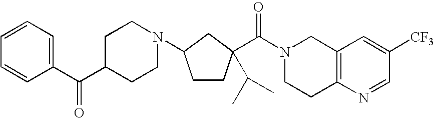

### Example II-18

### L-250442; C. Zhou

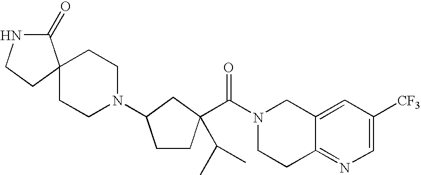

### Example II-19

### L-238241; S. Goble; 44292-063G

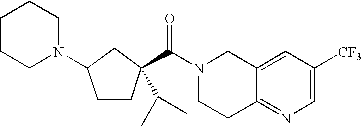

### Examples II-20 to II-28

Examples II-20 through II-28, in Table 8, below, are based on the formula:

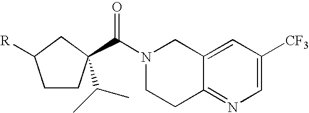

| | | | | Found |
|---|---|---|---|---|
| | | | Cal- | MW |
| Ex- | | Molecular | culated | [M + |
| ample | Structure | Formula | MW | H] |
| II-20 | 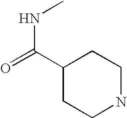 | C25H35F3N4O2 | 480.27 | 481 |
| II-21 | 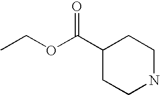 | C26H36F3N3O3 | 495.27 | 496 |
| II-22 | 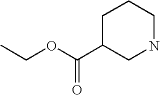 | C26H36F3N3O3 | 495.27 | 496 |
| II-23 | 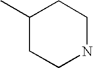 | C24H34F3N3O | 437.27 | 438 |
| II-24 | 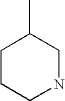 | C24H34F3N3O | 437.27 | 438 |
| II-25 | 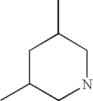 | C25H36F3N3O | 451.28 | 452 |
| II-26 | 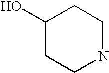 | C23H32F3N3O2 | 439.24 | 440 |
| II-27 | 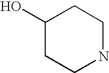 | C23H32F3N3O2 | 439.24 | 440 |
| II-28 | 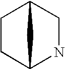 | C24H32F3N3O | 435.25 | 436 |

### Example II-29 and Example 11-30

### L-250911/913; S. Goble; 44292-075C-1/2

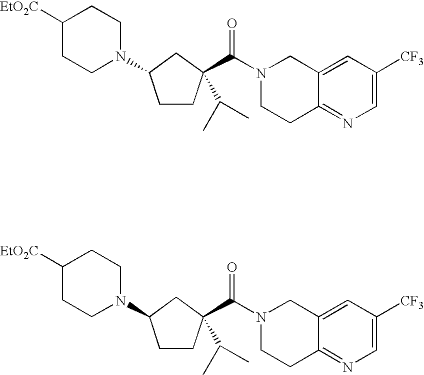

### Example II-31

### L-251644; S. Goble; 44292-079A

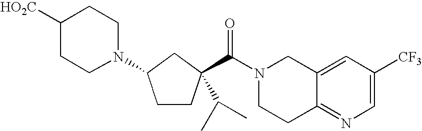

### Example II-32

### L-251638; S. Goble; 44292-079B

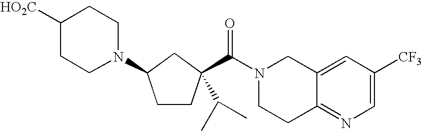

### Example II-33

### L-259996; S. Goble; 44292-080B

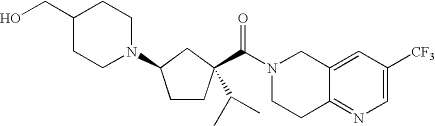

### Example II-34 and Example II-35

### L-896353/354; S. Goble; 44292-096-112

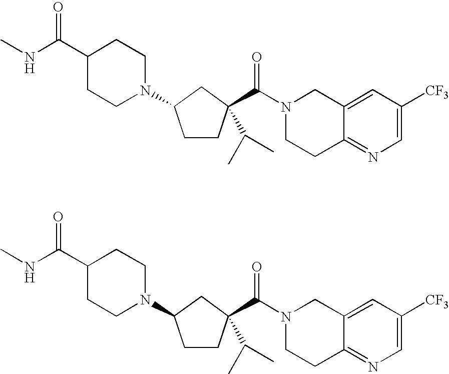

### Example II-36 and Example II-37

### L-251400/402; S. Goble; 44292-75B-1/2

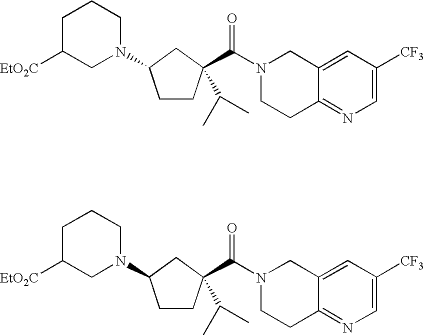

### Example II-38

### L-311529/628/743/748; S. Goble; 44292-75B-1/2

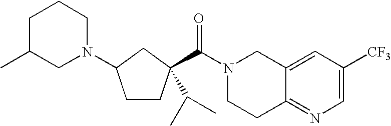

### Example II-42

### L-312021; S. Goble; 44292-75B-1/2

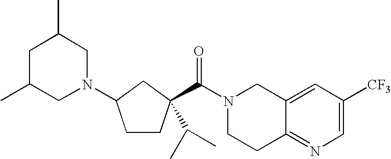

### Example II-47 and Example II-48

### L-330379/467; S. Goble; 44292-114

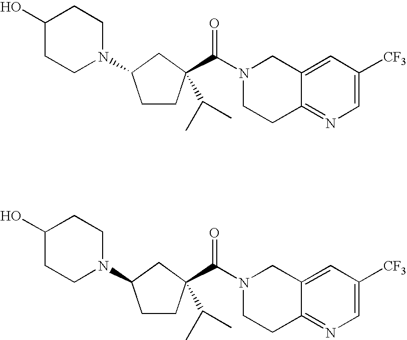

### Example II-49

### L-238242; S. Goble; 44292-0631

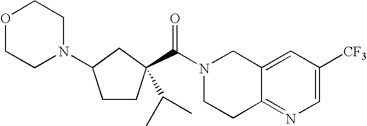

### Examples II-50 to II-53

Examples II-50 through II-53, in Table 9, below, are based on the formula:

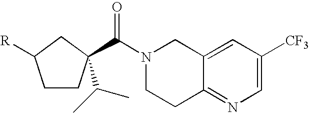

| | | Molecular | Calculated | Found MW |
|---|---|---|---|---|
| Example | Structure | Formula | MW | [M + H] |
| II-50 | 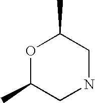 | C24H34F3N3O2 | 453.26 | 454 |
| II-51 | 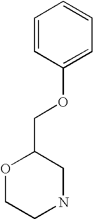 | C29H36F3N3O3 | 531.27 | 532 |
| II-52 | 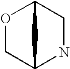 | C23H30F3N3O2 | 437.23 | 438 |

### Example II-53 and Example II-54

### L-250277/280; S. Goble; 44292-072

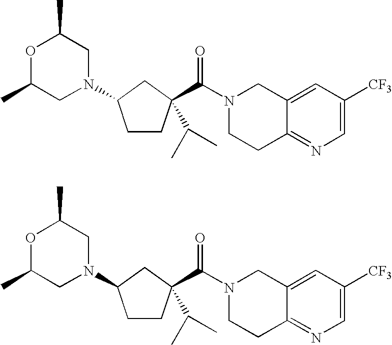

### Example II-55 and Example II-56

### L-250277/280; S. Goble; 44292-072

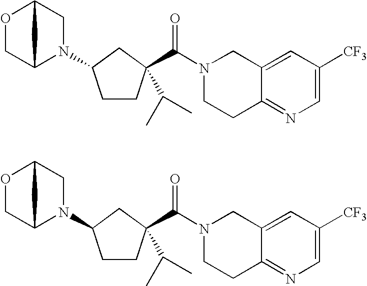

### Example II-57

### L-238248/246; S. Goble; 44292-063H

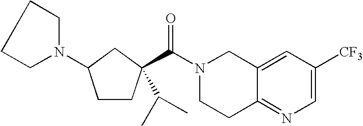

### Examples II-58 to II-62

Examples II-58 through II-62, in Table 10, below, are based on the formula:

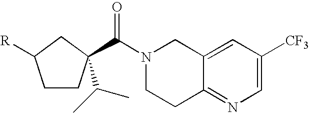

| | | | | Found |
|---|---|---|---|---|
| | | | Cal- | MW |
| Ex- | | Molecular | culated | [M + |
| ample | Structure | Formula | MW | H] |
| II-58 | 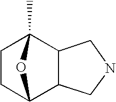 | C27H36F3N3O2 | 491.28 | 492 |
| II-59 | 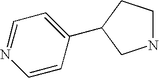 | C27H35F3N4O | 486.26 | 487 |
| II-60 | 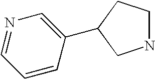 | C27H33F3N4O | 486.26 | 487 |
| II-61 | 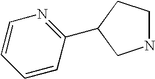 | C27H33F3N4O | 486.26 | 487 |
| II-62 | 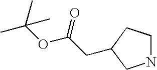 | C28H40F3N3O3 | 523.30 | 524 |

### Example II-63

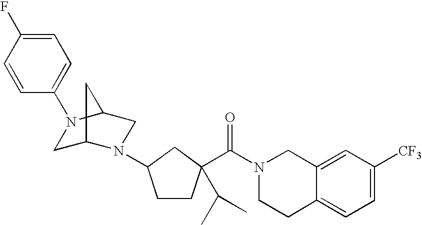

### Example II-64

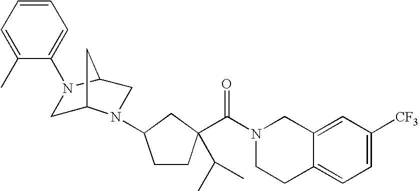

### Example II-65

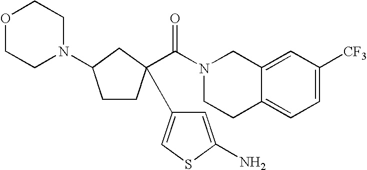

### Example II-66

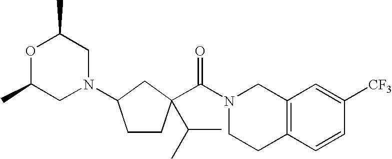

### Example II-67

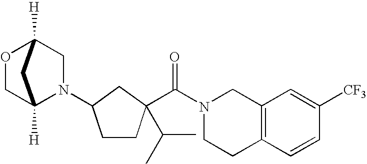

### Example II-68

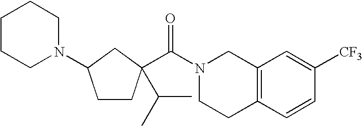

### Example II-69

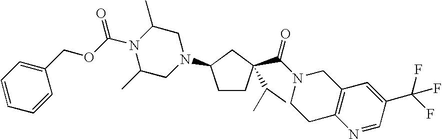

### Examples II-70 to II-72

Examples II-70 through II-72, in Table 11, below, are based on the formula:

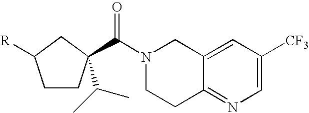

| | | | Cal- | Found |
|---|---|---|---|---|
| Ex- | | Molecular | culated | MW |
| ample | Structure | Formula | MW | [M + H] |
| II-70 | 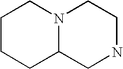 | C26H37F3N4O | 478.29 | 479 |
| II-71 | 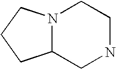 | C25H35F3N4O | 464.28 | 465 |
| II-72 | 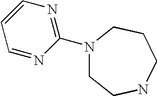 | C27H35F3N6O | 516.28 | 517 |

### Example II-73

### L-311207; S. Goble; 44292-89Q

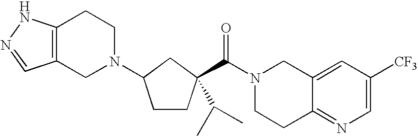

### Example II-74

### L-311211; S. Goble; 44292-89U

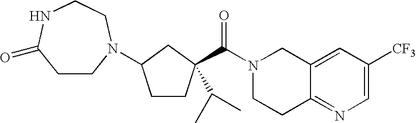

### Example II-75

### L-3103281299; S. Goble; 44292-89Y-1/2

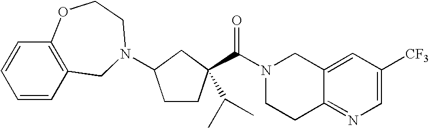

### Example II-76

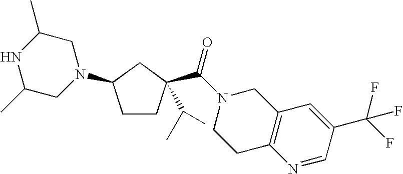

### Example II-77

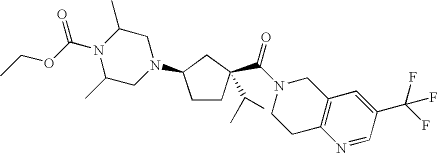

### Example II-78

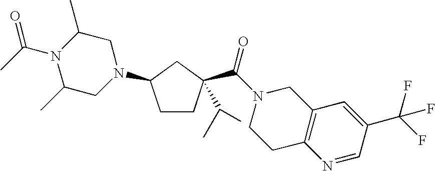

### Example II-79

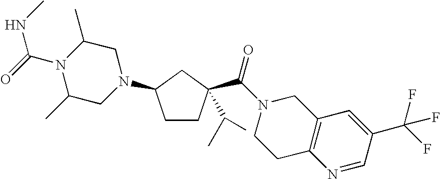

### Example II-80

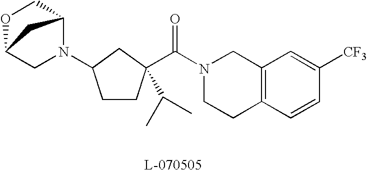

### Example II-81

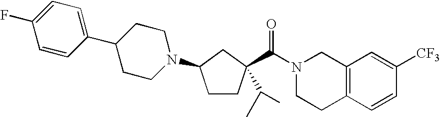

### Example II-82

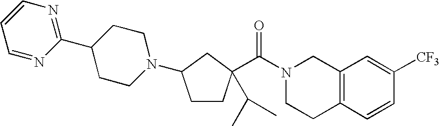

### Examples II-83 to II-91

Examples II-83 through II-91, in Table 12, below, are based on the formula:

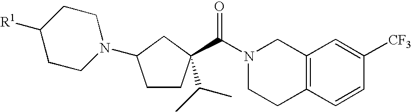

| | | Molecular | Calculated | Found |
|---|---|---|---|---|
| Example | R¹ | Formula | [M] | [M + H] |
| II-83 | 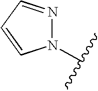 | C27H36F3N4O | 488.27 | 489 |
| II-84 | 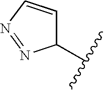 | C27H36F3N4O | 488.27 | 489 |
| II-85 | 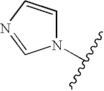 | C27H36F3N4O | 488.27 | 489 |
| II-86 | 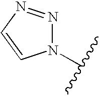 | C26H35F3N5O | 489.27 | 490 |
| II-87 | 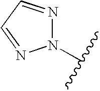 | C26H35F3N5O | 489.27 | 490 |
| II-88 | 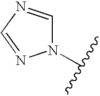 | C26H35F3N5O | 489.27 | 490 |
| II-89 | 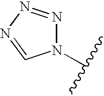 | C25H34F3N6O | 490.26 | 491 |
| II-90 | 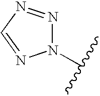 | C25H34F3N6O | 490.26 | 491 |
| II-91 | 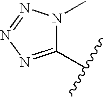 | C26H36F3N6O | 504.26 | 505 |

### Example II-92

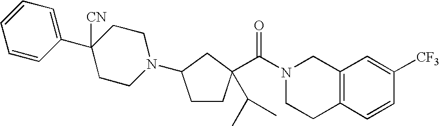

### Example II-93

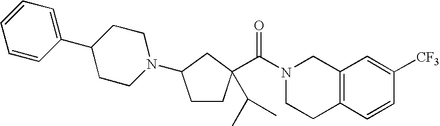

### Example II-94

### L-070188, L-070189

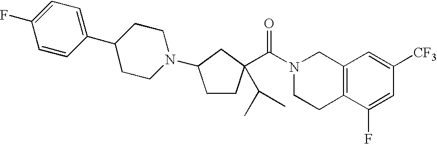

### Example II-95

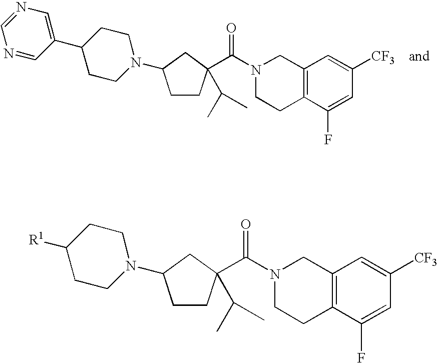

### Example II-105

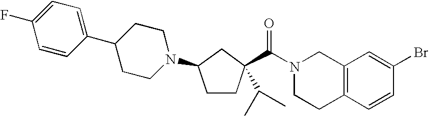

### Example II-106

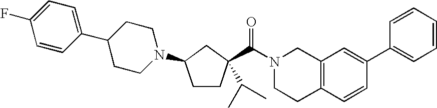

### Example II-107

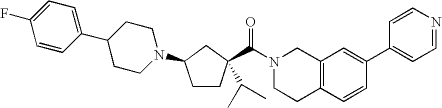

### Example II-108

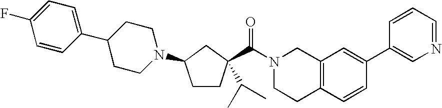

### Example II-109

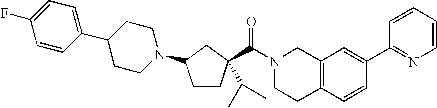

### Example II-110

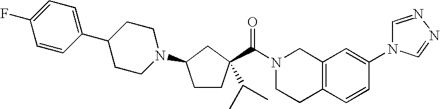

### Example II-111

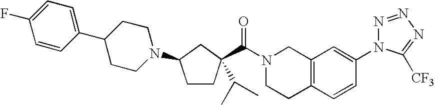

### Example II-112

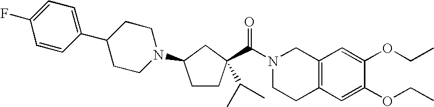

### Example II-113

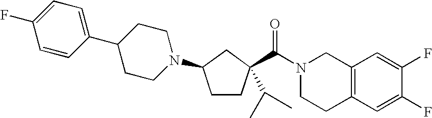

### Example II-114

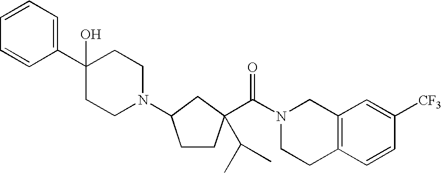

### Examples II-115 and II-116

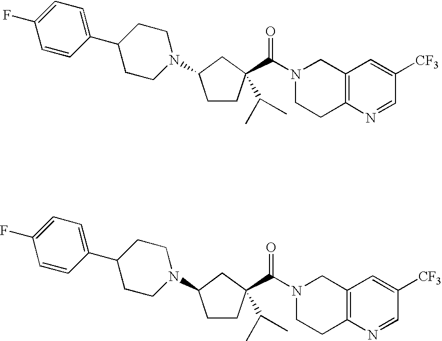

### Example II-117

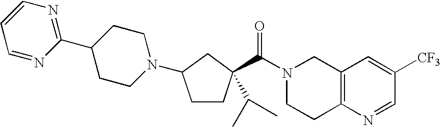

### Examples II-118 to II-129

Examples II-118 through II-129, in Table 13, below, are based on the formula:

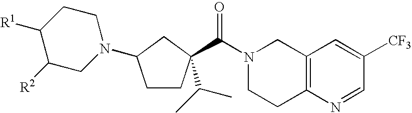

| | | | | Cal- | Found |
|---|---|---|---|---|---|
| Ex- | | | Molecular | culated | [M + |
| ample | R¹ | R² | Formula | [M] | H] |
| II-118 | 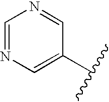 | H | C27H34F3N5O | 501.27 | 502 |
| II-119 | 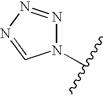 | H | C24H32F3N7O | 491.26 | 492 |
| II-120 | 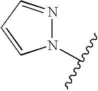 | H | C26H34F3N5O | 489.27 | 490 |
| II-121 | 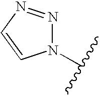 | H | C25H33F3N6O | 490.27 | 491 |
| II-122 | 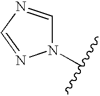 | H | C25H33F3N6O | 490.27 | 491 |
| II-123 | 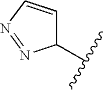 | H | C26H34F3N5O | 489.27 | 490 |
| II-124 | 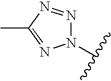 | H | C25H34F3N7O | 505.28 | 506 |
| II-125 | 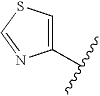 | H | C26H33F3N4OS | 506.23 | 507 |
| II-126 | 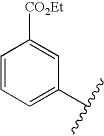 | H | C32H43F3N3O3 | 574.33 | 575 |
| II-127 | 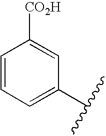 | H | C30H39F3N3O3 | 546.29 | 547 |
| II-128 | 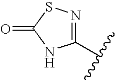 | H | C25H32F3N5O2S | 523.22 | 524 |
| II-129 | 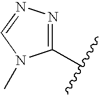 | H | C26H35F3N6O | 504.28 | 505 |

### Example II-130

### L-251172, L-251173, L-251174, L-251176, L-260261

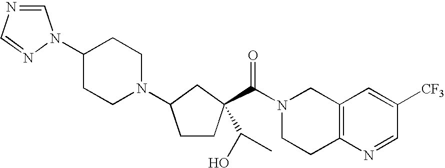

### Example II-131

### L-260661, L-260663, L-310458, L-896360, L-896361, L-896362

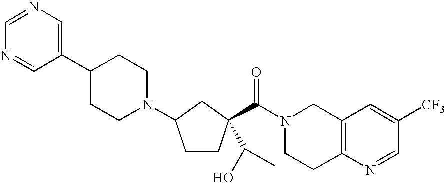

### Example II-132

### L-896358, L-896359

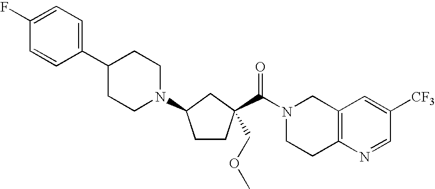

### Example II-133

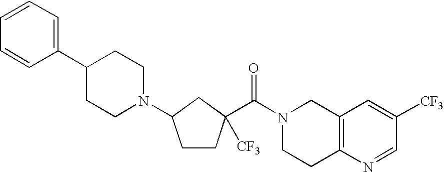

### Example II-134

### L-000400081

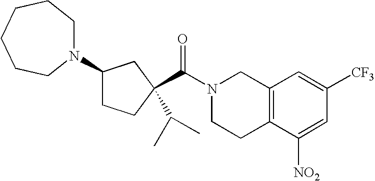

### Example II-135

### L-000400084

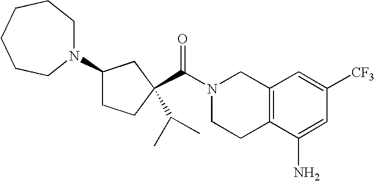

### Example II-136

### L-000401768

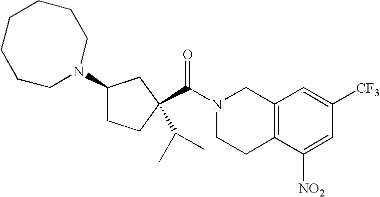

### Example II-137

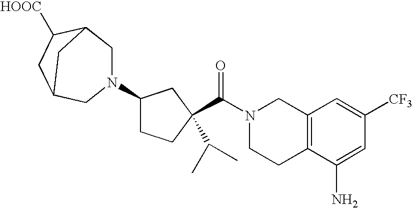

### Example II-138

### L-000392271

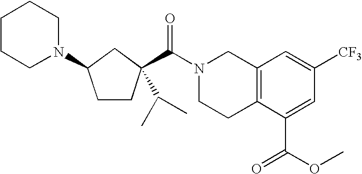

### Example II-139

### L-000392274

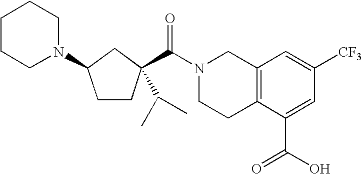

### Example II-140

### L-000392725

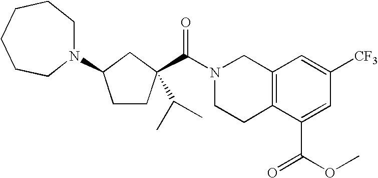

### Example II-141

### L-000392730

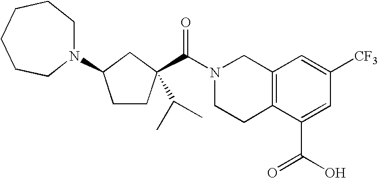

### Example II-142

### L-000436347

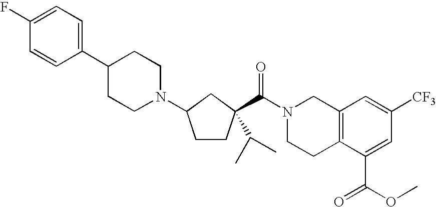

### Example II-143

### L-000436374

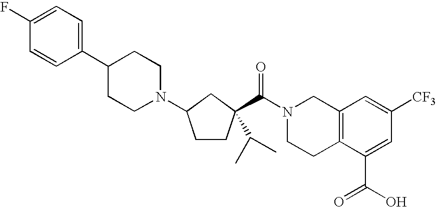

### Example II-144

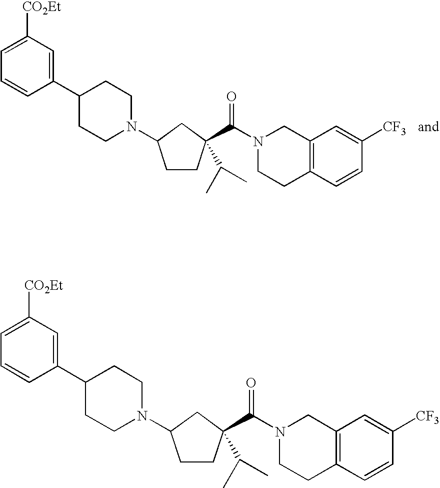

### Example II-145

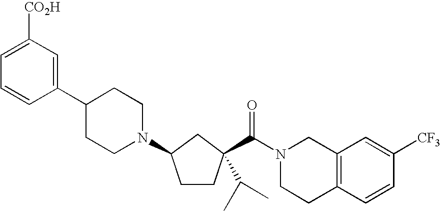

### Example II-146

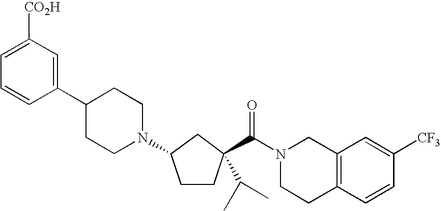

Additional CCR-2 antagonists useful in the inventive methods of the invention are those of Formulae IIIa and IIIb. 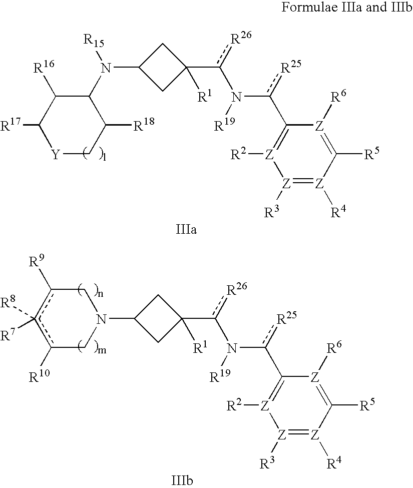
wherein:
X is selected from O, N, S, SO₂, or C.

Y is selected from:
—O—, NR¹²S—, —S—, —SO₂— and —CR¹²R¹²—, —NSO₂R¹⁴—, —NCOR¹³—, —CR¹²COR¹¹—, —CR¹²OCOR¹³—, —CO—,
R¹¹ is independently selected from: hydroxy, hydrogen,
   C₁₋₆ alkyl, —O—C₁₋₆alkyl, benzyl, phenyl, C₃₋₆ cycloalkyl where the alkyl, phenyl, benzyl, and cycloalkyl groups can be unsubstituted or substituted with 1-3 substituents where the substituents are independently-selected from: halo, hydroxy, C₁₋₃alkyl, C₁₋₃alkoxy, —CO₂H, —CO₂—C₁₋₆ alkyl, and trifluoromethyl;
   R¹² is selected from: hydrogen, C₁₋₆ alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl;
   R¹³ is selected from: hydrogen, C₁₋₆ alkyl, —O—C₁₋₆alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl;
   R¹⁴ is selected from: hydroxy, C₁₋₆ alkyl, —O—C₁₋₆alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl;

Z is independently selected from C or N, where at most two of the Z are N.

R¹ is selected from:
hydrogen, —C₁₋₆alkyl, —C₀₋₆alkyl-O—C₁₋₆alkyl, —C₀₋₆alkyl-S—C₁₋₆alkyl, —(C₀₋₆alkyl)-(C₃₋₇cycloalkyl)-(C₀₋₆alkyl), hydroxy, heterocycle, —CN, —NR¹²R¹², —NR¹²COR¹³, —NR¹²SO₂R¹⁴, —COR¹¹, —CONR¹²R¹², and phenyl;
   the alkyl and the cycloalkyl are unsubstituted or substituted with 1-7 substituents where the substituents are independently selected from:
   (a) halo,
   (b) hydroxy,
   (c) —O—C₁₋₃alkyl,
   (d) trifluoromethyl,
   (f) C₁₋₃alkyl,
   (g) —O—C₁₋₃alkyl,
   (h) —COR¹¹,
   (i) —SO₂R¹⁴,
   (j) —NHCOCH₃,
   (k) —NHSO₂CH₃,
   (l) -heterocycle,
   (m) ═O,
   (n) —CN,
and where the phenyl and heterocycle are unsubstituted or substituted with 1-3 substituents where the substituents are independently selected from: halo, hydroxy, —COR¹¹, C₁₋₃alkyl, C₁₋₃alkoxy and trifluoromethyl;

R² is selected from:
(a) hydrogen,
(b) C₁₋₃alkyl, optionally substituted with 1-3 fluoro,
(c) —O—C₁₋₃alkyl, optionally substituted with 1-3 fluoro,
(d) hydroxy,
(e) chloro,
(f) fluoro,
(g) bromo,
(h) phenyl,
(g) heterocycle, and
(h) nothing or O (when the Z bonded to R² is N);

R³ is selected from:
(a) hydrogen,
(b) C₁₋₃alkyl, optionally substituted with 1-3 fluoro,
(c) —O—C₁₋₃alkyl, optionally substituted with 1-3 fluoro,
(d) hydroxy,
(e) chloro,
(f) fluoro,
(g) bromo,
(h) phenyl,
(g) heterocycle, and
(h) nothing or O (when the Z bonded to R³ is N);

R⁴ is selected from:
(a) hydrogen,
(b) C₁₋₃alkyl, optionally substituted with 1-3 fluoro,
(c) —O—C₁₋₃alkyl, optionally substituted with 1-3 fluoro,
(d) hydroxy,
(e) chloro,
(f) fluoro,
(g) bromo,
(h) phenyl,
(g) heterocycle, and
(h) nothing or O (when the Z bonded to R⁴ is N);

R⁵ is selected from:
(a) C₁₋₆alkyl, where alkyl may be unsubstituted or substituted with 1-6 fluoro and optionally substituted with hydroxyl,
(b) —O—C₁₋₆alkyl, where alkyl may be unsubstituted or substituted with 1-6 fluoro,
(c) —CO—C₁₋₆alkyl, where alkyl may be unsubstituted or substituted with 1-6 fluoro,
(d) —S—C₁₋₆alkyl, where alkyl may be unsubstituted or substituted with 1-6 fluoro,
(e) -pyridyl, which may be unsubstituted or substituted with one or more substituents selected from: halo, trifluoromethyl, C₁₋₄alkyl, and COR¹¹,
(f) fluoro,
(g) chloro,
(h) bromo,
(i) —C₄₋₆cycloalkyl,
(j) —O—C₄₋₆cycloalkyl,
(k) phenyl, which may be unsubstituted or substituted with one or more substituents selected from: halo, trifluoromethyl, C₁₋₄alkyl, and COR¹¹,
(j) —O-phenyl, which may be unsubstituted or substituted with one or more substituents selected from: halo, trifluoromethyl, C₁₋₄alkyl, and COR¹¹,
(m) —C₃₋₆cycloalkyl, where alkyl may be unsubstituted or substituted with 1-6 fluoro,
(n) —O—C₃₋₆cycloalkyl, where alkyl may be unsubstituted or substituted with 1-6 fluoro,
(o) -heterocycle,
(p) —CN, and
(q) —COR¹¹;

R⁶ is selected from:
(a) hydrogen,
(b) C₁₋₃alkyl, optionally substituted with 1-3 fluoro,
(c) —O—C₁₋₃alkyl, optionally substituted with 1-3 fluoro,
(d) hydroxy,
(e) chloro,
(f) fluoro,
(g) bromo,
(h) phenyl,
(g) heterocycle, and
(h) nothing or O (when the Z bonded to R⁶ is N);

R⁷ is selected from:
hydrogen, (C₀₋₆alkyl)-phenyl, (C₀₋₆alkyl)-heterocycle, (C₀₋₆alkyl)-C₃₋₇cycloalkyl, (C₀₋₆alkyl)-COR¹¹, (C₀₋₆alkyl)-(alkene)-COR¹¹, (C₀₋₆alkyl)-SO₃H, (C₀₋₆alkyl)-W—C₀₋₄alkyl, (C₀₋₆alkyl)-CONR¹²-phenyl, (C₀₋₆alkyl)-CONR²O—V—COR¹¹, and nothing (when X is O, S, or SO₂), where W is selected from: a single bond, —O—, —S—, —SO—, —SO₂—, —CO—, —CO₂—, —CONR¹²— and —NR¹²—, and where V is selected from C₁₋₆alkyl or phenyl, and
   where the R²⁰ can be hydrogen, C₁₋₄alkyl, or where R²⁰ is joined via a 1-5 carbon tether to one of the carbons of V to form a ring, and
   where the C₀₋₆alkyl is unsubstituted or substituted with 1-5 substituents, where the substituents are independently selected from:
   (a) halo,
   (b) hydroxy,
   (c) —C₀₋₆alkyl
   (d) —O—C₁₋₃alkyl,
   (e) trifluoromethyl, and
   (f) —C₀₋₂alkyl-phenyl,
and where the phenyl, heterocycle, cycloalkyl, and C₀₋₄alkyl is unsubstituted or substituted with 1-5 substituents where the substituents are independently selected from:
   (a) halo,
   (b) trifluoromethyl,
   (c) hydroxy,
   (d) C₁₋₃alkyl,
   (e) —O—C₁₋₃alkyl,
   (f) —C₀₋₃—COR¹¹,
   (g) —CN,
   (h) —NR¹²R¹²,
   (i) —CONR¹²R¹², and
   (j) —C₀₋₃-heterocycle,
   or where the phenyl and heterocycle may be fused to another heterocycle, which itself may be unsubstituted or substituted with 1-2 substituents independently selected from hydroxy, halo, —COR11, and —C₁₋₃alkyl,
and where alkene is unsubstituted or substituted with 1-3 substituents which are independently selected from:
   (a) halo,
   (b) trifluoromethyl,
   (c) C₁₋₃alkyl,
   (d) phenyl, and
   (e) heterocycle;
R⁸ is selected from:
   (a) hydrogen,
   (b) nothing when X is either O, S, SO₂ or N or when a double bond joins the carbons to which R⁷ and R¹⁰ are attached,
   (c) hydroxy,
   (d) C₁₋₆alkyl,
   (e) C₁₋₆alkyl-hydroxy,
   (f) —O—C₁₋₃alkyl,
   (g) —COR¹¹,
   (h) —CONR¹²R¹², and
   (i) —CN;

or where R⁷ and R⁸ may be joined together to form a ring which is selected from:
(a) 1H-indene,
(b) 2,3-dihydro-1H-indene,
(c) 2,3-dihydro-benzofuran,
(d) 1,3-dihydro-isobenzofuran,
(e) 2,3-dihydro-benzothiofuran,
(f) 1,3-dihydro-isobenzothiofuran,
(g) 6H-cyclopenta[d]isoxazol-3-ol
(h) cyclopentane, and
(i) cyclohexane,
where the ring formed may be unsubstituted or substituted with 1-5 substituents independently selected from:
   (a) halo,
   (b) trifluoromethyl,
   (c) hydroxy,
   (d) C₁₋₃alkyl,
   (e) —O—C₁₋₃alkyl,
   (f) —C₀₋₃—COR¹¹,
   (g) —CN,
   (h) —NR¹²R¹²,
   (i) —CONR¹²R¹², and
   (j) —C₀₋₃-heterocycle,

or where R⁷ and R⁹ or R⁸ and R¹⁰ may be joined together to form a ring which is phenyl or heterocycle,
wherein the ring is unsubstituted or substituted with 1-7 substituents where the substituents are independently selected from:
   (a) halo,
   (b) trifluoromethyl,
   (c) hydroxy,
   (d) C₁₋₃alkyl,
   (e) —O—Cl₁₋₃alkyl,
   (f) —COR¹¹,
   (g) —CN,
   (h) —NR¹²R¹², and
   (i) —CONR¹²R¹²;

R⁹ and R¹⁰ are independently selected from:
(a) hydrogen,
(b) hydroxy,
(c) C₁₋₆alkyl,
(d) C₁₋₆alkyl-COR¹¹,
(e) C₁₋₆alkyl-hydroxy,
(f) —O—C₁₋₃alkyl,
(g) ═O, when R⁹ or R¹⁰ is connected to the ring via a double bond
(h) halo;

R¹⁵ is selected from:
(a) hydrogen, and
(b) C₁₋₆alkyl, which is unsubstituted or substituted with 1-3 substituents where the substituents are independently selected from: halo, hydroxy, —CO₂H, —CO₂C₁₋₆alkyl, and O—C₁₋₃alkyl;

R¹⁶ is selected from:
(a) hydrogen,
(b) C₁₋₆alkyl, where alkyl may be unsubstituted or substituted with 1-6 substituents where the substituents are selected from: fluoro, C₁₋₃alkoxy, hydroxy, —COR¹¹,
(c) fluoro,
(d) —O—C₁₋₃alkyl, where alkyl may be unsubstituted or substituted with 1-3 fluoro, and
(e) C₃₋₆ cycloalkyl,
(f) —O—C₃₋₆cycloalkyl,
(g) hydroxy,
(h) —COR¹¹,
(i) —OCOR¹³,
or R¹⁵ and R¹⁶ may be joined together via a C₂₋₄alkyl or a C₀₋₂alkyl-O—C₁₋₃alkyl chain to form a 5-7 membered ring;

R¹⁷ is selected from:
(a) hydrogen,
(b) C₁₋₆alkyl, where alkyl may be unsubstituted or substituted with 1-6 substituents where the substituents are selected from: fluoro, C₁₋₃alkoxy, hydroxy, —COR¹¹,
(c) COR¹¹,
(d) hydroxy, and
(e) —O—C₁₋₆alkyl, where alkyl may be unsubstituted or substituted with 1-6 substituents where the substituents are selected from: fluoro, C₁₋₃alkoxy, hydroxy, —COR¹¹,
or R¹⁶ and R¹⁷ may be joined together by a C₁₋₄alkyl chain or a C₀₋₃alkyl-O—C₀₋₃alkyl chain to form a 3-6 membered ring;

R¹⁸ is selected from:
(a) hydrogen, and
(b) C₁₋₆alkyl, where alkyl may be unsubstituted or substituted with 1-6 fluoro,
(c) fluoro,
(d) —O—C₃₋₆cycloalkyl, and
(e) —O—C₁₋₃alkyl, where alkyl may be unsubstituted or substituted with 1-6 fluoro,
or R¹⁶ and R¹⁸ may be joined together by a C₂₋₃alkyl chain to form a 5-6 membered ring, where the alkyl are unsubstituted or substituted with 1-3 substituents where the substituents are independently selected from: halo, hydroxy, —COR¹¹, C₁₋₃alkyl, and C₁₋₃alkoxy,
or R¹⁶ and R¹⁸ may be joined together by a C₁₋₂alkyl-O—C₁₋₂alkyl chain to form a 6-8 membered ring, where the alkyl are unsubstituted or substituted with 1-3 substituents where the substiuents are independently selected from: halo, hydroxy, —COR¹¹, C₁₋₃alkyl, and
C₁₋₃alkoxy,
or R¹⁶ and R¹⁸ may be joined together by a O—C₁₋₂alkyl-O-chain to form a 6-7 membered ring, where the alkyl are unsubstituted or substituted with 1-3 substituents where the substiuents are independently selected from: halo, hydroxy, —COR¹¹, C₁₋₃alkyl, and C₁₋₃alkoxy;

R¹⁹ is selected from:
(a) hydrogen,
(b) phenyl,
(c) C₁₋₆alkyl which may be substituted or unsubstituted with 1-6 of the following substituents: —COR¹¹, hydroxy, fluoro, chloro, —O—C₁₋₃alkyl; or

R² and R¹⁹ can also be joined together to form a heterocycle ring with a linker selected from the following list (with the left side of the linker being bonded to the amide nitrogen at R¹⁹):
(a) —CH₂(CR²⁸R²⁸)₁₋₃—,
(b) —CH₂NR²⁹—
(c) —NR²⁹CR²⁸R²⁸—,
(d) —CH₂O—,
(e) —CH₂SO₂—,
(f) —CH₂SO—,
(g) —CH₂S—,
(h) —CR²⁸R²⁸—,

where R²⁸ is selected from selected from:
(a) hydrogen,
(b) hydroxy,
(c) halo,
(d) C₁₋₃alkyl, where the alkyl is unsubstituted or substituted with 1-6 substituents independently selected from: fluoro, and hydroxy,
(e) —NR¹²R¹²,
(f) —COR¹¹,
(g) —CONR¹²R¹²,
(h) —NR¹²COR¹³,
(i) —OCONR¹²R¹²,
(j) —NR¹²CONR¹²R¹²,
(k) -heterocycle,
(l) —CN,
(m) —NR¹²—SO₂—NR¹²R¹²,
(n) —NR¹²—SO₂—R¹⁴,
(o) —SO₂—NR¹²R¹², and
(p) ═O, where R²⁸ is connected to the ring via a double bond (in which case the other R²⁸ at the same position is nothing, and
where R²⁹ is selected from:
(a) hydrogen,
(b) C₁₋₃alkyl, where the alkyl is unsubstituted or substituted with 1-6 substituents independently selected from: fluoro, and hydroxy,
(c) COR¹³,
(d) SO₂R¹⁴, and
(e) SO₂NR¹²R¹²;

R²⁵ and R²⁶ are independently selected from:
(a) ═O, where R²⁵ and/or R²⁶ is oxygen and is connected via a double bond.
(b) hydrogen,
(c) phenyl,
(d) C₁₋₆alkyl which may be substituted or unsubstituted with 1-6 of the following substituents: —COR¹¹, hydroxy, fluoro, chloro, —O—C₁₋₃alkyl;

m is selected from 0, 1, or 2;

n is selected from 1 or 2;

the dashed line represents a single or a double bond;

and pharmaceutically acceptable salts thereof and individual diastereomers thereof.

Examples of the compounds of Formulae IIIa and IIIb include the following:

### Formula III Compounds

### EXAMPLES

### Example III-1

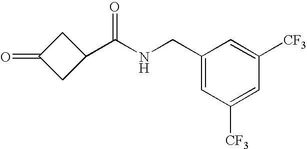

### Examples III-2 to III-10

Examples III-2 through III-10, in Table 14, below, are based on the formula:

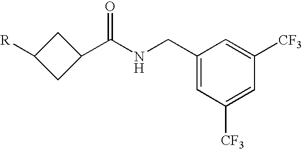

| Example | R | Molecular Formula | Calculated MW | Found M⁺H⁺ |
|---|---|---|---|---|
| III-2 | 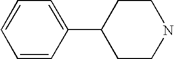 | C₂₅H₂₆F₆N₂O | 484.19 | 485.2 |
| III-3 | 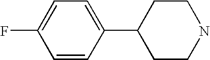 | C₂₅H₂₅F₇N₂O | 502.19 | 503.0 |
| III-4 | 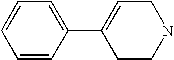 | C₂₅H₂₄F₆N₂O | 482.18 | 483.0 |
| III-5 | 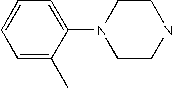 | C₂₅H₂₇F₆N₃O | 499.21 | 500.0 |
| III-6 | 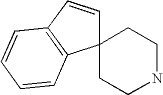 | C₂₇H₂₆F₆N₂O | 508.19 | 509.0 |
| III-7 | 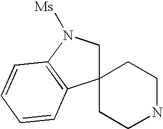 | C₂₇H₂₉F₆N₃O₃S₂ | 589.18 | 590.0 |
| III-8 | 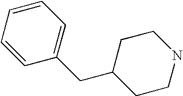 | C₂₆H₂₈F₆N₂O | 499.21 | 500.0 |
| III-9 | 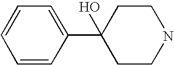 | C₂₅H₂₆F₆N₂O₂ | 500.19 | 501.0 |
| III-10 | 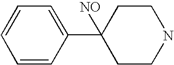 | C₂₆H₂₅F₆N₃O | 509.19 | 510.0 |

### Example III-11

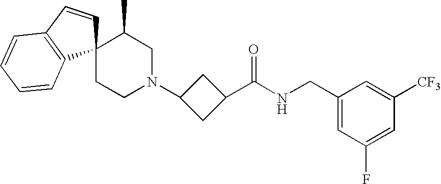

### Example III-12

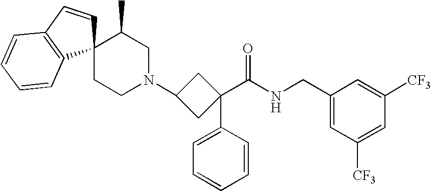

### Examples III-13 to III-40

Examples III-13 through III-40, in Table 15, below, are based on the formula:

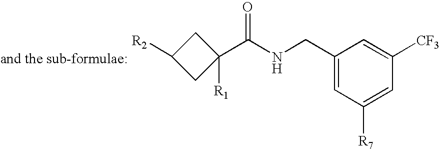

R1

| | | | | |
|---|---|---|---|---|
| 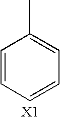 | 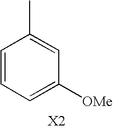 | 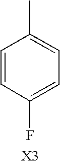 | 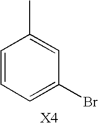 | 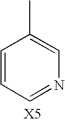 |

| | |
|---|---|
| 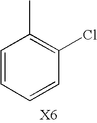 | 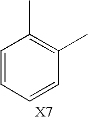 |

R2

| | |
|---|---|
| 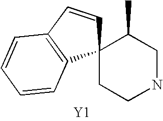 | 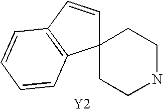 |

| | | |
|---|---|---|
| 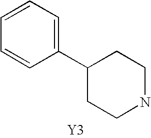 | 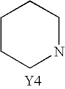 | 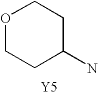 |

| Ex- | | | | Molecular | | Found |
|---|---|---|---|---|---|---|
| ample | R1 | R2 | R3 | Formula | Calculated MW | [M⁺H⁺] |
| III-13 | X1 | Y2 | CF3 | C33H30F6N2O | 584.23 | 585.25 |
| III-14 | X1 | Y3 | CF3 | C31H30F6N2O | 560.26 | 561.25 |
| III-15 | X1 | Y4 | CF3 | C25H26F6N2O | 484.48 | 485.20 |
| III-16 | X1 | Y5 | CF3 | C25H26F6N2O2 | 500.19 | 501.25 |
| III-17 | X1 | Y1 | F | C33H32F4N2O | 548.25 | 549.25 |
| III-18 | X1 | Y2 | F | C32H30F4N2O | 534.23 | 535.30 |
| III-19 | X1 | Y3 | F | C30H30F4N2O | 510.23 | 511.30 |
| III-20 | X1 | Y4 | F | C24H26F4N2O | 434.20 | 435.25 |
| III-21 | X1 | Y5 | F | C24H26F4N2O2 | 450.19 | 451.30 |
| III-22 | X2 | Y1 | F | C34H34F4N2O2 | 578.26 | 579.25 |
| III-23 | X2 | Y3 | F | C31H32F4N2O2 | 540.24 | 541.30 |
| III-24 | X2 | Y4 | F | C25H28F4N2O2 | 464.21 | 465.25 |
| III-25 | X3 | Y1 | F | C33H31F5N2O | 566.24 | 567.25 |
| III-26 | X3 | Y3 | F | C30H29F5N2O | 528.22 | 529.25 |
| III-27 | X3 | Y4 | F | C24H25F5N2O | 452.19 | 453.25 |
| III-28 | X4 | Y1 | F | C33H31BrF4N2O | 626.18 | 629.20 |
| III-29 | X4 | Y3 | F | C30H29BrF4N2O | 588.16 | 591.15 |
| III-30 | X4 | Y4 | F | C24H25BrF4N2O | 512.13 | 515.05 |
| III-31 | X5 | Y1 | F | C32H31F4N3O | 549.24 | 550.30 |
| III-32 | X5 | Y3 | F | C29H29F4N3O | 511.22 | 512.20 |
| III-33 | X5 | Y4 | F | C23H25F4N3O | 435.19 | 436.15 |
| III-34 | X5 | Y1 | CF3 | C33H31F6N3O | 599.24 | 600.25 |
| III-35 | X6 | Y1 | F | C33H31ClF4N2O | 582.21 | 583.3 |
| III-36 | X6 | Y3 | F | C30H29ClF4N2O | 544.19 | 545.20 |
| III-37 | X6 | Y4 | F | C24H25ClF4N2O | 468.16 | 469.15 |
| III-38 | X7 | Y1 | F | C34H34F4N2O | 562.26 | 563.25 |
| III-39 | X7 | Y3 | F | C31H32F4N2O | 524.25 | 525.25 |
| III-40 | X7 | Y4 | F | C25H28F4N2O | 448.21 | 449.15 |

### Example III-41

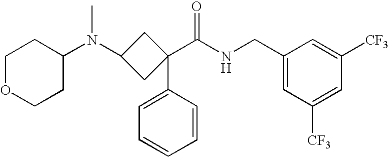

### Example III-42

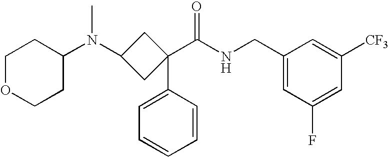

### Example III-43

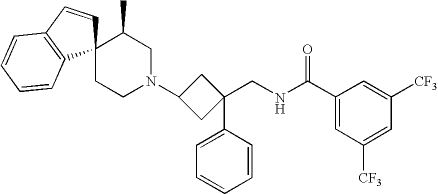

### Examples III-44 to III-53

Examples III-44 through III-53, in Table 16, below, are based on the formula:

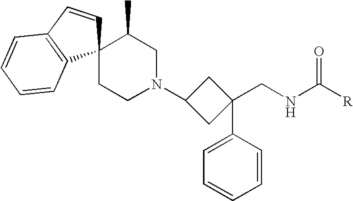

| Example | R | Molecular Formula | Calculated MW | Found M⁺H⁺ |
|---|---|---|---|---|
| III-44 | Me | C27H32N2O | 400.26 | 401.2 |
| III-45 | 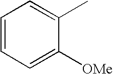 | C33H36N2O2 | 482.28 | 493.3 |
| III-46 | 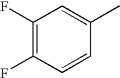 | C32H32F2N2O2 | 498.25 | 499.3 |
| III-47 | 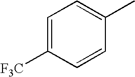 | C33H33F8N2O | 530.25 | 531.25 |
| III-48 | 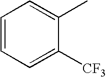 | C33H33F3N2O | 530.25 | 531 |
| III-49 | 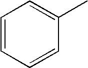 | C32H34N2O | 462.27 | 463.3 |
| III-50 | 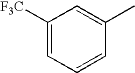 | C33H33F3N2O | 530.25 | 531.25 |
| III-51 | 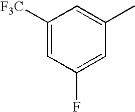 | C33H32F4N2O | 548.25 | 549.25 |
| III-52 | 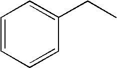 | C33H36N2O | 476.28 | 477.25 |
| III-53 | 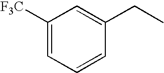 | C34H35F3N2O | 544.27 | 545.35 |

### Example III-54

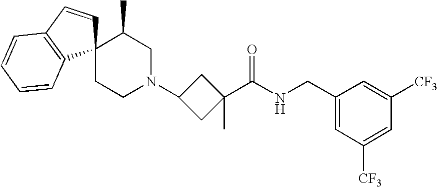

### Examples III-55 to III-63

Examples III-55 through III-63, in Table 17, below, are based on the formula:

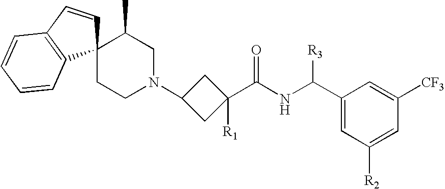

| | | | | | Calculated | Found |
|---|---|---|---|---|---|---|
| Example | R1 | R2 | R3 | Molecular Formula | MW | [M⁺H⁺] |
| III-55 | Me | F | H | C28H30F4N2O | 486.23 | 487.3 |
| III-56 | Et | CF3 | H | C30H32F6N2O | 550.24 | 551.2 |
| III-57 | Et | F | H | C29H32F4N2O | 500.24 | 501.25 |
| III-58 | Pr | CF3 | H | C31H34F6N2O | 564.26 | 565.3 |
| III-59 | Pr | F | H | C30H34F6N2O | 514.26 | 515.3 |
| III-60 | MeS | CF3 | H | C29H30F6N2OS | 568.20 | 569.2 |
| III-61 | MeS | F | H | C28H30F4N2OS | 518.20 | 519.25 |
| III-62 | Pr | H | Me | C31H37F3N2O | 510.29 | 511.3 |
| III-63 | Me | CF3 | Me | C32H36F6N2O | 578.27 | 579.25 |

### Example III-64

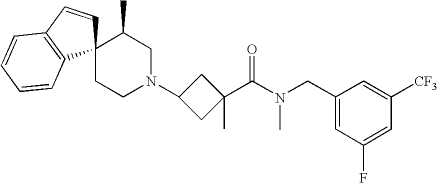

### Example III-65

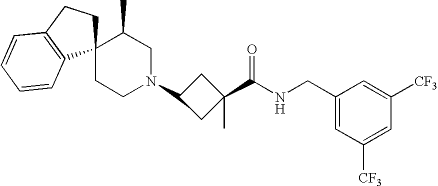

### Example III-66

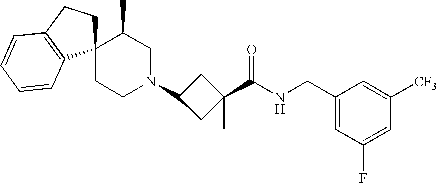

### Example III-67

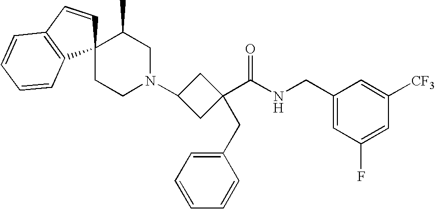

### Examples III-68 to III-76

Examples III-68 through III-76, in Table 18, below, are based on the formula:

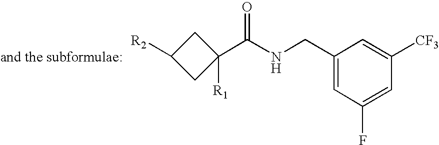

R1

| | | | |
|---|---|---|---|
| 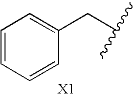 | 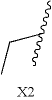 | 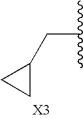 | 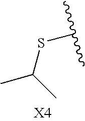 |

R2

| | |
|---|---|
| 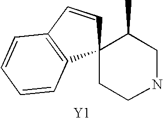 | 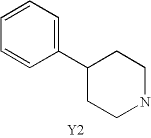 |

| | | |
|---|---|---|
| 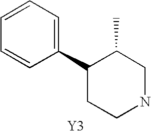 | 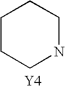 | 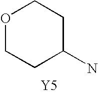 |

| Ex- | | | | | |
|---|---|---|---|---|---|
| ample | R1 | R2 | Molecular Formula | Calculated MW | Found [M⁺H⁺] |
| III-68 | X1 | Y2 | C31H32F4N2O | 524.25 | 525.25 |
| III-69 | X1 | Y4 | C25H28F4N2O | 448.21 | 449.2 |
| III-70 | X2 | Y2 | C26H30F4N2O | 462.23 | 463.3 |
| III-71 | X2 | Y4 | C20H26F4N2O | 386.20 | 387.2 |
| III-72 | X3 | Y1 | C31H34F4N2O | 526.26 | 527.3 |
| III-73 | X4 | Y1 | C30H34F4N2OS | 546.23 | 547.3 |
| III-74 | X2 | Y3 | C27H32F4N2O | 476.25 | 477.25 |
| III-75 | X2 | Y5 | C20H26F4N2O2 | 402.19 | 403.15 |
| III-76 | X1 | Y5 | C25H28F4N2O2 | 464.21 | 465.25 |

### Example III-77

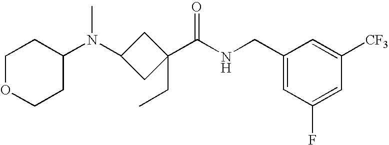

### Example III-78

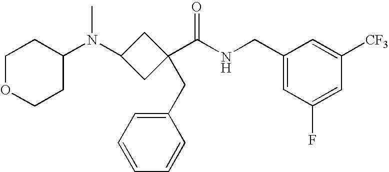

### Example III-79

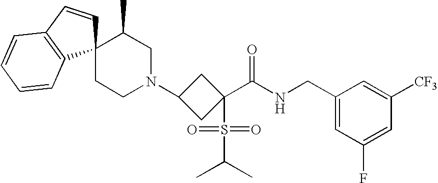

### Example III-80

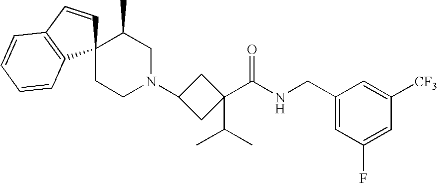

### Examples III-81 to III-116

Examples III-81 through III-116, in Table 19, below, are based on the formula:

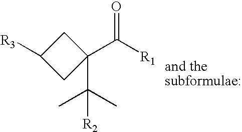

R1

| | |
|---|---|
| 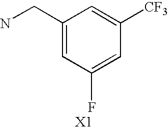 | 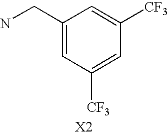 |

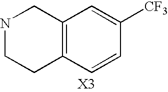

R3

| | |
|---|---|
| 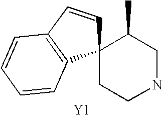 | 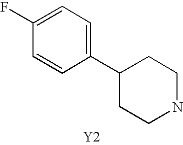 |

| | |
|---|---|
| 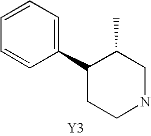 | 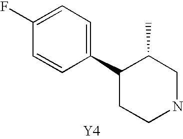 |

| | | |
|---|---|---|
| 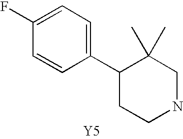 | 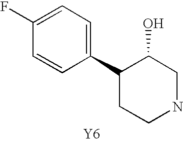 | 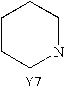 |

| | | |
|---|---|---|
| 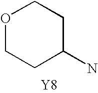 | 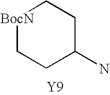 | 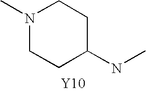 |

| | | |
|---|---|---|
| 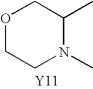 | 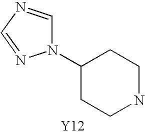 | 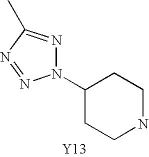 |

| | |
|---|---|
| 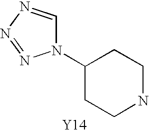 | 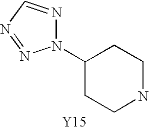 |

| | |
|---|---|
| 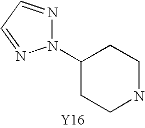 | 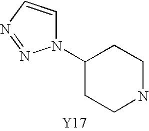 |

| | | | | | Calculated | Found |
|---|---|---|---|---|---|---|
| Example | R1 | R2 | R3 | Molecular Formula | MW | [M⁺H⁺] |
| III-81 | X1 | H | Y2 | C27H31F5N2O | 494 | 495 |
| III-82 | X1 | H | Y3 | C28H34F4N2O | 490 | 491 |
| III-83 | X1 | H | Y7 | C21H28F4N2O | 400 | 401 |
| III-84 | X1 | H | Y8 | C21H28F4N2O2 | 416 | 417 |
| III-85 | X1 | H | Y9 | C26H37F4N3O3 | 515 | 516 |
| III-86 | X1 | H | Y10 | C23H33F4N3O | 443 | 444 |
| III-87 | X2 | H | Y1 | C31H34F6N2O | 564 | 565 |
| III-88 | X2 | H | Y2 | C28H31F7N2O | 544.23 | 545.2 |
| III-89 | X2 | H | Y3 | C29H34F6N2O | 540 | 541 |
| III-90 | X2 | H | Y7 | C22H28F6N2O | 450 | 451 |
| III-91 | X2 | H | Y8 | C22H28F6N2O2 | 466 | 467 |
| III-92 | X2 | H | Y9 | C27H37F6N3O3 | 565 | 566 |
| III-93 | X2 | H | Y10 | C24H33F6N3O | 493 | 494 |
| III-94 | X1 | OH | Y1 | C30H34F4N2O2 | 530.26 | 531.25 |
| III-95 | X1 | OH | Y8 | C21H28F4N2O3 | 432.20 | 433.15 |
| III-96 | X2 | OH | Y1 | C31H34F6N2O2 | 580.25 | 581.2 |
| III-97 | X2 | OH | Y8 | C22H28F6N2O3 | 482.20 | 483.25 |
| III-98 | X2 | OH | Y2 | C28H31F7N2O2 | 560.23 | 561.25 |
| III-99 | X2 | H | Y12 | C24H29F6N5O | 517.23 | 518.2 |
| III-100 | X2 | H | Y13 | C24H30F6N6O | 532.24 | 533.2 |
| III-101 | X2 | H | Y14 | C23H28F6N2O | 518.22 | 519.25 |
| III-102 | X2 | H | Y15 | C23H28F6N6O | 518.22 | 519.25 |
| III-103 | X2 | H | Y16 | C24H29F6N5O | 517.23 | 518.2 |
| III-104 | X2 | H | Y17 | C24H29F6N5O | 517.23 | 518.2 |
| III-105 | X3 | H | Y1 | C32H37F3N2O | 522.29 | 523.45 |
| III-106 | X3 | H | Y8 | C23H31F3N2O2 | 424.23 | 525.35 |
| III-107 | X1 | OH | Y4 | C28H33F5N2O2 | 524.25 | 525.25 |
| III-108 | X2 | OH | Y4 | C29H33F7N2O2 | 574.24 | 575.2 |
| III-109 | X2 | H | Y5 | C30H35F7N2O | 572.25 | 573.25 |
| III-110 | X2 | H | Y4 | C29H33F7N2O | 558.25 | 559.3 |
| III-111 | X2 | H | Y6 | C28H31F7N2O3 | 576.22 | 577.3 |
| III-112 | X1 | OH | Y5 | C29H35F5N2O2 | 538.25 | 539.35 |
| III-113 | X1 | OH | Y6 | C27H31F5N2O3 | 526.23 | 527.3 |
| III-114 | X2 | OH | Y5 | C30H35F7N2O2 | 588.24 | 589.3 |
| III-115 | X2 | OH | Y6 | C28H31F7N2O2 | 560.23 | 561.25 |
| III-116 | X2 | OH | Y11 | C23H30F6N2O3 | 496.22 | 497.35 |

### Example III-117

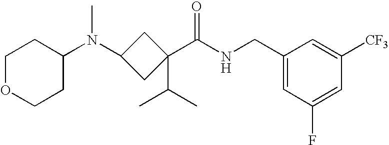

### Example III-118

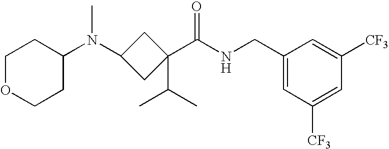

### Example III-119

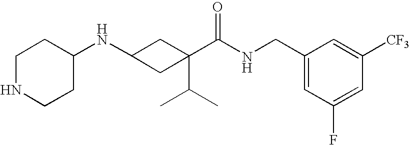

### Example III-120

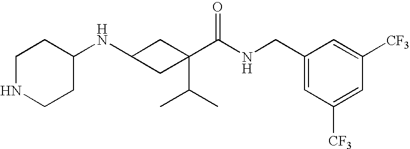

### Example III-121

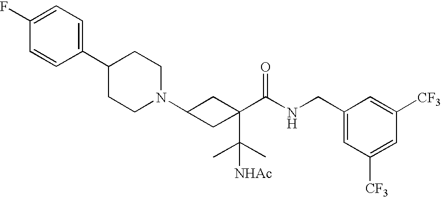

### Example III-122

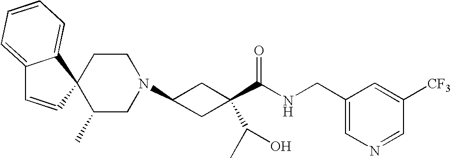

### Examples III-123 TO III-140

Examples III-123 through III-140, in Table 20, below, are based on the formula:

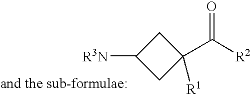

R1

| | | |
|---|---|---|
| 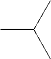 | 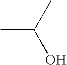 | 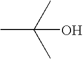 |

R2

| | |
|---|---|
| 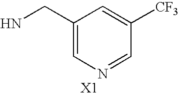 | 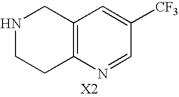 |

R3

| | | |
|---|---|---|
| 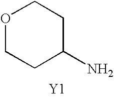 | 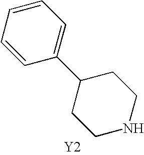 | 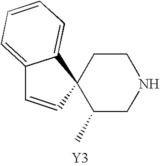 |

| | | | | | Cal- | Found |
|---|---|---|---|---|---|---|
| Ex- | | | | Molecular | culated | [M + |
| ample | R1 | R2 | R3 | formula | MW | H]⁺ |
| III-123 | i-Pr | X1 | Y1 | C20H28F3N3O2 | 399.21 | 400.2 |
| III-124 | i-Pr | X1 | Y2 | C26H32F3N3O | 459.25 | 460.5 |
| III-125 | i-Pr | X1 | Y3 | C29H34F3N3O | 497.27 | 498.2 |
| III-126 | i-Pr | X2 | Y1 | C22H30F3N3O2 | 425.23 | 426.2 |
| III-127 | i-Pr | X2 | Y2 | C28H34F3N3O | 485.27 | 486.3 |
| III-128 | i-Pr | X2 | Y3 | C31H36F3N3O | 523.28 | 524.3 |
| III-129 | CH(OH)CH₃ | X1 | Y1 | C19H26F3N3O3 | 401.19 | 402.1 |
| III-130 | CH(OH)CH₃ | X1 | Y2 | C25H30F3N3O2 | 461.23 | 462.5 |
| III-131 | CH(OH)CH₃ | X1 | Y3 | C28H32F3N3O2 | 499.24 | 500.25 |
| III-132 | CH(OH)CH₃ | X2 | Y1 | C21H28F3N3O3 | 427.21 | 428.2 |
| III-133 | CH(OH)CH₃ | X2 | Y2 | C27H32F3N3O2 | 487.24 | 488.15 |
| III-134 | CH(OH)CH₃ | X2 | Y3 | C30H34F3N3O2 | 525.26 | 526.3 |
| III-135 | C(OH)(CH₃)₂ | X1 | Y1 | C20H28F3N3O3 | 415.21 | 416.2 |
| III-136 | C(OH)(CH₃)₂ | X1 | Y2 | C26H32F3N3O2 | 475.24 | 476.5 |
| III-137 | C(OH)(CH₃)₂ | X1 | Y3 | C29H34F3N3O2 | 513.26 | 514.25 |
| III-138 | C(OH)(CH₃)₂ | X2 | Y1 | C22H30F3N3O3 | 441.22 | 442.2 |
| III-139 | C(OH)(CH₃)₂ | X2 | Y2 | C28H34F3N3O2 | 501.26 | 502.25 |
| III-140 | C(OH)(CH₃)₂ | X2 | Y3 | C31H36F3N3O2 | 539.28 | 540.3 |

Additional CCR-2 antagonists useful in the methods of the invention include those of Formula IV: 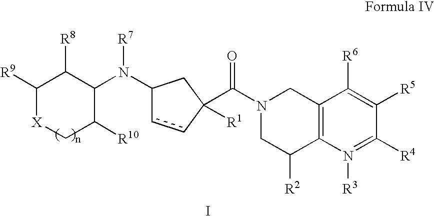
wherein:

X is selected from the group consisting of:
—O—, —NR²⁰—, —S—, —SO—, —SO₂—, and —CR²¹R²², —NSO₂R²⁰—, —NCOR²⁰—, —NCO₂R²⁰—, —CR²¹CO₂R²⁰—, —CR²¹OCOR²⁰—, —CO—,
where R²⁰ is selected from: hydrogen, C₁₋₆ alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl,
where R²¹ and R²² are independently selected from: hydrogen, hydroxy, C₁₋₆ alkyl, —O—C₁₋₆alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl;

R¹ is selected from:
—C₁₋₆alkyl, —C₀₋₆alkyl-O—C₁₋₆alkyl-, —C₀₋₆alkyl-S—C₁₋₆alkyl-, —(C₀₋₆alkyl)-(C₃₋₇cycloalkyl)-(C₀₋₆alkyl), hydroxy, —CO₂R²⁰, heterocycle, —CN, —NR²⁰R²⁶—, —NSO₂R²⁰—, —NCOR²⁰—, —NCO₂R²⁰—, —NCOR²⁰—, —CR²¹CO₂R²⁰—, —CR²¹OCOR²⁰—, phenyl and pyridyl,
where R²⁶ is selected from: hydrogen, C₁₋₆ alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl
where the alkyl and the cycloalkyl are unsubstituted or substituted with 1-7 substituents where the substituents are independently selected from:
   (a) halo,
   (b) hydroxy,
   (c) —O—C₁₋₃alkyl,
   (d) trifluoromethyl,
   (f) C₁₋₃alkyl,
   (g) —O—C₁₋₃alkyl,
   (h) —CO₂R²⁰,
   (i) —SO₂R₂₀,
   (j) —NHCOCH₃,
   (k) —NHSO₂CH₃,
   (l) -heterocycle,
   (m) ═O,
   (n) —CN,
and where the phenyl and pyridyl are unsubstituted or substituted with 1-3 substituents where the substituents are independently selected from: halo, hydroxy, C₁₋₃alkyl, C₁₋₃alkoxy and trifluoromethyl;

R² is selected from:
(a) hydrogen,
(b) hydroxy,
(c) halo,
(d) C₁₋₃alkyl, where the alkyl is unsubstituted or substituted with 1-6 substituents independently selected from: fluoro, and hydroxy,
(e) —NR²⁰R²⁶,
(f) —CO₂R²⁰,
(g) —CONR²⁰R²⁶,
(h) —NR²⁰COR²¹,
(i) —OCONR²OR²⁶,
(j) —NR²⁰CONR²⁰R²⁶,
(k) -heterocycle,
(l) —CN,
(m) —NR²⁰—SO₂—NR²⁰R²⁶,
(n) —NR²⁰—SO₂—R²⁶,
(o) —SO₂—NR²⁰R²⁶, and
(p) ═O, where R² is connected to the ring via a double bond;

R³ is oxygen or is absent;

R⁴ is selected from:
(a) hydrogen,
(b) C₁₋₆alkyl,
(c) trifluoromethyl,
(d) trifluoromethoxy,
(e) chloro,
(f) fluoro,
(g) bromo, and
(h) phenyl;

R⁵ is selected from:
(a) C₁₋₆alkyl, where alkyl may be unsubstituted or substituted with 1-6 fluoro and optionally substituted with hydroxyl,
(b) —O—C₁₋₆alkyl, where alkyl may be unsubstituted or substituted with 1-6 fluoro,
(c) —O—C₁₋₆alkyl, where alkyl may be unsubstituted or substituted with 1-6 fluoro,
(d) —S—C₁₋₆alkyl, where alkyl may be unsubstituted or substituted with 1-6 fluoro,
(e) -pyridyl, which may be unsubstituted or substituted with one or more substituents selected from the group consisting of: halo, trifluoromethyl, C₁₋₄alkyl, and CO₂R²⁰,
(f) fluoro,
(g) chloro,
(h) bromo,
(i) —C₄₋₆cycloalkyl,
(j) —O—C₄₋₆cycloalkyl,
(k) phenyl, which may be unsubstituted or substituted with one or more substituents selected from the group consisting of: halo, trifluoromethyl, C₁₋₄alkyl, and CO₂R²⁰,
(l) —O-phenyl, which may be unsubstituted or substituted with one or more substituents selected from the group consisting of: halo, trifluoromethyl, C₁₋₄alkyl, and CO₂R²⁰,
(m) —C₃₋₆cycloalkyl, where alkyl may be unsubstituted or substituted with 1-6 fluoro,
(n) —O—C₃₋₆cycloalkyl, where alkyl may be unsubstituted or substituted with 1-6 fluoro,
(o) -heterocycle,
(p) —CN, and
(q) —CO₂R²⁰;

R⁶ is selected from:
(a) hydrogen,
(b) C₁₋₆alkyl, and
(c) trifluoromethyl
(d) fluoro
(e) chloro, and
(f) bromo;

R⁷ is selected from:
(a) hydrogen, and
(b) C₁₋₆alkyl, which is unsubstituted or substituted with 1-3 substituents where the substituents are independently selected from: halo, hydroxy, —CO₂H, —CO₂C₁₋₆alkyl, and —O—C₁₋₃alkyl;

R⁸ is selected from:
(a) hydrogen,
(b) C₁₋₆alkyl, where alkyl may be unsubstituted or substituted with 1-6 substituents where the substituents are chosen from the group: fluoro, C₁₋₃alkoxy, hydroxy, —CO₂R²⁰,
(c) fluoro,
(d) —O—C₁₋₃alkyl, where alkyl may be unsubstituted or substituted with 1-3 fluoro, and
(e) C₃₋₆ cycloalkyl,
(f) —O—C₃₋₆cycloalkyl,
(g) hydroxy,
(h) —CO₂R²⁰,
(i) —OCOR²⁰,
or R⁷ and R⁸ may be joined together via a C₂₋₄alkyl or a C₀₋₂alkyl-O—C₁₋₃alkyl chain to form a 5-7 membered ring;

R⁹ is selected from:
(a) hydrogen,
(b) C₁₋₆alkyl, where alkyl may be unsubstituted or substituted with 1-6 substituents where the substituents are chosen from the group: fluoro, C₁₋₃alkoxy, hydroxy, —CO₂R²⁰,
(c) CO₂R²⁰,
(d) hydroxy, and
(e) —O—C₁₋₆-alkyl, where alkyl may be unsubstituted or substituted with 1-6 substituents where the substituents are chosen from the group: fluoro, C₁₋₃alkoxy, hydroxy, —CO₂R²⁰,
or R⁸ and R⁹ may be joined together by a C₁₋₄alkyl chain or a C₀₋₃alkyl-O—C₀₋₃alkyl chain to form a 3-6 membered ring;

R¹⁰ is selected from:
(a) hydrogen, and
(b) C₁₋₆alkyl, where alkyl may be unsubstituted or substituted with 1-6 fluoro,
(c) fluoro,
(d) —O—C₃₋₆cycloalkyl, and
(e) —O—C₁₋₃alkyl, where alkyl may be unsubstituted or substituted with 1-6 fluoro,
or R⁸ and R¹⁰ may be joined together by a C₂₋₃alkyl chain to form a 5-6 membered ring, where the alkyl are unsubstituted or substituted with 1-3 substituents where the substiuents are independently selected from: halo, hydroxy, —CO₂R²⁰, C₁₋₃alkyl, and C₁₋₃alkoxy,
or R⁸ and R¹⁰ may be joined together by a C₁₋₂alkyl-O—C₁₋₂alkyl chain to form a 6-8 membered ring, where the alkyl are unsubstituted or substituted with 1-3 substituents where the substiuents are independently selected from: halo, hydroxy, —CO₂R²⁰, C₁₋₃alkyl, and
C₁₋₃alkoxy,
or R⁸ and R¹⁰ may be joined together by a —C₁₋₂alkyl-O-chain to form a 6-7 membered ring, where the alkyl are unsubstituted or substituted with 1-3 substituents where the substiuents are independently selected from: halo, hydroxy, —CO₂R²⁰, C₁₋₃alkyl, and
C₁₋₃alkoxy;

n is selected from 0, 1 and 2;

the dashed line represents a single or a double bond;

and pharmaceutically acceptable salts thereof and individual diastereomers thereof.

### Formula IV Compounds

### EXAMPLES

Examples of the compounds of Formula IV include the following:

### Example IV-1

### L-070824

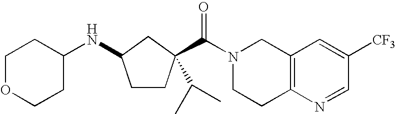

### Example IV-2

### L-070957

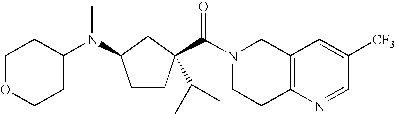

### Example IV-3

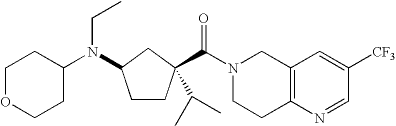

### Example IV-4

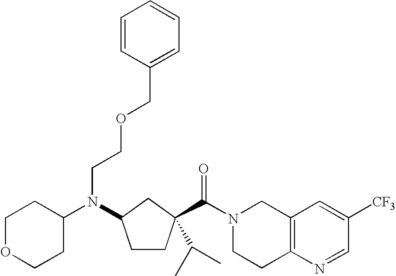

### Example IV-5

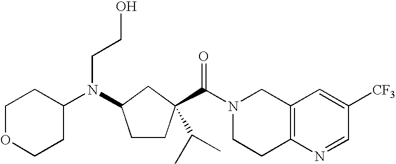

### Example IV-6

### L-383564

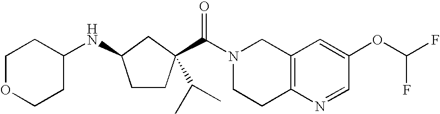

### Example IV-7

### L-385420

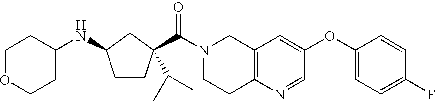

### Example IV-8

### L-384866

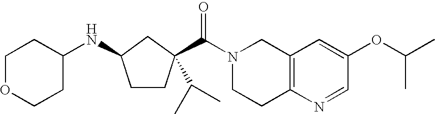

### Example IV-9

### L-385474

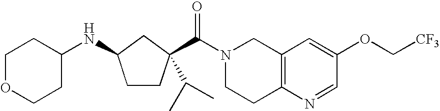

### Example IV-10

### L-385425

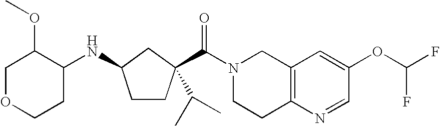

### Example IV-11

### L-385425

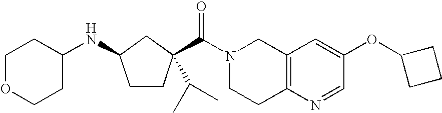

### Example IV-12

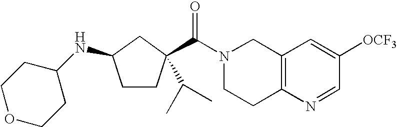

### Example IV-13

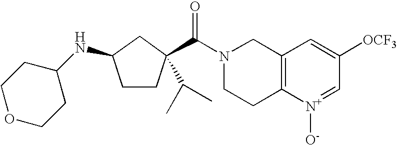

### Example IV-14

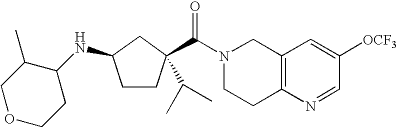

### Example IV-15

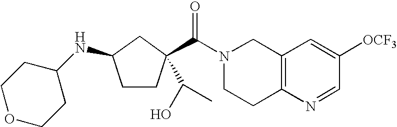

### Example IV-16

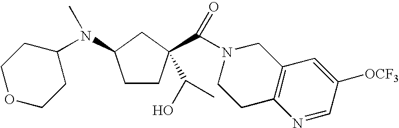

### Example IV-17

### L-071081, L-122051,L-122055, L-122056

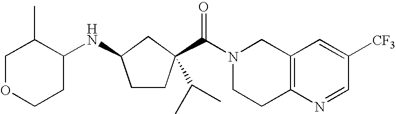

### Example IV-18

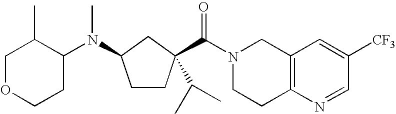

### Example IV-19

### L-384291,L-384292, L-384294

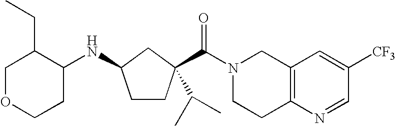

### Example IV-20

### L-071112

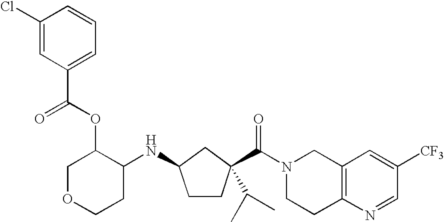

### Example IV-21

### L-071113

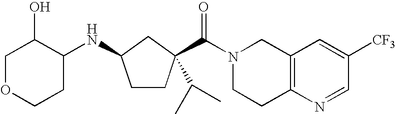

### Example IV-22

### L-220426

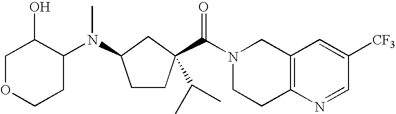

### Example IV-23

### L-124464, L-124466, L-124467, L-124469

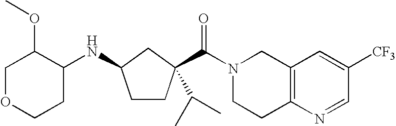

### Example IV-24

### L-330098, L-330100

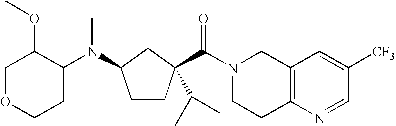

### Example IV-25

### L-383580, L-383581, L-383582

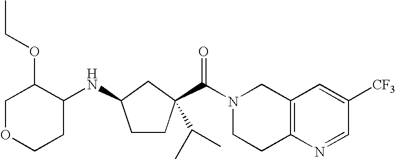

### Example IV-26

### L-233994, L-233995, L-233996, L-233997

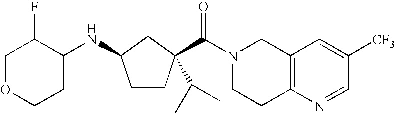

### Example IV-27

### L-251447, L-251450

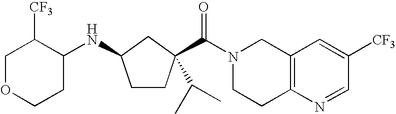

### Example IV-28

### L-070948

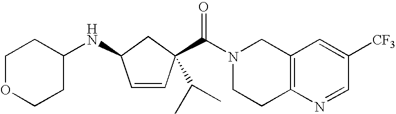

### Example IV-29

### L-237169, L-237171

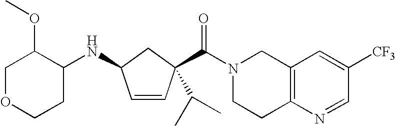

### Example IV-30

### L-071040

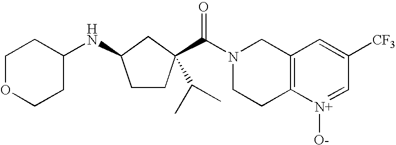

### Example IV-31

### L-220288

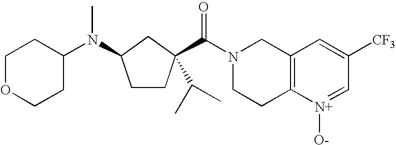

### Example IV-32

### L-071117, L-114785, L-114787, L-114790, L-114793

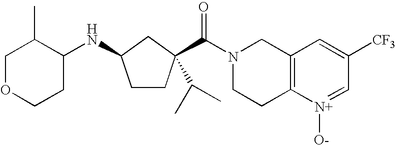

### Example IV-33

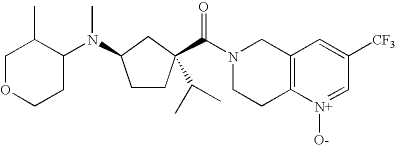

### Example IV-34

### L-384261, L-384263, L-384264

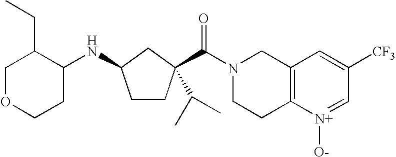

### Example IV-35

### L-330023, L-330027, L-330030, L-330032

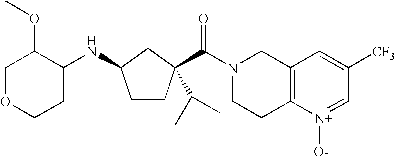

### Example IV-36

### L-346122, L-346124

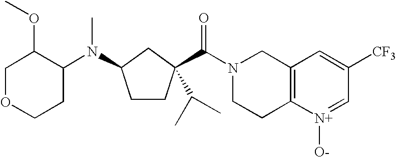

### Example IV-37

### L-075726

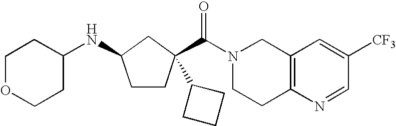

### Example IV-38

### L-121151

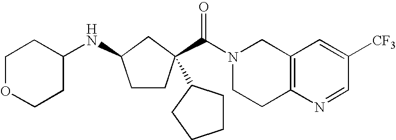

### Example IV-39

### L-121158

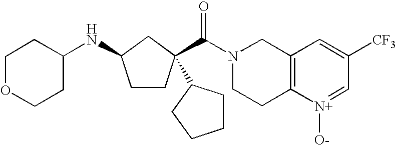

### Example IV-40

### L-114746

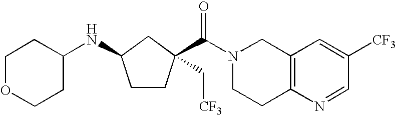

### Example IV-41

### L-220280

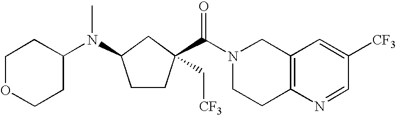

### Example IV-42

### L-220284, L-221962, L-221965, L-221966, L-221969

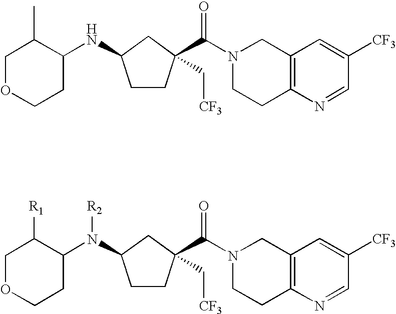

### Example IV-43 to IV47

Examples IV-43 through IV-47, in Table 21, below, are based on the following formula:

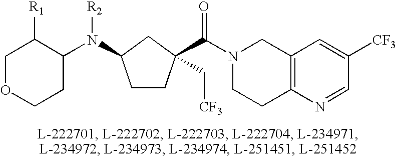

| EX- | | | | FW: formula/ |
|---|---|---|---|---|
| AMPLE | R1 | R2 | Column and eluant | found [M + H]⁺ |
| IV-43 | CH₃ | CH₃ | Single isomers obtained | C₂₄H₃₁F₆N₃O₂ |
| | | | from Example 31 | 508.2 |
| IV-44 | OMe | H | Preparative ChiralCel OD | C₂₃H₂₉F₆N₃O₃ |
| | | | 93% Hexane:7% Ethanol | 510.2 |
| IV-45 | OMe | CH₃ | Single isomers obtained | C₂₄H₃₁F₆N₃O₃ |
| | | | from Example 34 | 524.2 |
| IV-46 | F | H | Preparative ChiralCel OD | C₂₂H₂₆F₇N₃O₂ |
| | | | 90% Hexane:10% | 498.1 |
| | | | Ethanol | |
| IV-47 | CF3 | H | Preparative ChiralCel OD | C₂₃H₂₆F₉N₃O₂ |
| | | | 97% Hexane:3% Ethanol | 548.3 |

### Example IV-48

### L-123133

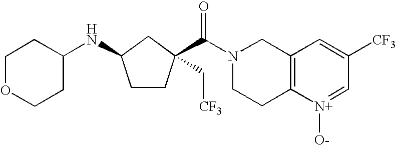

### Example IV-49

### L-221002

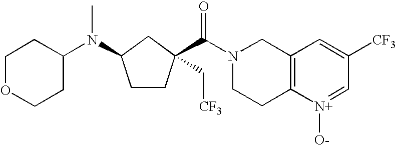

### Example IV-50

### L-123134

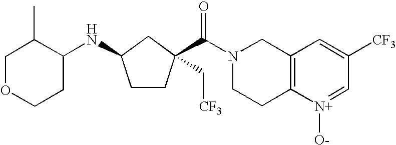

### Example IV-51

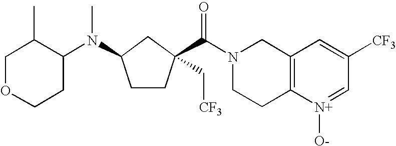

### Example IV-52

### L-223917

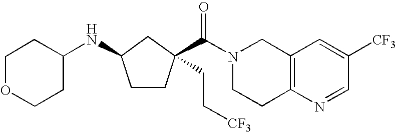

### Example IV-53

### L-234189, L-234197, L-234216, L-234226

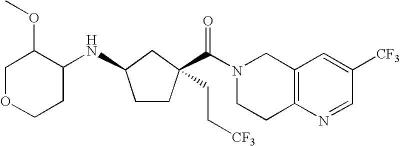

### Example IV-54

### L-235604, L235605, L-235606, L-235608

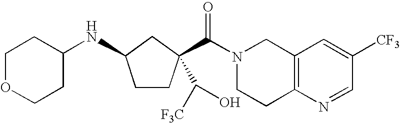

### Example IV-55

### L-071090, L-071091

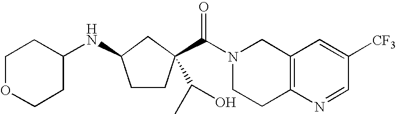

### Example IV-56

### L-071120, L-220990

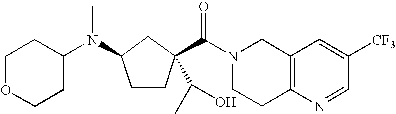

### Example IV-57

### L-0711510, L-074362, L-074363

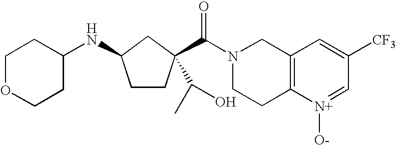

### Example IV-58

### L-071149, L-071150

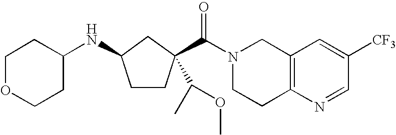

### Example IV-59

### L-071128, L-071129, L-071130, L-071131

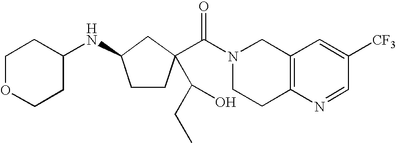

### Example IV-63

### L-385477, L-385479, L-385477, L-385479

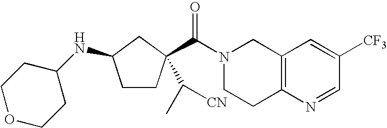

### Example IV-64

### L-071031, L-071032

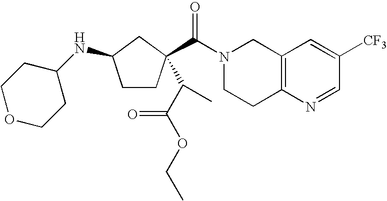

### Example IV-65

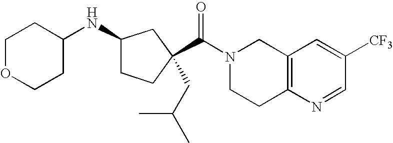

### Example IV-66

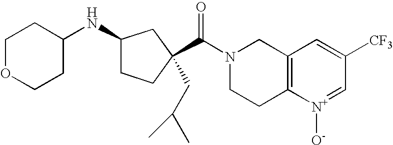

### Example IV-67

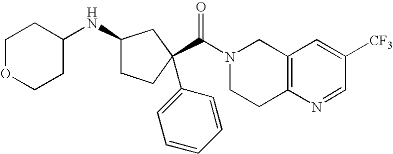

### Example IV-68

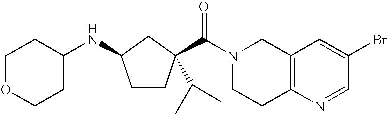

### Example IV-69

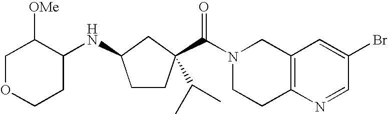

### Example IV-70

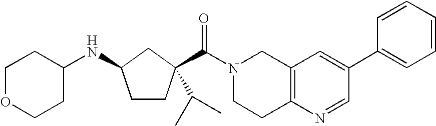

### Example IV-71 to IV-82

The phenyl group from Example 70 can be replaced by other substituents as shown in Table 22:

| | | | Cal- | |
|---|---|---|---|---|
| Ex- | | Molecular | culated | Found |
| ample | substituent | Formula | [M] | [M + H]⁺ |
| IV-71 | 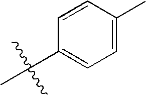 | C₂₉H₃₉N₃O₂: | 461.30 | 462.3 |
| IV-72 | 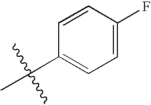 | C₂₈H₃₆N₃O₂F | 465.27 | 466.3 |
| IV-73 | 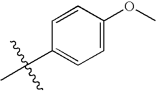 | C₂₉H₃₉N₃O₃ | 477.30 | 478.3 |
| IV-74 | 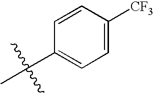 | C₂₉H₃₆N₃O₂F₃ | 515.24 | 516.3 |
| IV-75 | 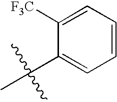 | C₂₉H₃₆N₃O₂F₃ | 515.24 | 516.3 |
| IV-76 | 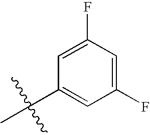 | C₂₈H₃₅N₃O₂F₂ | 483.26 | 484.3 |
| IV-77 | 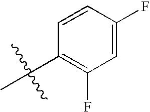 | C₂₈H₃₅N₃O₂F₂ | 483.26 | 484.3 |
| IV-78 | 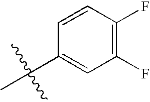 | C₂₈H₃₅N₃O₂F₂ | 483.26 | 484.3 |
| IV-79 | 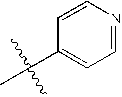 | C₂₇H₃₆N₄O₂ | 448.27 | 449.3 |
| IV-80 | 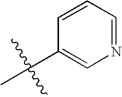 | C₂₇H₃₆N₄O₂ | 448.27 | 449.3 |
| IV-81 | 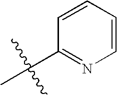 | C₂₇H₃₆N₄O₂ | 448.27 | 449.3 |
| IV-82 | 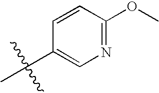 | C₂₈H₃₈N₄O₃ | 478.28 | 479.3 |

### Example IV-83

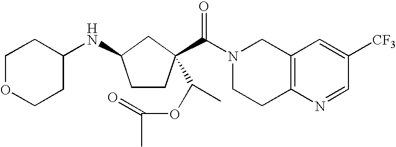

### Example IV-84

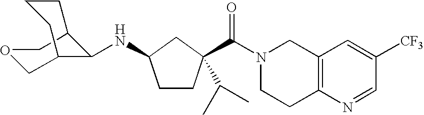

### Example IV-85

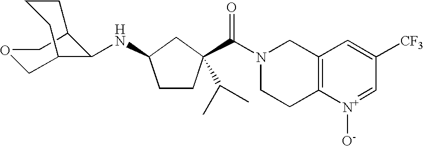

### Example IV-86

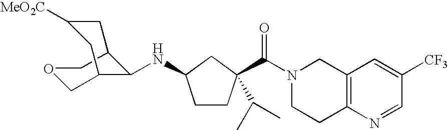

### Example IV-87

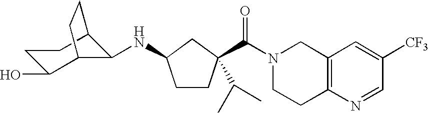

### Example IV-88

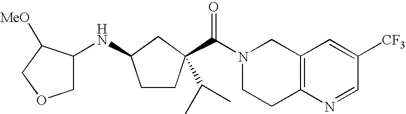

### Example IV-89

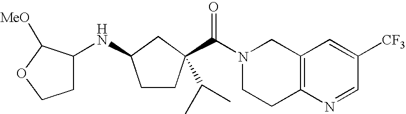

### Example IV-90

### L-224150; S. Goble; 44292-013

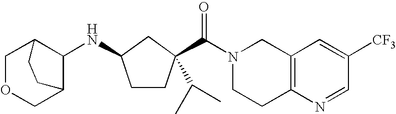

### Example IV-91

### L-224567; S. Goble; 44292-020

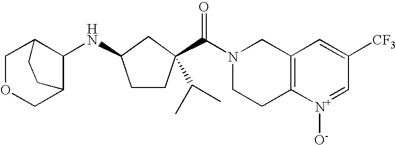

### Example IV-92

### L-234682; S. Goble; 44292-039

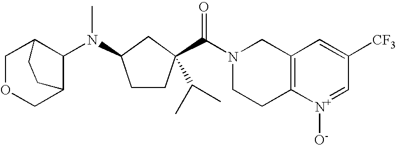

### Example IV-93

### L-233387, S. Goble; 44292-031

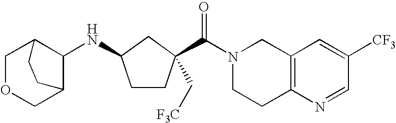

### Example IV-94

### L-233979, S. Goble; 44292-036

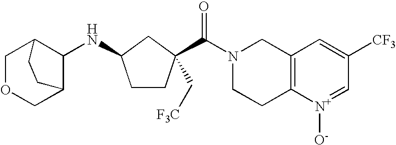

### Example IV-95

### L-234673/236874/876; S. Goble; 44292-037/059

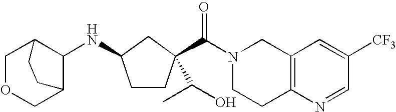

### Example IV-96

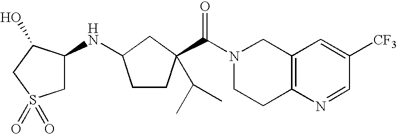

### Example IV-97

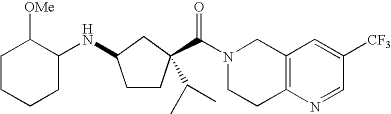

### Example IV-98

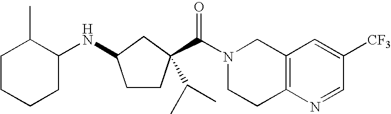

### Example IV-99

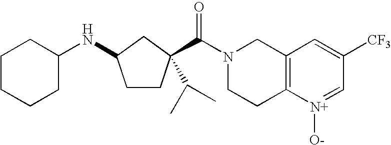

### Example IV-100

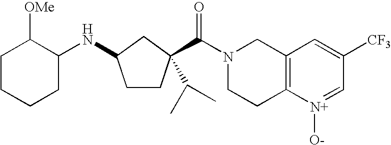

### Example IV-101

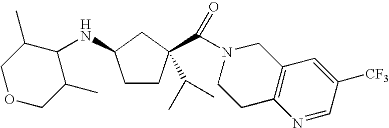

### Example IV-102

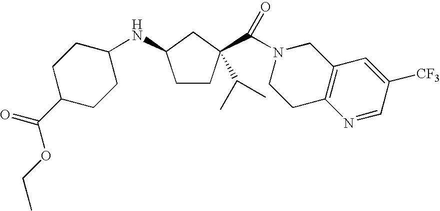

### Example IV-103

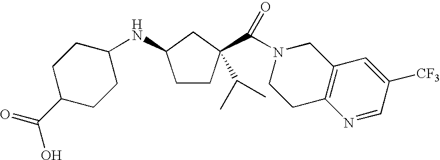

### Example IV-104

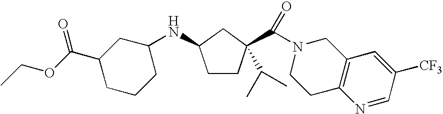

### Example IV-105

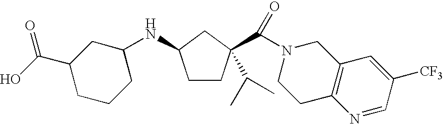

### Example IV-106

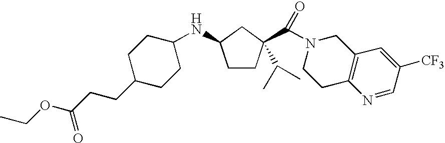

### Example IV-107

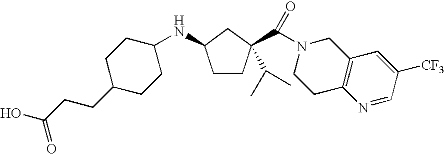

### Example IV-108

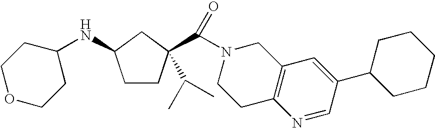

### Example IV-109

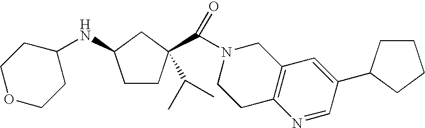

### Example IV-110

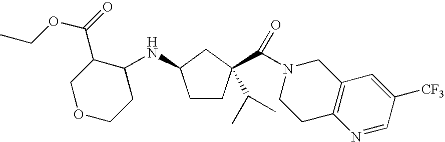

Additional CCR-2 useful in the inventive methods are those of formula V: 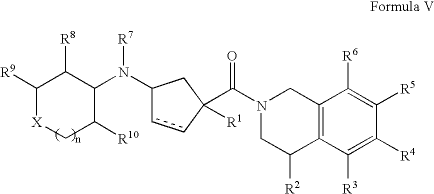
wherein:

X is selected from the group consisting of:
—O—, —NR₂₀—, —S—, —SO—, —SO₂—, and —CR²¹R²²—, —NSO₂R²⁰—,
—NCOR²⁰—, —NCO₂R²⁰—, —CR²¹CO₂R²⁰—, —CR²¹OCOR²⁰—, —CO—,
where R²⁰ is selected from: hydrogen, C₁₋₆ alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl,
where R²¹ and R²² are independently selected from: hydrogen, hydroxy, C₁₋₆ alkyl, —O—C₁₋₆alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl;

R¹ is selected from:
—C₁₋₆alkyl, —C₀₋₆alkyl-O—C₁₋₆alkyl-, —C₀₋₆alkyl-S—C₁₋₆alkyl-,
—(C₀₋₆alkyl)-(C₃₋₇cycloalkyl)-(C₀₋₆alkyl), hydroxy, —CO₂R²⁰, heterocycle,
—CN, —NR²⁰R²⁶—, —NSO₂R²⁰—, —NCOR²⁰—, —NCO₂R²⁰—, —NCOR²⁰—,
—CR²¹CO₂R²⁰—, —CR²¹OCOR²⁰—, phenyl and pyridyl,
where R²⁶ is selected from: hydrogen, C₁₋₆ alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl
where the alkyl and the cycloalkyl are unsubstituted or substituted with 1-7 substituents where the substituents are independently selected from:
   (a) halo,
   (b) hydroxy,
   (c) —O—C₁₋₃alkyl,
   (d) trifluoromethyl,
   (f) C₁₋₃alkyl,
   (g) —O—C₁₋₃alkyl,
   (h) —CO₂R²⁰,
   (i) —SO₂R²⁰,
   (j) —NHCOCH₃,
   (k) —NHSO₂CH₃,
   (l) -heterocycle,
   (m) ═O,
   (n) —CN,
and where the phenyl and pyridyl are unsubstituted or substituted with 1-3 substituents where the substituents are independently selected from: halo, hydroxy, C₁₋₃alkyl, C₁₋₃alkoxy and trifluoromethyl;

R² is selected from:
(a) hydrogen,
(b) hydroxy,
(c) halo,
(d) C₁₋₃alkyl, where the alkyl is unsubstituted or substituted with 1-6 substituents independently selected from: fluoro, and hydroxy,
(e) —NR²⁰R²⁶,
(f) —CO₂R²⁰,
(g) —CONR²OR²⁶,
(h) —NR²OCOR²¹,
(i) —OCONR²⁰R²⁶,
(j) —NR²⁰CONR₂OR²⁶,
(k) -heterocycle,
(l) —CN,
(m) —NR²⁰—SO₂—NR²⁰R²⁶,
(n) —NR²⁰—SO₂—R²⁶,
(o) —SO₂—NR²⁰R²⁶, and
(p) ═O, where R² is connected to the ring via a double bond;

R³ is selected from:
(a) hydrogen,
(b) hydroxy,
(c) halo,
(d) C₁₋₆alkyl,
(e) —O—C₁₋₆alkyl,
(f) —NR²⁰R²¹,
(g) —NR²⁰CO₂R²¹,
(h) —NR²⁰CONR²⁰R²¹,
(i) —NR²⁰—SO₂—NR²⁰R²¹,
(j) —NR²⁰—SO₂—R²¹,
(k) heterocycle,
(l) —CN,
(m) —CONR²⁰R²¹,
(n) —CO₂R₂₀,
(o) —NO₂,
(p) —S—R²⁰,
(q) —SO—R²⁰,
(r) —SO₂—R²⁰, and
(s) —SO₂—NR²⁰R²¹;

R⁴ is selected from:
(a) hydrogen,
(b) C₁₋₆alkyl,
(c) trifluoromethyl,
(d) trifluoromethoxy,
(e) chloro,
(f) fluoro,
(g) bromo, and
(h) phenyl;

R⁵ is selected from:
(a) C₁₋₆alkyl, where alkyl may be unsubstituted or substituted with 1-6 fluoro and optionally substituted with hydroxyl,
(b) —O—C₁₋₆alkyl, where alkyl may be unsubstituted or substituted with 1-6 fluoro,
(c) —CO—C₁₋₆alkyl, where alkyl may be unsubstituted or substituted with 1-6 fluoro,
(d) —S—C₁₋₆alkyl, where alkyl may be unsubstituted or substituted with 1-6 fluoro,
(e) -pyridyl, which may be unsubstituted or substituted with one or more substituents selected from the group consisting of: halo, trifluoromethyl, C₁₋₄alkyl, and CO₂R²⁰,
(f) fluoro,
(g) chloro,
(h) bromo,
(i) —C₄₋₆cycloalkyl,
(j) —O—C₄₋₆cycloalkyl,
(k) phenyl, which may be unsubstituted or substituted with one or more substituents selected from the group consisting of: halo, trifluoromethyl, C₁₋₄alkyl, and CO₂R²⁰,
(l) —O-phenyl, which may be unsubstituted or substituted with one or more substituents selected from the group consisting of: halo, trifluoromethyl, C₁₋₄alkyl, and CO₂R²⁰,
(m) —C₃-cycloalkyl, where alkyl may be unsubstituted or substituted with 1-6 fluoro,
(n) —O—C₃₋₆cycloalkyl, where alkyl may be unsubstituted or substituted with 1-6 fluoro,
(o) -heterocycle,
(p) —CN, and
(q) —CO₂R²⁰;

R⁶ is selected from:
(a) hydrogen,
(b) C₁₋₆alkyl, and
(c) trifluoromethyl
(d) fluoro
(e) chloro, and
(f) bromo;

R⁷ is selected from:
(a) hydrogen, and
(b) C₁₋₆alkyl, which is unsubstituted or substituted with 1-3 substituents where the substituents are independently selected from: halo, hydroxy, —CO₂H, —CO₂C₁₋₆alkyl, and —O—C₁₋₃alkyl;

R⁸ is selected from:
(a) hydrogen,
(b) C₁₋₆alkyl, where alkyl may be unsubstituted or substituted with 1-6 substituents where the substituents are chosen from the group: fluoro, C₁₋₃alkoxy, hydroxy, —CO₂R²⁰,
(c) fluoro,
(d) —O—C₁₋₃alkyl, where alkyl may be unsubstituted or substituted with 1-3 fluoro, and
(e) C₃₋₆ cycloalkyl,
(f) —O—C₃₋₆cycloalkyl,
(g) hydroxy,
(h) —CO₂R²⁰,
(i) —OCOR²⁰,
or R⁷ and R⁸ may be joined together via a C₂₋₄alkyl or a C₀₋₂alkyl-O—C₁₋₃alkyl chain to form a 5-7 membered ring;

R⁹ is selected from:
(a) hydrogen,
(b) C₁₋₆alkyl, where alkyl may be unsubstituted or substituted with 1-6 substituents where the substituents are chosen from the group: fluoro, C₁₋₃alkoxy, hydroxy, —CO₂R²⁰,
(c) CO₂R²⁰,
(d) hydroxy, and
(e) —O—C₁₋₆alkyl, where alkyl may be unsubstituted or substituted with 1-6 substituents where the substituents are chosen from the group: fluoro, C₁₋₃alkoxy, hydroxy, —CO₂R²⁰,
or R⁸ and R⁹ may be joined together by a C₁₋₄alkyl chain or a C₀₋₃alkyl-O—C₀₋₃alkyl chain to form a 3-6 membered ring;

R¹⁰ is selected from:
(a) hydrogen, and
(b) C₁₋₆alkyl, where alkyl may be unsubstituted or substituted with 1-6 fluoro,
(c) fluoro,
(d) —O—C₃₋₆cycloalkyl, and
(e) —O—C₁₋₃alkyl, where alkyl may be unsubstituted or substituted with 1-6 fluoro,
or R⁸ and R¹⁰ may be joined together by a C₂₋₃alkyl chain to form a 5-6 membered ring, where the alkyl are unsubstituted or substituted with 1-3 substituents where the substiuents are independently selected from: halo, hydroxy, —CO₂R²⁰, C₁₋₃alkyl, and C₁₋₃alkoxy,
or R⁸ and R¹⁰ may be joined together by a C₁₋₂alkyl-O—C₁₋₂alkyl chain to form a 6-8 membered ring, where the alkyl are unsubstituted or substituted with 1-3 substituents where the substiuents are independently selected from: halo, hydroxy, —CO₂R²⁰, C₁₋₃alkyl, and C₁₋₃alkoxy,
or R⁸ and R¹⁰ may be joined together by a —O—C₁₋₂alkyl-O-chain to form a 6-7 membered ring, where the alkyl are unsubstituted or substituted with 1-3 substituents where the substiuents are independently selected from: halo, hydroxy, —CO₂R²⁰, C₁₋₃alkyl, and C₁₋₃alkoxy;

n is selected from 0, 1 and 2;

the dashed line represents a single or a double bond;

and pharmaceutically acceptable salts thereof and individual diastereomers thereof.

### Formula V Compounds

### EXAMPLES

Examples of compounds of Formula V include the following:

### Example V-1

### L-070370, L-070371, L-070320, L-070321

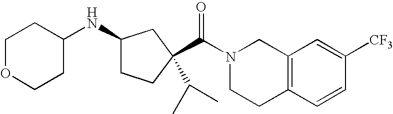

### Example V-2

### L-070675, L-070676, L-070677, L-070678

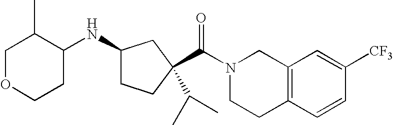

### Example V-3

### L-070575

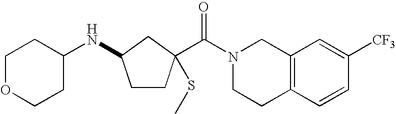

### Example V-4

### L-070578, L-070579

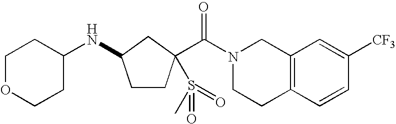

### Example V-5

### L-383766

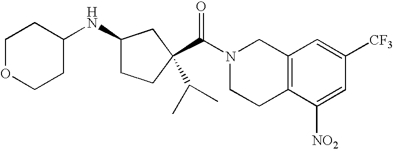

### Example V-6

### L-384176

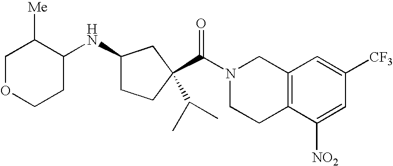

### Example V-7

### L-383767, L-383769

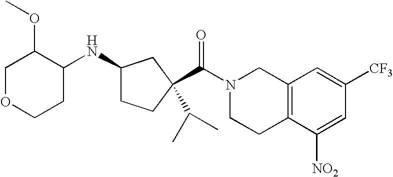

### Example V-8

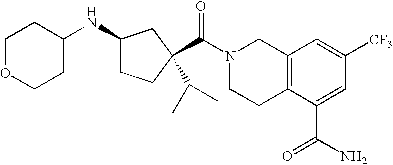

### Example V-9

### L-114593

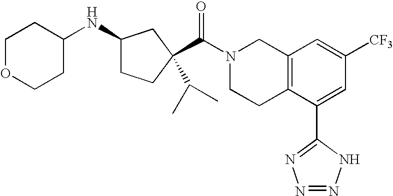

### Example V-10

### L-074303

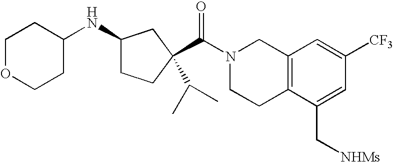

### Example V-11

### L-073260

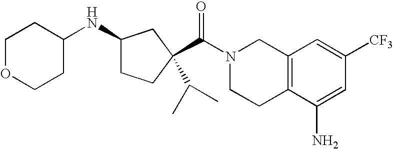

### Example V-12

### L-120189

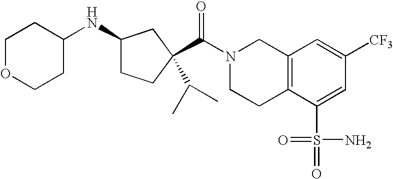

### Example V-13

### L-070942, L-070943

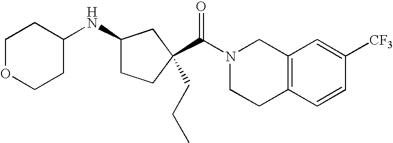

### Example V-14

### L-070963

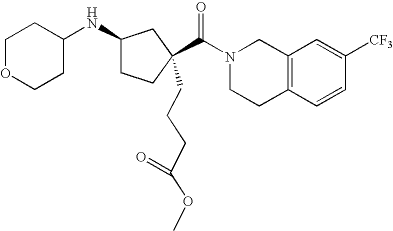

### Example V-15

### L-070964

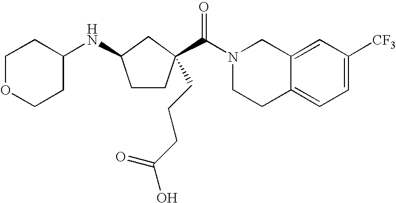

### Example V-16

### L-070287, L-070662, L-070670

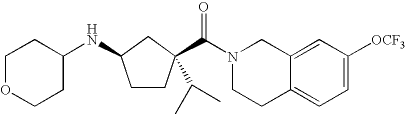

### Example V-17

### L-070422

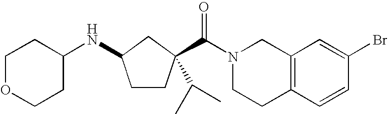

### Example V-18

### L-070825

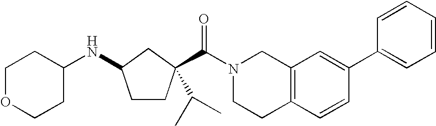

### Example V-19

### L-070237

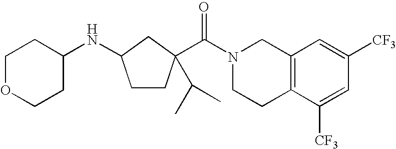

### Example V-20

### L-070379, L-070380, L-070435, L-070436

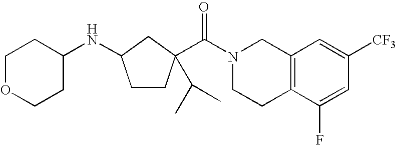

### Example V-21

### L-070728, L-070729

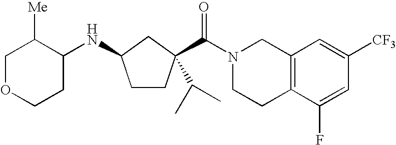

### Example V-22

### L-070755, L-070757

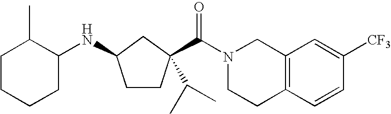

### Example V-23

### L-070730, L-070731, L-070732

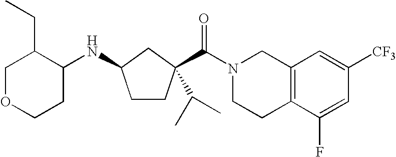

### Example V-24

### L-070733, L-070734, L-070735

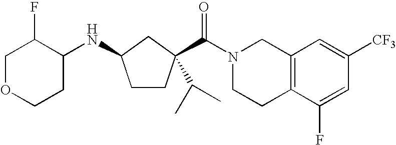

### Example V-25

### L-070421

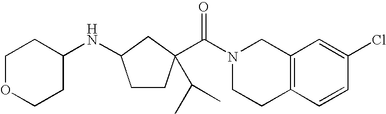

### Example V-26

### L-234913

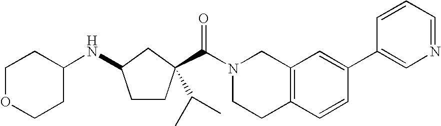

### Example V-27

### L-260680

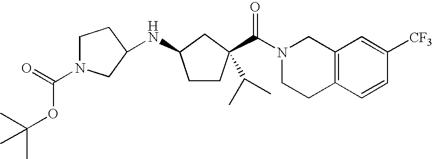

### Example V-28

### L-260683

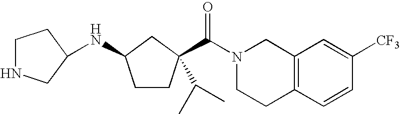

### Example V-29

### L-310391

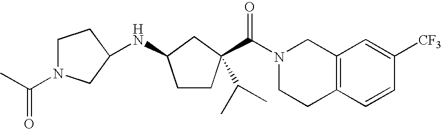

### Example V-30 to V-39

Examples V-30 through V-39, in Table 23, below, are based on the Formula:

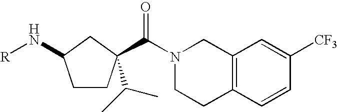

| | | Molecular | Calculated | Found |
|---|---|---|---|---|
| Example | R | Formula | [M⁺H⁺] | [M⁺H⁺] |
| V-30 L-070757 | 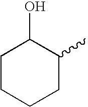 | C₂₅H₃₆F₃N₂O₂ | 453.27 | 453.25 |
| V-31 L-070771 | 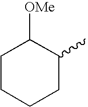 | C₂₆H₃₈F₃N₂O₂ | 467.28 | 467.35 |
| V-32 L-070772 | 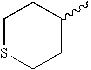 | C₂₄H₃₄F₃N₂OS | 455.23 | 455.2 |
| V-33 L-070773 | 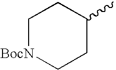 | C₂₉H₄₃F₃N₃O₃ | 538.32 | 538.3 |
| V-34 L-070774 | 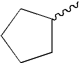 | C₂₄H₃₄F₃N₂O | 423.25 | 423.25 |
| V-35 L-070775 | 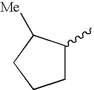 | C₂₅H₃₆F₃N₂O | 437.27 | 437.35 |
| V-36 L-070776 | 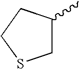 | C₂₃H₃₂F₃N₂OS | 441.21 | 441.25 |
| V-37 L-070778 | 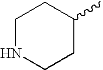 | C₂₄H₃₄F₃N₃O | 437.27 | 437.25 |
| V-38 L-070813 | 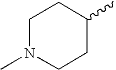 | C₂₅H₃₇F₃N₃O | 452.28 | 452.35 |
| V-39 L-070816 | 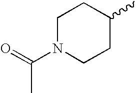 | C₂₆H₃₇F₃N₃O₂ | 480.28 | 480.25 |

### Example V-40

### L-250553

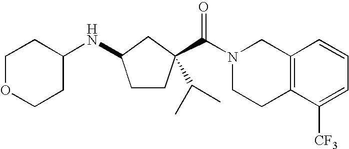

### Example V-41

### L-236892

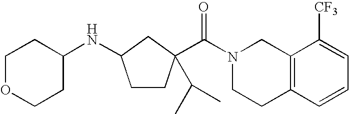

### Example V-42

### L-236378

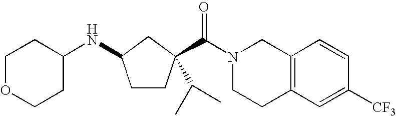

### Example V-43

### Alex NB 30766-81, L-071002

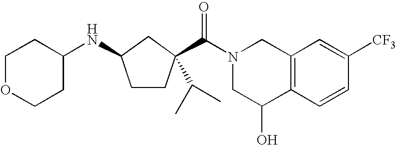

### Example V-44

### Alex NB 30766-110, L-071001

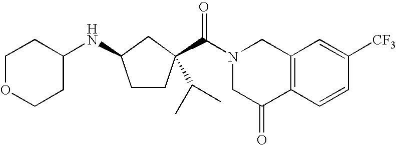

### Example V-45

### Alex NB 30766-115, L-071067

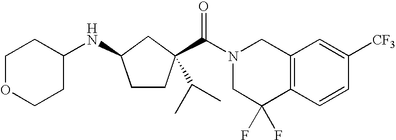

### Example V-46

### Alex NB 30767-73, L-1 14771 and L-1 14773

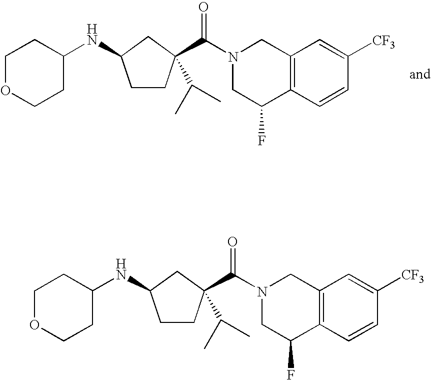

### Example V-47

### Alex NB 30767-45, L-120416 and L-120421

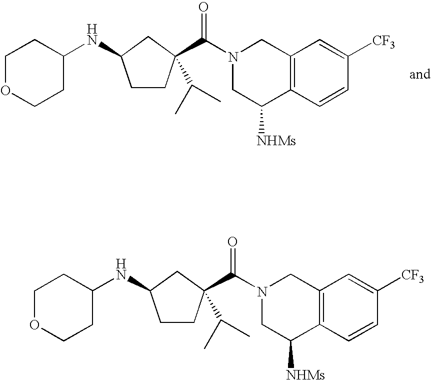

### Example V-48

### Alex NB 3076746, L-120425

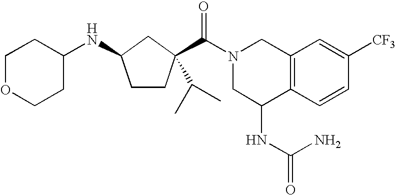

### Example V-49

### Alex NB 30767-47, L-120430

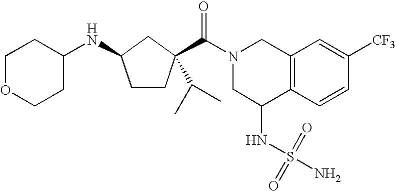

### Example V-50

### Alex NB 30767-72, L-123597

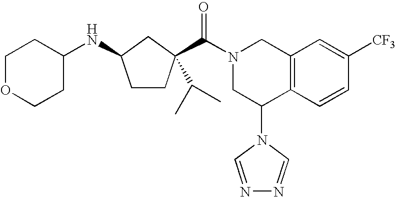

### Example V-51

### Alex NB 30767-89, L-221505, L-221506

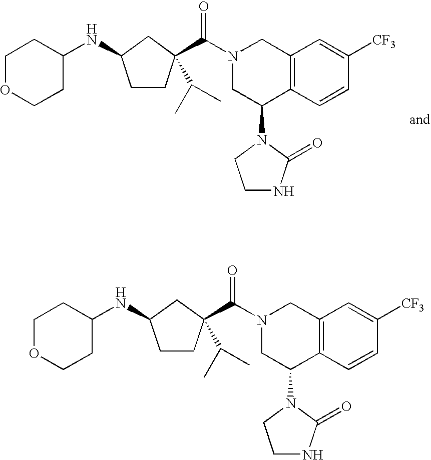

### Example V-52

### Alex NB 44362-52, L-311982, L-311985

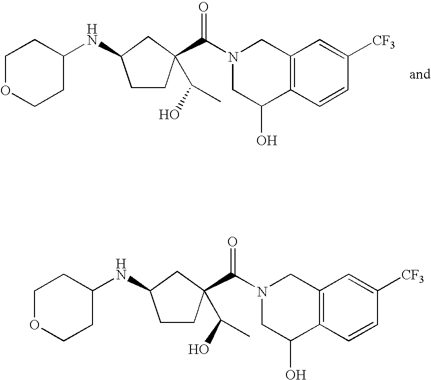

### Example V-53

### Alex NB 44362-70, L-383026, L-383032, L-383038, L-383089

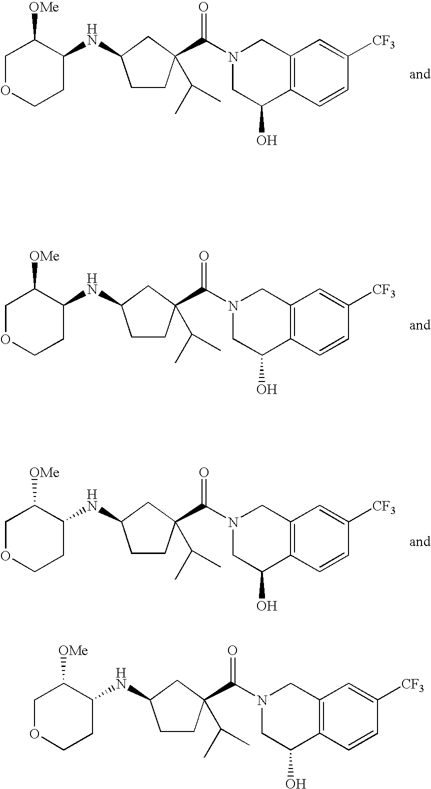

### Example V-54

### L-070949; S. Goble; 30708-110

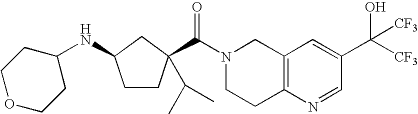

### Example V-55

### L-070977; S. Goble; 30708-127A

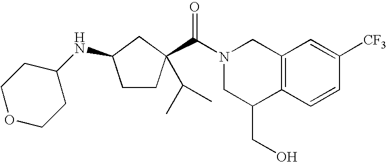

### Example V-56

### L-070992; S. Goble; 43899-018

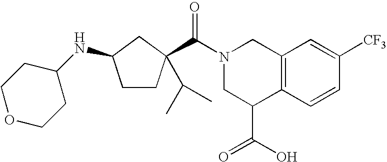

### Example V-57

### L-071088; S. Goble; 43899-027

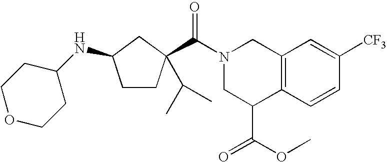

### Example V-58

### L-121449; S. Goble; 43899-113

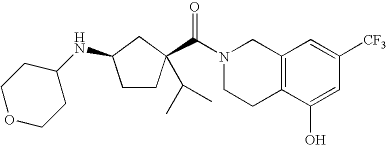

### Example V-59

### L-122515; S. Goble; 43899-127

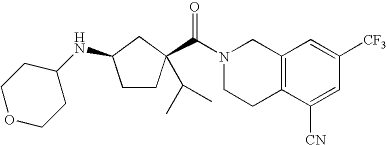

### Example V-60

### L-221934, S. Goble; 43899-128

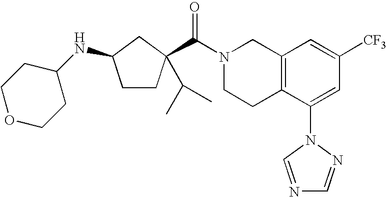

### Example V-61

### L-1923280; S. Goble; 43899-125

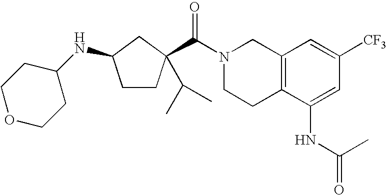

### Example V-62

### L-223615; S. Goble; 44292-015

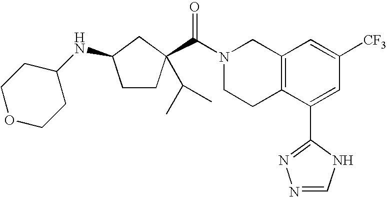

### Example V-63

### L-224164; S. Goble; 44292-017

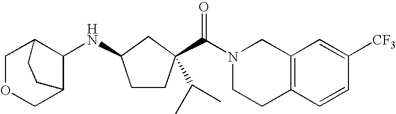

### Example V-64

### L-124089

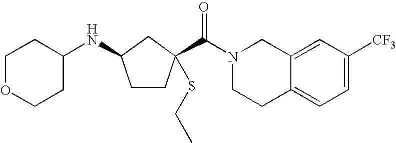

### Example V-65

### L-220436

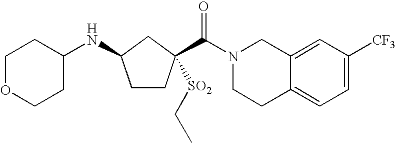

### Example V-66

### L-221632

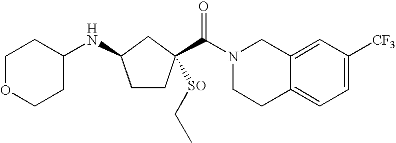

### Example V-67

### L-311515

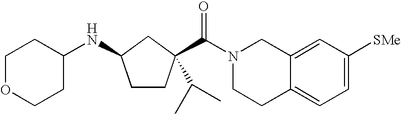

### Example V-68

### L-311518

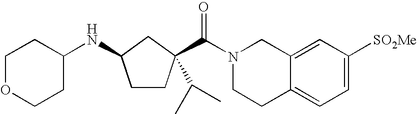

### Example V-69

### L-074185

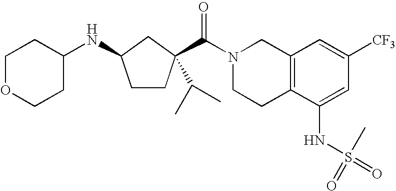

### Example V-70

### L-074197

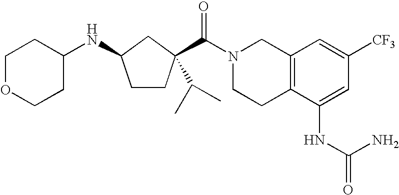

### Example V-71

### L-074302

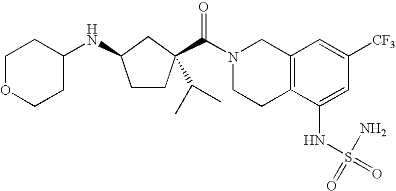

### Example V-72

### L-235567

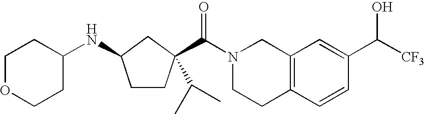

### Example V-73

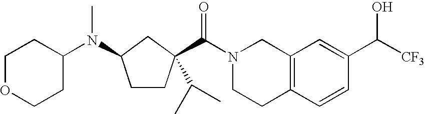

### Example V-74

### L-236107

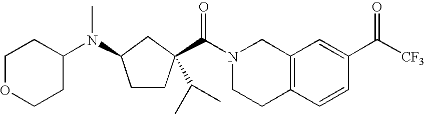

### Example V-75

### L-071029

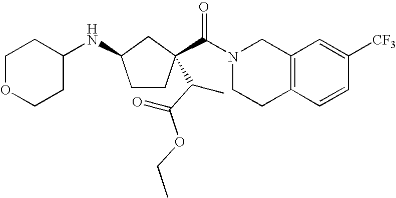

### Example V-76

### L-071028

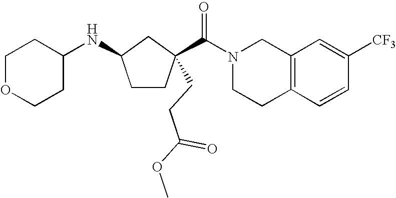

### Example V-77

### L-070967

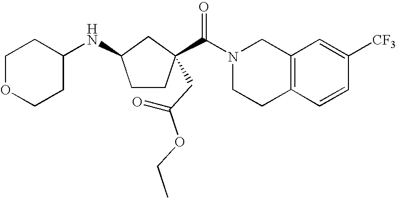

### Example V-78

### L-070887

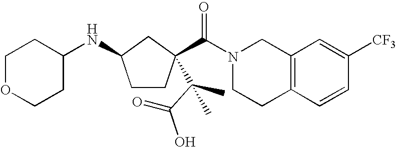

### Example V-79

### L-070838

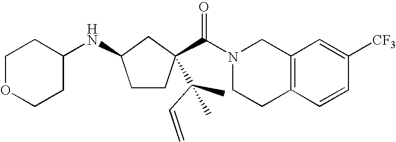

### Example V-80

### L-071054, L-071055, L-071056, L-071059, L-071061

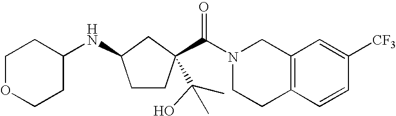

### Example V-80

### L-071075, L-071074

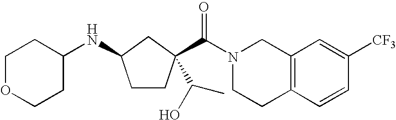

### Example V-81

### L-075638

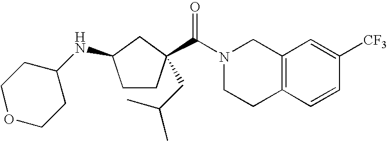

### Example V-82

### L-071148

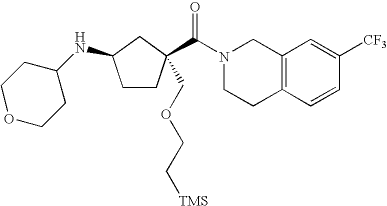

### Example V-83

### L-075404

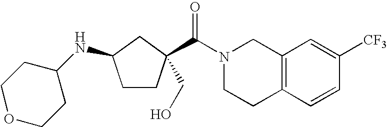

### Example V-84

### L-120222

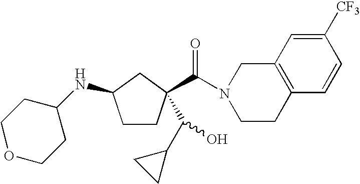

### Example V-85

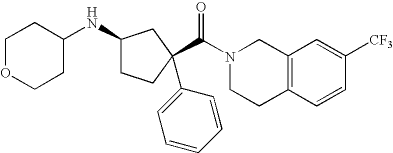

### Example V-86

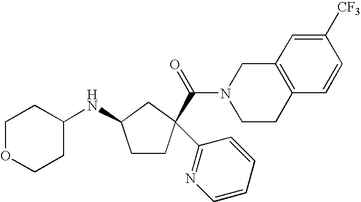

### Example V-87

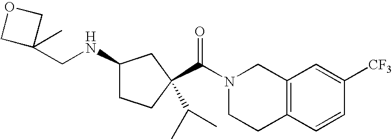

### Example V-88

### L-311887

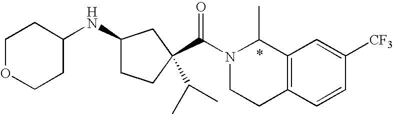

### Example V-89

### L-075595

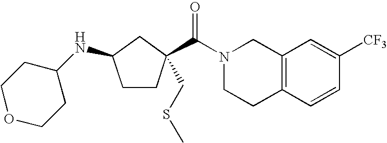

### Example V-90

### L-120400

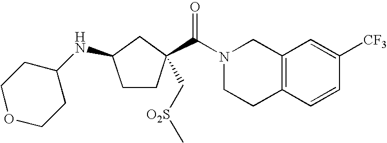

### Example V-91

### L-124984

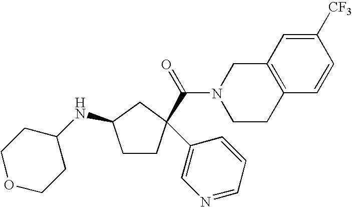

### Example V-92

### L-070513

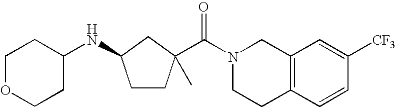

### Example V-93

### L-070756

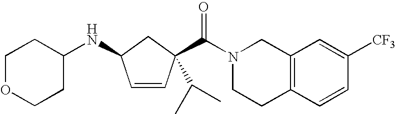

### Example V-94

### L-070686

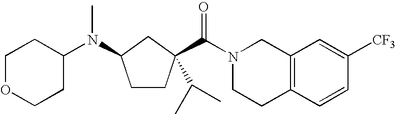

### Example V-95

### L-070720, L-070721

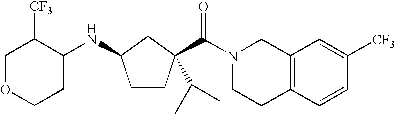

### Example V-96

### L-070722, L-070788, L-070789, L-070790, L-070791

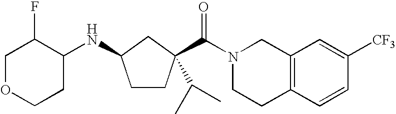

### Example V-97

### L-070723, L-070792, L-070793, L-070794

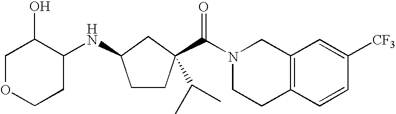

### Example V-98

### L-070514

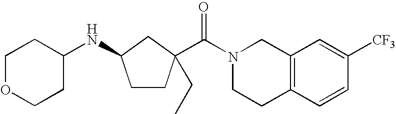

### Example V-99

### L-070872, L-070937, L-070938

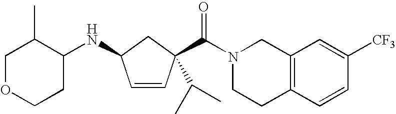

### Example V-100

### L-070873

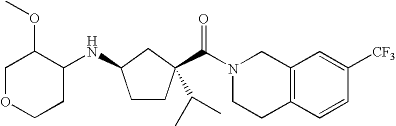

### Example V-101

### L-070855

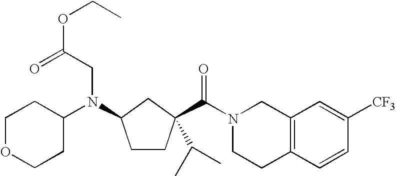

### Example V-102

### L-070856

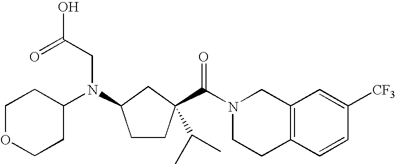

### Example V-103

### L-070898

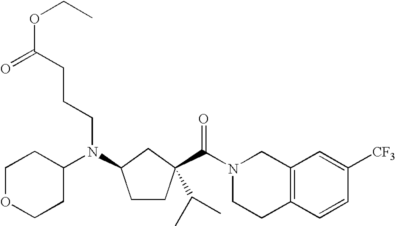

### Example V-104

### L-070899

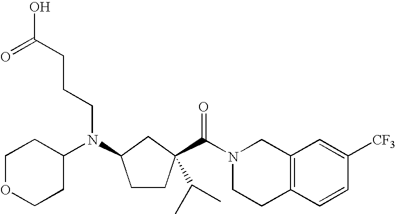

### Example V-105

### L-070858

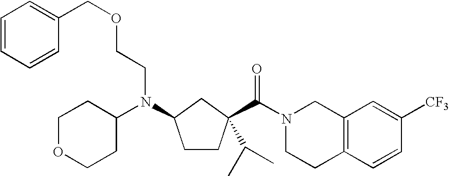

### Example V-106

### L-070859

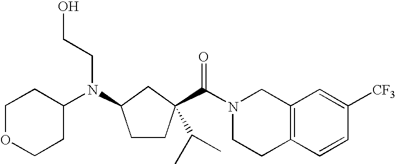

### Example V-107

### L-070857

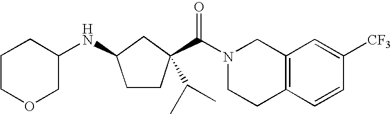

### Example V-108

### L-070830, L-070860, L-070861

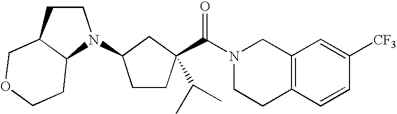

### Example V-109

### L-070831

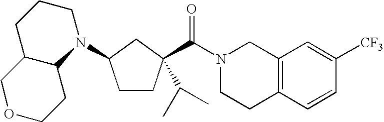

### Example V-110

### L-121458

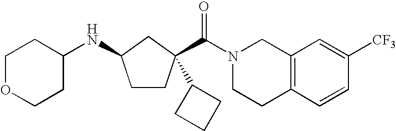

### Example V-111 and V-112

### L-071037 and L-071038

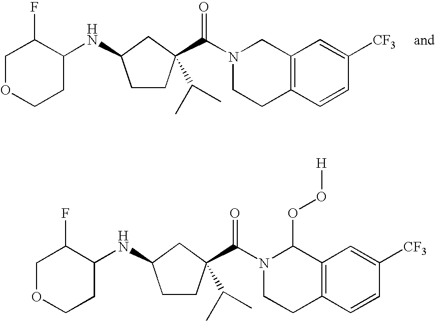

### Example V-113

### L-070843

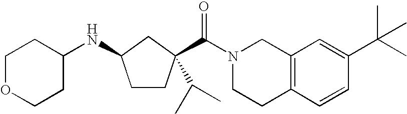

### Example V-114

### L-071141

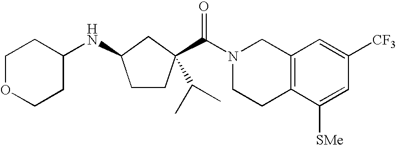

### Example V-115

### L-071159

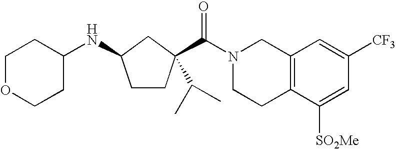

### Example V-116

### L-071160

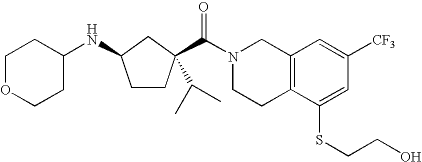

### Example V-117

### L-071160

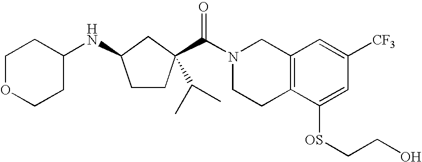

### Example V-118

### L-071161

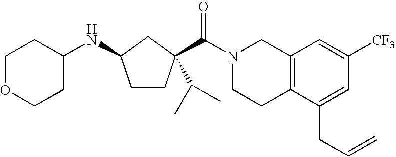

### Example V-119

### L-071163

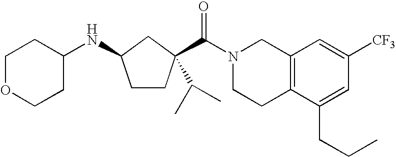

### Example V-120

### L-071164

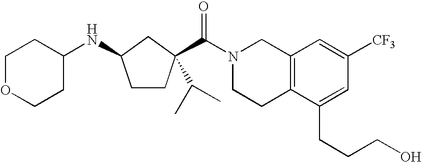

### Example V-121

### L-390277

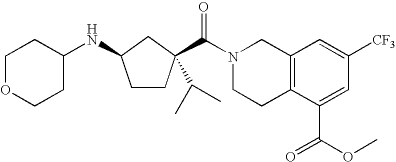

### Example V-122

### L-390278

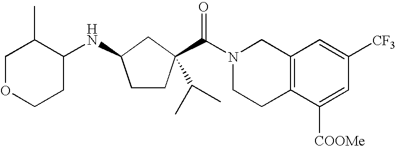

### Example V-123

### L-390280

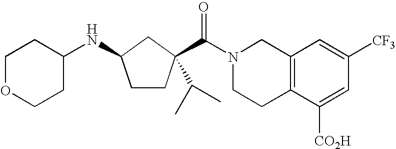

Additional CCR-2 angtagonists useful in the methods of the invention include those of Formula VI: 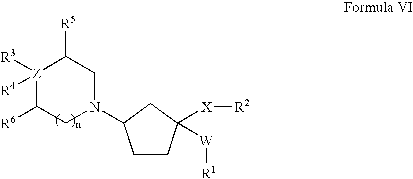
wherein:

X is selected from the group consisting of:
—NR¹⁰—, —O—, —CH₂O—, —CONR¹⁰—, —NR¹⁰CO—, —CO₂—, —OCO—,
—CH₂(NR¹⁰)CO—, —N(COR¹⁰)—, —CH₂N(COR¹⁰)—, phenyl, and
C₃₋₆ cycloalkyl,
where R¹⁰ is independently selected from: hydrogen, C₁₋₆ alkyl, benzyl, phenyl, and C₁₋₆ alkyl-C₃₋₆ cycloalkyl,
   which is unsubstituted or substituted with 1-3 substituents where the substituents are independently selected from: halo, C₁₋₃alkyl, C₁₋₃alkoxy and trifluoromethyl;

W is selected from:
phenyl and heterocycle, which is unsubstituted or substituted with 1-3 substituents where the substituents are independently selected from: halo, C₁₋₃alkoxy and trifluoromethyl;

Z is selected from:
C, N, and —O—, wherein when Z is N, then R⁴ is absent, and when W is —O—, then both R³ and R⁴ are absent;

n is an integer selected from 0, 1, 2, 3 and 4;

R¹ is selected from:
(a) halo,
(b) trifluoromethyl,
(c) trifluoromethoxy,
(d) hydroxy,
(e) C₁₋₆alkyl,
(f) C₃₋₇cycloalkyl,
(g) —O—C₁₋₆alkyl,
(h) —O—C₃₋₇cycloalkyl,
(i) —SCF₃,
(j) —S—C₁₋₆alkyl,
(k) —SO₂—C₁₋₆alkyl,
(l) phenyl,
(m) heterocycle,
(n) —CO₆R⁹,
(o) —CN,
(p) —NR⁹R¹⁰,
(q) —NR⁹—SO₂—R¹⁰,
(r) —SO₂—NR⁹R¹⁰, and
(s) —CONR⁹R¹⁰
(t) —NHC(═NH)NH₂, and
(u) hydrogen,

R² is selected from:
(C₀₋₆alkyl)-phenyl and (C₀₋₆alkyl)-heterocycle, where the alkyl is unsubstituted or substituted with 1-7 substituents where the substituents are independently selected from:
   (a) halo,
   (b) hydroxy,
   (c) —O—C₁₋₃alkyl,
   (d) trifluoromethyl, and
   (e) —C₁₋₃alkyl,
and where the phenyl and the heterocycle is unsubstituted or substituted with 1-5 substituents where the substituents are independently selected from:
   (a) halo,
   (b) trifluoromethyl,
   (c) trifluoromethoxy,
   (d) hydroxy,
   (e) C₁₋₆alkyl,
   (f) C₃₋₇cycloalkyl,
   (g) —O—C₁₋₆alkyl,
   (h) —O—C₃₋₇cycloalkyl,
   (i) —SCF₃,
   (j) —S—C₁₋₆alkyl,
   (k) —SO₂—C₁₋₆alkyl,
   (l) phenyl,
   (m) heterocycle,
   (n) —CO₂R⁹,
   (o) —CN,
   (p) —NR⁹R¹⁰,
   (q) —NR⁹—SO₂—R¹⁰,
   (r) —SO₂—NR⁹R¹⁰, and
   (s) —CONR⁹R¹⁰;

R³ is —(C₀₋₆alkyl)-phenyl,
where the alkyl is unsubstituted or substituted with 1-5 substituents where the substituents are independently selected from:
(a) halo,
(b) hydroxy,
(c) —O—C₁₋₃alkyl, and
(d) trifluoromethyl,

and where the phenyl is unsubstituted or substituted with 1-5 substituents where the substituents are independently selected from:
(a) halo,
(b) trifluoromethyl,
(c) hydroxy,
(d) C₁₋₃alkyl,
(e) —O—C₁₋₃alkyl,
(f) —CO₂R⁹,
(g) —CN,
(h) —NR⁹R¹⁰, and
(i) —CONR⁹R¹⁰;

R⁴ is selected from:
(a) hydrogen,
(b) hydroxy,
(c) C₁₋₆alkyl,
(d) C₁₋₆alkyl-hydroxy,
(e) —O—C₁₋₃alkyl,
(f) —CO₂R⁹,
(g) —CONR⁹R¹⁰, and
(h) —CN;

or where R³ and R⁴ may be joined together to form a ring which is selected from:
(a) 1H-indene,
(b) 2,3-dihydro-1H-indene,
(c) 2,3-dihydro-benzofuran,
(d) 1,3-dihydro-isobenzofuran,
(e) 2,3-dihydro-benzothiofuran, and
(f) 1,3-dihydro-isobenzothiofuran,

or where R³ and R⁵ or R⁴ and R⁶ may be joined together to form a ring which is phenyl,
wherein the ring is unsubstituted or substituted with 1-7 substituents where the substituents are independently selected from:
   (a) halo,
   (b) trifluoromethyl,
   (c) hydroxy,
   (d) C₁₋₃alkyl,
   (e) —O—C₁₋₃alkyl,
   (f) —CO₂R₉,
   (g) —CN,
   (h) —NR⁹R¹⁰, and
   (i) —CONR⁹R¹⁰;

R⁵ and R⁶ are independently selected from:
(a) hydrogen,
(b) hydroxy,
(c) C₁₋₆alkyl,
(d) C₁₋₆alkyl-hydroxy,
(e) —O—C₁₋₃alkyl,
(f) oxo, and
(g) halo;

and pharmaceutically acceptable salts thereof and individual diastereomers thereof.

### Formula VI Compounds

### EXAMPLES

Examples of the compounds of Formula VI include the following:

### Example VI-1

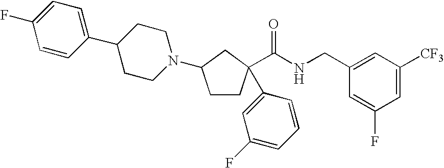

### Example VI-2

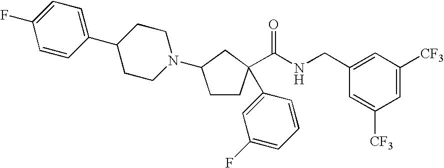

### Example VI-11

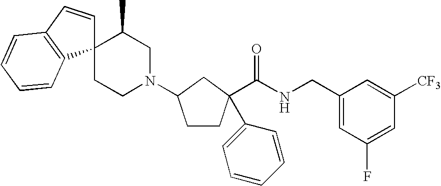

### Example VI-24

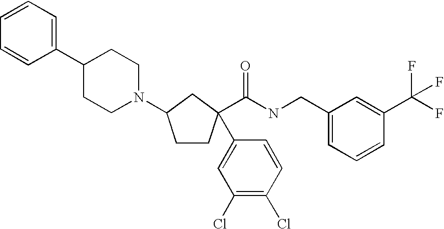

### Example VI-45

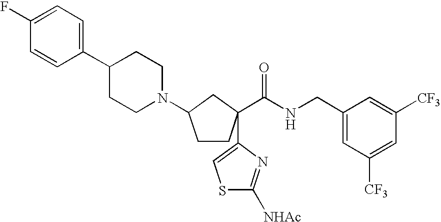

### Example VI-46

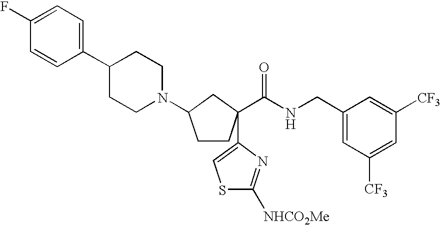

### Example VI-47

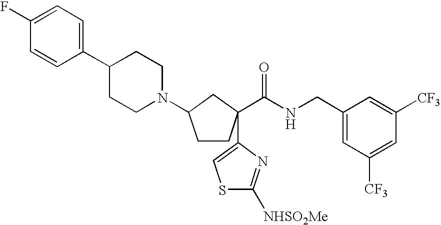

### Example VI-48

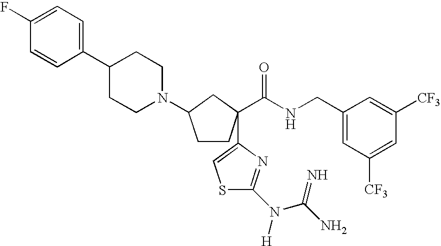

### Example VI-49

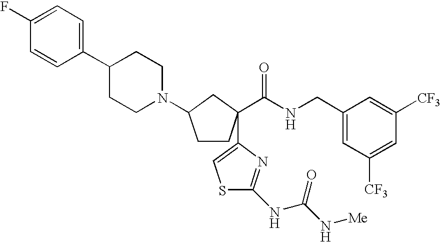

### Example VI-50

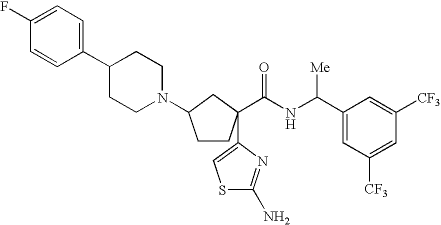

### Example VI-51

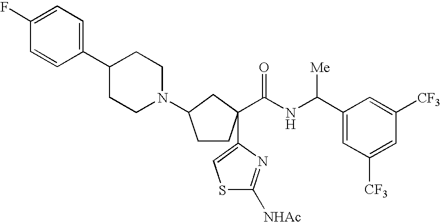

### Example VI-80

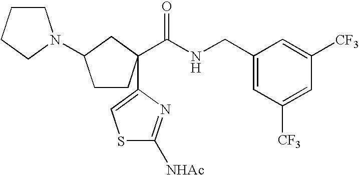

### Example VI-81

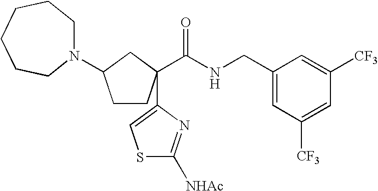

### Example VI-82

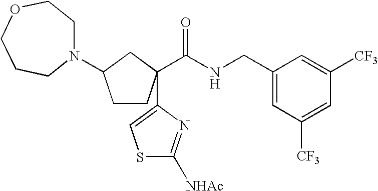

### Example VI-83

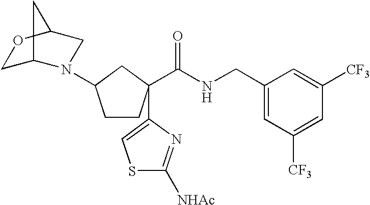

### Example VI-84

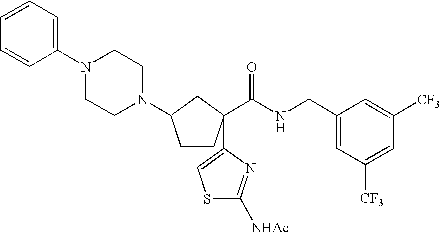

Additional CCR-2 antagonists useful in the methods of the invention include theose of Formula VII. 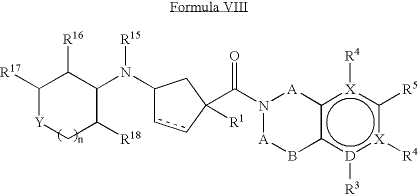
wherein:

A, B, X, and D are defined as follows with the exceptions that A, B, X, and D cannot be simultaneously CR⁸R⁸, CR²R², CR⁴, and CR³, respectively, and that D can only be N when at least one of A, B, or X is not CR⁸R⁸, CR²R², or CR⁴, respectively (where R⁸, R², R⁴, and R³ are defined below;

A is independently selected from the group consisting of —CR⁸R⁸—, —CO—, —NR⁸—, and —O—, where
R⁸ is independently selected from hydrogen, C₁₋₆alkyl, CO₄alkylCOR¹¹, and
   where R¹¹ is selected from: hydroxy, hydrogen, C₁₋₆ alkyl, —O—C₁₋₆alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl;

B is selected from the group consisting of —CR²R²—, —O—, —SO—, —SO₂—, —NSO₂R¹⁴—, —NCOR¹³—,
—NCONR¹²R¹²— and —CO—, where R² is independently selected from hydrogen, C₁₋₆alkyl, fluoro, hydroxy, heterocycle, —NHCOR¹³, —NHSO₂R¹⁴, and —O—C₁₋₆alkyl, and
   where R¹² is selected from: hydrogen, C₁₋₆ alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl, and
   where R¹³ is selected from: hydrogen, C₁₋₆ alkyl, —O—C₁₋₆alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl, and
   where R¹⁴ is selected from: hydroxy, C₁₋₆ alkyl, —O—C₁₋₆alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl, and
where the heterocycle is unsubstituted or substituted with 1-3 substituents where the substituents are independently selected from: halo, hydroxy, —COR¹¹, C₁₋₃alkyl, C₁₋₃alkoxy and trifluoromethyl;

X is independently selected from a carbon atom, or a nitrogen atom;

D can be a carbon atom, and when one of B, X, or D is not CR²R², a carbon atom, and a carbon atom, respectively, then D can also be a nitrogen atom;

Y is selected from the group consisting of:
—O—, —NR¹²—, —S—, —SO—, —SO₂—, and —CR¹¹R¹¹—, —NSO₂R¹⁴—,
—NCOR¹³—, —NCONR¹²R¹²—, —CR¹¹COR¹¹—, —CR¹¹OCOR¹³— and —CO—;

R¹ is selected from:
hydrogen, —C₁₋₆alkyl, —C₀₋₆alkyl-O—C₁₋₆alkyl, —C₀₋₆alkyl-S—C₁₋₆alkyl,
—(C₀₋₆alkyl)-(C₃₋₇cycloalkyl)-(C₀₋₆alkyl), hydroxy, heterocycle,
—CN, —NR¹²R¹², —NR¹²COR¹³, —NR¹²SO₂R¹⁴, —COR¹¹, —CONR¹²R¹², and phenyl,
where the alkyl and the cycloalkyl are unsubstituted or substituted with 1-7 substituents where the substituents are independently selected from:
   (a) halo,
   (b) hydroxy,
   (c) —O—C₁₋₃alkyl,
   (d) trifluoromethyl,
   (f) C₁₋₃alkyl,
   (g) —O—C₁₋₃alkyl,
   (h) —COR¹¹,
   (i) —SO₂R¹⁴,
   (j) —NHCOCH₃,
   (k) —NHSO₂CH₃,
   (l) -heterocycle,
   (m) ═O,
   (n) —CN,
and where the phenyl and heterocycle are unsubstituted or substituted with 1-3 substituents where the substituents are independently selected from: halo, hydroxy, —COR¹¹, C₁₋₃alkyl, C₁₋₃alkoxy and trifluoromethyl;

R³ is selected from:
(a) hydrogen,
(b) C₁₋₃alkyl, optionally substituted with 1-3 fluoro,
(c) —O—C₁₋₃alkyl, optionally substituted with 1-3 fluoro,
(d) hydroxy,
(e) chloro,
(f) fluoro,
(g) bromo,
(h) phenyl,
(g) heterocycle, and
(h) nothing, O, or hydrogen (when the Z bonded to R³ is N);

R⁴ is selected from:
(a) hydrogen,
(b) C₁₋₃alkyl, optionally substituted with 1-3 fluoro,
(c) —O—C₁₋₃alkyl, optionally substituted with 1-3 fluoro,
(d) hydroxy,
(e) chloro,
(f) fluoro,
(g) bromo,
(h) phenyl,
(g) heterocycle, and
(h) nothing, O, or hydrogen (when the Z bonded to R⁴ is N);

R⁵ is selected from:
(a) C₁₋₆alkyl, where alkyl is unsubstituted or substituted with 1-6 fluoro and optionally substituted with hydroxyl,
(b) —O—C₁₋₆alkyl, where alkyl is unsubstituted or substituted with 1-6 fluoro,
(c) —CO—C₁₋₆alkyl, where alkyl is unsubstituted or substituted with 1-6 fluoro,
(d) —S—C₁₋₆alkyl, where alkyl is unsubstituted or substituted with 1-6 fluoro,
(e) -pyridyl, which is unsubstituted or substituted with one or more substituents selected from the group consisting of: halo, trifluoromethyl, C₁₋₄alkyl, and COR¹¹,
(f) fluoro,
(g) chloro,
(h) bromo,
(i) —C₄₋₆cycloalkyl,
(j) —O—C₄₋₆cycloalkyl,
(k) phenyl, which is unsubstituted or substituted with one or more substituents selected from the group consisting of: halo, trifluoromethyl, C₁₋₄alkyl, and COR¹¹,
(l) —O-phenyl, which is unsubstituted or substituted with one or more substituents selected from the group consisting of: halo, trifluoromethyl, C₁₋₄alkyl, and COR¹¹,
(m) —C₃₋₆cycloalkyl, where alkyl is unsubstituted or substituted with 1-6 fluoro,
(n) —O—C₃₋₆cycloalkyl, where alkyl is unsubstituted or substituted with 1-6 fluoro,
(o) -heterocycle,
(p) —CN, and
(q) —COR¹¹;

R¹⁵ is selected from:
(a) hydrogen, and
(b) C₁₋₆alkyl, which is unsubstituted or substituted with 1-3 substituents where the substituents are independently selected from: halo, hydroxy, —CO₂H, —CO₂C₁₋₆alkyl, and —O—C₁₋₃alkyl;

R¹⁶ is selected from:
(a) hydrogen,
(b) C₁₋₆alkyl, where alkyl is unsubstituted or substituted with 1-6 substituents where the substituents are chosen from the group: fluoro, C₁₋₃alkoxy, hydroxy, —COR¹¹,
(c) fluoro,
(d) —O—C₁₋₃alkyl, where alkyl is unsubstituted or substituted with 1-3 fluoro, and
(e) C₃₋₆ cycloalkyl,
(f) —O—C₃₋₆cycloalkyl,
(g) hydroxy,
(h) —COR¹¹,
(i) —OCOR¹³,
or R¹⁵ and R¹⁶ are joined together via a C₂₋₄alkyl or a C₀₋₂alkyl-O—C₁₋₃alkyl chain to form a 5-7 membered ring;

R¹⁷ is selected from:
(a) hydrogen,
(b) C₁₋₆alkyl, where alkyl is unsubstituted or substituted with 1-6 substituents where the substituents are chosen from the group: fluoro, C₁₋₃alkoxy, hydroxy, —COR¹¹,
(c) COR¹¹,
(d) hydroxy, and
(e) —O—C₁₋₆alkyl, where alkyl is unsubstituted or substituted with 1-6 substituents where the substituents are chosen from the group: fluoro, C₁₋₃alkoxy, hydroxy, —COR¹¹,
or R¹⁶ and R¹⁷ are joined together by a C₁₋₄alkyl chain or a C₀₋₃alkyl-O—C₀₋₃alkyl chain to form a 3-6 membered ring;

R¹⁸ is selected from:
(a) hydrogen, and
(b) C₁₋₆alkyl, where alkyl is unsubstituted or substituted with 1-6 fluoro,
(c) fluoro,
(d) —O—C₃₋₆cycloalkyl, and
(e) —O—C₁₋₃alkyl, where alkyl is unsubstituted or substituted with 1-6 fluoro, or R¹⁶ and R¹⁸ are joined together by a C₂₋₃alkyl chain to form a 5-6 membered ring, where the alkyl are unsubstituted or substituted with 1-3 substituents where the substiuents are independently selected from: halo, hydroxy, —COR¹¹, C₁₋₃alkyl, and C₁₋₃alkoxy,
or R¹⁶ and R¹⁸ are joined together by a C₁₋₂alkyl-O—C₁₋₂alkyl chain to form a 6-8 membered ring, where the alkyl are unsubstituted or substituted with 1-3 substituents where the substiuents are independently selected from: halo, hydroxy, —COR¹¹, C₁₋₃alkyl, and C₁₋₃alkoxy,
or R¹⁶ and R¹⁸ are joined together by a —O—C₁₋₂alkyl-O-chain to form a 6-7 membered ring, where the alkyl are unsubstituted or substituted with 1-3 substituents where the substiuents are independently selected from: halo, hydroxy, —COR¹¹, C₁₋₃alkyl, and C₁₋₃alkoxy;

n is selected from 0, 1 and 2;

the dashed line represents a single or a double bond;

and pharmaceutically acceptable salts thereof and individual diastereomers thereof.

### Formula VII Compounds

### EXAMPLES

Example of the compounds of Formula VII include the following:

### Example VII-1

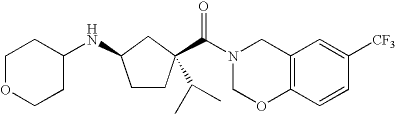

### Examples VII-2

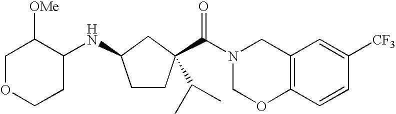

### Example VII-3

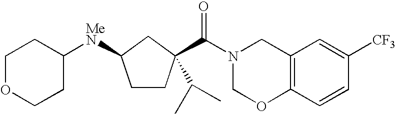

### Examples VII-4

### L-222681 and L-222682 Alex NB 30767-105

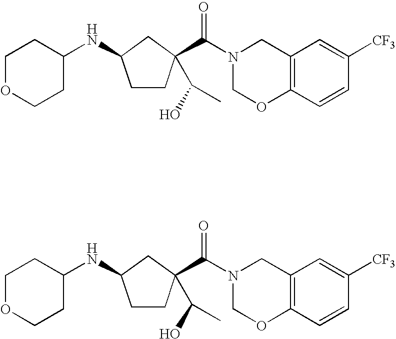

### Example VII-5

### Alex NB 30766 p 141, L-000071104-001R

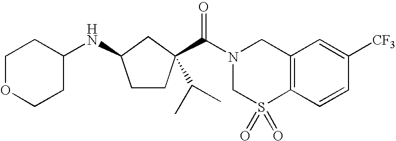

### Example VII-6

### Alex NB 307661p 142, L-000071105, L-000071106

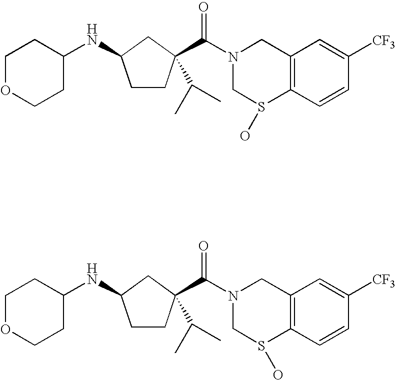

### Example VII-7

### Alex NB 30766 p 140, L-000071107

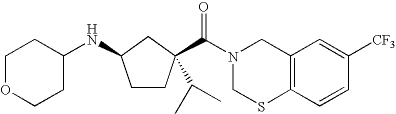

### Example VII-8

### Alex NB 30767 p 102, L-000222364, L-000222365

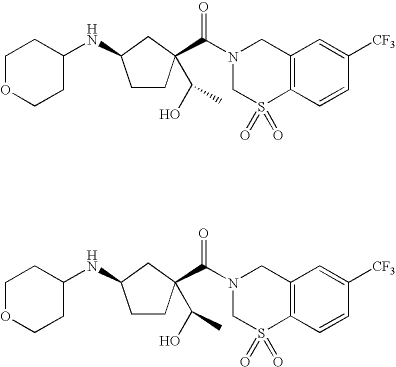

### Example VII-9

### Belinda NB 44364-, L-000234920

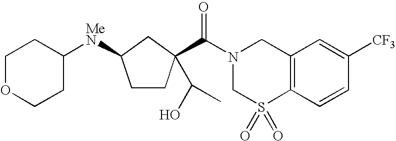

### Example VII-10

### Belinda L-234921, NB 44364

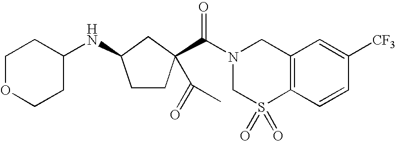

### Example VII-11

### Alex NB 44362 p 21, L-238754, L-238753

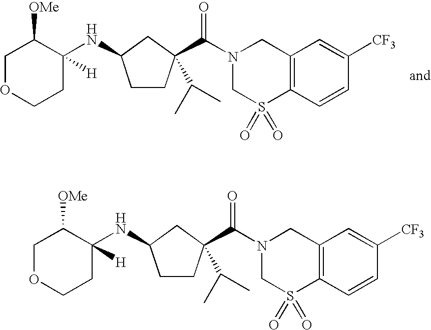

### Example VII-12

### Alex NB 30767-13, L-071127

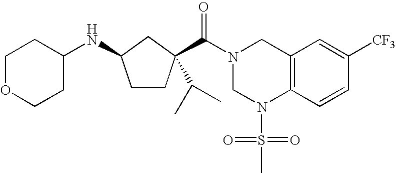

### Example VII-13

### Alex NB 30767-18, L-071140

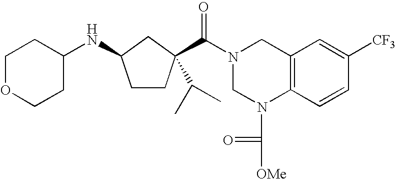

### EXAMPLE VII-14

### Alex NB 30767-141, L-235510

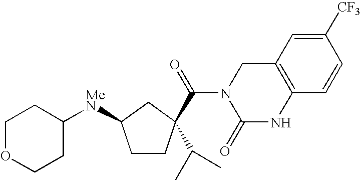

### Example VII-15

### Alex NB 30767-37, L-071154

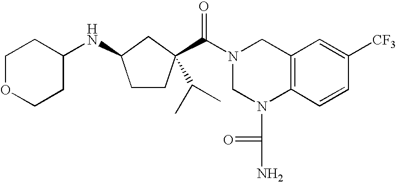

### Example VII-16

### Alex NB 30767-34, L-071155

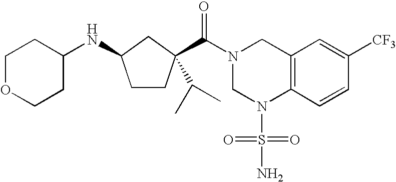

### Example VII-17

### Alex NB 30767-111, L-224750

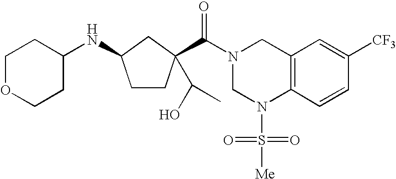

### Example VII-18

### Alex NB 30767-133, L-234924

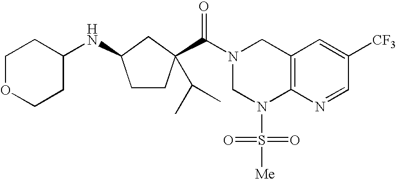

### Example VII-19

### Belinda NB 33364-39, L-250439

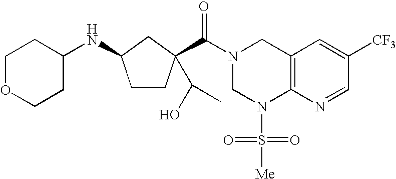

### Example VII-20

### 344432; S. Goble; 44292-115

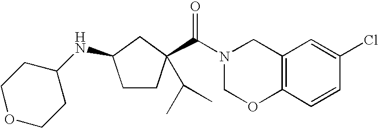

### Example VII-21

### L-070946

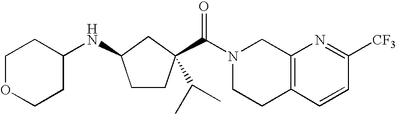

### Example VII-22

### L-071027

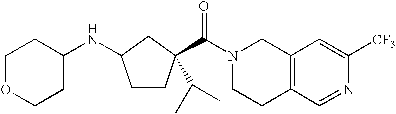

### Example VII-23

### L-071108

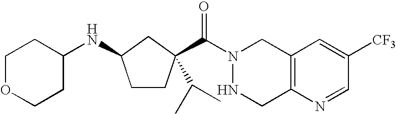

### Example VII-24

### L-121572

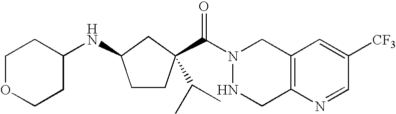

### Example VII-25

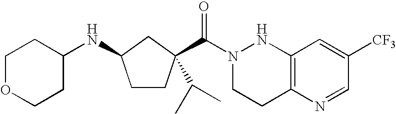

### Example VII-26

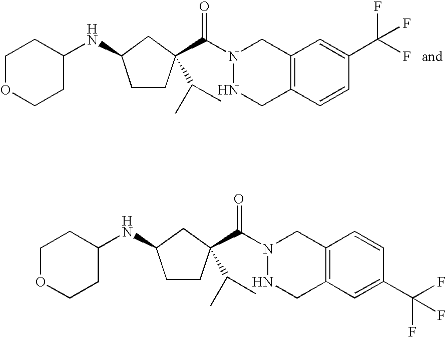

### Example VII-27

### L-224792

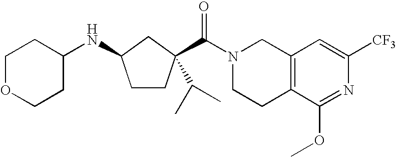

### Example VII-28

### L-224967

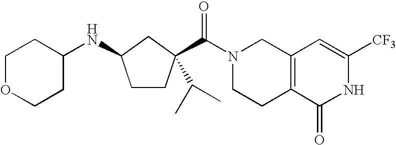

### Example VII-29

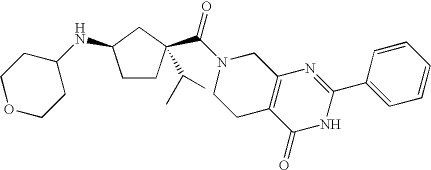

Additional CCR-2 antagonists useful in the methods of the invention include those of Formula VIII:

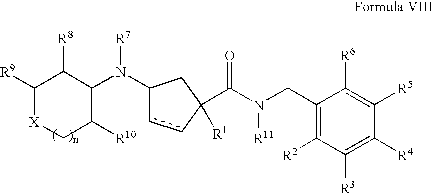

X is selected from the group consisting of:
—O—, NR²⁰—, —S—, —SO—, —SO₂—, and —CR²¹R²²—, —NSO₂R²⁰—,
—NCOR²⁰—, —NCO₂R²⁰—, —CR²¹CO₂R²⁰—, —CR²¹OCOR²⁰—, —CO—,
where R²⁰ is selected from: hydrogen, C₁₋₆ alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl,
where R²¹ and R²² are independently selected from: hydrogen, hydroxy, C₁₋₆ alkyl, —O—C₁₋₆alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl;

R¹ is selected from:
—C₁₋₆alkyl, —C₀₋₆alkyl-O—C₁₋₆alkyl-, —C₀₋₆alkyl-S—C₁₋₆alkyl-,
—(C₀₋₆alkyl)-(C₃₋₇cycloalkyl)-(C₀₋₆alkyl), hydroxy, —CO₂R²⁰, heterocycle,
—CN, —NR²⁰R²⁶—, —NSO₂R²⁰—, —NCOR²⁰—, —NCO₂R²⁰—, —NCOR²⁰—,
—CR²¹CO₂R²⁰—, —CR²¹OCOR²⁰—, phenyl and pyridyl,
where R²⁶ is selected from: hydrogen, C₁₋₆ alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl
where the alkyl and the cycloalkyl are unsubstituted or substituted with 1-7 substituents where the substituents are independently selected from:
   (a) halo,
   (b) hydroxy,
   (c) —O—C₁₋₃-alkyl,
   (d) trifluoromethyl,
   (f) C₁₋₃alkyl,
   (g) —O—C₁₋₃alkyl,
   (h) —CO₂R²⁰,
   (i) —SO₂R²⁰,
   (j) —NHCOCH₃,
   (k) —NHSO₂CH₃,
   (l) -heterocycle,
   (m) ═O,
   (n) —CN,
and where the phenyl and pyridyl are unsubstituted or substituted with 1-3 substituents where the substituents are independently selected from: halo, hydroxy, C₁₋₃alkyl, C₁₋₃alkoxy and trifluoromethyl;

R² is selected from:
(a) hydrogen,
(b) C₁₋₆alkyl,
(c) trifluoromethyl,
(d) trifluoromethoxy,
(e) chloro,
(f) bromo, and
(g) phenyl;

R³ is selected from:
(a) hydrogen,
(b) hydroxy,
(c) halo,
(d) C₁₋₆alkyl,
(e) —O—C₁₋₆alkyl,
(f) —NR²⁰R²¹,
(g) —NR²⁰CO₂R²¹,
(h) —NR²⁰CONR²⁰R²¹,
(i) —NR²⁰—SO₂—NR²⁰R²¹,
(j) —NR²⁰—SO₂—R²¹,
(k) heterocycle,
(l) —CN,
(m) —CONR²⁰R²¹,
(n) —CO₂R²⁰,
(o) —NO₂,
(p) —S—R²⁰,
(q) —SO—R²⁰,
(r) —SO₂—R²⁰, and
(s) —SO₂—NR²⁰R²¹;

R⁴ is selected from:
(a) hydrogen,
(b) C₁₋₆alkyl,
(c) trifluoromethyl,
(d) trifluoromethoxy,
(e) chloro,
(f) bromo, and
(g) phenyl;

R⁵ is selected from:
(a) C₁₋₆alkyl substituted with 1-6 fluoro and optionally substituted with hydroxyl,
(b) —O—C₁₋₆alkyl substituted with 1-6 fluoro,
(c) —CO—C₁₋₆alkyl substituted with 1-6 fluoro,
(d) —S—C₁₋₆alkyl,
(e) -pyridyl,
(f) fluoro,
(g) chloro,
(h) bromo, and
(i) phenyl;

R⁶ is selected from:
(a) hydrogen,
(b) C₁₋₆alkyl,
(c) trifluoromethyl,
(d) trifluoromethoxy,
(e) chloro,
(f) bromo, and
(g) phenyl;

R⁷ is selected from:
(a) hydrogen,
(b) C₁₋₆alkyl, and
(c) trifluoromethyl;

R⁸ is selected from:
(a) hydrogen,
(b) C₁₋₆alkyl, where alkyl may be unsubstituted or substituted with 1-6 substituents where the substituents are chosen from the group: fluoro, C₁₋₃alkoxy, hydroxy, —CO₂R²⁰,
(c) fluoro,
(d) —O—C₁₋₃alkyl, where alkyl may be unsubstituted or substituted with 1-3 fluoro, and
(e) C₃₋₆ cycloalkyl,
(f) —O—C₃₋₆cycloalkyl,
(g) hydroxy,
(h) —CO₂R²⁰,
(i) —OCOR²⁰,
or R⁷ and R⁸ may be joined together via a C₂₋₄alkyl or a
C₀₋₂alkyl-O—C₁₋₃alkyl chain to form a 5-7 membered ring;

R⁹ is selected from:
(a) hydrogen,
(b) C₁₋₆alkyl, where alkyl may be unsubstituted or substituted with 1-6 substituents where the substituents are chosen from the group: fluoro, C₁₋₃alkoxy, hydroxy, —CO₂R²⁰,
(c) CO₂R²⁰,
(d) hydroxy, and
(e) —O—C₁₋₆alkyl, where alkyl may be unsubstituted or substituted with 1-6 substituents where the substituents are chosen from the group: fluoro, C₁₋₃alkoxy, hydroxy, —CO₂R²⁰,
or R⁸ and R⁹ may be joined together by a C₁₋₄alkyl chain or a C₀₋₃alkyl-O—C₀₋₃alkyl chain to form a 3-6 membered ring;

R¹⁰ is selected from:
(a) hydrogen, and

(b) C₁₋₆alkyl,
or R⁸ and R¹⁰ may be joined together by a C₂₋₃alkyl chain to form a 5-6 membered ring;
(a) hydrogen, and
(b) C₁₋₆alkyl, where alkyl may be unsubstituted or substituted with 1-6 fluoro,
(c) fluoro,
(d) —O—C₃₋₆Cycloalkyl, and
(e) —O—C₁₋₃alkyl, where alkyl may be unsubstituted or substituted with 1-6 fluoro,
or R⁸ and R¹⁰ may be joined together by a C₂₋₃alkyl chain to form a 5-6 membered ring, where the alkyl are unsubstituted or substituted with 1-3 substituents where the substiuents are independently selected from: halo, hydroxy, —CO₂R²⁰, C₁₋₃alkyl, and C₁₋₃alkoxy,
or R⁸ and R¹⁰ may be joined together by a C₁₋₂alkyl-O—C₁₋₂alkyl chain to form a 6-8 membered ring, where the alkyl are unsubstituted or substituted with 1-3 substituents where the substiuents are independently selected from: halo, hydroxy, —CO₂R²⁰, C₁₋₃alkyl, and C₁₋₃alkoxy,
or R⁸ and R¹⁰ may be joined together by a O-C₁₋₂alkyl-O-chain to form a 6-7 membered ring, where the alkyl are unsubstituted or substituted with 1-3 substituents where the substiuents are independently selected from: halo, hydroxy, —CO₂R²⁰, C₁₋₃alkyl, and C₁₋₃alkoxy;

R¹¹ is selected from:
(a) hydrogen,
(b) C₁₋₆alkyl, and
(c) trifluoromethyl;

n is selected from 0, 1 and 2;

the dashed line represents a single or a double bond;

and pharmaceutically acceptable salts thereof and individual diastereomers thereof.

### Formula VIII Compounds

### EXAMPLES

Examples of the compounds of Formula VIII include the following:

### Example VIII-1

### L-059471, L-059730, L-059,731

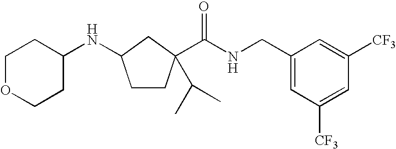

### Example VIII-2

### L-059501, L-059695, L-059696

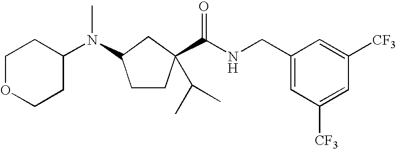

### Example VIII-3

### L-059675

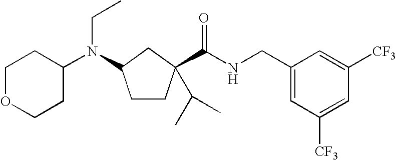

### Example VIII-4

### L-059708

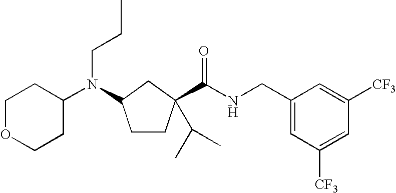

### Example VIII-5

### L-059709

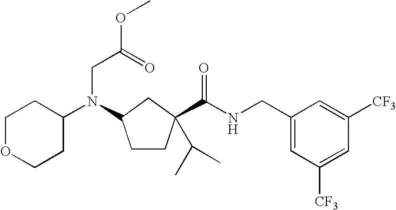

### Example VIII-6

### L-059707

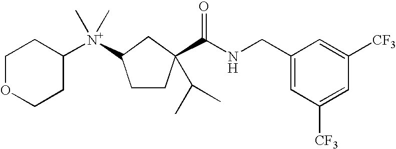

### Example VIII-7

### L-059724

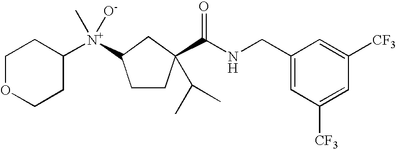

### Example VIII-8

### L-059676

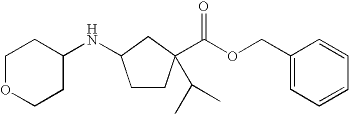

### Example VIII-9

### L-059944

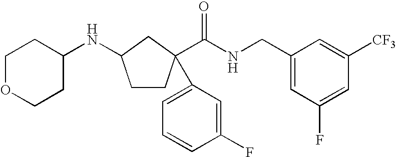

### Example VIII-10

### L-059946

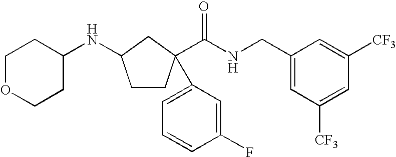

### Examples VIII-11 to VIII-18

Examples VIII-11 through VIII-18, in Table 24, below, are based on the Formula:

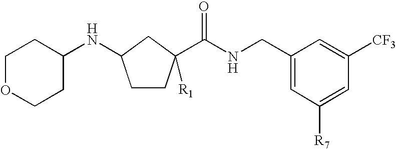

| | | | | | Found |
|---|---|---|---|---|---|
| Ex- | | | Molecular | Calculated | [M + |
| ample | R₁ | R₇ | Formula | [M + H⁺] | H⁺] |
| VIII-11 L- 059948 | 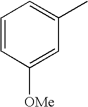 | F | C₂₆H₃₁F₄N₂O₃ | 495.22 | 495.22 |
| VIII-12 L- 059950 | 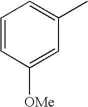 | CF₃ | C₂₇H₃₁F₆N₂O₃ | 545.22 | 545.20 |
| VIII-13 L- 070139 | 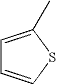 | F | C₂₃H₂₇F₄N₂O₂S | 471.17 | 471.25 |
| VIII-14 L- 070141 | 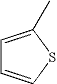 | CF₃ | C₂₇H₃₁F₆N₂O₂S | 521.16 | 521.15 |
| VIII-15 L- 070143 | 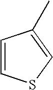 | F | C₂₃H₂₇F₄N₂O₂S | 471.17 | |
| VIII-16 L- 070145 | 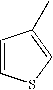 | CF₃ | C₂₄H₂₇F₆N₂O₂S | 521.16 | 521.20 |
| VIII-17 L- 059952 | 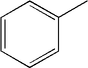 | F | C₂₅H₂₉F₆N₂O₂ | 465.21 | 465.25 |
| VIII-18 L- 059954 | 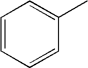 | CF₃ | C₂₆H₂₉F₆N₂O₂ | 515.21 | 515.20 |

### Example VIII-19

### L-070392

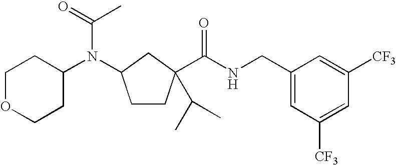

### Example VIII-20

### EX 13: L-070208

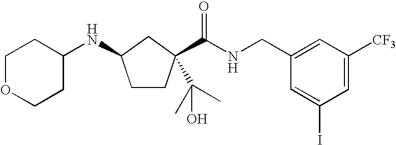

### Example VIII-21 to VIII-37

### Examples VIII-21 through VII-37, in Table 25, below, are based on the Formula:

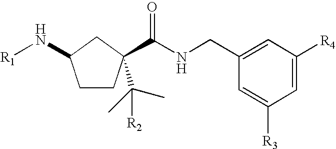

| Ex. | R1 | R2 | R3 | R4 | Molecular Formula | Calc'd [M⁺H⁺] | Found [M⁺H⁺] |
|---|---|---|---|---|---|---|---|
| VIII-21 L-070209 | 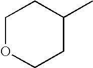 | OH | Cl | CF₃ | C₂₂H₃₁ClF₃N₂O₃ | 463.19 | 463.15 |
| VIII-22 L-070328 | 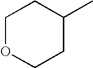 | OH | H | Ph | C₂₇H₃₇N₂O₃ | 437.27 | 437.35 |
| VIII-23 L-070329 | 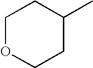 | OH | H | OCF₃ | C₂₂H₃₂F₂N₂O₄ | 445.22 | 445.3 |
| VIII-24 L-070330 | 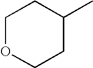 | OH | H | 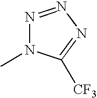 | C₂₂H₃₂F₃N₆O₃ | 497.24 | 497.2 |
| VIII-25 L-070331 | 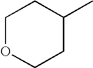 | OH | F | CF₃ | C₂₂H₃₁F₄N₂O₃ | 447.22 | 445.25 |
| VIII-26 L-070332 | 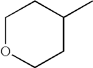 | OH | Cl | Cl | C₂₁H₃₁Cl₂N₂O₃ | 429.16 | 429.25 |
| VIII-27 L-070619 L-070620 | 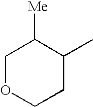 | OH | F | CF₃ | C₂₃H₃₃F₄N₂O₃ | 461.23 | 461.25 |
| VIII-28 L-070718 | 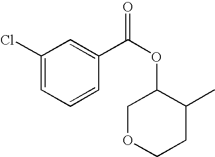 | OH | F | CF₃ | C₂₉H_{34Cl}F₄N₂O₅ | 601.20 | 601.3 |
| VIII-29 L-070719 | 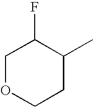 | OH | F | CF₃ | C₂₂H₃₀F₅N₂O₃ | 465.21 | 465.25 |
| VIII-30 L-070721 L-070803 L-070804 | 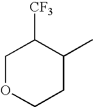 | OH | F | CF₃ | C₂₃H₃₀F₇N₂O₃ | 515.21 | 515.2 |
| VIII-31 L-070754 | 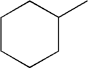 | OH | F | CF₃ | C₂₃H₃₃F₄N₂O₄ | 445.24 | 445.3 |
| VIII-32 L-070762 L-070768 L-070777 | 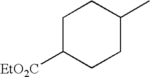 | H | CF₃ | CF₃ | C₂₇H₃₇F₆N₂O₃ | 551.26 | 551.35 |
| VIII-33 L-070769 | 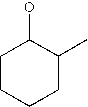 | H | CF₃ | CF₃ | C₂₄H₃₃F₆N₂O₂ | 495.24 | 495.25 |
| VIII-34 L-070705 | 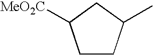 | H | CF₃ | CF₃ | C₂₅H₃₃F₆N₂O₃ | 523.23 | 523.3 |
| VIII-35 | 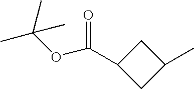 | H | CF₃ | CF₃ | C₂₇H₃₇F₆N₂O₃ | 551.26 | 551.2 |
| VIII-36 L-070813 | 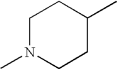 | H | CF₃ | CF₃ | C₂₄H₃₄F₆N₃O | 494.25 | 494.3 |
| VIII-37 L-070814 | 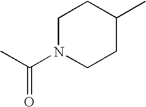 | H | CF₃ | CF₃ | C₂₅H₃₄F₆N₃O₂ | 522.25 | 522.25 |

### Example VIII-38

### L-070802

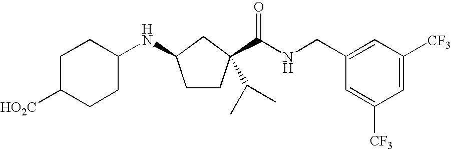

### Example VIII-39

### L-070847

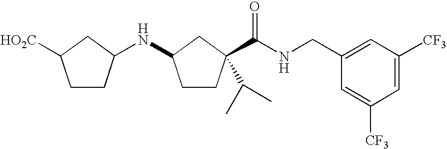

### Example VIII-40

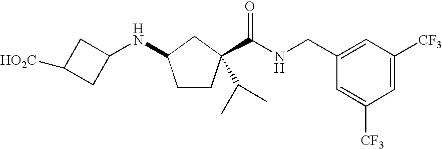

### Example VIII-41

### L-070882

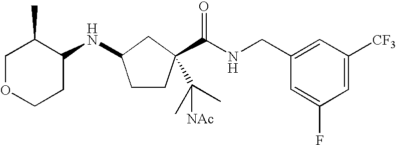

### Example VIII-42

### L-070333, L-070334, L-070335

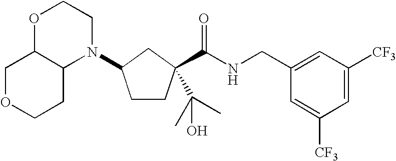

### Example VIII-43

### L070235

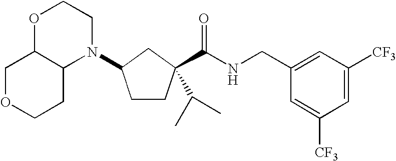

### Example VIII-44

### L-070658

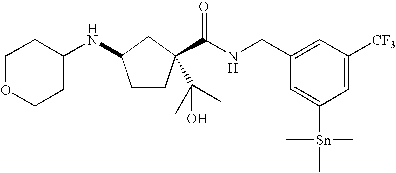

### Example VIII-45

### L-070659

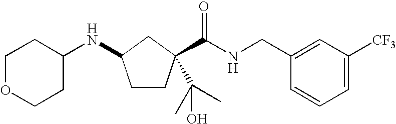

### Example VIII-46

### L-070725

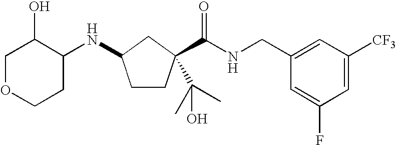

### Example VIII-47

### L-070671

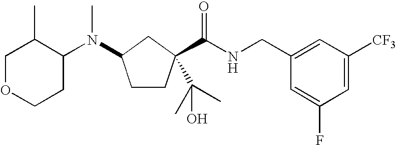

### Example VIII-48

### L-070706, L-070707, L-070708

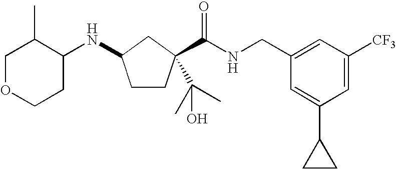

### Example VIII-49

### L-070572

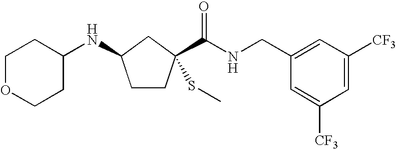

### Example VIII-50

### L-070576, L-070577

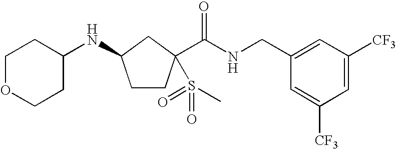

### Example VIII-51

### L-070616

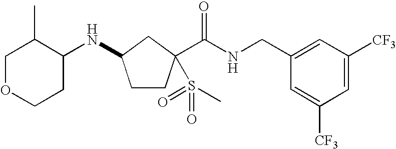

### Example VIII-52

### L-070621

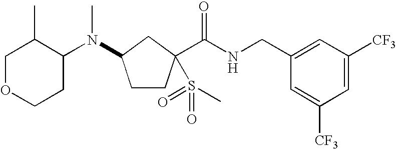

### Example VIII-53

### L-070687, L-070688

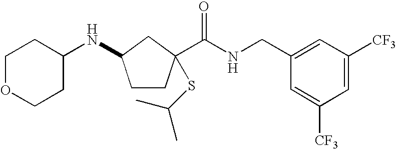

### Example VIII-54

### L-070689, L-070690

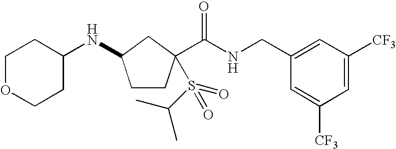

### Example VIII-55

### L-070669

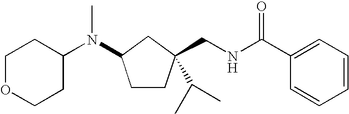

### Examples VIII-56 to VIII-61

Examples VIII-56 through VIII-61, in Table 26, below, are based on the Formula:

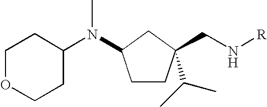

| | | | Cal- | |
|---|---|---|---|---|
| | | Molecular | culated | Found |
| Ex. | R | Formula | [M⁺H⁺] | [M⁺H⁺] |
| VIII-56 L-070970 | 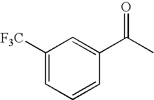 | C₂₃H₃₃F₃N₂O₂ | 427.25 | 427.3 |
| VIII-57 L-070971 | 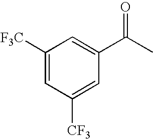 | C₂₄H₃₂F₆N₂O₂ | 495.24 | 495.25 |
| VIII-58 L-070972 | 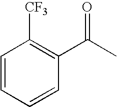 | C₂₃H₃₃F₃N₂O₂ | 427.25 | 427.3 |
| VIII-59 L-070973 | 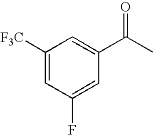 | C₂₃H₃₂F₄N₂O₂ | 445.24 | 445.3 |
| VIII-60 L-070974 | 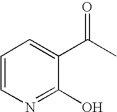 | C₂₁H₃₃N₃O₃ | 376.25 | 376.3 |
| VIII-61 L-070975 | 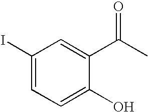 | C₂₂H₃₃IN₂O₃ | 501.15 | 501.25 |

### Example VIII-62

### L-070976

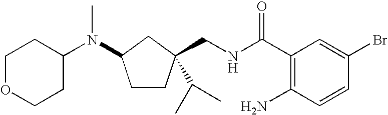

### Example VIII-63

### L-059959, L-059960

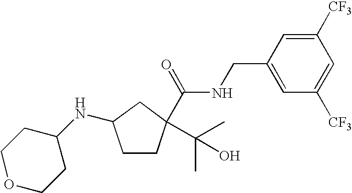

### Example VIII-64

### L-059980

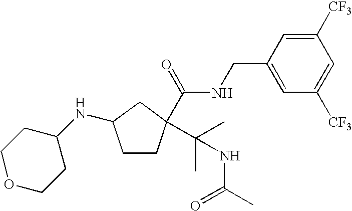

### Example VIII-65

### L-070151, L-070152, L-070153, L-070154, L-070155, L-070156

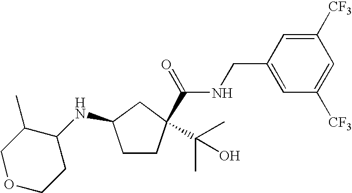

### Example VIII-66

### L-070506

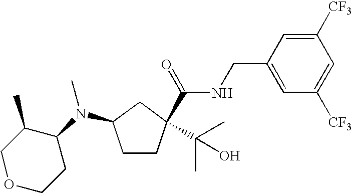

### Example VIII-67

### L-070716

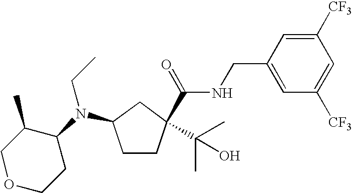

### Example VIII-68

### L-070758

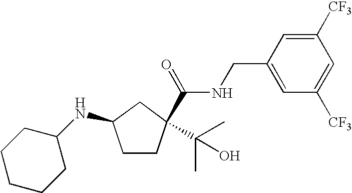

### Example VIII-69

### L-070763, L-070764, L-070765

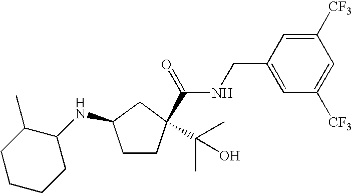

### Example VIII-70

### L-070798

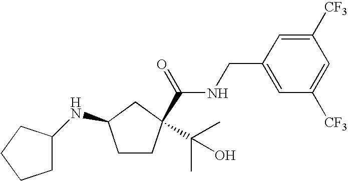

### Example VIII-71

### L-070423

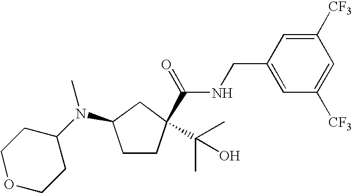

### Example VIII-72

### L-070343, L-070344

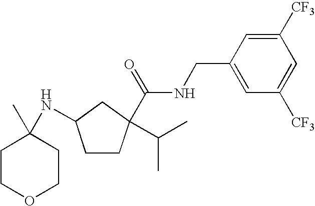

### Example VIII-73

### L-070345, L-070346, L-070347

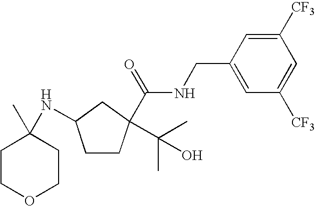

### Example VIII-74

### L-070373

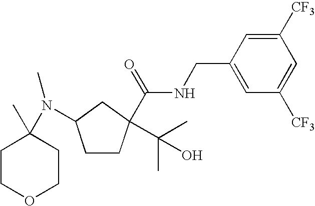

### Example VIII-75

### L-059442, L-059441

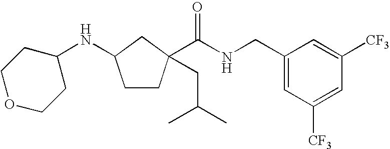

### Example VIII-76

### L-070046, L-070093, L-070094

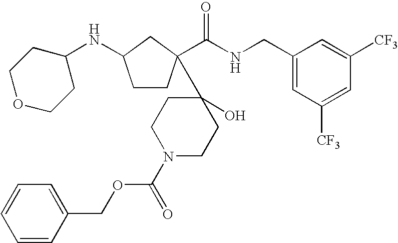

### Example VIII-77

### L-070150

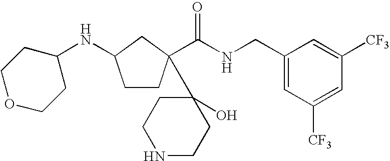

### Example VIII-78

### L-070091, L-070092

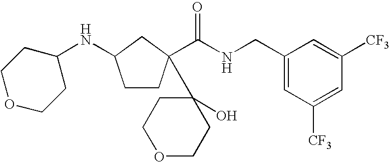

### Example VIII-79

### L-070135

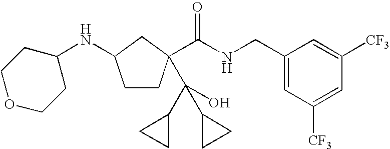

### Example VIII-80

### L-070095

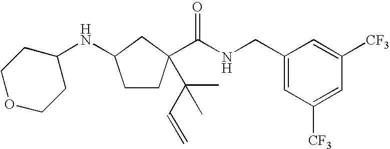

### Example VIII-81

### L-070175, L-070176, L-070177, L-070178

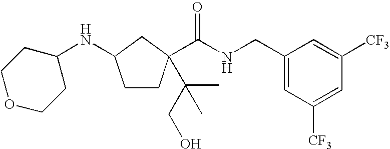

### Example VIII-82

### L-070214

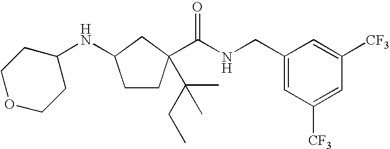

### Example VIII-83

### L-070908

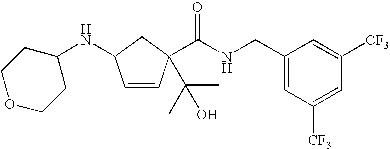

### Example VIII-84

### L-070910

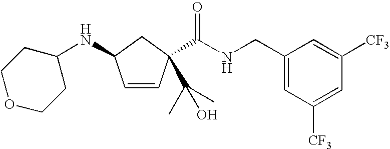

### Example VIII-85

### L-070909, L-070921

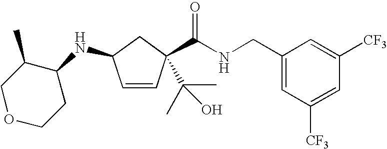

### Example VIII-86

### L-070888, L-070889, L-070917

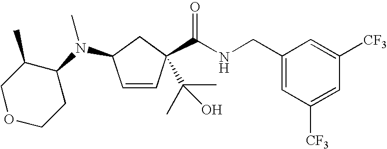

### Example VIII-87

### L-070072, L-070073

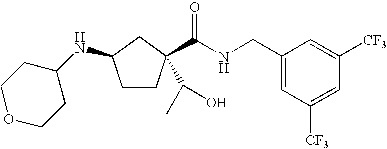

### Example VIII-88

### L-070740,L-070741

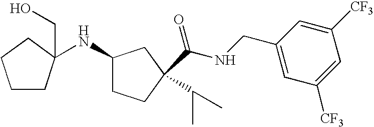

### Example VIII-89

### L-070672

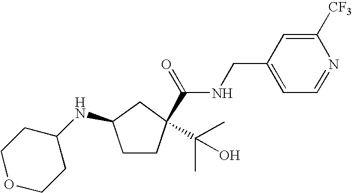

### Example VIII-90

### L-070048

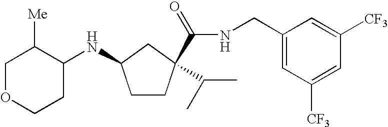

### Examples VIII-90 to 131

Examples VIII-90 through VIII-131, in Table 27, below, are based on the Formula:

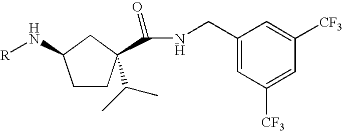

| | | | Cal- | |
|---|---|---|---|---|
| | | | culated | |
| | | Molecular | [M + | Found |
| Ex. | R | Formula | H⁺] | [M + H⁺] |
| VIII-90 L-070048 | 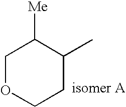 | C₂₄H₃₂F₆N₂O₂ | 495.24 | 495.30 |
| VIII-91 L-070049 | 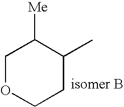 | C₂₇H₃₁F₆N₂O₂ | 495.24 | 495.30 |
| VIII-92 L-070050 | 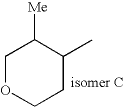 | C₂₄H₃₂F₆N₂O₂ | 495.24 | 495.30 |
| VIII-93 L-070637 | 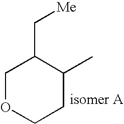 | C₂₅H₃₄F₆N₂O₂ | 509.26 | 509.40 |
| VIII-94 L-070638 | 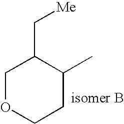 | C₂₅H₃₄F₆N₂O₂ | 509.26 | 509.40 |
| VIII-95 L-070639 | 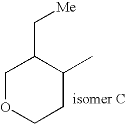 | C₂₅H₃₄F₆N₂O₂ | 509.26 | 509.40 |
| VIII-96 L-070404 | 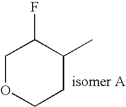 | C₂₃H₂₉F₇N₂O₂ | 499.22 | 499.20 |
| VIII-97 L-070405 | 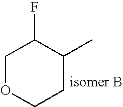 | C₂₃H₂₉F₇N₂O₂ | 499.22 | 499.20 |
| VIII-98 L-070406 | 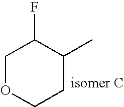 | C₂₃H₂₉F₇N₂O₂ | 499.22 | 499.20 |
| VIII-99 L-070531 | 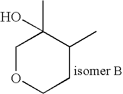 | C₂₄H₂₉F₉N₂O₃ | 565.21 | 565.30 |
| VIII-100 L-070530 | 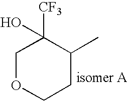 | C₂₄H₂₉F₉N₂O₃ | 565.21 | 565.30 |
| VIII-101 L-070406 | 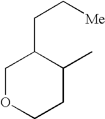 | C₂₆H₃₆F₆N₂O₂ | 523.28 | 523.30 |
| VIII-102 L-070297 | 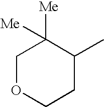 | C₂₅H₃₄F₆N₂O₃ | 509.26 | 509.20 |
| VIII-103 L-070338 | 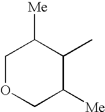 | C₂₅H₃₄F₉N₂O₂ | 509.26 | 509.20 |
| VIII-104 L-070608 | 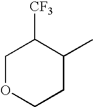 | C₂₄H₂₉F₉N₂O₃ | 549.26 | 549.40 |
| VIII-105 L-070534 | 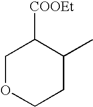 | C₂₆H₃₄F₆N₂O₄ | 553.25 | 553.40 |
| VIII-106 L-070607 | 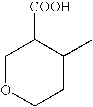 | C₂₆H₃₄F₆N₂O₄ | 525.22 | 525.30 |
| VIII-107 L-070110 | 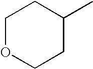 | C₂₃H₃₀F₆N₂O₂ | 481.23 | 481.20 |
| VIII-108 L-070024 | 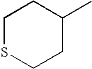 | C₂₃H₃₀F₆N₂OS | 497.21 | 497.20 |
| VIII-109 L-070109 | 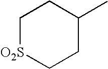 | C₂₃H₃₀F₆N₂O₃S | 529.20 | 529.20 |
| VIII-110 L-070660 | 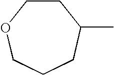 | C₂₄H₃₂F₆N₂O₂ | 495.24 | 495.30 |
| VIII-112 L-070025 | 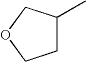 | C₂₂H₂₉F₆N₂O₂ | 497.21 | 467.20 |
| VIII-113 L-070372 | 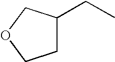 | C₂₂H₂₉F₆N₂O₂ | 467.21 | 467.20 |
| VIII-114 L-070110 | 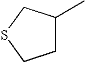 | C₂₂H₃₀F₆N₂OS | 483.19 | 483.20 |
| VIII-115 L-238096 | 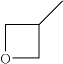 | C₂₁H₂₆F₆N₂O₂ | 453.20 | 453.15 |
| VIII-116 L-070191 | 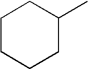 | C₂₄H₃₂F₆N₂O | 479.25 | 479.30 |
| VIII-117 L-070064 | 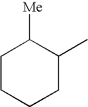 | C₂₅H₃₄F₆N₂O | 493.27 | 493.30 |
| VIII-118 L-070190 | 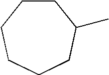 | C₂₅H₃₄F₆N₂O | 493.27 | 493.30 |
| VIII-119 L-070193 | 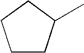 | C₂₃H₃₀F₆N₂O | 453.20 | 453.15 |
| VIII-120 L-070194 | 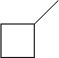 | C₂₂H₂₈F₆N₂O | 451.22 | 451.30 |
| VIII-121 L-070295 | 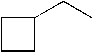 | C₂₃H₃₀F₆N₂O | 465.23 | 465.30 |
| VIII-122 L-070027 | 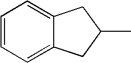 | C₂₇H₃₀F₆N₂O | 513.23 | 513.30 |
| VIII-123 L-872374 | 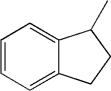 | C₂₇H₃₀F₆N₂O | 513.23 | 513.40 |
| VIII-124 L-872371 | 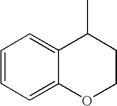 | C₂₇H₃₀F₆N₂O₂ | 529.23 | 529.30 |
| VIII-125 L-872372 | 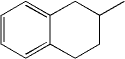 | C₂₈H₃₂F₆N₂O | 527.25 | 527.30 |
| VIII-126 L-070192 | 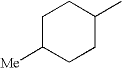 | C₂₅H₃₄F₆N₂O | 493.27 | 493.30 |
| VIII-127 L-070685 | 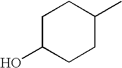 | C₂₄H₃₂F₆N₂O₂ | 495.24 | 495.40 |
| VIII-128 L-070003 | 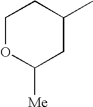 | C₂₄H₃₂F₆N₂O₂ | 495.24 | 495.40 |
| VIII-129 L-070498 | 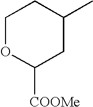 | C₂₅H₃₂F₆N₂O₄ | 539.23 | 539.30 |
| VIII-130 L-070900 | 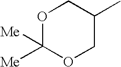 | C₂₄H₃₂F₆N₂O₃ | 511.24 | 511.30 |
| VIII-131 L-070161 | 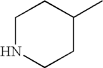 | C₂₃H₃₂F₆N₂O | 480.24 | 480.30 |

### Examples VIII-132 to 140

Examples VIII-132 through VIII-140, in Table 28, below, are based on the Formula:

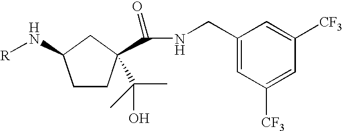

| | | | Cal- | |
|---|---|---|---|---|
| | | | culated | |
| | | Molecular | [M + | Found |
| Ex. | R | Formula | H⁺] | [M + H⁺] |
| VIII-132 L-070682 | 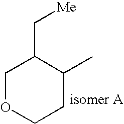 | C₂₅H₃₄F₆N₂O₃ | 525.26 | 525.40 |
| VIII-133 L-070683 | 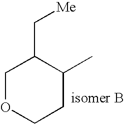 | C₂₅H₃₄F₆N₂O₃ | 525.26 | 525.40 |
| VIII-134 L-070482 | 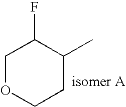 | C₂₃H₂₉F₇N₂O₃ | 515.21 | 515.40 |
| VIII-135 L-070483 | 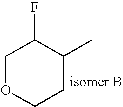 | C₂₃H₂₉F₇N₂O₃ | 515.21 | 515.40 |
| VIII-136 L-070784 | 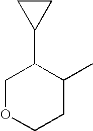 | C₂₆H₃₄F₆N₂O₃ | 537.26 | 537.40 |
| VIII-137 L-070895 | 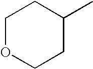 | C₂₃H₃₀F₆N₂O₃ | 497.22 | 497.20 |
| VIII-138 L-070785 | 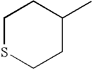 | C₂₃H₃₀F₆N₂O₂S | 513.20 | 513.20 |
| VIII-139 L-070901 | 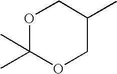 | C₂₄H₃₂F₆N₂O₄ | 527.23 | — |
| VIII-140 L-070818 | 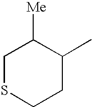 | C₂₄H₃₂F₆N₂O₂S | 527.22 | 527.40 |

### Examples VIII-141 to 144

Examples VIII-141 through VIII-144, in Table 29, below, are based on the Formula:

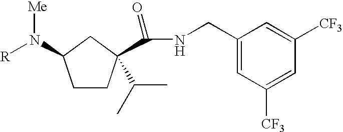

| | | Molecular | Calculated | Found |
|---|---|---|---|---|
| Ex. | R | Formula | [M + H⁺] | [M + H⁺] |
| VIII-141 L-070293 | 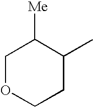 | C₂₅H₃₄F₆N₂O₂ | 509.26 | 509.30 |
| VIII-142 L-070296 | 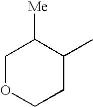 | C₂₅H₃₄F₆N₂O₂ | 509.26 | 509.30 |
| VIII-143 L-070571 | 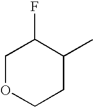 | C₂₄H₃₁F₇N₂O₂ | 513.24 | 513.30 |
| VIII-144 L-070570 | 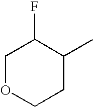 | C₂₄H₃₁F₇N₂O₂ | 513.24 | 513.30 |

### Example VIII-145

### L-070727

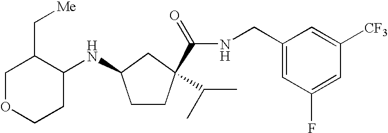

### Example VIII-146

### L-251768

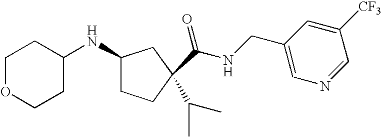

### Example VIII-147

### L-260857, L-260858, L-260860, L-260862, L-251769

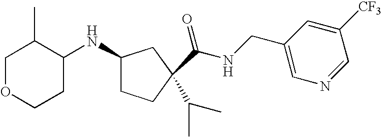

### Example VIII-148

### L-260225

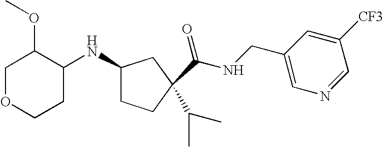

### Example VIII-149

### L-070673

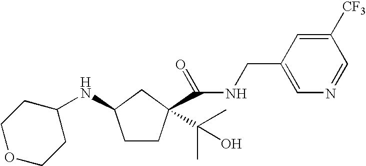

### Example VIII-150

### L-070196, L-070197, L-070198

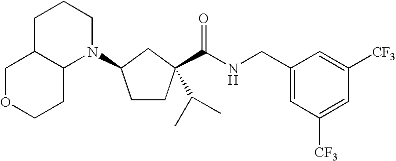

### Example VIII-151

### L-070215, L-070216, L-070217, L-070218

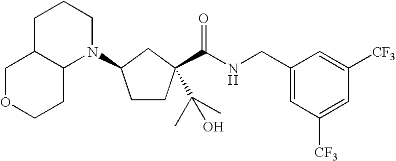

### Example VIII-152

### L-070183, L-070184

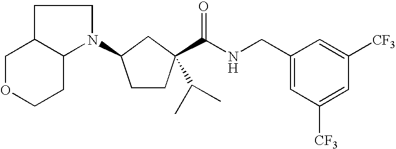

### Example VIII-153

### L-070258, L-070259

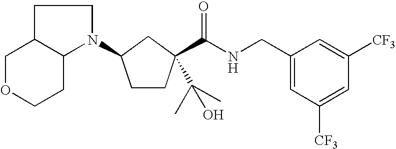

### Example VIII-154

### L-070717, L-070712

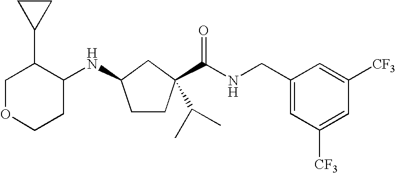

### Example VIII-155

### L-059847

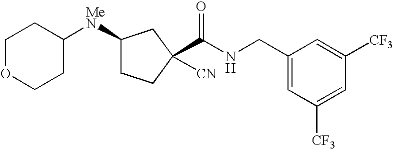

### Example VIII-156

### L-059961

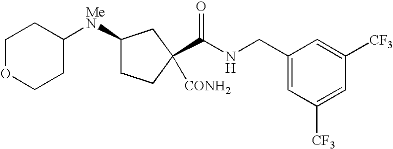

### Example VIII-157

### L-059963

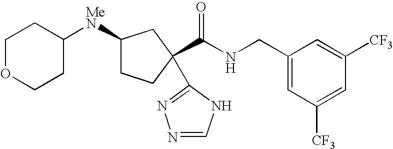

### Example VIII-158

### L-070023

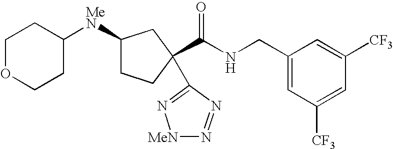

### Example VIII-159

### L-070539

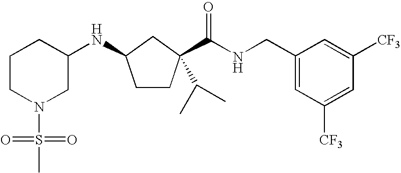

### Example VIII-160

### L-070679, L-070680, L-070681

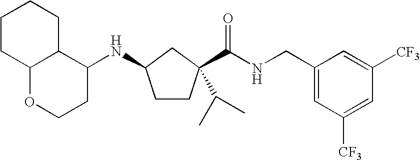

### Example VIII-161

### L-070779

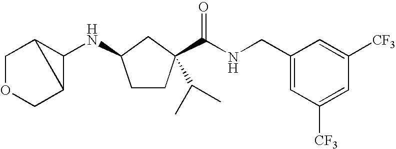

### Example VIII-162

### L-070124, L-070125, L-070199, L-070200, L-070201, L-070202

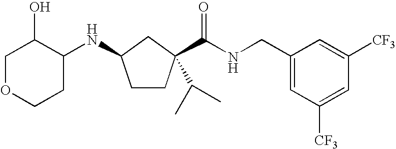

### Example VIII-163

### L-070130

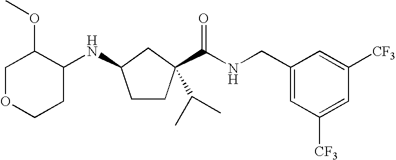

### Example VIII-164

### L-070213, L-070131, L-070132, L-070133

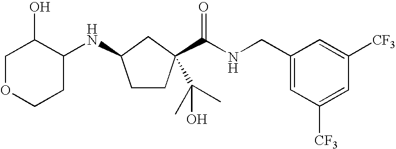

### Example VIII-165

### L-070275, L-070276

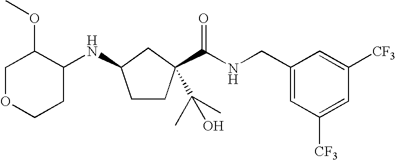

### Example VIII-166

### L-070336

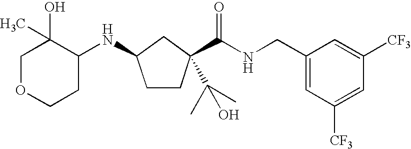

### Example VIII-167

### L-070511

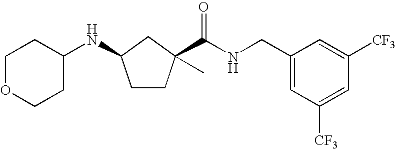

### Example VIII-168

### L-070512

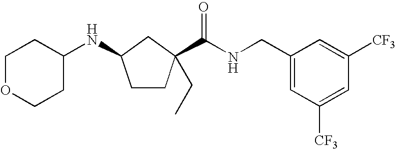

### Example VIII-169

### L-070627, L-070628

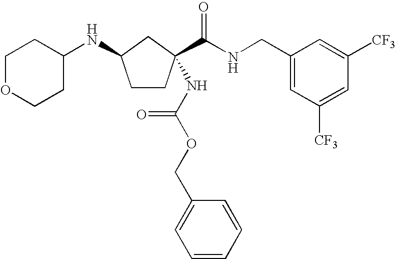

### Example VIII-170

### L-070629, L-070630

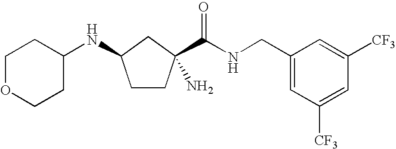

### Example VIII-171

### L-070569, L-070617, L-070618

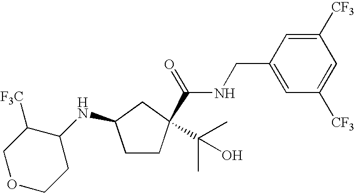

### Example VIII-172

### L-070203, L-070204

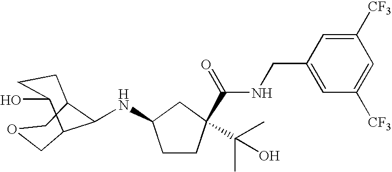

### Example VIII-173

### L-070614

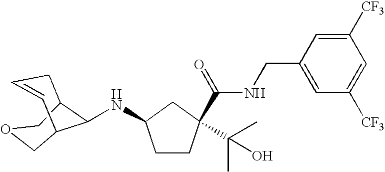

### Example VIII-174

### L-070654, L-070655

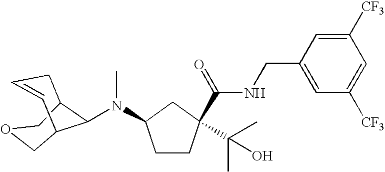

### Example VIII-175

### L-070430, L-070431

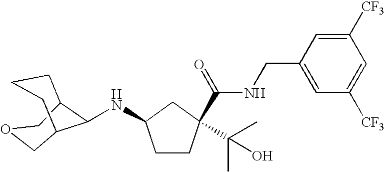

### Example VIII-176

### L-070656, L-070657

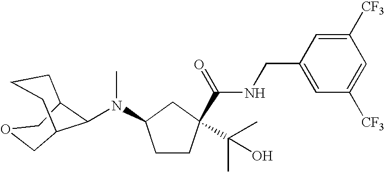

### Example VIII-177

### L-070702, L-070703, L-070704, L-070705

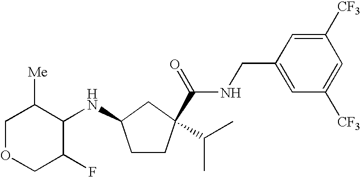

### Example VIII-178

### L-070031, L-070032

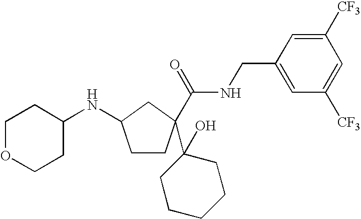

### Example VIII-179

### L-070030, L-070057, L-070058

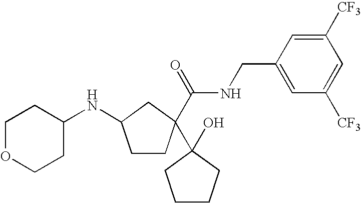

### Example VIII-180

### L-059, 975, L-059997, L-059998, L-07055, L-070056

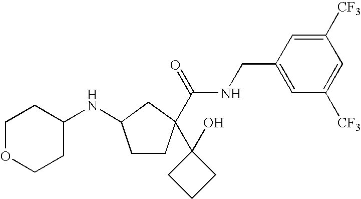

### Example VIII-181

### L-070759, L-070763

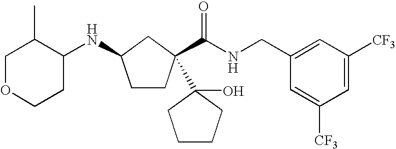

### Example VIII-182

### L-070186, L-070187

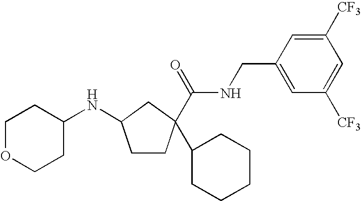

### Example VIII-183

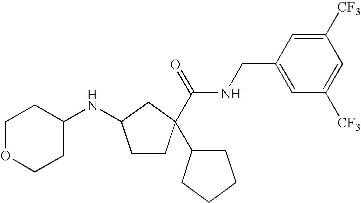

### L-070098, L-070099, L-070105

### Example VIII-184

### L-070134, L-070136, L-070137, L-070120

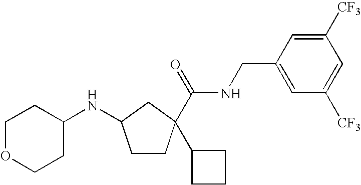

### Example VIII-185

### L-070205, L-070206, L-070207

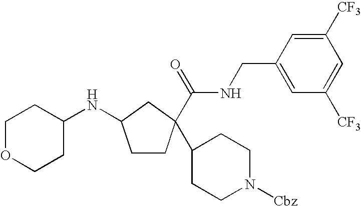

### Example VIII-186

### L-070238

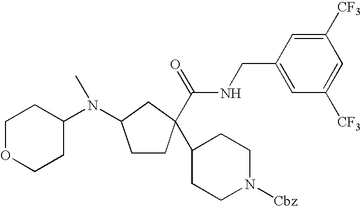

### Example VIII-187

### L-070239

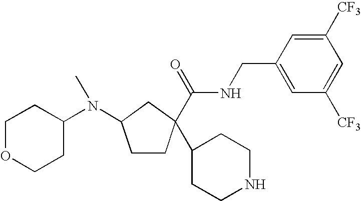

### Example VIII-188

### L-070285

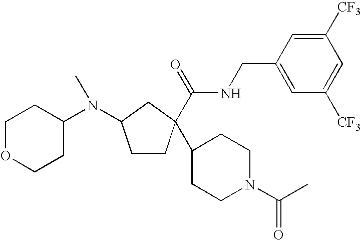

### Example VIII-189

### L-070286

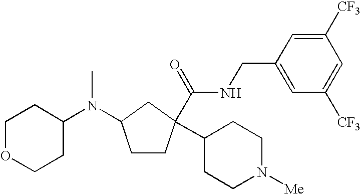

### Example VIII-190

### L-070062

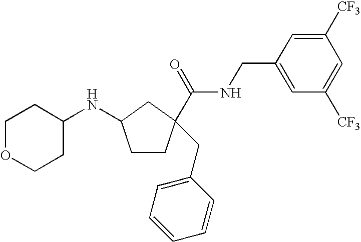

### Example VIII-191

### L-070063

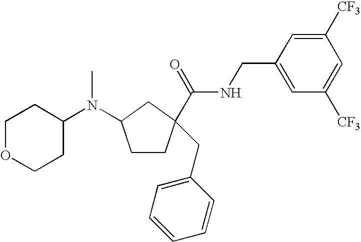

### Example VIII-192

### L-059681

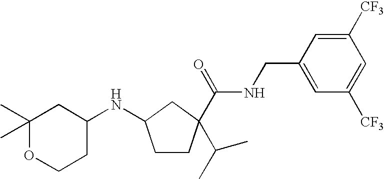

### Example VIII-193

### L-070157

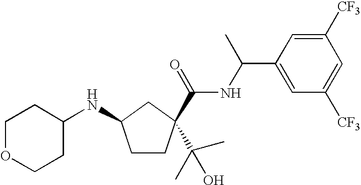

### Example VIII-194

### L-070941

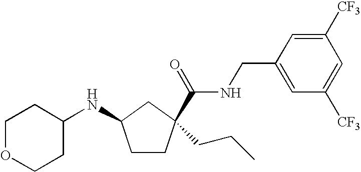

### Example VIII-195

### L-059539, L-059706, L-059723, L-059749, L-059751

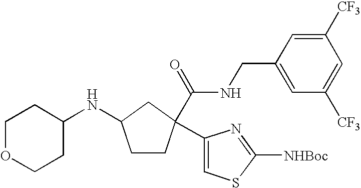

### Example VIII-196

### L-059541

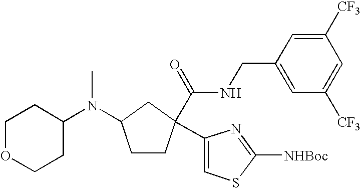

### Example VIII-197

### L-059542, L-059771

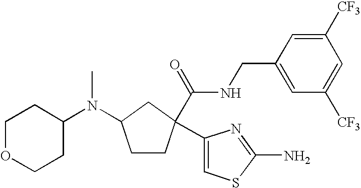

### Example VIII-198

### L-059543, L-059772

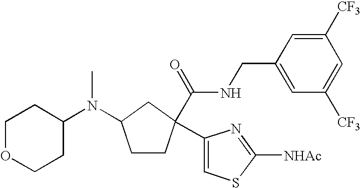

### Example VIII-199

### L-059515

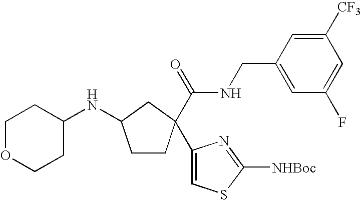

### Example VIII-200

### L-059519

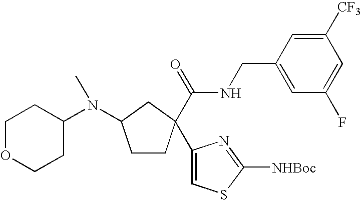

### Example VIII-201

### L-059520

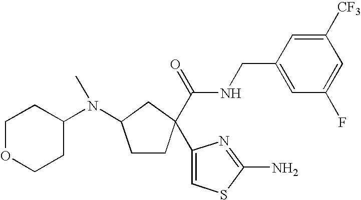

### Example VIII-202

### L-059521

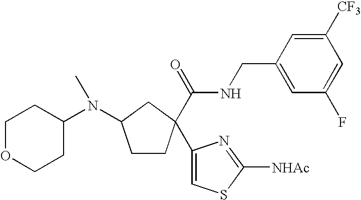

### Example VIII-203

### L-059836, L-059837

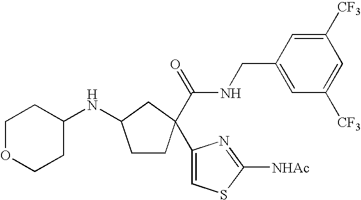

### Example VIII-204

### L-059582

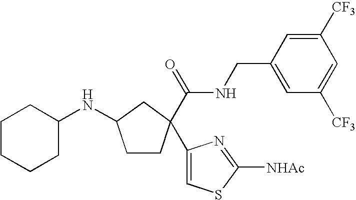

### Example VIII-205

### L-059991, L-059992

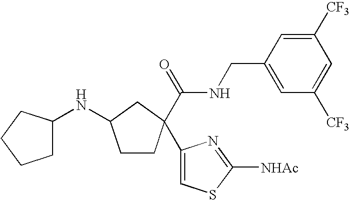

### Example VIII-206

### L-059834, L-059835

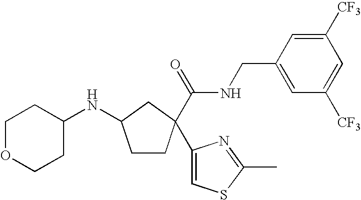

### Example VIII-207

### L-070028

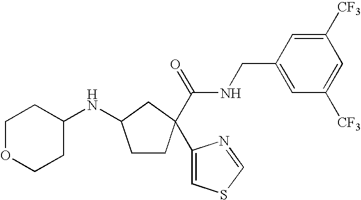

### Example VIII-208

### L-070395

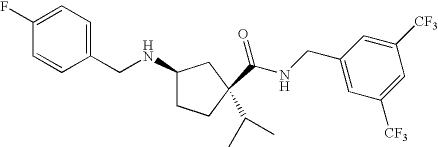

### Examples VIII-209 to 221

Examples VIII-209 through VIII-221, on Table 30, below, are based on the Formula:

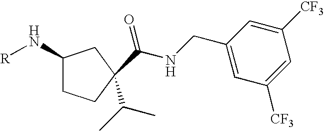

| Ex. | R | Molecular Formula | Calc'd [M + H]⁺ | Found [M + H]⁺ |
|---|---|---|---|---|
| VIII-209 L-070474 | 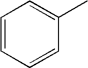 | C₂₅H₂₈F₆N₂O | 487.21 | 487 |
| VIII-210 L-070397 | 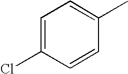 | C₂₅H₂₈ClF₆N₂O | 521.17 | 521 |
| VIII-211 L-070441 | 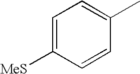 | C₂₆H₃₀F₆N₂OS | 533.20 | 533 |
| VIII-212 L-070442 | 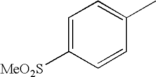 | C₂₆H₃₀F₆N₂O₃S | 565.19 | 565 |
| VIII-213 L-070844 | 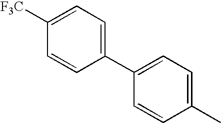 | C₃₂H₃₁F₉N₂O | 631.23 | 631 |
| VIII-214 L-070475 | 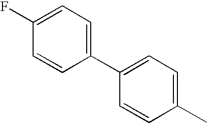 | C₃₁H₃₁F₇N₂O | 481.23 | 581 |
| VIII-215 L-070781 | 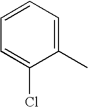 | C₂₅H₂₇ClF₆N₂O | 521.17 | 521 |
| VIII-216 L-070782 | 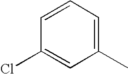 | C₂₅H₂₇ClF₆N₂O | 521.17 | 521 |
| VIII-217 L-070471 | 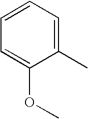 | C₂₆H₃₀F₆N₂O₂ | 517.22 | 517 |
| VIII-218 L-070472 | 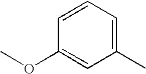 | C₂₆H₃₀F₆N₂O | 501.23 | 517 |
| VIII-219 L-070398 | 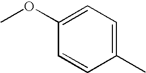 | C₂₆H₃₀F₆N₂O | 501.23 | 517 |
| VIII-220 L-070801 | 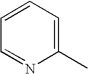 | C₂₄H₂₇F₆N₃O | 488.21 | 488 |
| VIII-221 L-070800 | 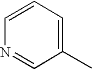 | C₂₄H₂₇F₆N₃O | 488.21 | 488 |

### Example VIII-222

### L-059429

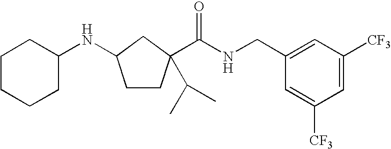

### Example VIII-223

### L-070298

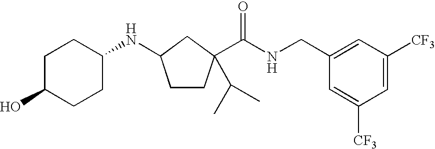

### Example VIII-224

### L-070299

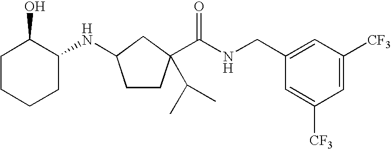

### Example VIII-226

### L-059873

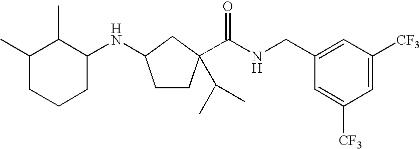

### Example VIII-227

### L-059874

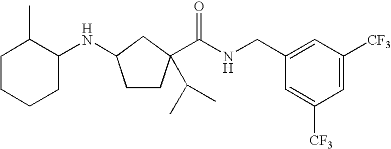

### Example VIII-228

### L-070820

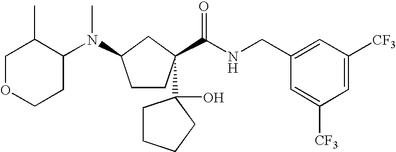

### Example VIII-229

### L-070797

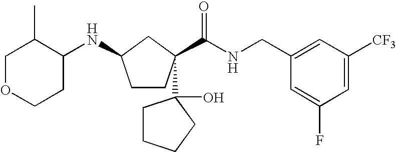

### Example VIII-230

### L-070796

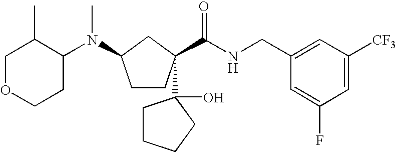

### Example VIII-231

### L-070625, L-070626

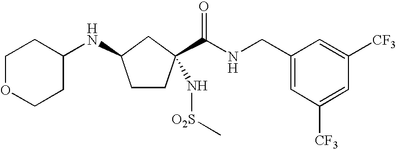

### Example VIII-232

### L-070623, L-070624

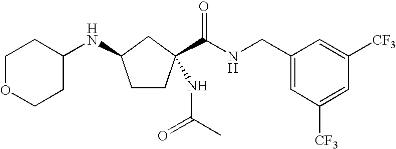

### Example VIII-233

### L-236155

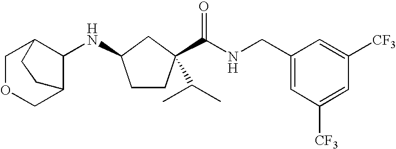

### Example VIII-234

### L-070745

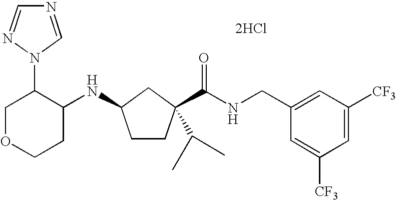

### Example VIII-235

### L-070751

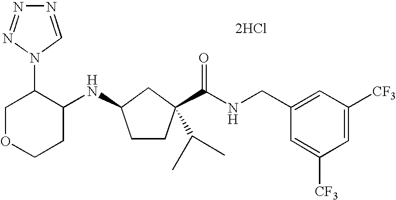

### Example VIII-236

### L-059759, L-059760

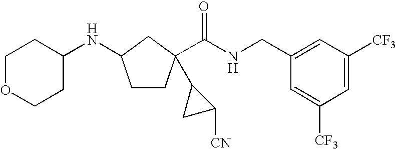

### Example VIII-237

### L-059774

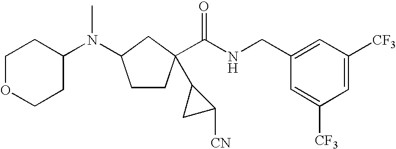

### Example VIII-238

### L-070494, L-070495

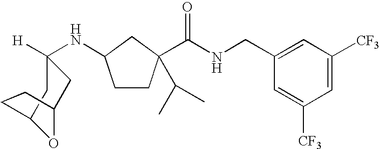

### Example VIII-239

### L-070368

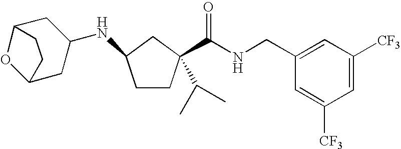

### Example VIII-240

### L-070597

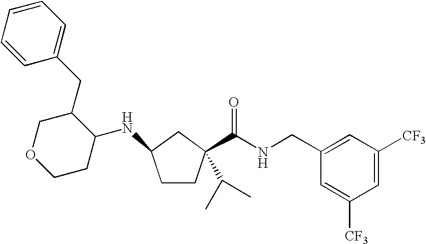

### Example VIII-241

### L-070645, L-070646, L-070647, L-070648

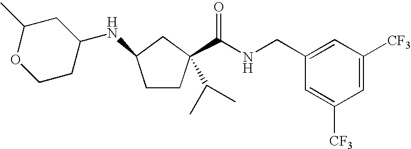

### Example VIII-242

### L-070742, L-070743, L-070653

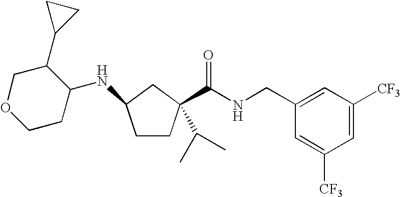

### Example VIII-243

### L-070744

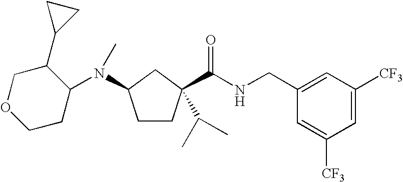

### Example VIII-244

### L-070746

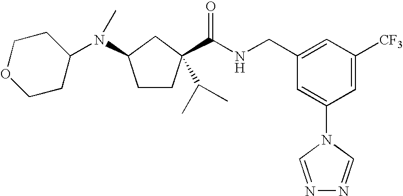

### Example VIII-245

### L-070748

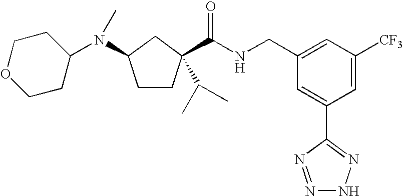

### Example VIII-246

### L-070747

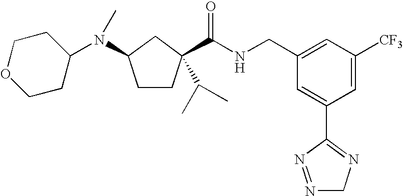

### Example VIII-247

### L-070749

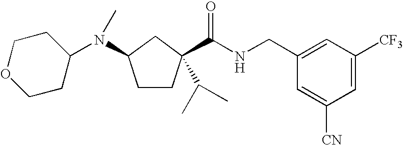

### Example VIII-248

### L-070750

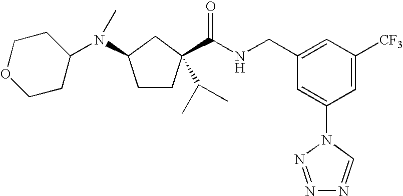

### Example VIII-249

### L-070905

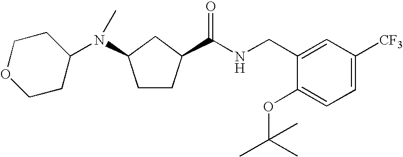

### Example VIII-250

### L-070906

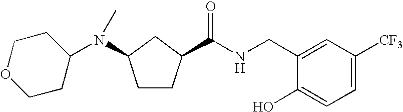

### Example VIII-252

### L-070978

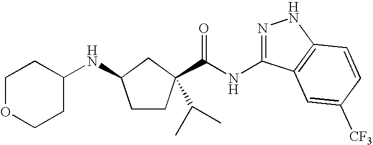

### Example VIII-253

### L-077657

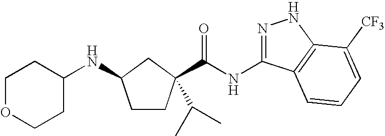

Additional CCR-2 antagonists useful in the methods of the invention include those of Formula IX: 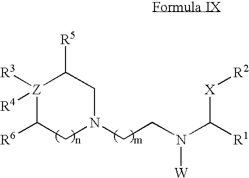
wherein:

X is selected from the group consisting of:
—NR¹⁰—, —O—, —CH₂O—, —CONR¹⁰—, —NR¹⁰CO—, —CO₂—, —OCO—,
—CH₂(NR₁₀)CO—, —N(COR¹⁰)—, —CH₂N(COR¹⁰)—, phenyl, and
C₃₋₆ cycloalkyl,
where R¹⁰ is independently selected from: hydrogen, C₁₋₆ alkyl, benzyl, phenyl, and C₁₋₆ alkyl-C₃₋₆ cycloalkyl,
   which is unsubstituted or substituted with 1-3 substituents where the substituents are independently selected from: halo, C₁₋₃ alkyl,
   C₁₋₃ alkoxy and trifluoromethyl;

W is selected from:
hydrogen and C₁₋₆ alkyl, which is unsubstituted or substituted with 1-3 substituents where the substituents are independently selected from: halo, C₁₋₃alkoxy and trifluoromethyl;

Z is selected from:
C, N, and —O—, wherein when Z is N, then R⁴ is absent, and when W is —O—, then both R³ and R⁴ are absent;

n is an integer selected from 0, 1, 2, 3 and 4;

n is an integer selected from 1, 2, 3 and 4;

R¹ is selected from:
hydrogen, —C₀₋₆alkyl-, —(C₀₋₆alkyl)-alkenyl-, —(C₀₋₆alkyl)-C₃₋₆cycloalkyl, —(C₀₋₆alkyl)-phenyl, and —(C₀₋₆alkyl)-heterocycle,
where the alkyl is unsubstituted or substituted with 1-7 substituents where the substituents are independently selected from:
(a) halo,
(b) hydroxy,
(c) —O—C₁₋₃alkyl,
(d) trifluoromethyl, and
(e) —C₁₋₃alkyl,
and where the phenyl and the heterocycle is unsubstituted or substituted with 1-5 substituents where the substituents are independently selected from:
(a) halo,
(b) hydroxy; alkoxy
(c) amino; acylamino;sulfonylamino; alkoxycarbonylamino
(d) carboxylic acid; carbamide; sulfonamide

or wherein W and R¹ may be joined together to form a ring by a group selected from:
—(C₁₋₆alkyl)-, —C₀₋₆alkyl-Y—(C₁₋₆alkyl)-, and —(C₀₋₆alkyl)-Y—(C₀₋₆alkyl)-(C₃₋₇cycloalkyl)-(C₀₋₆alkyl),
   where Y is selected from:
   a single bond, —O—, —S—, —SO—, —SO₂—, and —NR¹⁰—,
   and where the alkyl and the cycloalkyl are unsubstituted or substituted with 1-7 substituents where the substituents are independently selected from:
   (a) halo,
   (b) hydroxy,
   (c) —O—C₁₋₃alkyl, and
   (d) trifluoromethyl,
   (e) C₁₋₃alkyl,
   (f) —O—C₁₋₃alkyl,
   (g) —CO₂R⁹, wherein R⁹ is independently selected from: hydrogen, C₁₋₆ alkyl, C₅₋₆ cycloalkyl, benzyl or phenyl, which is unsubstituted or substituted with 1-3 substituents where the substituents are independently selected from: halo, C₁₋₃alkyl, C₁₋₃alkoxy and trifluoromethyl,
   (h) —CN,
   (i) —NR⁹R¹⁰,
   (j) —NR⁹COR¹⁰,
   (k) —NR⁹SO₂R¹⁰, and
   (l) —CONR⁹R¹⁰;

R² is selected from:
(C₀₋₆alkyl)-phenyl and (C₀₋₆alkyl)-heterocycle,
   where the alkyl is unsubstituted or substituted with 1-7 substituents where the substituents are independently selected from:
   (a) halo,
   (b) hydroxy,
   (c) —O—C₁₋₃alkyl,
   (d) trifluoromethyl, and
   (e) —C₁₋₃alkyl,
and where the phenyl and the heterocycle is unsubstituted or substituted with 1-5 substituents where the substituents are independently selected from:
   (a) halo,
   (b) trifluoromethyl,
   (c) trifluoromethoxy,
   (d) hydroxy,
   (e) C₁₋₆alkyl,
   (f) C₃₋₇cycloalkyl,
   (g) —O—C₁₋₆alkyl,
   (h) —O—C₃₋₇cycloalkyl,
   (i) —SCF₃,
   (j) —S—C₁₋₆alkyl,
   (k) —SO₂—C₁₋₆alkyl,
   (l) phenyl,
   (m) heterocycle,
   (n) —CO₂R₉,
   (o) —CN,
   (p) —NR⁹R¹⁰,
   (q) —NR⁹—SO₂—R¹⁰,
   (r) —SO₂—NR⁹R¹⁰, and
   (s) —CONR⁹R¹⁰;

R³ is —(C₀₋₆alkyl)-phenyl,
where the alkyl is unsubstituted or substituted with 1-5 substituents where the substituents are independently selected from:
(a) halo,
(b) hydroxy,
(c) —O—C₁₋₃alkyl, and
(d) trifluoromethyl,
and where the phenyl is unsubstituted or substituted with 1-5 substituents where the substituents are independently selected from:
(a) halo,
(b) trifluoromethyl,
(c) hydroxy,
(d) C₁₋₃alkyl,
(e) —O—C₁₋₃alkyl,
(f) —CO₂R⁹,
(g) —CN,
(h) —NR⁹R¹⁰, and
(i) —CONR⁹R¹⁰;

R⁴ is selected from:
(a) hydrogen,
(b) hydroxy,
(c) C₁₋₆alkyl,
(d) C₁₋₆alkyl-hydroxy,
(e) —O—C₁₋₃alkyl,
(f) —CO₂R⁹,
(g) —CONR⁹R¹⁰, and
(h) —CN;

or where R³ and R⁴ may be joined together to form a ring which is selected from:
(a) 1H-indene,
(b) 2,3-dihydro-1H-indene,
(c) 2,3-dihydro-benzofuran,
(d) 1,3-dihydro-isobenzofuran,
(e) 2,3-dihydro-benzothiofuran, and
(f) 1,3-dihydro-isobenzothiofuran,

or where R³ and R⁵ or R⁴ and R⁶ may be joined together to form a ring which is phenyl,
wherein the ring is unsubstituted or substituted with 1-7 substituents where the substituents are independently selected from:
   (a) halo,
   (b) trifluoromethyl,
   (c) hydroxy,
   (d) C₁₋₃alkyl,
   (e) —O—C₁₋₃alkyl,
   (f) —CO₂R⁹,
   (g) —CN,
   (h) —NR⁹R¹⁰, and
   (i) —CONR⁹R¹⁰;

R⁵ and R⁶ are independently selected from:
(a) hydrogen,
(b) hydroxy,
(c) C₁₋₆alkyl,
(d) C₁₋₆alkyl-hydroxy,
(e) —O—C₁₋₃alkyl,
(f) oxo, and
(g) halo;

and pharmaceutically acceptable salts thereof and individual diastereomers thereof.

### Formula IX Compounds

### EXAMPLES

Examples of the compounds of Formula IX include the following:

### Example IX-1

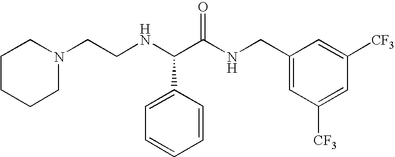

### Example IX-21

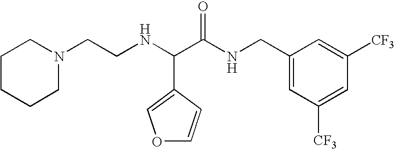

### Example IX-22

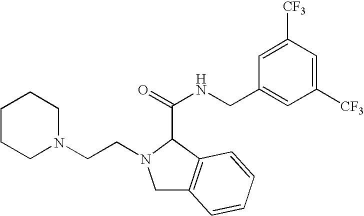

### Example IX-34

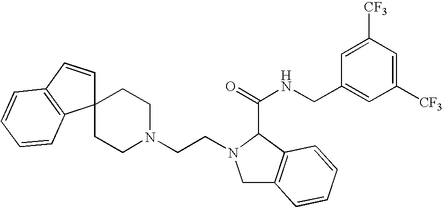

### Example IX-51

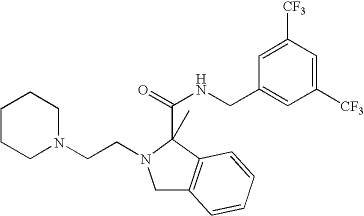

### Example IX-52

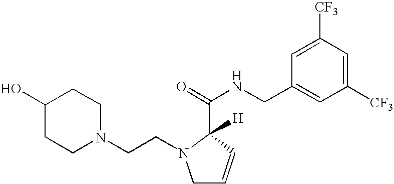

### Example IX-78

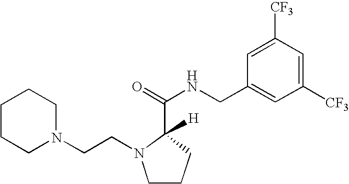

### Example IX-79

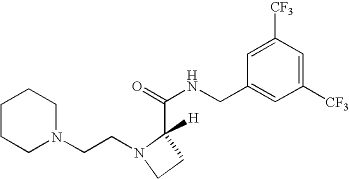

### Example IX-80

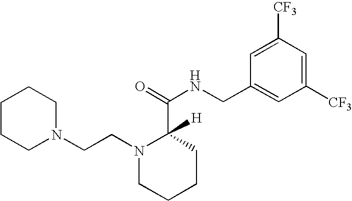

### Example IX-81

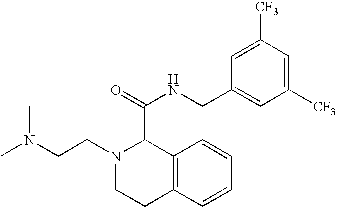

### Example IX-82

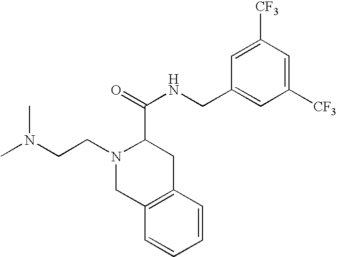

### Example IX-83

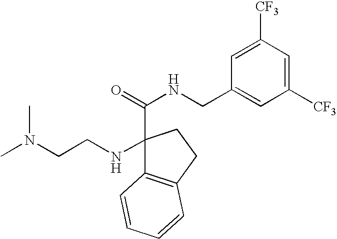

Additional CCR-2 antagonists useful in the methods of the inventors include those of Formula Xae and Xb. 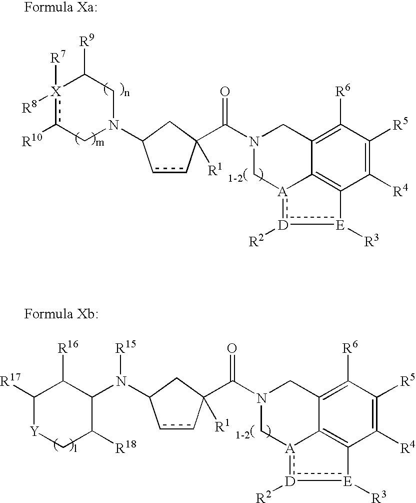
wherein:

A is selected from C or N;

D and E are independently selected from C, N, O, —SO— and —SO₂— to make a fused carbocycle (if A, D and E are all C) or a heterocycle (if at least one of A, D, or E is N, O, or S). The dashed lines represent either single or double bonds, where the dashed lines between A-D-E represent either one single and one double bond in either of the 2 possible configurations, or represent 2 single bonds;

X is selected from O, N, S, SO₂, or C.

Y is selected from the group consisting of:
—O—, —NR¹²—, —S—, —SO—, —SO₂—, and —CR¹²R¹²—, —NSO₂R¹⁴—, —NCOR¹³—, —CR¹²COR¹¹—, —CR¹²OCOR¹³— and —CO—,
where R¹¹ is independently selected from: hydroxy, hydrogen, C₁₋₆ alkyl, —O—C₁₋₆alkyl, benzyl, phenyl and C₃₋₆ cycloalkyl, where the alkyl, phenyl, benzyl, and cycloalkyl groups can be unsubstituted or substituted with 1-3 substituents, and where the substituents are independently selected from: halo, hydroxy, C₁₋₃alkyl, C₁₋₃alkoxy, —CO₂H, —CO₂—C₁₋₆ alkyl, and trifluoromethyl,
where R¹² is selected from: hydrogen, C¹⁻⁶ alkyl, benzyl, phenyl, and C₃₋₆ cycloalkyl, where the alkyl, phenyl, benzyl, and cycloalkyl groups can be unsubstituted or substituted with 1-3 substituents, and where the substituents are independently selected from: halo, hydroxy, C₁₋₃alkyl, C₁₋₃alkoxy, —CO₂H, —CO₂—C₁₋₆ alkyl, and trifluoromethyl,
where R¹³ is selected from: hydrogen, C₁₋₆ alkyl, —O—C₁₋₆alkyl, benzyl, phenyl, C₃₋₆ cycloalkyl, where the alkyl, phenyl, benzyl, and cycloalkyl groups can be unsubstituted or substituted with 1-3 substituents, and where the substituents are independently selected from: halo, hydroxy, C₁₋₃alkyl, C₁₋₃alkoxy, —CO₂H, —CO₂—C₁₋₆ alkyl, and trifluoromethyl, and
where R¹⁴ is selected from: hydroxy, C₁₋₆ alkyl, —O—C₁₋₆alkyl, benzyl, phenyl, C₃₋₆ cycloalkyl, where the alkyl, phenyl, benzyl, and cycloalkyl groups can be unsubstituted or substituted with 1-3 substituents, and where the substituents are independently selected from: halo, hydroxy, C₁₋₃alkyl, C₁₋₃alkoxy, —CO₂H, —CO₂—C₁₋₆ alkyl, and trifluoromethyl;

R¹ is selected from:
hydrogen, —C₁₋₆alkyl, —C₀₋₆alkyl-O—C₁₋₆alkyl, —C₀₋₆alkyl-S—C₁₋₆alkyl, —(C₀₋₆alkyl)-(C₃₋₇cycloalkyl)-(C₀₋₆alkyl), hydroxy, heterocycle, —CN, —NR¹²R¹², —NR¹²COR¹³, —NR¹²SO₂R¹⁴, —COR¹¹, —CONR¹²R¹², and phenyl,
where the alkyl and the cycloalkyl are unsubstituted or substituted with 1-7 substituents, where the substituents are independently selected from:
   (a) halo,
   (b) hydroxy,
   (c) —O—C₁₋₃alkyl,
   (d) trifluoromethyl,
   (f) C₁₋₃alkyl,
   (g) —O—C₁₋₃alkyl,
   (h) —COR¹¹,
   (i) —SO₂R¹⁴,
   (j) —NHCOCH₃,
   (k) —NHSO₂CH₃,
   (l) -heterocycle,
   (m) ═O, and
   (n) —CN,
and where the phenyl and heterocycle are unsubstituted or substituted with 1-3 substituents, where the substituents are independently selected from: halo, hydroxy, C₁₋₃alkyl, C₁₋₃alkoxy and trifluoromethyl;

if D is C, R² is selected from:
(a) hydrogen,
(b) C₁₋₃alkyl, optionally substituted with 1-3 fluoro,
(c) —O—C₁₋₃alkyl, optionally substituted with 1-3 fluoro,
(d) hydroxy,
(e) chloro,
(f) fluoro,
(g) bromo, and
(h) phenyl, and
(g) ═O (where R³ forms a double bond to E);

if D is N, R² is selected from:
(a) hydrogen,
(b) C₁₋₃alkyl, optionally substituted with 1-3 fluoro,
(c) —O—C₁₋₃alkyl, optionally substituted with 1-3 fluoro,
(d) phenyl, and
(e) O (to give an N-oxide).

if D is O, SO, or SO₂, R² is nothing;

if E is C, R³ is selected from:
(a) hydrogen,
(b) C₁₋₃alkyl, optionally substituted with 1-3 fluoro,
(c) —O—C₁₋₃alkyl, optionally substituted with 1-3 fluoro,
(d) hydroxy,
(e) chloro,
(f) fluoro,
(g) bromo, and
(h) phenyl, and
(g) ═O (where R³ forms a double bond to E);

if E is N, R³ is selected from:
(a) hydrogen,
(b) C₁₋₃alkyl, optionally substituted with 1-3 fluoro,
(c) —O—C₁₋₃alkyl, optionally substituted with 1-3 fluoro,
(d) phenyl,
(e) O (to give an N-oxide).

if E is O, SO, or SO₂, R³ is nothing;

R⁴ is selected from:
(a) hydrogen,
(b) C₁₋₃alkyl, optionally substituted with 1-3 fluoro,
(c) —O—C₁₋₃alkyl, optionally substituted with 1-3 fluoro,
(d) hydroxy,
(e) chloro,
(f) fluoro,
(g) bromo, and
(h) phenyl;

R₅ is selected from:
(a) C₁₋₆alkyl, where alkyl may be unsubstituted or substituted with 1-6 fluoro and optionally substituted with hydroxyl,
(b) —O—C₁₋₆alkyl, where alkyl may be unsubstituted or substituted with 1-6 fluoro,
(c) —CO—C₁₋₆alkyl, where alkyl may be unsubstituted or substituted with 1-6 fluoro,
(d) —S—C₁₋₆alkyl, where alkyl may be unsubstituted or substituted with 1-6 fluoro,
(e) -pyridyl, which may be unsubstituted or substituted with one or more substituents selected from the group consisting of: halo, trifluoromethyl, C₁₋₄alkyl, and COR¹¹,
(f) fluoro,
(g) chloro,
(h) bromo,
(i) —C₄₋₆cycloalkyl,
(j) —O—C₄₋₆cycloalkyl,
(k) phenyl, which may be unsubstituted or substituted with one or more substituents selected from the group consisting of: halo, trifluoromethyl, C₁₋₄alkyl, and COR¹¹,
(l) —O-phenyl, which may be unsubstituted or substituted with one or more substituents selected from the group consisting of: halo, trifluoromethyl, C₁₋₄alkyl, and COR¹¹,
(m) —C₃₋₆cycloalkyl, where alkyl may be unsubstituted or substituted with 1-6 fluoro,
(n) —O—C₃₋₆cycloalkyl, where alkyl may be unsubstituted or substituted with 1-6 fluoro,
(o) -heterocycle,
(p) —CN, and
(q) —COR¹¹;

R⁶ is selected from:
(a) hydrogen,
(b) alkyl, optionally substituted with 1-3 fluoro,
(c) —O—C₁₋₃alkyl, optionally substituted with 1-3 fluoro,
(d) hydroxy,
(e) chloro,
(f) fluoro,
(g) bromo, and
(h) phenyl;

R⁷ is selected from:
hydrogen, (C₀₋₆alkyl)-phenyl, (C₀₋₆alkyl)-heterocycle, (C₀₋₆alkyl)-C₃₋₇cycloalkyl, (C₀₋₆alkyl)-COR¹¹, (C₀₋₆alkyl)-(alkene)-COR¹¹, (C₀₋₆alkyl)-SO₃H, (C₀₋₆alkyl)-W-C₀₋₄alkyl, (C_{0 6}alkyl)-CONR¹²-phenyl, (C₀₋₆alkyl)-CONR²⁰-V-COR¹¹, and nothing (when X is O, S, or SO₂), where V is selected from C₁₋₆alkyl or phenyl, and
where W is selected from: a single bond, —O—, —S—, —SO—, —SO₂—, —CO—, —CO₂—, —CONR¹²— and —NR¹²—,
where the R²⁰ can be hydrogen, C₁₋₄alkyl, or where R²⁰ is joined via a 1-5 carbon tether to one of the carbons of V to form a ring, where the C₀₋₆alkyl is unsubstituted or substituted with 1-5 substituents,
where the substituents are independently selected from:
(a) halo,
(b) hydroxy,
(c) —C₀₋₆alkyl
(d) —O—C₁₋₃alkyl,
(e) trifluoromethyl, and
(f) —C₀₋₂alkyl-phenyl,
where the phenyl, heterocycle, cycloalkyl, and C₀₋₄alkyl is unsubstituted or substituted with 1-5 substituents where the substituents are independently selected from:
   (a) halo,
   (b) trifluoromethyl,
   (c) hydroxy,
   (d) C₁₋₃alkyl,
   (e) —O—C₁₋₃alkyl,
   (f) —C₀₋₃—COR¹¹,
   (g) —CN,
   (h) —NR¹²R¹²,
   (i) —CONR¹²R¹², and
   (j) —C₀₋₃-heterocycle,
   or where the phenyl and heterocycle may be fused to another heterocycle, which itself may be unsubstituted or substituted with 1-2 substituents independently selected from hydroxy, halo, —COR11, and C₁₋₃alkyl,
and where alkene is unsubstituted or substituted with 1-3 substituents which are independently selected from:
   (a) halo,
   (b) trifluoromethyl,
   (c) C₁₋₃alkyl,
   (d) phenyl, and
   (e) heterocycle;

R⁸ is selected from:
(a) hydrogen,
(b) nothing when X is either O, S, SO₂ or N or when a double bond joins the carbons to which R⁷ and R¹⁰ are attached,
(c) hydroxy,
(d) C₁₋₆alkyl,
(e) C₁₋₆alkyl-hydroxy,
(f) —O—C₁₋₃alkyl,
(g) —COR¹¹,
(h) —CONR¹²R¹², and
(i) —CN;

or where R⁷ and R⁸ may be joined together to form a ring which is selected from:
(a) 1H-indene,
(b) 2,3-dihydro-1H-indene,
(c) 2,3-dihydro-benzofuran,
(d) 1,3-dihydro-isobenzofuran,
(e) 2,3-dihydro-benzothiofuran,
(f) 1,3-dihydro-isobenzothiofuran,
(g) 6H-cyclopenta[d]isoxazol-3-ol
(h) cyclopentane, and
(i) cyclohexane,
where the ring formed may be unsubstituted or substituted with 1-5 substituents independently selected from:
   (a) halo,
   (b) trifluoromethyl,
   (c) hydroxy,
   (d) C₁₋₃alkyl,
   (e) —O—C₁₋₃alkyl,
(f) —C₀₋₃—COR¹¹,
(g) —CN,
(h) —NR¹²R¹²,
(i) —CONR¹²R¹², and
(j) —C₀₋₃-heterocycle,

or where R⁷ and R⁹ or R⁸ and R¹⁰ may be joined together to form a ring which is phenyl or heterocycle,
wherein the ring is unsubstituted or substituted with 1-7 substituents where the substituents are independently selected from:
   (a) halo,
   (b) trifluoromethyl,
   (c) hydroxy,
   (d) C₁₋₃alkyl,
   (e) —O—C₁₋₃alkyl,
   (f) —COR¹¹,
   (g) —CN,
   (h) —NR¹²R¹², and
   (i) —CONR¹²R¹²;

R⁹ and R¹⁰ are independently selected from:
(a) hydrogen,
(b) hydroxy,
(c) C₁₋₆alkyl,
(d) C₁₋₆alkyl-COR¹¹,
(e) C₁₋₆alkyl-hydroxy,
(f) —O—C₁₋₃alkyl,
(g) ═O, when R⁹ or R¹⁰ is connected to the ring via a double bond
(h) halo;

R¹⁵ is selected from:
(a) hydrogen, and
(b) C₁₋₆alkyl, which is unsubstituted or substituted with 1-3 substituents where the substituents are independently selected from: halo, hydroxy, —. CO₂H, —CO₂C₁₋₆alkyl, and —O—C₁₋₃alkyl;

R¹⁶ is selected from:
(a) hydrogen,
(b) C₁₋₆alkyl, where alkyl may be unsubstituted or substituted with 1-6 substituents where the substituents are chosen from the group: fluoro, C₁₋₃alkoxy, hydroxy, —COR¹¹,
(c) fluoro,
(d) —O—C₁₋₃alkyl, where alkyl may be unsubstituted or substituted with 1-3 fluoro, and
(e) C₃₋₆ cycloalkyl,
(f) —O—C₃₋₆cycloalkyl,
(g) hydroxy,
(h) —COR¹¹, and
(i) —OCOR¹³,
or R¹⁵ and R¹⁶ may be joined together via a C₂₋₄alkyl or a C₀₋₂alkyl-O—C₁₋₃alkyl chain to form a 5-7 membered ring;

R¹⁷ is selected from:
(a) hydrogen,
(b) C₁₋₆alkyl, where alkyl may be unsubstituted or substituted with 1-6 substituents where the substituents are chosen from the group: fluoro, C₁₋₃alkoxy, hydroxy, —COR¹¹,
(c) COR¹¹,
(d) hydroxy, and
(e) —O—C₁₋₆alkyl, where alkyl may be unsubstituted or substituted with 1-6 substituents where the substituents are chosen from the group: fluoro, C₁₋₃alkoxy, hydroxy, and —COR¹¹,
or R¹⁶ and R¹⁷ may be joined together by a C₁₋₄alkyl chain or a C₀₋₃alkyl-O—C₀₋₃alkyl chain to form a 3-6 membered ring;

R¹⁸ is selected from:
(a) hydrogen,
(b) C₁₋₆alkyl, where alkyl may be unsubstituted or substituted with 1-6 fluoro,
(c) fluoro,
(d) —O—C₃₋₆cycloalkyl, and
(e) —O—C₁₋₃alkyl, where alkyl may be unsubstituted or substituted with 1-6 fluoro,
or R¹⁶ and R¹⁸ may be joined together by a C₂₋₃alkyl chain to form a 5-6 membered ring, where the alkyl are unsubstituted or substituted with 1-3 substituents where the substiuents are independently selected from: halo, hydroxy, —COR¹¹, C₁₋₃alkyl, and C₁₋₃alkoxy,
or R¹⁶ and R¹⁸ may be joined together by a C₁₋₂alkyl-O—C₁₋₂alkyl chain to form a 6-8 membered ring, where the alkyl are unsubstituted or substituted with 1-3 substituents where the substiuents are independently selected from: halo, hydroxy, —COR¹¹, C₁₋₃alkyl, and C₁₋₃alkoxy,
or R¹⁶ and R¹⁸ may be joined together by a —O—C₁₋₂alkyl-O-chain to form a 6-7 membered ring, where the alkyl are unsubstituted or substituted with 1-3 substituents where the substiuents are independently selected from: halo, hydroxy, —COR¹¹C₁₋₃alkyl, and C₁₋₃alkoxy;

R¹⁹ selected from:
(a) hydrogen,
(b) phenyl, and
(c) C₁₋₆alkyl which may be substituted or unsubstituted with 1-6 of the following substituents: —COR¹¹, hydroxy, fluoro, chloro and —O—C₁₋₃alkyl;

1, m, and n are each selected from 0, 1 and 2.

and pharmaceutically acceptable salts thereof and individual diastereomers thereof.

### Formula X Compounds

### EXAMPLES

Examples of the compounds of Formula X include the following:

### Example X-1

### (L-071142; S. Goble, 44292-048A)

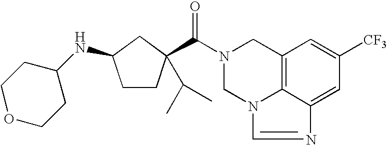

### Example X-2

### (L-071156; S. Goble; 43899-084B/092B)

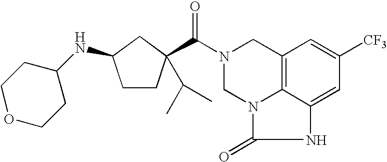

### Example X-3

### (L-114895; S. Goble; 43899-103B)

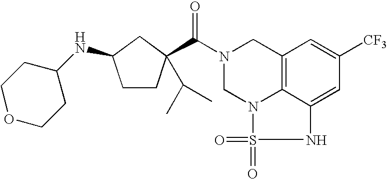

### Example X-4

### (L-221392: S. Goble; 43899-147B)

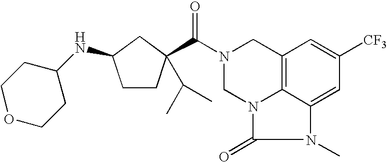

### Example X-5

### (L-075642; C. Tang)

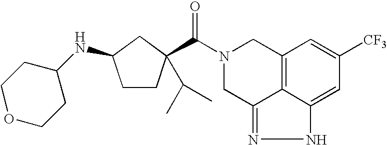

Additional CCR-2 antagonists useful in the methods of the inventors include those of Formula XI: 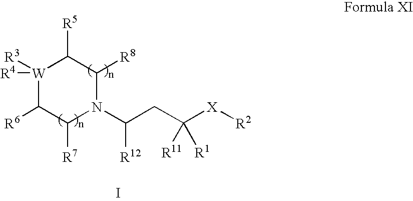
wherein:

W is selected from the group consisting of:
C, N, and —O—, wherein when W is N, then R⁴ is absent, and when W is —O—, then both R³ and R⁴ are absent;

X is selected from the group consisting of:
—NR¹⁰—, —O—, —CH₂O—, —CONR¹⁰—, —NR¹⁰CO—, —CO₂—, —OCO—, —CH₂(NR¹⁰)CO—, —N(COR¹⁰)—, and —CH₂N(COR¹⁰)—,
and where R¹⁰ is independently selected from: hydrogen, C₁₋₆ alkyl, benzyl, phenyl, and C₁₋₆ alkyl-C₃₋₆ cycloalkyl,
   which is unsubstituted or substituted with 1-3 substituents where the substituents are independently selected from: halo, C₁₋₃alkyl,
   C₁₋₃alkoxy and trifluoromethyl;
or where R¹⁰ and R² may be joined together to form a 5- or 6-membered ring,

R¹ is selected from:
hydrogen, —C₀₋₆alkyl-Y-phenyl-, —C₀₋₆alkyl-Y-heterocycle-, —C₀₋₆alkyl-Y-(C₁₋₆alkyl)-, and
—(C₀₋₆alkyl)-Y-(C₀₋₆alkyl)-(C₃₋₇cycloalkyl)-(C₀₋₆alkyl),
   where Y is selected from:
   a single bond, —O—, —S—, —SO—, —SO₂—, and —NR¹⁰—,
   and where the phenyl, heterocycle, alkyl and the cycloalkyl are unsubstituted or substituted with 1-7 substituents where the substituents are independently selected from:
   (a) halo,
   (b) hydroxy,
   (c) —O—C₁₋₃alkyl,
   (d) trifluoromethyl,
   (e) C₁₋₃alkyl,
   (f) —C₃₋₆cycloalkyl
   (g) —CO₂R⁹, wherein R⁹ is independently selected from: hydrogen, C₁₋₆ alkyl, C₅₋₆cycloalkyl, benzyl or phenyl, which is unsubstituted or substituted with 1-3 substituents where the substituents are independently selected from: halo, C₁₋₃alkyl, C₁₋₃alkoxy and trifluoromethyl,
   (h) —CN,
   (i) —NR⁹R¹⁰,
   (j) —NR⁹COR¹⁰,
   (k) —NR⁹SO₂R¹⁰,
   (l) —NR⁹CO₂R¹⁰,
   (m) —NR⁹CONR⁹R¹⁰,
   (n) —CONR⁹R¹⁰,
   (o) heterocycle,
   (p) phenyl;

R² is selected from:
(C₀₋₆alkyl)-phenyl and (C₀₋₆alkyl)-heterocycle,
   where the alkyl is unsubstituted or substituted with 1-7 substituents where the substituents are independently selected from:
   (a) halo,
   (b) hydroxy,
   (c) —O—C₁₋₃alkyl,
   (d) trifluoromethyl,
   (e) —C₁₋₃alkyl,
   (f) —CO₂R⁹, and
   (g) oxo;
and where the phenyl and the heterocycle may be unsubstituted or substituted with 1-5 substituents where the substituents are independently selected from:
   (a) halo,
   (b) trifluoromethyl,
   (c) trifluoromethoxy,
   (d) hydroxy,
   (e) C₁₋₆alkyl,
   (f) C₃₋₇cycloalkyl,
   (g) —O—C₁₋₆alkyl,
   (h) —O—C₃₋₇cycloalkyl,
   (i) —SCF₃,
   (j) —S—C₁₋₆alkyl,
   (k) —SO₂-C₁₋₆alkyl,
   (l) phenyl,
   (m) heterocycle,
   (n) —CO₂R⁹,
   (o) —CN,
   (p) —NR⁹R¹⁰,
   (q) —NR⁹—SO₂—R¹⁰,
   (r) —SO₂—NR⁹R¹⁰,
   (s) —CONR⁹R¹⁰, and
   (t) —O-phenyl;

R³ is selected from:
hydrogen, (C₀₋₆alkyl)-phenyl, (C₀₋₆alkyl)-heterocycle, C₁₋₆alkyl, CF₃, C₃₋₇cycloalkyl, —NR⁹R¹⁰, —CO₂R⁹, —NR⁹—SO₂—R¹⁰, —NR⁹CONR⁹R¹⁰, and —CONR⁹R¹⁰,
   where the alkyl is unsubstituted or substituted with 1-5 substituents where the substituents are independently selected from:
   (a) halo,
   (b) hydroxy,
   (c) —O—C₁₋₃alkyl, and
   (d) trifluoromethyl,
and where the phenyl, heterocycle, and cycloalkyl are unsubstituted or substituted with 1-5 substituents where the substituents are independently selected from:
   (a) halo,
   (b) trifluoromethyl,
   (c) hydroxy,
   (d) C₁₋₃alkyl,
   (e) —O—C₁₋₃alkyl,
   (f) —CO₂R⁹,
   (g) —CN,
   (h) —NR⁹R¹⁰, and
   (i) —CONR⁹R¹⁰
   (j) NR⁹SO₂R¹⁰,
   (k) SO₂NR⁹R¹⁰
   (l) phenyl,
   (m) heterocycle;
and where the phenyl, heterocycle, and cycloalkyl may or may not be fused to another phenyl or heterocycle;

R⁴ is selected from:
(a) hydrogen,
(b) hydroxy,
(c) C₁₋₆alkyl,
(d) C₁₋₆alkyl-hydroxy,
(e) —O—C₁₋₃alkyl,
(f) C₀₋₆CO₂R⁹,
(g) —CONR⁹R¹⁰, and
(h) —CN;

or R³ and R⁴ may be joined together to form a ring which is selected from:
(a) 1H-indene,
(b) 2,3-dihydro-1H-indene,
(c) 2,3-dihydro-benzofuran,
(d) 1,3-dihydro-isobenzofuran,
(e) 2,3-dihydro-benzothiofuran, and
(f) 1,3-dihydro-isobenzothiofuran,
where the 1H-indene, 2,3-dihydro-1H-indene, 2,3-dihydro-benzofuran, 1,3-dihydro-isobenzofuran, 2,3-dihydro-benzothiofuran, and 1,3-dihydro-isobenzothiofuran may be unsubstituted or substituted with 1-5 substituents where the substituents are independently selected from:
   (i) halo,
   (ii) trifluoromethyl,
   (iii) hydroxy,
   (iv) C₁₋₃alkyl,
   (v) —O—C₁₋₃alkyl,
   (vi) C₀₋₄CO₂R⁹,
   (vii) —CN,
   (viii) —NR⁹R¹⁰, and
   (ix) —CONR⁹R¹⁰
   (x) NR⁹SO₂R¹⁰,
   (xi) SO₂₉R₁₀
   (xii) phenyl,
   (xiii) heterocycle;

R⁵, R⁶, R⁷ and R⁸ are independently selected from:
(a) hydrogen,
(b) hydroxy,
(c) C₁₋₆alkyl,
(d) C₁₋₆alkyl-hydroxy,
(e) —O—C₁₋₃alkyl,
(f) oxo, and
(g) halo,
(h) C₀₋₄CO₂R⁹, and
(i) CF₃,
or where R⁵ and R⁶, or R⁷ and R⁸ may be joined together via a C₂₋₃alkyl chain to form a ring, or where R³ and R⁵, or R⁴ and R⁶ may be joined together to form a ring which is phenyl, heterocycle, or cycloalkyl, wherein the ring is unsubstituted or substituted with 1-7 substituents where the substituents are independently selected from:
   (i) halo,
   (ii) trifluoromethyl,
   (iii) hydroxy,
   (iv) C₁₋₃alkyl,
   (v) —O—C₁₋₃alkyl,
   (vi) CO₂R⁹,
   (vii) —CN,
   (viii) —NR⁹R¹⁰,
   (ix) —CONR⁹R¹⁰, and
   (x) phenyl;

R¹¹ is selected from:
(a) hydrogen,
(b) halo
(c) C₁₋₆alkyl,
(d) hydroxy,
(e) CO₂R⁹,
(f) —O—C₁₋₃alkyl, and
(g) —NR⁹R¹⁰;

R¹² is selected from:
(a) hydrogen,
(b) C₁₋₆alkyl, and
(c) CO₂R⁹;

n is an integer selected from 0, 1, 2 and 3;

and pharmaceutically acceptable salts thereof and individual diastereomers thereof.

### Formula XI Compounds

### EXAMPLES

Examples of the compounds of Formula XI include the following:

### Example XI-1

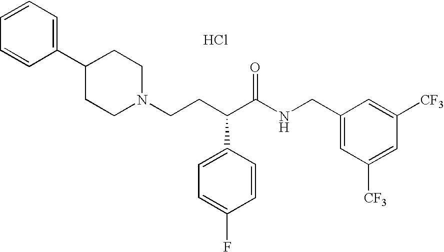

### Example XI-2

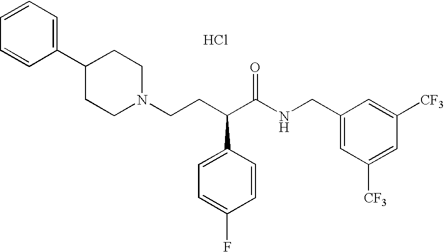

### Example XI-3

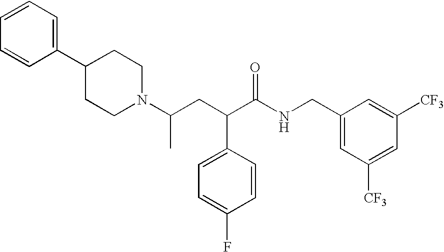

### Example XI-4

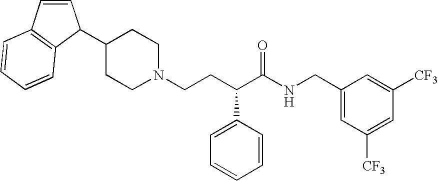

### Example XI-5

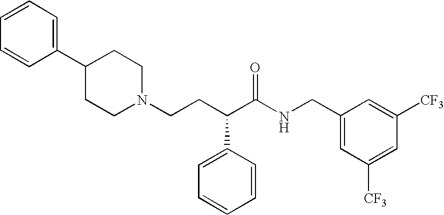

### Example XI-6

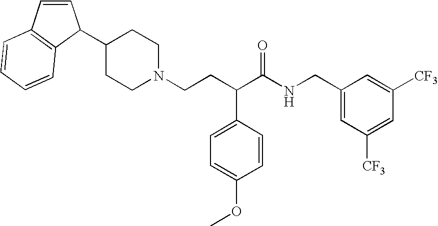

### Example XI-7

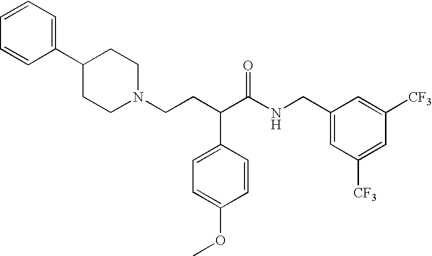

### Example XI-8

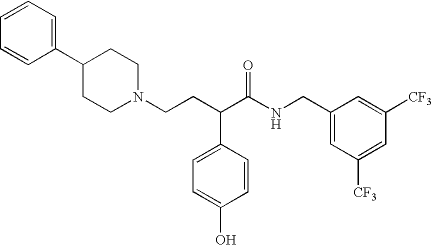

### Example XI-9

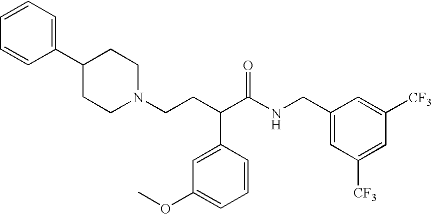

### Example XI-10

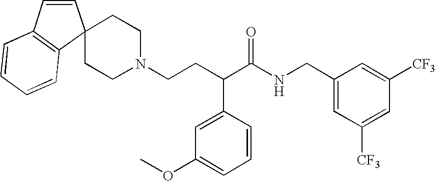

### Example XI-11

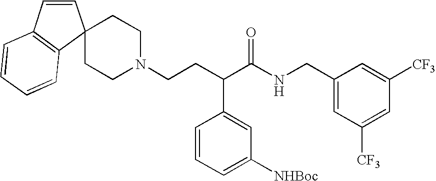

### Example XI-12

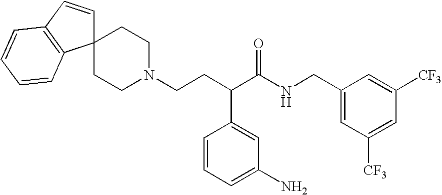

### Example XI-13

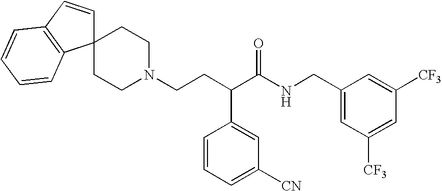

### Example XI-14

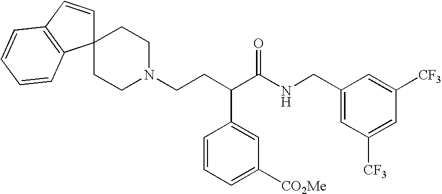

### Example XI-15

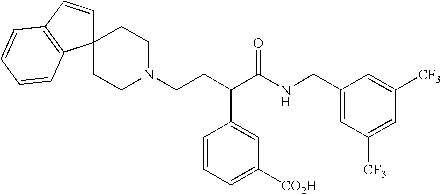

### Example XI-16

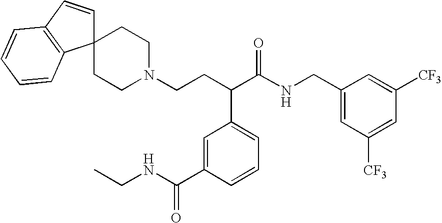

### Example XI-18

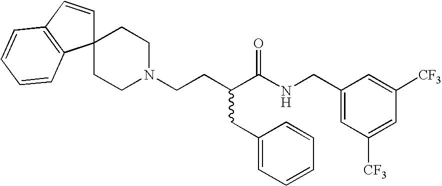

### Example XI-19

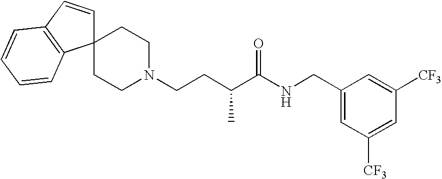

### Example XI-20

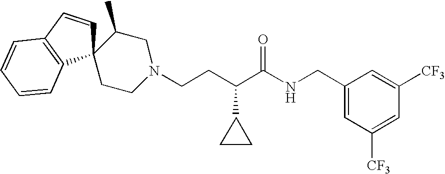

### Example XI-21

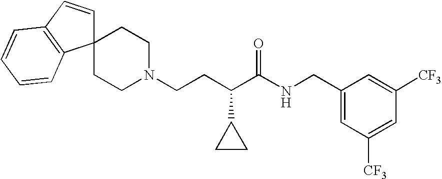

### Example XI-22

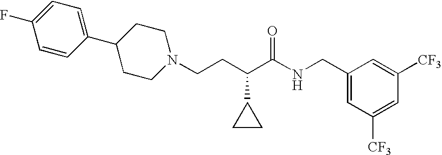

### Example XI-23

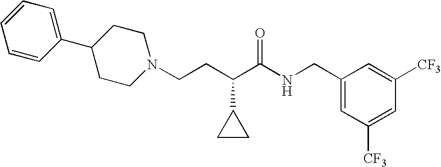

### Example XI-24

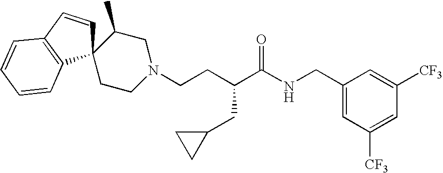

### Example XI-25

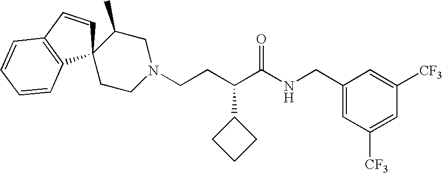

### Example XI-26

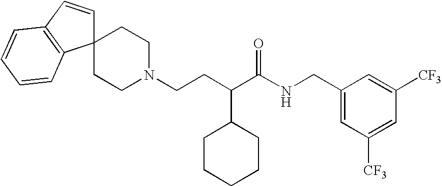

### Example XI-27

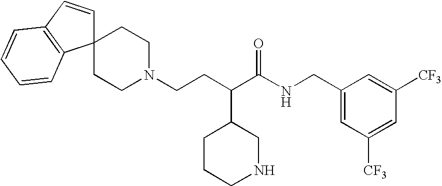

### Example XI-28

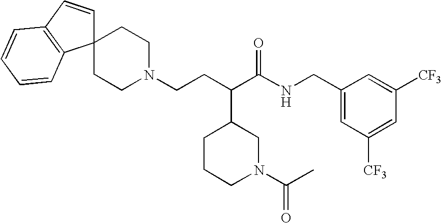

### Example XI-29

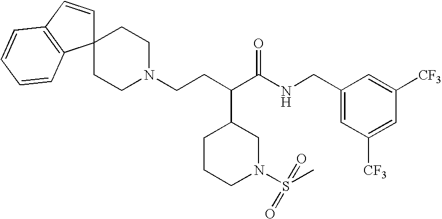

### Example XI-30

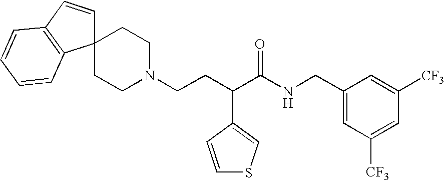

### Example XI-31

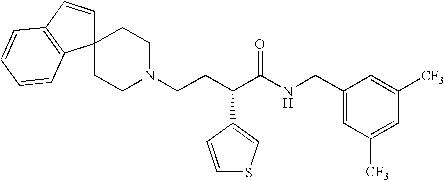

### Example XI-32

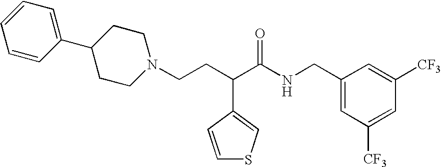

### Example XI-33

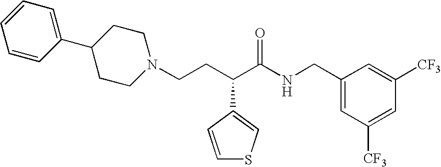

### Example XI-34

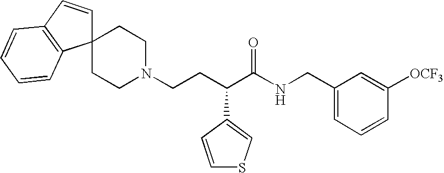

### Example XI-35

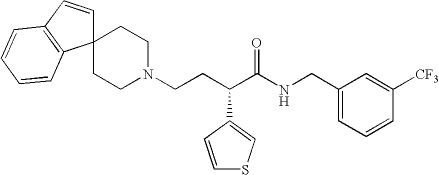

### Example XI-36

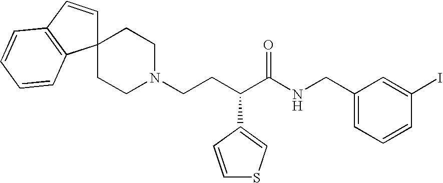

### Example XI-37

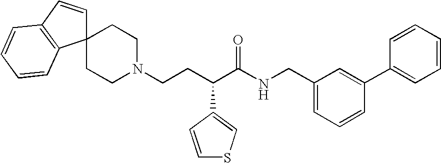

### Example XI-38

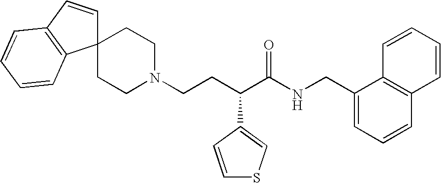

### Example XI-39

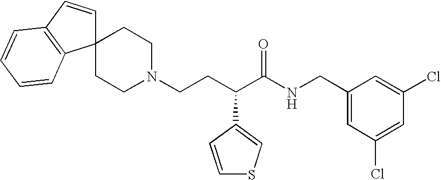

### Example XI-40

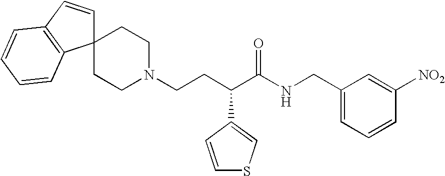

### Example XI-41

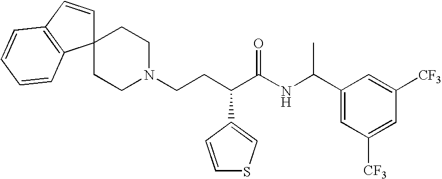

### Example XI-42

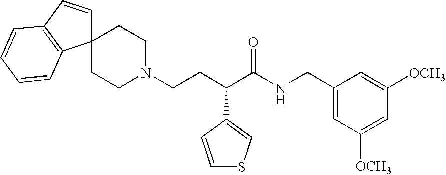

### Example XI-43

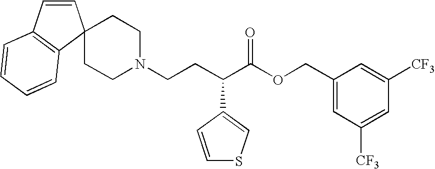

### Example XI-44

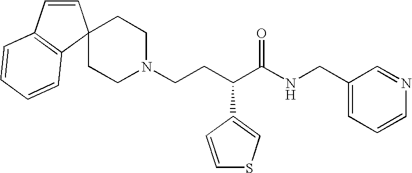

### Example XI-45

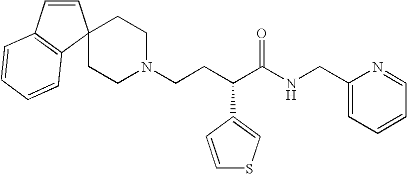

### Example XI-46

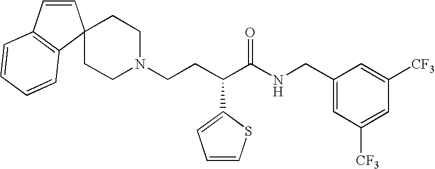

### Example XI-47

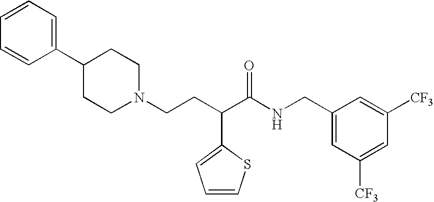

### Example XI-48

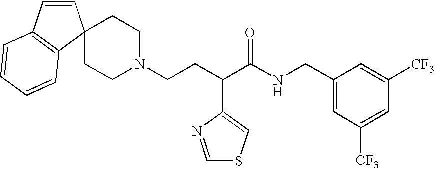

### Example XI-49

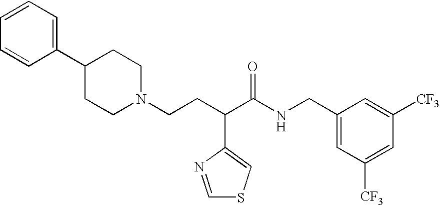

### Example XI-50

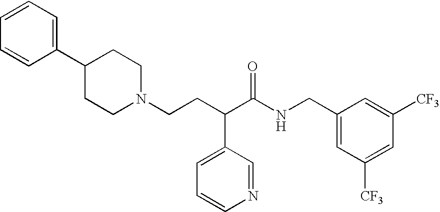

### Example XI-51

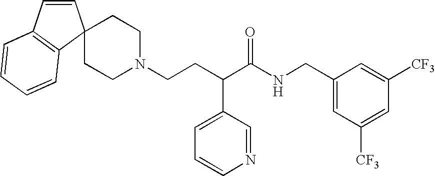

### Example XI-52

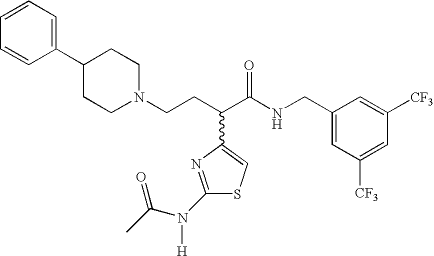

### Example XI 52A-N

Examples XI-52A through N, on Table 31, below, are based on the Formula:

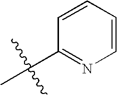

| | | | | m/z |
|---|---|---|---|---|
| EXAMPLE | Amine | R1 | R2 | (M + 1) |
| XI-52A | 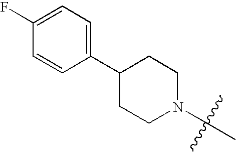 | H | H | 631 |
| IX-52B | 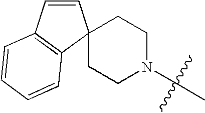 | H | H | 637 |
| IX-52C | 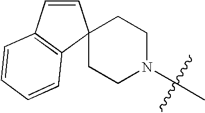 | H | Me | 651 |
| IX-52D | 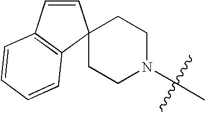 | Me | H | 651 |
| IX-52E | 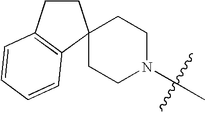 | H | H | 639 |
| IX-52F | 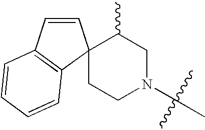 | H | H | 651 |
| IX-52G | 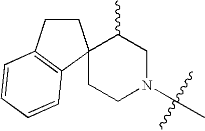 | H | H | 653 |
| IX-52H | 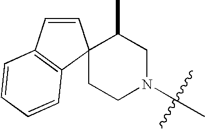 | H | H | 651 |
| IX-52I | 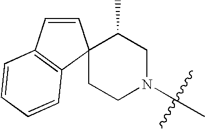 | H | H | 651 |
| IX-52J | 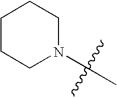 | H | H | 537 |
| IX-52K | 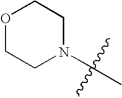 | H | H | 539 |
| IX-52L | 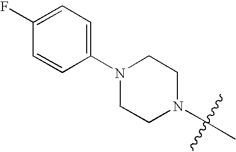 | H | H | 632 |
| IX-52M | 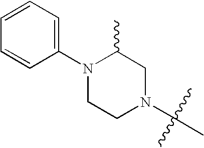 | H | H | 628 |
| IX-52N | 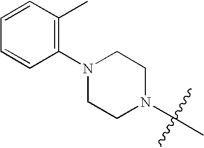 | H | H | 628 |

### Example XI-53

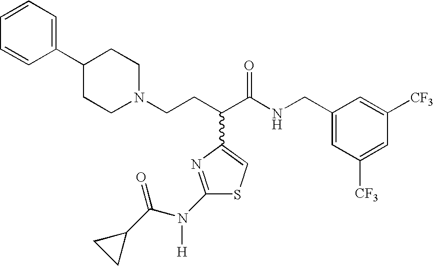

### Examples XI 54-70

Examples XI-54 through XI-70, on Table 32, below, are based on the Formula:

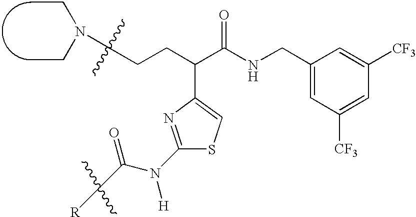

| EXAMPLE | Amine | R | m/z (M + 1) | Note |
|---|---|---|---|---|
| XI-54 | 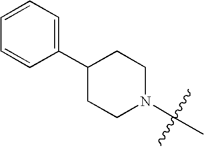 | 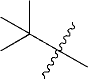 | 655 | |
| XI-55 | 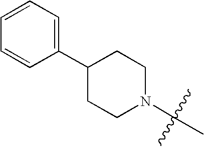 | 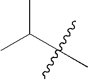 | 641 | |
| XI-56 | 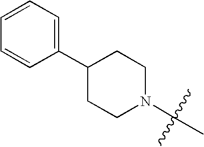 | 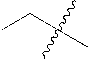 | 627 | |
| XI-57 | 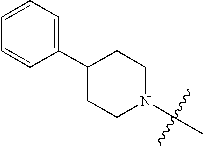 | 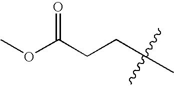 | 685 | |
| XI-58 | 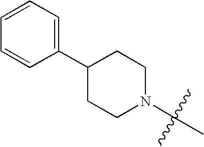 | 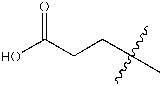 | 671 | From Hydrolysis of EXAMPLE XI-57 |
| XI-59 | 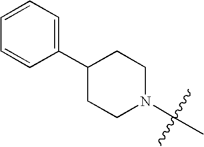 | 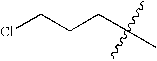 | 675/677 | |
| XI-60 | 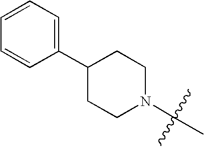 | 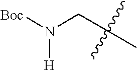 | 728 | |
| XI-61 | 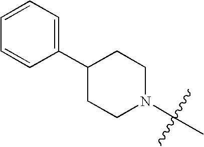 | 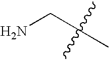 | 628 | From TFA Treatment of EXAMPLE XI-60 |
| XI-62 | 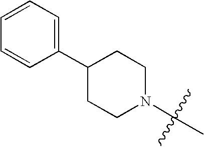 | 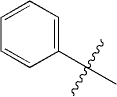 | 675 | |
| XI-63 | 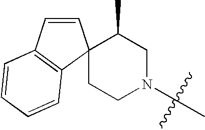 | 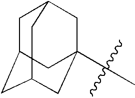 | 785 | |
| XI-64 | 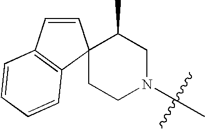 | 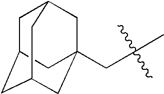 | 771 | |
| XI-65 | 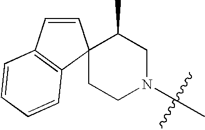 | 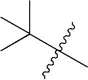 | 693 (hold) | |
| XI-66 | 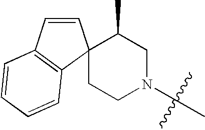 | 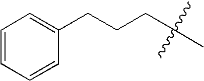 | 755 | |
| XI-67 | 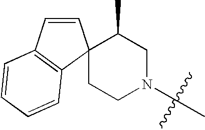 | 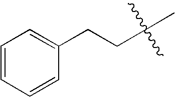 | 741 | |
| XI-68 | 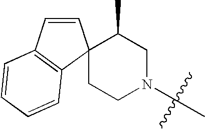 | 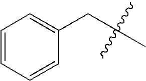 | 727 | |
| XI-69 | 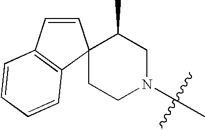 | 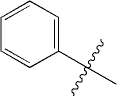 | 713 | |
| XI-70 | 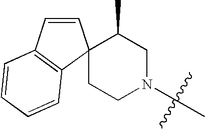 | 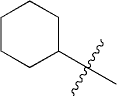 | 719 | |

### Example XI-71

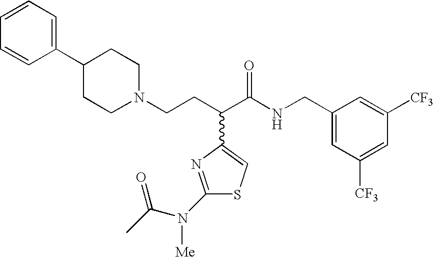

### Example XI-72

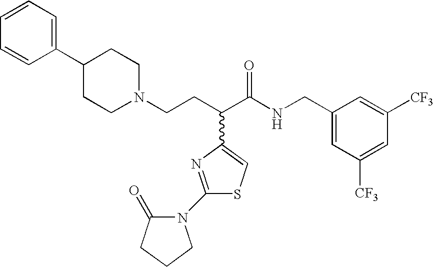

### Example XI-73

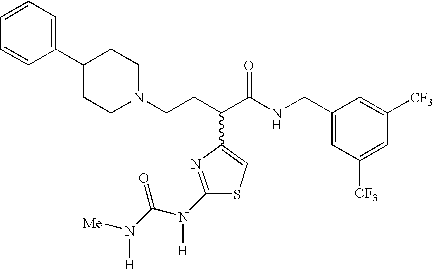

### Examples XI 74-79

Examples XI-74 through XI-79, on Table 33, below, are based on the Formula:

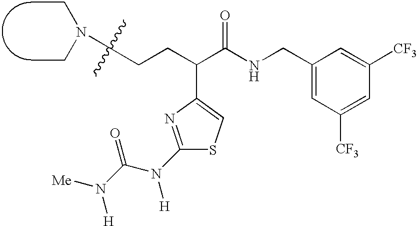

| EXAMPLE | Amine | (M + 1) |
|---|---|---|
| XI-74 | 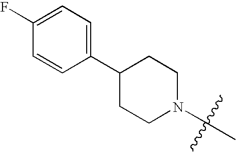 | 646 |
| XI-75 | 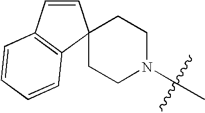 | 637 |
| XI-76 | 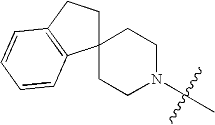 | 654 |
| XI-77 | 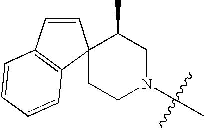 | 666 |
| XI-78 | 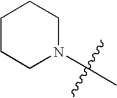 | 552 |
| XI-79 | 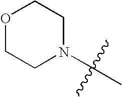 | 554 |

### Example XI-80

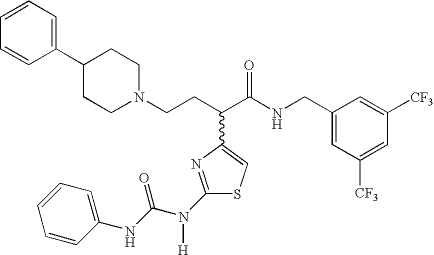

### Example XI-81

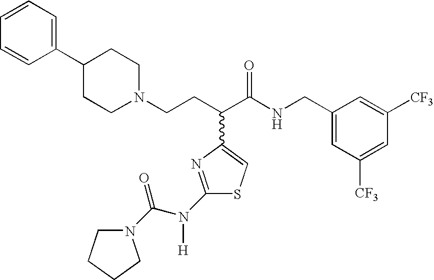

### Example XI-82

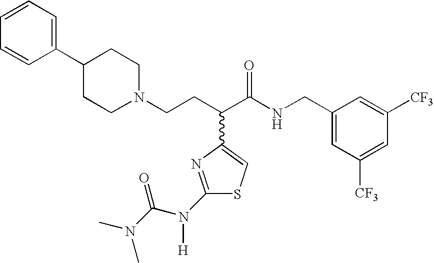

### Example XI-83

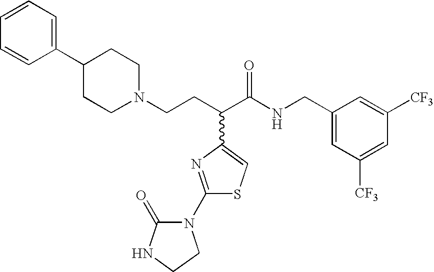

### Example XI-84

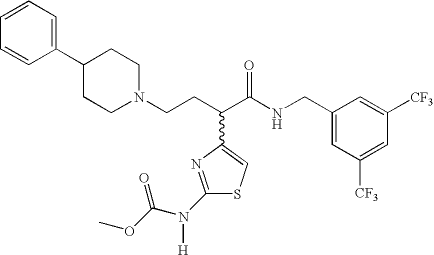

### Example XI-87

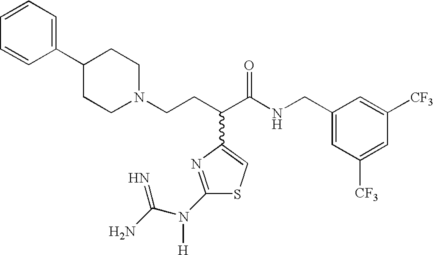

### Examples XI 88-92

Examples XI-88 through XI-92, on Table 34, below, are based on the Formula:

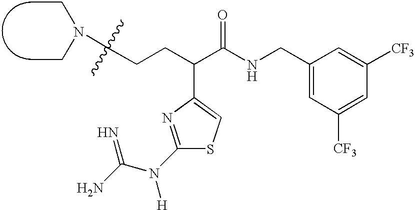

| EX. | | |
|---|---|---|
| XI- | Amine | (M + 1) |
| XI-88 | 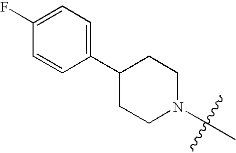 | 631 |
| XI-89 | 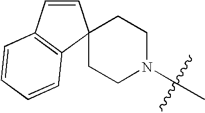 | 637 |
| XI-90 | 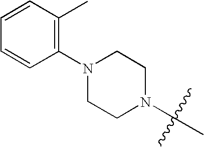 | 628 |
| XI-91 | 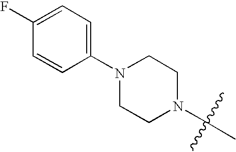 | 632 |
| XI-92 | 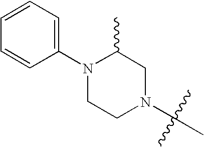 | 628 |

### Example XI-93

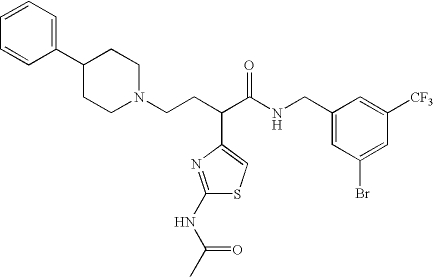

### Example XI-94

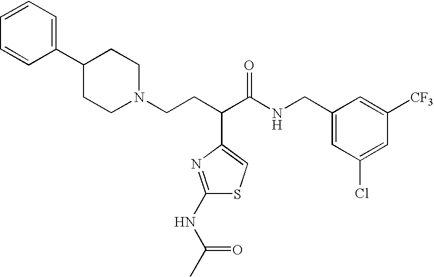

### Example XI-95

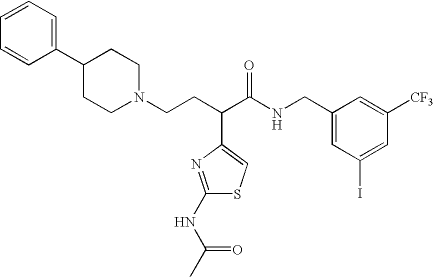

### Example XI-96

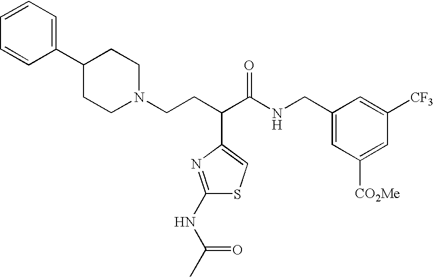

### Example XI-97

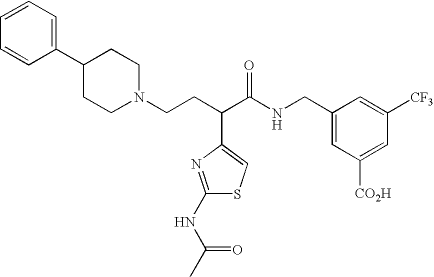

### Example XI-98

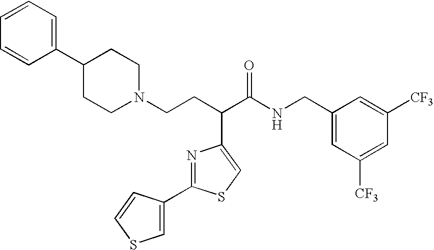

### Example XI-99

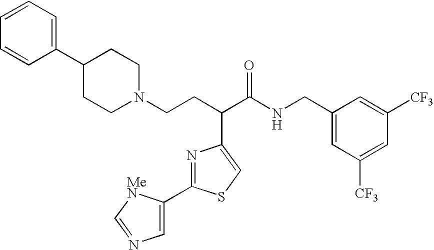

### Example XI-111

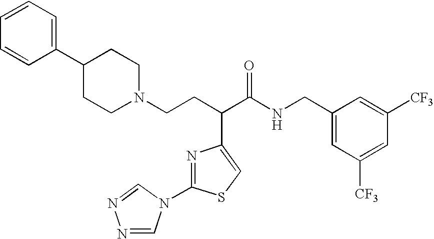

### Example XI-112

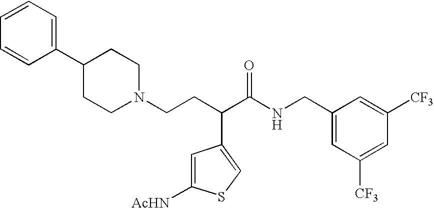

### Example XI-113

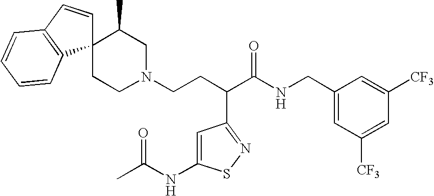

### Example XI-114

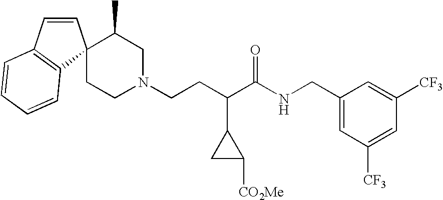

### Example XI-115

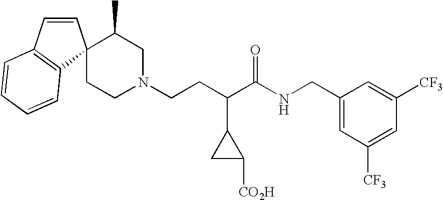

### Example XI-116

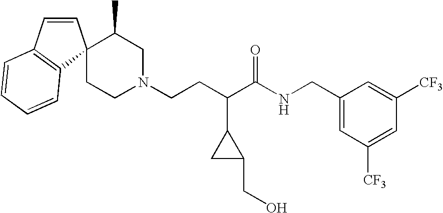

### Example XI-117

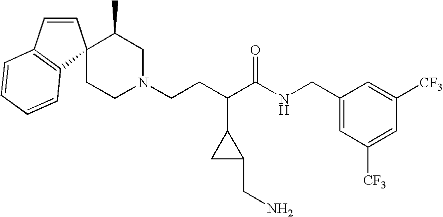

### Example XI-118

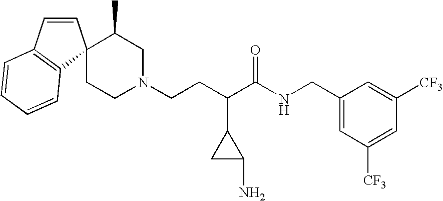

### Example XI-160

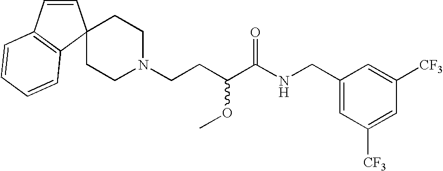

### Example XI-161

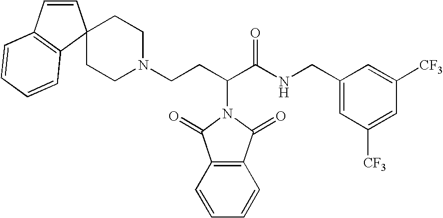

### Example XI-162

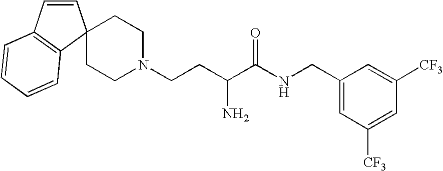

### Example XI-163

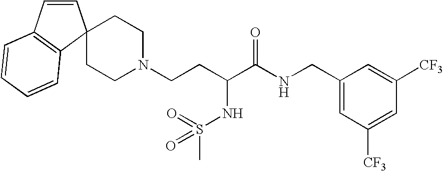

Additional CCR-2 antagonists useful in the methods of the invention include these of Formula XII: 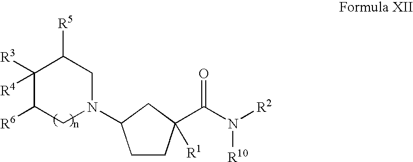
wherein:

R¹ is selected from:
hydrogen,
—C₀₋₆alkyl-Y—(C₁₋₆alkyl)-, and
—(C₀₋₆alkyl)-Y—(C₀₋₆alkyl)-(C₃₋₇cycloalkyl)-(C₀₋₆alkyl),
   where Y is selected from:
   a single bond, —O—, —S—, —SO—, —SO₂—, and —NR¹⁰—, and where the alkyl and the cycloalkyl are unsubstituted or substituted with 1-7 substituents where the substituents are independently selected from:
   (a) halo,
   (b) hydroxy,
   (c) —O—C₁₋₃alkyl, and
   (d) trifluoromethyl,
   (e) C₁₋₃alkyl,
   (f) —O—C₁₋₃alkyl,
   (g) —CO₂R⁹, wherein R⁹ is independently selected from: hydrogen, C₁₋₆ alkyl, C₅₋₆ cycloalkyl, benzyl or phenyl, which is unsubstituted or substituted with 1-3 substituents where the substituents are independently selected from: halo, C₁₋₃alkyl, C₁₋₃alkoxy and trifluoromethyl,
   (h) —CN,
   (i) heterocycle,
   (j) —NR⁹R¹⁰,
   (k) —NR⁹COR¹⁰,
   (l) —NR⁹SO₂R¹⁰, and
   (m) —CONR⁹R¹⁰;

R² is selected from:
(C₀₋₆alkyl)-phenyl and (C₀₋₆alkyl)-heterocycle,
   where the alkyl is unsubstituted or substituted with 1-7 substituents where the substituents are independently selected from:
   (a) halo,
   (b) hydroxy,
   (c) —O—C₁₋₃alkyl,
   (d) trifluoromethyl, and
   (e) —C₁₋₃alkyl,
and where the phenyl and the heterocycle is unsubstituted or substituted with 1-5 substituents where the substituents are independently selected from:
   (a) halo,
   (b) trifluoromethyl,
   (c) trifluoromethoxy,
   (d) hydroxy,
   (e) C₁₋₆alkyl,
   (f) C₃₋₇cycloalkyl,
   (g) —O—C₁₋₆alkyl,
   (h) —O—C₃₋₇cycloalkyl,
   (i) —SCF₃,
   (j) —S—C₁₋₆alkyl,
   (k) —SO₂—C₁₋₆alkyl,
   (l) phenyl,
   (m) heterocycle,
   (n) —CO₂R⁹,
   (o) —CN,
   (p) —NR⁹R¹⁰,
   (q) —NR⁹—SO₂—R¹⁰,
   (r) —SO₂—NR⁹R¹⁰, and
   (s) —CONR⁹R¹⁰;

R³ is selected from:
(C₀₋₆alkyl)-heterocycle,
   where the alkyl is unsubstituted or substituted with 1-5 substituents where the substituents are independently selected from:
   (a) halo,
   (b) hydroxy,
   (c) —O—C₁₋₃alkyl, and
   (d) trifluoromethyl,
and where the heterocycle is unsubstituted or substituted with 1-5 substituents where the substituents are independently selected from:
   (a) halo,
   (b) trifluoromethyl,
   (c) hydroxy,
   (d) C₁₋₃alkyl,
   (e) —O—C₁₋₃alkyl,
   (f) —CO₂R⁹,
   (g) —CN,
   (h) —NR⁹R¹⁰, and
   (i) —CONR⁹R¹⁰;

R⁴ is selected from:
(a) hydrogen,
(b) hydroxy,
(c) C₁₋₆alkyl,
(d) C₁₋₆alkyl-hydroxy,
(e) —O—C₁₋₃alkyl,
(f) —CO₂R⁹,
(g) —CONR⁹R¹⁰, and
(h) —CN;

or where R³ and R⁴ may be joined together to form a ring which is selected from:
(a) 1H-indene,
(b) 2,3-dihydro-1H-indene,
(c) 2,3-dihydro-benzofuran,
(d) 1,3-dihydro-isobenzofuran,
(e) 2,3-dihydro-benzothiofuran, and
(f) 1,3-dihydro-isobenzothiofuran,

or where R³ and R⁵ or R⁴ and R⁶ may be joined together to form a ring which is phenyl,
wherein the ring is unsubstituted or substituted with 1-7 substituents where the substituents are independently selected from:
   (a) halo,
   (b) trifluoromethyl,
   (c) hydroxy,
   (d) C₁₋₃alkyl,
   (e) —O—C₁₋₃alkyl,
   (f) —CO₂R⁹,
   (g) —CN,
   (h) —NR⁹R¹⁰, and
   (i) —CONR⁹R¹⁰;

R⁵ and R⁶ are independently selected from:
(a) hydrogen,
(b) hydroxy,
(c) C₁₋₆alkyl,
(d) C₁₋₆alkyl-hydroxy,
(e) —O—C₁₋₃ alkyl,
(f) oxo, and
(g) halo;

R¹⁰ is independently selected from:
hydrogen, C₁₋₆ alkyl, benzyl, phenyl, and C₁₋₆ alkyl-C₃₋₆ cycloalkyl, which is unsubstituted or substituted with 1-3 substituents where the substituents are independently selected from: halo, C₁₋₃alkyl, C₁₋₃alkoxy and trifluoromethyl;

n is an integer which is 0 or 1;

and pharmaceutically acceptable salts thereof and individual diastereomers thereof.

### Formula XII Compounds

### EXAMPLES

Examples of the compounds of Formula XIII include the following:

### Example XII-1

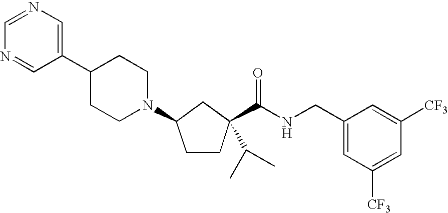

### Example XII-2

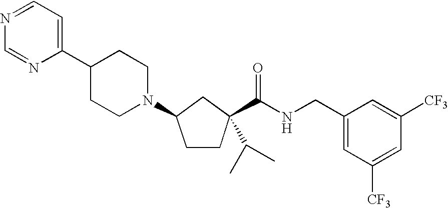

### Example XII-3

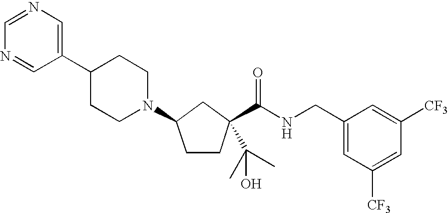

### Examples XII 1-3

Examples XII-4 through XII-62, on Table 35, below, are based on the Formula:

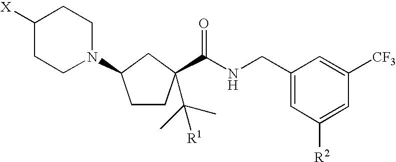

| | | | | | Ob- |
|---|---|---|---|---|---|
| | | | | | served |
| | | | | | M + H |
| Ex- | | | | Calc. | by |
| ample | X | R¹ | R² | MW | ESI-MS |
| XII-4 | 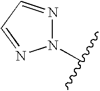 | H | CF₃ | 531 | 532 |
| XII-5 | 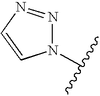 | H | CF₃ | 531 | 532 |
| XII-6 | 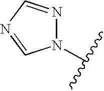 | H | CF₃ | 531 | 532 |
| XII-7 | 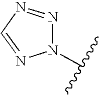 | H | CF₃ | 532 | 533 |
| XII-8 | 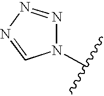 | H | CF₃ | 532 | 533 |
| XII-9 | 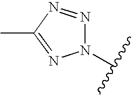 | H | CF₃ | 546 | 547 |
| XII-10 | 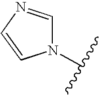 | H | CF₃ | 530 | 531 |
| XII-11 | 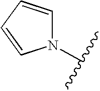 | H | CF₃ | 529 | 530 |
| XII-12 | 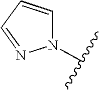 | H | CF₃ | 530 | 531 |
| XII-13 | 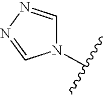 | H | CF₃ | 531 | 532 |
| XII-14 | 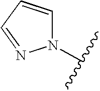 | OH | CF₃ | 546 | 547 |
| XII-15 | 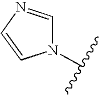 | OH | CF₃ | 546 | 547 |
| XII-16 | 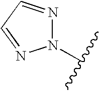 | OH | CF₃ | 547 | 548 |
| XII-17 | 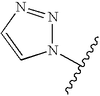 | OH | CF₃ | 547 | 548 |
| XII-18 | 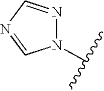 | OH | CF₃ | 547 | 548 |
| XII-19 | 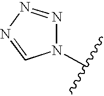 | OH | CF₃ | 548 | 549 |
| XII-20 | 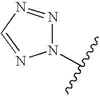 | OH | CF₃ | 548 | 549 |
| XII-21 | 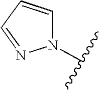 | H | F | 480 | 481 |
| XII-22 | 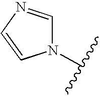 | H | F | 480 | 481 |
| XII-23 | 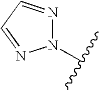 | H | F | 481 | 482 |
| XII-24 | 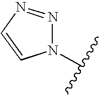 | H | F | 481 | 482 |
| XII-25 | 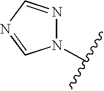 | H | F | 481 | 482 |
| XII-26 | 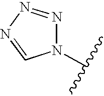 | H | F | 482 | 483 |
| XII-27 | 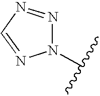 | H | F | 482 | 483 |
| XII-28 | 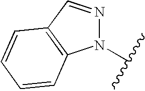 | H | CF₃ | 580 | 581 |
| XII-29 | 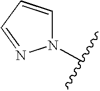 | OH | F | 496 | 497 |
| XII-30 | 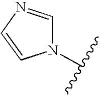 | OH | F | 496 | 497 |
| XII-31 | 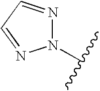 | OH | F | 497 | 498 |
| XII-32 | 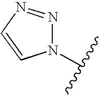 | OH | F | 497 | 498 |
| XII-33 | 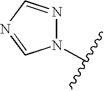 | OH | F | 497 | 498 |
| XII-34 | 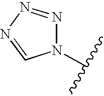 | OH | F | 498 | 499 |
| XII-35 | 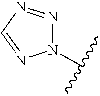 | OH | F | 498 | 499 |
| XII-36 | 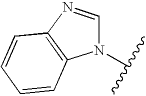 | H | CF₃ | 580 | 581 |
| XII-37 | 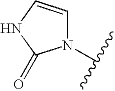 | H | CF₃ | 546 | 547 |
| XII-38 | 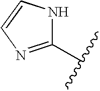 | H | CF₃ | 530 | 531 |
| XII-39 | 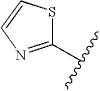 | H | CF₃ | 547 | 548 |
| XII-40 | 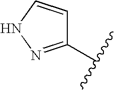 | H | CF₃ | 530 | 531 |
| XII-41 | 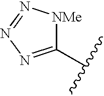 | OH | CF₃ | 562 | 563 |
| XII-42 | 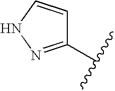 | H | F | 480 | 481 |
| XII-43 | 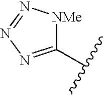 | H | F | 496 | 497 |
| XII-44 | 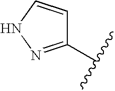 | OH | F | 496 | 497 |
| XII-45 | 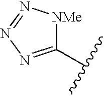 | OH | F | 512 | 513 |
| XII-46 | 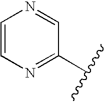 | H | CF₃ | 542 | 543 |
| XII-47 | 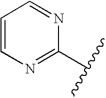 | H | CF₃ | 542 | 543 |
| XII-48 | 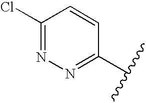 | H | CF₃ | 577 | 578 |
| XII-49 | 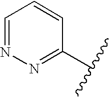 | H | CF₃ | 542 | 543 |
| XII-50 | 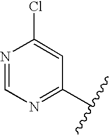 | H | CF₃ | 577 | 578 |
| XII-51 | 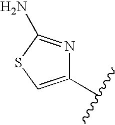 | H | CF₃ | 562 | 563 |
| XII-52 | 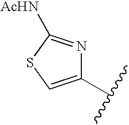 | H | CF₃ | 604 | 605 |
| XII-53 | 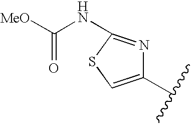 | H | CF₃ | 620 | 621 |
| XII-54 | 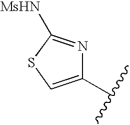 | H | CF₃ | 640 | 641 |
| XII-55 | 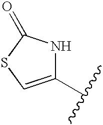 | H | CF₃ | 564 | 565 |
| XII-56 | 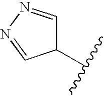 | H | CF₃ | 530 | 531 |
| XII-57 | 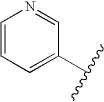 | H | CF₃ | 541 | 542 |
| XII-58 | 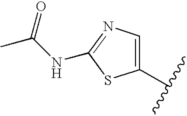 | H | CF₃ | 604 | 605 |
| XII-59 | 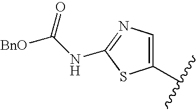 | H | CF₃ | 696 | 697 |
| XII-60 | 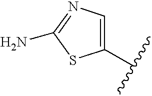 | H | CF₃ | 562 | 563 |
| XII-61 | 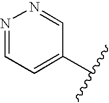 | H | CF₃ | 542 | 543 |
| XII-62 | 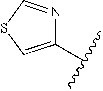 | H | CF₃ | 547 | 548 |

### Example XII-63

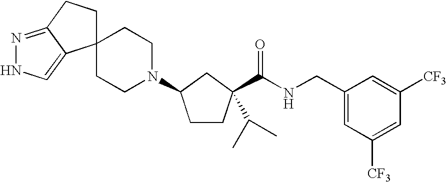

### Example XII-64

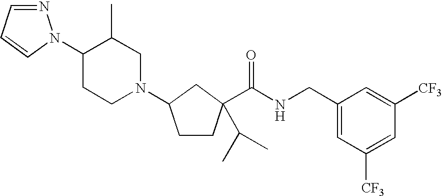

The subject treated in the present methods is generally a mammal, preferably a human being, male or female, in whom antagonism of CCR2 receptor activity for treating neuropathic pain is desired. The term “therapeutically effective amount” means the amount of the subject compound that will elicit the biological or medical response of a tissue, system, animal or human that is being sought by the researcher, veterinarian, medical doctor or other clinician. As used herein, the term “treatment” refers both to the treatment and to the prevention or prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

Methods of the present invention include administration of a CCR-2 antagonist via oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients.

In the treatment of conditions involving neutropathic pain an appropriate dosage level will generally be about 0.01 to 500 mg per kg patient body weight per day which can be administered in single or multiple doses. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

When treating conditions involving neuropathic pain, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligrams to about 50 milligrams. In the case of a 70 kg adult human, the total daily dose will generally be from about 7 milligrams to about 350 milligrams. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Biological Examples

### Example B-1

### Binding Assays

The utility of the compounds in accordance with the present invention as modulators of chemokine receptor activity may be demonstrated by methodology known in the art, such as the assay for chemokine binding as disclosed by Van Riper, et al., J. Exp. Med., 177, 851-856 (1993) which may be readily adapted for measurement of CCR-2 binding.

Receptor affinity in a CCR-2 binding assay was determined by measuring inhibition of ¹²⁵I-MCP-1 to the endogenous CCR-2 receptor on various cell types including monocytes, THP-1 cells, or after heterologous expression of the cloned receptor in eukaryotic cells. The cells were suspended in binding buffer (50 mM HEPES, pH 7.2, 5 mM MgCl₂, 1 mM CaCl₂, and 0.50% BSA) with and added to test compound or DMSO and ¹²⁵I-MCP-1 at room temperature for 1 h to allow binding. The cells were then collected on GFB filters, washed with 25 mM HEPES buffer containing 500 mM NaCl and cell bound ¹²⁵I-MCP-1 was quantified.

In a chemotaxis assay chemotaxis was performed using T cell depleted PBMC isolated from venous whole or leukophoresed blood and purified by Ficoll-Hypaque centrifugation followed by rosetting with neuraminidase-treated sheep erythrocytes. Once isolated, the cells were washed with HBSS containing 0.1 mg/ml BSA and suspended at 1×10⁷ cells/ml. Cells were fluorescently labeled in the dark with 2 μM Calcien-AM (Molecular Probes), for 30 min at 37° C. Labeled cells were washed twice and suspended at 5×10⁶ cells/ml in RPMI 1640 with L-glutamine (without phenol red) containing 0.1 mg/ml BSA. MCP-1 (Peprotech) at 10 ng/ml diluted in same medium or medium alone were added to the bottom wells (27 μl). Monocytes (150,000 cells) were added to the topside of the filter (30 μl) following a 15 min preincubation with DMSO or with various concentrations of test compound. An equal concentration of test compound or DMSO was added to the bottom well to prevent dilution by diffusion. Following a 60 min incubation at 37° C., 5% CO₂, the filter was removed and the topside was washed with HBSS containing 0.1 mg/ml BSA to remove cells that had not migrated into the filter. Spontaneous migration (chemokinesis) was determined in the absence of chemoattractant

In particular, useful compounds have activity in binding to the CCR-2 receptor in the aforementioned assays, with an IC₅₀ of less than about 1 μM. Such a result is indicative of the intrinsic activity of the compounds in use as modulators of chemokine receptor activity.

The animal studies described in the examples which follow establish that CCR-2 plays a significant role in neuropathic nociception.

### Example B-2

### Animals Used in Studies

Mice—Mice lacking CCR2 (CCR2 −/−) were generated by homologous recombination. Both CCR2 −/− and wild-type mice were of the genetic background C57BL/6Jx129P3/J (Taconic). The CCR2 −/− mouse was a random intercross on the C57BL/6x129/Ola background, and wild-type mice were of the genetic background C57BL/6x129SvEvTacF1 (Taconic).

Rats—Certain studies (as specified below) employed male Sprague-Dawley rats (Taconic, Germantown, N.Y.) weighing 200-300 grams. Other studies (specified below) employed Male Sprague-Dawley rats (Charles River, Kent, UK) weighing 145-160 g. Finally, the post-herpetic neuralgia model employed male Wistar rats (Charles River) weighing 200-300 g.

### Example B-3

### Test Methods, Procedures and Apparatus

### Mouse Studies

Rota-Rod: Mice were trained on the rota-rod for 3 minutes at a speed of 10 rpm. For testing, the speed was set at 10 rpm for 60 seconds and subsequently accelerated to 600 rpm. The time taken for mice to fall after the beginning of the acceleration was recorded.

Hot plate: Mice were habituated to the hot-plate apparatus with temperature set at 45° C. for 2 minutes. Subsequently, mice were placed on the hot-plate and the temperature was sequentially changed to 52.5 and 55.5° C. (cut off set up at 30 seconds) each and then to 58.5° C. (cut off set up at 20 seconds). The time when mice either licked their paws or jumped was recorded.

Formalin Test: For 4 days prior to testing, mice were acclimated for 2 hours every day on the test platform. On the day of study, mice were placed for 1 hour on the test platform, and subsequently were administered 10 μl of 2% formalin in the plantar surface of the left paw. The time mice spent either licking or lifting the injected paw was recorded over 2-minute periods at 5-minute intervals for 50 minutes. Following formalin injection, mice displayed a biphasic response. Phase 1 (0-10 min post-injection) is considered to reflect acute pain, whereas phase 2 (10-50 min post-injection) reflects chronic, inflammatory pain. The formalin test: a quantitative study of the analgesic effects of morphine, meperidine, and brain stem stimulation in rats and cats. Pain. December 1977;4(2):161-74)

To quantify the magnitude of the inflammatory response, paw diameters were measured with calipers 90 minutes after formalin injection.

MCP-1 Intraplantar Test: To investigate if MCP-1 evokes hyperalgesia, MCP-1 (150 or 500 ng in 5 μl, Research Diagnostics Inc, Flanders, N.J.) was injected subcutaneously and mechanical sensitivity assessed with von Frey filaments at various times after MCP-1 administration.

Thermal and Mechanical Stimulation Tests: Thermal sensitivity was assessed by measuring paw withdrawal latencies to a radiant heat stimulus (Hargreaves K, Dubner R, Brown F, Flores C, Joris J. A new and sensitive method for measuring thermal nociception in cutaneous hyperalgesia. Pain. January 1988;32(1):77-88.) Mechanical sensitivity was determined with calibrated von Frey filaments using the up-and-down paradigm. (Chaplan S R, Bach F W, Pogrel J W, Chung J M, Yaksh T L. Quantitative assessment of tactile allodynia in the rat paw. J Neurosci Methods. July 1994;53(1):55-63.)

Complete Freund's Adjuvant: Mice received a unilateral 30 μl intraplantar injection of CFA (0.5 mg/ml, Sigma, St. Louis, Mo.) into the left paw. Thermal and mechanical paw thresholds were determined before and up to 2 weeks after CFA administration.

Nerve injury: Mice were anesthetized with a mixture of ketamine (50 mg/kg, i.m., Pfizer Animal Health) and medetomidine (1 mg/kg, i.m., Pfizer Animal Health). An incision was made just below the hip bone, parallel to the sciatic nerve. The nerve was exposed, and any adhering tissue removed from the nerve. A tight ligature with 6-0 silk suture thread around ⅓ to ½ of the diameter of the sciatic nerve was made. Muscles were closed with suture thread and the wound with wound clips. The response of the mice to mechanical stimulation was tested before and up to 15 days after nerve injury. Mechanical sensitivity was determined with calibrated von Frey filaments.

Intragastrical administration by gavage: Compound and vehicle were given via a 18 G Gavage needle at 0.2 ml/30 g of the mouse body weight.

Real-time PCR analysis: Real-time PCR was used to assess CCR2 mRNA regulation after injury. Various tissues were dissected ipsilateral to the injury (plantar paw skin, sciatic nerve, DRG: L4, L5 and L6 and lumbar spinal cord) in naïve mice, in mice 2 days after CFA administration and in sciatic nerve ligated mice 2, 4 and 7 days, and 2, 3 and 4 weeks after ligation. Tissues were homogenized using a polytron in Ultraspec reagent (Biotecx Laboratories Inc, Houston, Tex.). RNA was isolated using Ultraspec RNA isolation system according to the manufacturer's protocol. mRNA was isolated using Qiagen oligotex kit (Valencia, Calif.). Reverse transcription (RT) was performed in a 100 μl reaction mixture containing 1× RT-PCR buffer, 5.5 mM MgC12, 500 μM dNTP Mix, 2.5 μM random hexamers, 0.8 units of RNAse inhibitor and 3.75 units of multiscribe RTase (Applied Biosystem, Foster City, Calif.). The reaction mixture was incubated for 10 minutes at 25° C., then 30 minutes at 48° C. and at 95° C. for 5 minutes and then stored at −20° C. until further PCR analysis.

Real-time quantitative PCR: Quantitation of mRNA for CCR2 and GAPDH was performed using an Applied Biosystems (Foster City, Calif.) PRISM 7700 sequence detection system. Samples of cDNA from control, inflamed and neuropathic groups or samples from neuropathic groups at different times were analyzed simultaneously by real-time PCR, with each sample run in duplicate. The PCR mixture was prepared using the multiplex real-time PCR protocol according to the manufacturer's instructions and the PCR and data analysis were run using the system software. Five μl of RT product for each sample was used as the template in a 50 μl reaction mixture. The primers and the TaqMan probe for CCR2 were as follows: 5′-AACAGTGCCCAGTTTTCTATAGG-3′, 5′-CGAGACCTCTTGCTCCCCA-3′ and 5′-6FAM-ACAGCAGATCGAGTGAGCTCTACATTCACTCC-TAMRA-3′. The primers and TaqMan probe for GAPDH were as follows: 5′-TGCACCACCAACTGCTTAG-3′, 5′-GGATGCAGGGATGATGTTC-3′ and 5′-VIC-CAGAAGACTGTGGATGGCCCCTC-TAMRA-3′. At the completion of the PCR reaction (total of 40 cycles), the amount of a target message in each sample was estimated from a threshold cycle number (Ct). Average Ct values were normalized to average Ct values for GAPDH mRNA from the same cDNA preparations. Results presented are expressed as fold increases over control values.

Immunohistochemistry: Mice were deeply anesthetized with sodium pentobarbital (100 mg/kg i.p.) and perfused through the ascending aorta with 4% formaldehyde (in 0.1 M phosphate buffer (PB), pH=7.4). The spinal cords, dorsal root ganglia, sciatic nerves and hind-paw skin were removed and placed in 4% formaldehyde for 4 hrs and then cryoprotected in 30% sucrose (in 0.1M PB). Tissues were sectioned (20-40 μm) on a freezing microtome (Leica SM 2000R, Nussloch, Germany) and collected into 0.1 M PB. Sections were incubated for 60 minutes at room temperature in 3% normal goat serum in PB with 0.9% sodium chloride and 0.3% Triton-X. Sections were then incubated overnight in CCR2 antiserum at 1:400 (4.25 μg/ml). This antibody raised against the C-terminal part (365-373) was raised and tested in house on CCR2 and CCR5 transfected CHO cells via immunocytochemistry, and western blots. The antibody was shown to have minimal cross-reactivity to murine CCR5, and no reactivity to non-transfected CHO cells was observed. Moreover in CCR2 −/− mice tissues, no specific labeling was detected. After the primary antiserum incubation, tissue sections were washed 3 times in 0.1 M PB and then incubated in CY-2 or Cy-3™ conjugated goat anti-rabbit IgG (1:600 in 0.1 M PB; Jackson ImmunoResearch, West Grove, Pa.) for 2 hours at room temperature. The sections were washed 3 times in 0.1 M PB, mounted on gelatin-coated slides, dried, and coverslipped with DPX (Aldrich, Milwaukee, Wis.).

In order to identify CCR2 positive cells in the skin, DRG and sciatic nerve F4/80 (1:100; Serotec, Raleigh, N.C.) was used as a monocyte/macrophage marker. For cells expressing CCR2 in the spinal cord, either the neuronal markers, MAP-2 or synaptophysin, (both 1:200; Sigma, St Louis, Mo.) or glial markers for astrocytes (GFAP: 1:20000, Sigma), oligodendrocytes (CNPase; 1:25, Chemicon, Temecula, Calif.) and microglia (OX-42; 1:4000; Cedarlane, Ontario, Canada) were used. Phospho p38 mitogen-activated protein kinase (pp38; 1:200, SantaCruz, Calif.) was used as a marker for glial activation. Double labeling studies with monoclonal antibodies in mouse spinal cord presented very poor staining therefore rat spinal cord was used for those studies (FIG. 3F-I). The secondary antibody was Cy-2™ conjugated goat anti-mouse IgG (1:600 in 0.1 M PB; Jackson ImmunoResearch).

### Rat Studies

Male Sprague-Dawley rats (Charles River, 145-160 g) were used in the paw pressure, hot plate and tail pinch rat models. Baselines values in each model were taken. Three baselines 20 min apart in hot plate (52.2 deg C.) and two baselines 1 hr apart in tail pinch and paw pressure (Ugo Basile apparatus) tests were taken prior to compound administration (n=5 per group). CCR-2 Antagonist C was diluted in 5% EtOH: 95% water. The vehicle group received 5% EtOH: 95% Water. Diclofenac (30 mg/kg p.o., diluted in 0.5% methylcellulose) and morphine (5 mg/kg s.c. diluted in saline) were used as the positive controls. All groups were dosed at 2 ml/kg.

Intragastrical administration by gavage: Compound and vehicle were given via a 15 G Gavage needle at 1 ml/100 g of the rat body weight.

Intrathecal administration by intrathecal catheter: Using Hamilton syringe to inject each rat: 5 μl compound or vehicle, 1 μl air and 9 μl vehicle.

Complete Freund's Adjuvant (CFA): Male Sprague-Dawley rats (Charles River) were injected with CFA (150 μl) intraplantar into their left paw. This study included 3 groups: (1) CCR-2 Antagonist C at 3 mg/kg bid started 2 hours before CFA injection, (2) vehicle group and (3) CCR-2 Antagonist C at 10 mg/kg given on day 3 post-CFA (rats received vehicle on day 0-2)(n=6 per group). Rats were dosed for 3.5 days bid. Before the morning dose and two hours after it, weight bearing and paw size were measured. On the final day of the study (day 3 post-CFA) in addition to weight bearing, paw pressure threshold was also evaluated at 2 hr post dose.

Carrageenan: Male Sprague-Dawley rats (Charles River, 150-200 g) were injected with carrageenan (5 mg in 150 μl saline) intraplantar into their left paw. Three hours after carrageenan, their withdrawal latency to mechanical pressure was measured (Ugo Basile apparatus). Two measures were taken for each paw, 35 min apart. Rats were then dosed with the test compounds. At 1 and 2 hours after drug administration, their mechanical threshold was measured (n=8 per group), but if rats do not display hyperalgesia (i.e. threshold higher than 80% of contralateral paw) they were not included in the results (hence n=6-7 per group).

L5-L6 Spinal Nerve Ligation (Chung): Male Sprague-Dawley rats (Taconic) were anesthetized with 2% gaseous isofluorane (For induction 3-5% and O₂ 500-700 μl, for maintenance 2-3% and O₂ 400-500 μl). Following dorsal skin incision and muscle separation, the posterior interarticular traverse process of L/S1 was exposed and carefully removed with a mircro Rongeur. The L5 and L6 spinal nerves were tightly ligated by a square knot with 6-0 silk thread. The muscles were closed with 4-0 absorbable sutures and the skin was closed with wound clips. Rats that exhibited motor deficiency (such as paw dragging) were excluded from further testing (less than 5% of the animals were excluded). Animals were pre-tested and non-sensitive rats (50% paw withdrawal threshold above 3 g) were also excluded from compound testing. The results were expressed either as 50% paw withdrawal threshold, or in % maximal possible effect (MPE). MPE was calculated as follows:

$$\% MPE = \frac{Post \text{-} treatment value - Pretreatment value}{Pre \text{-} operation cut \text{-} off value - Pretreatment value}$$

Pre-operation cut-off value is 15 grams.

Intrathecal catheterization. After shaving the back of the head and neck, the rats were placed in a stereotaxic headholder with the head flexed forward. A 8-cm saline filled polyethylene tube (PE5) was placed into the subarachnoid space through a small puncture and threaded caudally so that the caudal tip rested on the rostral edge of the lumbar enlargement. The rats were allowed to recover for a minimum of 2-3 days prior to further study. Only animals exhibiting normal motor behavior upon recovery from anesthesia were employed in the study. Animals with impaired motor function (e.g. hind limb paralysis) were euthanized.

Post-Herpetic Neuralgia: Rats were injected subcutaneously in the footpad with approximately 4×10⁶ wild-type varicella zoster virus (VZV) cells/animal in 50 μl PBS, as previously described (Fleetwood-Walker et al., 1999). Rats were tested for mechanical allodynia. (von Frey filaments) and thermal hyperalgesia (Hargreaves' infra-red apparatus) ipsi- and contralateral side of the injection. Time course studies showed that allodynia developed within one week, peaked 4-7 weeks post-injection and rats recovered at 11-12 weeks. Gabapentin, Lamotrigine and Mexiletine (100 mg/kg, p.o.; used in the clinic for PHN) were used as positive controls. All drugs were administered 34 weeks post-VZV injection). Test compound was administered bid for 3 days.

Compounds: A CCR-2 antagonist having the formula: 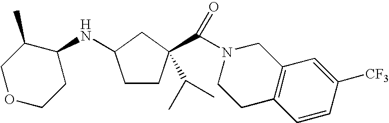
(CCR-2 Antagonist “A”) was tested in the formalin test and the mouse nerve injury model. A second CCR-2 antagonist: 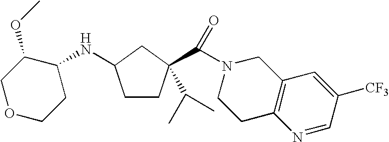
(CCR-2 Antagonist “B”) was tested in the formalin test only. Both compounds were diluted in 0.5% methylcellulose and were dosed p.o. at a volume of 0.2 ml per 30 g body-weight. For the formalin test, compounds were administered 60 min before the formalin injection. For the nerve injury model, Compound A was tested 4-5 days after surgery. A third CCR-2 antagonist having the formula: 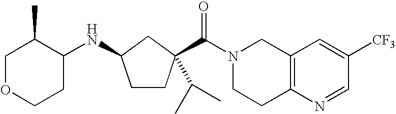
(CCR-2 Antagonist “C”) was tested in the rat nerve model, MCP-1 co-administration model, the carrageenan model and the CFA model. The compound was dissolved into ethanol/H₂O=9/95 prior to testing.

### Example B4

Mouse Rota-Rod Results

CCR2 −/− mice did not exhibit any impairment of motor coordination. Thus, retention times using the rota-rod test were 23.6±2.4 seconds for CCR2 −/− mice and 24.1±3.8 seconds for CCR2 +/+ mice (t-test p=0.89, n=18-19/group).

[image]

### Example B-5

Mouse Acute Nociception, Hot Plate Test Results

In the hot plate test no differences in latency period were found at the 3 tested temperatures (52.5, 55.5 and 58.5° C.) between the 2 groups of mice.

[image]

### Example B-6

Mouse Formalin Test Results

[image]

CCR2 −/− mice displayed a markedly attenuated behavior, compared with CCR2 +/+ mice, in their responses to formalin injection. Thus, phase 1 (0-10 minutes) responses were decreased by 24% in the CCR2 −/− mice compared to the CCR2 +/+ mice and phase 2 (15-50 minutes) responses were significantly (p=0.0285; n=9/group) decreased by 70% in the CCR2 −/− mice compared to CCR2 +/+ mice. Paw edema, measured 90 minutes after formalin injection, was not different in the 2 groups.

The effects of intraplantar injection of MCP-1 (150 and 500 ng) on mechanical allodynia were assessed in C57BL/6 mice. At a dose of 150 ng moderate allodynia (20-40% decrease in mechanical threshold) was observed. However, 500 ng of MCP-1 significantly decreased mechanical threshold (Kruskal-Wallis followed by Dunn's test, p<0.01; n=7-9/group).

[image]

### Example B-7

### Rat Persistent Pain, CFA Test Results

After inflammation induced by CFA administration, CCR2 knockout mice developed attenuated mechanical allodynia as compared to the wild type group (n=15-16/group). This decreased response (20-30%) was observed from 6 hours to 2 days after CFA. No differences between genotypes were evident in the development of thermal hyperalgesia.

[image]

Development of mechanical allodynia is characteristic of the response to nerve injury. CCR2 +/+ mice showed a significant (Kruskal-Wallis p<0.001, followed by Dunn's test) decrease in mechanical threshold starting 3 days after surgery until the last time point tested, 2 weeks after the nerve ligation. In contrast, CCR2 −/− mice did not develop mechanical allodynia following partial sciatic nerve injury. Mechanical thresholds in CCR2 −/− mice were equivalent before and after nerve injury (p=0.96). Furthermore, mechanical thresholds were significantly (Kruskal-Wallis followed by Dunn's test, p<0.001 at day 3, 5,

[image]

7, 11 and 15) different between CCR2 −/− and CCR2 +/+ mice at all time points except baseline and day 1.

### Example B-8

### Mouse CCR2 mRNA Regulation

Real time PCR was performed in various tissue after CFA and nerve injury of C57BL/6 mice. Basal levels of mCCR2 expression were detected as indicated by Ct values ranging from 33.7 to 28.2. A large increase in CCR2 mRNA expression was found in the paw skin following CFA injection, whereas levels in the sciatic nerve and spinal cord only increased two-fold. Following nerve injury, CCR2 mRNA up-regulation in the sciatic nerve and dorsal root ganglia was rapid, marked and sustained; in the paw skin there was a transient upregulation of CCR2 mRNA following ligation and no change was detected in the spinal cord.

CCR2 mRNA in various tissues during chronic pain states. Results are expressed as mean ± s.d. fold over control:

| | CFA | Nerve injury | | | | | |
|---|---|---|---|---|---|---|---|
| | 2 days | 2 days | 4 days | 1 week | 2 weeks | 3 weeks | 4 weeks |
| Paw skin | 21.1 ± 4.7 | 4.8 ± 0.2 | 2.8 ± 0.2 | 1.5 ± 0.1 | 1.9 ± 0.2 | 0.8 ± 0.1 | 1.0 ± 0.1 |
| Sciatic nerve | 2.4 ± 2.4 | 6.6 ± 0.1 | 8.3 ± 0.5 | 3.0 ± 0.7 | 5.0 ± 0.8 | 1.7 ± 0.1 | 3.4 ± 0.4 |
| DRG | 2.8 ± 0.4 | 5.4 ± 0.2 | 6.0 ± 0.6 | 4.3 ± 0.5 | 6.3 ± 0.0 | 3.2 ± 0.1 | 5.6 ± 0.5 |
| Spinal cord | 0.5 ± 0.1 | 1.4 ± 0.1 | 1.4 ± 0.1 | 1.1 ± 0.7 | 0.5 ± 0.1 | 0.9 ± 0.1 | 0.6 ± 0.1 |

### Example B-9

### Mouse CCR Protein Distribution after Chronic Injury

In the absence of inflammation or injury, only a few or no CCR2-like immunoreactive (-LI) monocytes/macrophages were observed. Consistent with the PCR data, in the CFA-inflamed paw skin, numerous monocytes/macrophages were CCR2 positive in the dermis and around blood vessels. Macrophages were identified by immunoreactivity for F4/80; about ⅔ of the F4/80 positive cells were CCR2 positive. No CCR2 positive cells in the skin were detected one week following nerve injury. In the sciatic nerve, after CFA a few CCR2 positive macrophages were present in the perineurium only, whereas in the neuropathic model, numerous macrophages were detected not only in the neuroma but also distant from the neuroma, in the perineurium as well as the endoneurium. In the DRG, as observed in the sciatic nerve, a few CCR2-LI cells were detected in response to CFA administration. In contrast, and consistent with PCR data, numerous CCR2-LI macrophages were present after nerve injury both in the perineurium and surrounding neuronal cells. In the spinal cord following nerve injury cells staining positive for CCR2 were identified as microglia (double labeled with OX-42). CCR2-LI cells did not double label for neuronal, astrocytes or oligodendrocyte markers. No CCR2-LI staining was detected on neurons in either the DRGs or the spinal cord.

Since microglia were shown to express CCR2 in the spinal cord and as glial cells reportedly are activated during chronic pain states, astrocytes and microglia were compared in the CCR2 −/− and CCR2 +/+ mice one week after partial nerve ligation. The number of astrocytes in the superficial laminae of the spinal cord was reduced in CCR2 −/− as compared to CCR2 +/+ mice. Furthermore, activated p38 mitogen-activated protein kinase, as detected with a phospho-specific p38 antibody, was at lower levels in microglia of the CCR2 knockout mice as compared to the wild-type.

### Example B-10

### CCR-2 Antagonist in Mouse, Formalin

CCR-2 Antagonist A significantly decreased mouse pain behavior in the formalin test (50% at 3 mg/kg p.o.). CCR-2 Antagonist B decreased pain behavior in the formalin test (30% at 30 mg/kg p.o.).

More specifically, CCR-2 Antagonist A had no effect on phase 1, but significantly decreased phase 2 times at 3 and 30 mg/kg. (ANOVA p=0.0182, followed by a Dunnett's test, n=5-7/group). No difference with the vehicle group was observed at 1 mg/kg. CCR-2 Antagonist B decreased phase 2 by 20% at 10 mg/kg and by 30% at 30 mg/kg.

[image]

### Example B-11

### CCR-2 Antagonist in Mouse, Neuropathic Pain

Compound A at 30 mg/kg p.o. reversed mechanical allodynia in mouse induced by nerve injury (Kruskal-Wallis p=0.0136, followed by a Dunn's test, p<0.05 at 4.5 hr time point, n=10).

[image]

### Example B-12

### MCP-1 Upregulation (In Spinal Cord, DRG)

The following experiments show that MCP-1 mRNA was persistently upregulated in the spinal cord 8-16 fold starting 2 days post spinal nerve ligation. In additiona CCR2 mRNA was persistently upregulated in the spinal cord 6-10 fold starting 2 days post spinal nerve ligation.

Spinal nerve ligation and drug administration: Male Sprague-Dawley rats (Taconic). Spinal nerve ligation (SNL) injury was induced using the procedure of Kim and Chung (Kim and Chung, 1992). Anesthesia was induced with 2% gaseous isofluorane (For induction 3-5% and O₂ 500-700 μl, for maintenance 2-3% and O₂ 400-500 μl). Following dorsal skin incision and muscle separation, the posterior interarticular transverse process of L/S1 was exposed and carefully removed with a micro Rongeur. The L5 and L6 spinal nerves were tightly ligated by a square knot with 6-0 silk thread. The muscles were closed with 4-0 absorbable sutures and the skin was closed with wound clips. Rats that exhibited motor deficiency (such as paw dragging) or failure to exhibit subsequent tactile allodynia were excluded from further testing (less than 5% of the animals were excluded). Sham control rats underwent the same operation and handling as the experimental animals but without spinal nerve ligation.

Tissue dissection and RNA preparation: Rat dorsal root ganglia (DRG) and spinal cord were dissected and rapidly frozen in liquid nitrogen. The spinal cord tissue was then partially thawed and further dissected on an ice-cold metal plate. Total RNA from each sample was prepared using Trizol™ (Life Technologies, Gaithersburg, Md.), followed by RNEasy™ (Qiagen, Hilden Germany). RNA samples were analyzed by denatured gel electrophoresis. In addition, total RNA quality was assessed by capillary electrophoresis (Bioanalyzer 2100 Agilent, Palo Alto, Calif.) to ensure that the 28S:18S rRNA ratio was >1.0 for each sample.

Quantitative Real-Time PCR (QRT-PCR): Total RNA was treated with DNase I, Amplification Grade (Invitrogen, Carlsbad, Calif.) to remove DNA contamination before cDNA synthesis. cDNA was synthesized with oligo (dT)12-18 using Superscript First-Strand Synthesis System for RT-PCR (Invitrogen, Carlsbad, Calif.). Real-time PCR analysis was performed on a Applied Biosystems ABI Prism7700 Sequence Detection System. Matching primers and fluorescence probes were designed for each of the genes using the Primer Express program provided by Applied Biosystems. Both forward and reverse primers were used at 900 nM. In all cases, the final probe concentration was 250 nM. The PCR reaction was performed in a final volume of 50 μl using TaqMan Universal PCR Master Mix containing AmpliTaq Gold DNA Polymerase, AmpErase UNG, dNTPs (with dUTP), Passive Reference 1, optimized buffer components (proprietary formulation) and 1 μl of cDNA template.

QRT-PCR Data Analysis: Average Cₜ values from triplicate PCR reactions were normalized to average Cₜ values for GAPDH RNA from the same cDNA preparations. The ratio of expression of a pair of samples was calculated as: 2^{-(meanΔΔCt)}·Cₜ represents the threshold cycle and ΔΔCₜ represents the difference C_{t(test gene)}−C_{t(GAPDH RNA)} for sample#l minus contralateral sample #2. Using the ANOVA method, 95% confidence intervals were determined for each ratio as:

$${2}^{- ( mean ΔΔ Ct ) \pm {t}_{0.975 , N - m} s } \sqrt{\frac{1}{{n}_{i}} + \frac{1}{{n}_{j}}}$$

where t_{0.975} is the 97.5^{th} percentile of the t-distribution with N-m degrees of freedom, N is the total pooled sample size for a gene, m is the number of treatments including control, s is the pooled standard deviation, nᵢ and nⱼ are the number of two samples, respectively, being compared.

Regulation of MCP-1 and CCR2 expression in the DRG in the Chung model as revealed by QRT-PCR. The fold change of expression between the ipsilateral and contralateral DRG is determined at 8, 24, 48, 72 hours, and at 3, 7, 14, 28, and 120 days post spinal nerve ligation surgery.

[image]

Regulation of MCP-1 and CCR2 expression in the spinal cord in the Chung model as revealed by QRT-PCR. The fold change of expression between the ipsilateral and contralateral DRG is determined at 8, 24, 48, 72 hours, and at 3, 7, 14, 28, and 120 days post spinal nerve ligation surgery.

[image]

### Example B-13

### CCR-2 Antagonist in Rat, & MCP-1 Co-Administration

Example B-12 demonstrated that MCP-1 mRNA was persistently upregulated 8-16 fold starting 2 days post spinal nerve ligation. Consistent with the up-regulation of MCP-1 in the spinal cord in the Chung model, MCP-1 intrathecal injection (225-750 ng/rat) induced a chronic mechanical allodynia, behaviorally comparable to that in the Chung model. Co-injection of MCP-1 with CCR-2. Antagonist C inhibited and delayed the development of mechanical allodynia (further establishing that CCR2 is involved in the development of allodynia induced by MCP-1).

Intrathecal injection of MCP-1 (750 ng/rat) to naïve rats induces bilateral mechanical allodynia. (only the left paw results are shown in the graph but right paw results are similar to the left paw). At Day 0, 1, 3, 4, 7, 10, 11, 15, 18 and 21 post dosing, 50% paw withdrawal threshold was determined. Co-injection of 20 μg/rat CCR-2 Antagonist C (via intrathecal catheter) with MCP-1 has partial preemptive anti-allodynic effect on day 4, 7, 10 and 11.

[image]

### Example B-14

### CCR-2 Antagonist in Rat, Chronic Dosing

CCR-2 Antagonist C was evaluated in a multiple dosing study for 5 days. (3 mg/kg, b.i.d), and demonstrated significant efficacy using this chronic dosing regimen. 50% paw withdrawal threshold following multiple dosing (3 mg/kg, PO, b.i.d.) of CCR-2 Antagonist C. Five days post spinal nerve ligation, the animals were tested before and 1 hr after dosing at 7 a.m. each day for 5 days. Data=Mean±SEM, n=10 rats. Efficacy: % MPE. Five days post spinal nerve ligation, the animals were tested before and 1 hr after dosing at 7 a.m. each day for 5 days. Significant efficacy was observed starting at day 3.

[image]

### Example B-15

### CCR-2 Antagonist in Rat, Weight Bearing Test

CCR-2 Antagonist C at 3 mg/kg bid significantly reversed weight bearing on day 2 and 3 post-dose. CCR-2 Antagonist C at 10 mg/kg also significantly reversed weight bearing on the affected limb on day 3.

[image]

[image]

The syntheses of CCR-2 Antagonists A, B, and C disclosed in WO 03/093321 published Nov. 13, 2003.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. Therefore, the invention is defined by the claims which follow and not limited by the examples.

What is claimed is:

1. A method for treating neuropathic pain comprising administering to a patient in need of such treatment a therapeutically effective amount of a CCR-2 antagonist.

2. A method for treating neuropathic pain comprising administering to a patient in need of such treatment a therapeutically effective amount of a compound of the formula: 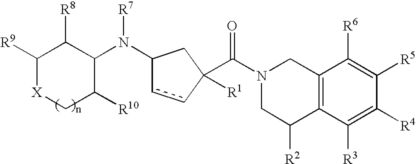
wherein:
X is selected from the group consisting of:
—O—, —NR²⁰—, —S—, —SO—, —SO₂—, and —CR²¹R²²—, —NSO₂R²⁰—, —NCOR²⁰—, —NCO₂R²⁰—, —CR²¹CO₂R²⁰—, —CR²¹OCOR²⁰—, —CO—,
where R²⁰ is selected from: hydrogen, C₁₋₆ alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂-C₁₋₆ alkyl, and trifluoromethyl,
where R²¹ and R²² are independently selected from: hydrogen, hydroxy, C₁₋₆ alkyl, —O—C₁₋₆alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl;
R¹ is selected from:
—C₁₋₆alkyl, —C₀₋₆alkyl-O—C₁₋₆alkyl-, —C₀₋₆alkyl-S—C₁₋₆alkyl-, —(C₀₋₆alkyl)-(C₃₋₇cycloalkyl)-(C₀₋₆alkyl), hydroxy, —CO₂R²⁰, heterocycle, —CN, —NR²⁰R²⁶—, —NSO₂R²⁰—, —NCOR²⁰—, —NCO₂R²⁰—, —NCOR²⁰—, —CR²¹CO₂R²⁰—, —CR²¹OCOR²⁰—, phenyl and pyridyl,
where R²⁶ is selected from: hydrogen, C₁₋₆ alkyl, benzyl, phenyl, C₃₋₆ cycloalkyl where the alkyl, phenyl, benzyl, and cycloalkyl groups can be unsubstituted or substituted with 1-3 substituents where the substituents are independently selected from: halo, hydroxy, C₁₋₃alkyl, C₁₋₃alkoxy, —CO₂H, —CO₂—C₁₋₆ alkyl, and trifluoromethyl
where the alkyl and the cycloalkyl are unsubstituted or substituted with 1-7 substituents where the substituents are independently selected from:
(a) halo,
(b) hydroxy,
(c) —O—C₁₋₃alkyl,
(d) trifluoromethyl,
(f) C₁₋₃alkyl,
(g) —O—C₁₋₃alkyl,
(h) —CO₂R²⁰,
(i) —SO₂R²⁰,
(j) —NHCOCH₃,
(k) —NHSO₂CH₃,
(l) -heterocycle,
(m) ═O,
(n) —CN,
and where the phenyl and pyridyl are unsubstituted or substituted with 1-3 substituents where the substituents are independently selected from: halo, hydroxy, C₁₋₃alkyl, C₁₋₃alkoxy and trifluoromethyl;
R² is selected from:
(a) hydrogen,
(b) hydroxy,
(c) halo,
(d) C₁₋₃alkyl, where the alkyl is unsubstituted or substituted with 1-6 substituents independently selected from: fluoro, and hydroxy,
(e) —NR²⁰R²⁶,
(f) —CO₂R²⁰,
(g) —CONR²⁰R²⁶,
(h) —NR²⁰COR²¹,
(i) —OCONR²⁰R²⁶,
(j) —NR²⁰CONR²⁰R²⁶,
(k) -heterocycle,
(l) —CN,
(m) —NR²⁰—SO₂—NR²⁰R²⁶,
(n) —NR²⁰—SO₂—R²⁶,
(o) —SO₂—NR²⁰R²⁶, and
(p) ═O, where R² is connected to the ring via a double bond;
R³ is selected from:
(a) hydrogen,
(b) hydroxy,
(c) halo,
(d) C₁₋₆alkyl,
(e) —O—C₁₋₆alkyl,
(f) —NR²⁰R²¹,
(g) —NR²⁰CO₂R²¹,
(h) —NR²⁰CONR²⁰R²¹,
(i) —NR²⁰—SO₂—NR²⁰R²¹,
(j) —NR²⁰—SO₂—R²¹,
(k) heterocycle,
(l) —CN,
(m) —CONR²⁰R²¹,
(n) —CO₂R²⁰,
(o) —NO₂,
(p) —S—R²⁰,
(q) —SO—R²⁰,
(r) —SO₂—R²⁰, and
(s) —SO₂—NR²⁰R²¹;
R⁴ is selected from:
(a) hydrogen,
(b) C₁₋₆alkyl,
(c) trifluoromethyl,
(d) trifluoromethoxy,
(e) chloro,
(f) fluoro,
(g) bromo, and
(h) phenyl;
R⁵ is selected from:
(a) C₁₋₆alkyl, where alkyl may be unsubstituted or substituted with 1-6 fluoro and optionally substituted with hydroxyl,
(b) —O—C₁₋₆alkyl, where alkyl may be unsubstituted or substituted with 1-6 fluoro,
(c) —CO—C₁₋₆alkyl, where alkyl may be unsubstituted or substituted with 1-6 fluoro,
(d) —S—C₁₋₆alkyl, where alkyl may be unsubstituted or substituted with 1-6 fluoro,
(e) -pyridyl, which may be unsubstituted or substituted with one or more substituents selected from the group consisting of: halo, trifluoromethyl, C₁₋₄alkyl, and CO₂R²⁰,
(f) fluoro,
(g) chloro,
(h) bromo,
(i) —C₄₋₆cycloalkyl,
(j) —O—C₄₋₆cycloalkyl,
(k) phenyl, which may be unsubstituted or substituted with one or more substituents selected from the group consisting of: halo, trifluoromethyl, C₁₋₄alkyl, and CO₂R²⁰,
(l) —O-phenyl, which may be unsubstituted or substituted with one or more substituents selected from the group consisting of: halo, trifluoromethyl, C₁₋₄alkyl, and CO₂R²⁰,
(m) —C₃₋₆cycloalkyl, where alkyl may be unsubstituted or substituted with 1-6 fluoro,
(n) —O—C₃₋₆cycloalkyl, where alkyl may be unsubstituted or substituted with 1-6 fluoro,
(o) -heterocycle,
(p) —CN, and
(q) —CO₂R²⁰;
R⁶ is selected from:
(a) hydrogen,
(b) C₁₋₆alkyl, and
(c) trifluoromethyl
(d) fluoro
(e) chloro, and
(f) bromo;
R⁷ is selected from:
(a) hydrogen, and
(b) C₁₋₆alkyl, which is unsubstituted or substituted with 1-3 substituents where the substituents are independently selected from: halo, hydroxy, —CO₂H, —CO₂C₁₋₆alkyl, and —O—C₁₋₃alkyl;
R⁸ is selected from:
(a) hydrogen,
(b) C₁₋₆alkyl, where alkyl may be unsubstituted or substituted with 1-6 substituents where the substituents are chosen from the group: fluoro, C₁₋₃alkoxy, hydroxy, —CO₂R²⁰,
(c) fluoro,
(d) —O—C₁₋₃alkyl, where alkyl may be unsubstituted or substituted with 1-3 fluoro, and
(e) C₃₋₆ cycloalkyl,
(f) —O—C₃₋₆cycloalkyl,
(g) hydroxy,
(h) —CO₂R²⁰,
(i) —OCOR²⁰,
or R⁷ and R⁸ may be joined together via a C₂₋₄alkyl or a C₀₋₂ alkyl-O—C₁₋₃alkyl chain to form a 5-7 membered ring;
R⁹ is selected from:
(a) hydrogen,
(b) C₁₋₆alkyl, where alkyl may be unsubstituted or substituted with 1-6 substituents where the substituents are chosen from the group: fluoro, C₁₋₃alkoxy, hydroxy, —CO₂R²⁰,
(c) CO₂R²⁰,
(d) hydroxy, and
(e) —O—C₁₋₆alkyl, where alkyl may be unsubstituted or substituted with 1-6 substituents where the substituents are chosen from the group: fluoro, C₁₋₃alkoxy, hydroxy, —CO₂R²⁰,
or R⁸ and R⁹ may be joined together by a C₁₋₄alkyl chain or a C₀₋₃alkyl-O—C₀₋₃alkyl chain to form a 3-6 membered ring;
R¹⁰ is selected from:
(a) hydrogen, and
(b) C₁₋₆alkyl, where alkyl may be unsubstituted or substituted with 1-6 fluoro,
(c) fluoro,
(d) —O—C₃₋₆cycloalkyl, and
(e) —O—C₁₋₃alkyl, where alkyl may be unsubstituted or substituted with 1-6 fluoro,
or R⁸ and R¹⁰ may be joined together by a C₂₋₃alkyl chain to form a 5-6 membered ring, where the alkyl are unsubstituted or substituted with 1-3 substituents where the substiuents are independently selected from: halo, hydroxy, —CO₂R²⁰, C₁₋₃alkyl, and C_{1-3 l alkoxy,}
or R⁸ and R¹⁰ may be joined together by a C₁₋₂alkyl-O—C₁₋₂alkyl chain to form a 6-8 membered ring, where the alkyl are unsubstituted or substituted with 1-3 substituents where the substiuents are independently selected from: halo, hydroxy, —CO₂R²⁰, C₁₋₃alkyl, and C₁₋₃alkoxy,
or R⁸ and R¹⁰ may be joined together by a —O—C₁₋₂alkyl-O-chain to form a 6-7 membered ring, where the alkyl are unsubstituted or substituted with 1-3 substituents where the substiuents are independently selected from: halo, hydroxy, —CO₂R²⁰, C₁₋₃alkyl, and C₁₋₃alkoxy;
n is selected from 0, 1 and 2;
the dashed line represents a single or a double bond;
and pharmaceutically acceptable salts thereof and individual diastereomers thereof.

3. A method of claim 2, wherein X is oxygen.

4. A method for treating neuropathic pain comprising administering to a patient in need of such treatment a therapeutically effective amount of a compound of the formula: [chemical structure]
